(19) United States
(12) Patent Application Publication
(10) Pub. No.: US 2026/0364091 A1
(43) Pub. Date: Nov. 19, 2026

(54) ANTICANCER THERAPY

(71) Applicant: Boehringer Ingelheim International GmbH, Ingelheim am Rhein (DE)

(72) Inventors: Sabine JURADO, Wien (AT); Marco Hans HOFMANN, Vienna (AT); Simone LIEB, Wien (AT); Philipp SCHMALHORST, Vienna (AT); Krzyzstof ZAK, Wien (AT)

(21) Appl. No.: 19/652,379

(22) Filed: Apr. 20, 2026

(30) Foreign Application Priority Data
Apr. 23, 2025 (EP) 25171874.8

(51) Int. Cl. A61K 31/553; A61K 31/52; A61K 31/5386; A61P 35/00

(52) U.S. Cl. CPC A61K 31/553; A61K 31/52; A61K 31/5386; A61P 35/00

(57) ABSTRACT

The present invention relates to the use of an inhibitor binding to the switch II pocket of KRAS; preferably a KRAS G12D switch II inhibitor, in the treatment of cancer that is resistant to treatment with a tri-complex inhibitor, particularly of cancer where the cancer cells exhibit a primary KRAS G12D mutation and further a secondary KRAS mutation Y71X, preferably Y71H.

### FIELD OF THE INVENTION

The present invention relates to the use of an inhibitor binding to the switch II pocket of KRAS; preferably a KRAS G12D switch II inhibitor, in the treatment of cancer that is resistant to treatment with a tri-complex inhibitor, particularly of cancer where the cancer cells exhibit a primary KRAS G12D mutation and further a secondary KRAS mutation Y71X, preferably Y71H.

### BACKGROUND OF THE INVENTION

V-Ki-ras2 Kirsten rat sarcoma viral oncogene homolog (KRAS) is a small GTPase of the Ras family of proteins that exists in cells in either GTP-bound or GDP-bound states (McCormick et al., J. Mol. Med. (Berl)., 2016, 94(3):253-8; Nimnual et al., Sci. STKE., 2002, 2002(145):pe36).

KRAS mutations (e.g. amino acids G12, G13, Q61, A146) are found in a variety of human cancers including lung cancer, colorectal cancer and pancreatic cancer (Cox et al., Nat. Rev. Drug Discov., 2014, 13(11):828-51). Alterations (e.g. mutation, over-expression, gene amplification) in Ras family proteins/Ras genes have also been described as a resistance mechanism against cancer drugs such as the EGFR antibodies cetuximab and panitumumab (Leto et al., J. Mol. Med. (Berl). 2014 July; 92(7):709-22) and the EGFR tyrosine kinase inhibitor osimertinib/AZD9291 (Ortiz-Cuaran et al., Clin. Cancer Res., 2016, 22(19):4837-47; Eberlein et al., Cancer Res., 2015, 7 5(12):2489-500).

Genetic alterations affecting e.g. codon 12 of KRAS substitute the glycine residue naturally occurring at this position for different amino acids such as aspartic acid (the G12D mutation or KRAS G12D), cysteine (the G12C mutation or KRAS G12C), valine (the G12V mutation or KRAS G12V) among others. Similarly, mutations within codons 13, 61 and 146 of KRAS are commonly found in the KRAS gene. Altogether KRAS mutations are detectable in 35% of lung, 45% of colorectal and up to 90% of pancreatic cancers (Herdeis et al., Curr Opin Struct Biol., 2021, 71:136-147). The KRAS G12D mutation was associated with shorter survival in lung cancer and pancreatic cancer (Ricciuti, B. et al. Ann. Oncol., 2022, 33:1029-1040; Rachakonda, P. S. et al., PLoS One. 2013; 8(4): e60870).

For patients with advanced solid tumors harboring a KRAS G12D mutation, several selective inhibitors have entered clinical trials (MRTX1133, RMC-9805 (Zoldonrasib), QTX3034/46, LY3962673) and RMC-6236 (Daraxonrasib), a pan-RAS inhibitor from Revolution Medicines, has recently shown promising early clinical data. Further KRAS inhibitors either mutant specific or targeting multiple KRAS aberrations are described in WO2021041671, WO2022173678, WO2023060253, WO2023099624, WO2023064857 and Tanada, M. et al. J. Am. Chem. Soc. 2023, 145 (30), 16610-16620.

(K)RAS inhibitors can inhibit KRAS by binding to its switch II pocket or by forming a tri-complex with KRAS and cyclophilin A (Tanaka N. and Ebi H., Cancer Sci. 2025, 116:600-612). These later inhibitors will thereafter be referred to as tri-complex inhibitors.

However, alongside the great hopes placed in (K)RAS targeting therapy, resistance is likely to occur, as it was already observed in patients relapsing in response to the covalent switch II KRAS G12C inhibitors sotorasib (AMG510) and adagrasib (MRTX849), which have already received accelerated approval (Awad M. M., et al., N Engl J Med 2021; 384:2382-93; Tanaka N. et al Cancer Discov 2021; 11:1913-22; and Holderfield, M., et al., Nature 2024, 629, 919-926).

Multiple mechanisms have been identified pre-clinically and/or clinically to occur in the context of acquired resistance KRAS inhibitors, such as on-target mutations, reactivation of the MAPK or alternative pathways, and histological changes (Tanaka N. and Ebi H., Cancer Sci. 2025, 116:600-612; Riedl J. M., et al., Ann Oncol. 2025, 36, 682-692). In the case of resistance mediated through on-target mutation in the switch II pocket, a modulator binding to a different part of the protein could still be effective (Tanaka N., et al., Cancer Discov. 2021; 11:1913-22). Resistance to tri-complex inhibitors adressing KRAS is likely to also be observed clinically. Thus, there is the need to provide inhibitors that allow for a secondary treatment option for cancers with acquired resistance to tri-complex inhibitors.

### SUMMARY OF THE INVENTION

The colorectal cancer cell line GP2d, expressing a primary KRAS G12D mutation, was continuously treated with tri-complex inhibitor to generate resistance. Once the resistance was confirmed, individual outgrowing clones were subsequently profiled in a series of assays to identify potential mechanism(s) and cross-tested with other inhibitors. Cells were cross resistant to tri-complex inhibitors Daraxonrasib (RMC-6236) and/or Zoldonrasib (RMC-9805), highlighting a common mechanism of action, while remaining sensitive to other KRAS switch II inhibitors, preferably KRAS G12D switch II inhibitors. This provides an option for a second-line therapy to overcome an acquired resistance caused by secondary KRAS mutations as can be expected to emerge in the course of treatment with tri-complex inhibitors.

According to a first aspect a KRAS switch II inhibitor, as described herein above and below, is provided for use in the treatment and/or prevention of cancer, wherein the cancer is resistant to treatment with a tri-complex inhibitor.

According to another aspect a KRAS G12D switch II inhibitor, as described herein above and below, is provided for use in the treatment and/or prevention of cancer, wherein the cancer is resistant to treatment with a tri-complex inhibitor.

In an embodiment, the cancer has become resistant to treatment with a tri-complex inhibitor after earlier treatment with a tri-complex inhibitor.

In an embodiment, the cancer is resistant to treatment with Daraxonrasib (RMC-6236) and/or Zoldonrasib (RMC-9805).

In preferred embodiments, the cancer has become resistant to treatment with Daraxonrasib (RMC-6236) and/or Zoldonrasib (RMC-9805) after earlier treatment with Daraxonrasib (RMC-6236) and/or Zoldonrasib (RMC-9805).

In an embodiment, the cancer cells of the cancer exhibit a primary KRAS G12D mutation and further exhibit a secondary KRAS Y71X mutation, preferably Y71H.

In an embodiment, the cancer cells of the cancer have been determined to exhibit a primary KRAS G12D mutation and further exhibit a secondary KRAS Y71X mutation, preferably Y71H.

In embodiments the KRAS switch II inhibitor is selected from the group consisting of
KRAS inhibitors generically described/or specifically disclosed in WO2023064857,
any KRAS inhibitor of example 1 to 73 as disclosed in WO2023064857,
preferably KRAS inhibitor of example 22 and/or 26 as disclosed in WO2023064857 the disclosure being incorporated by reference in its entirety herein, and with the respective synthesis and properties.

In embodiments the KRAS G12D switch II inhibitor is selected from the group consisting of
KRAS inhibitors generically described/or specifically disclosed in WO2023064857,
any KRAS inhibitor of example 1 to 73 as disclosed in WO2023064857,
KRAS G12D inhibitors generically described/or specifically disclosed in WO2022173678
any KRAS inhibitor of example 1 to 51 as disclosed in WO2022173678
preferably KRAS inhibitor of example 21 and/or 28 as disclosed in WO2022173678
MRTX1133 (Wang, X. et al. Journal of Medicinal Chemistry 2022 65 (4), 3123-3133) the disclosure being incorporated by reference in its entirety herein, and with the respective synthesis and properties.

In one embodiment the KRAS G12D switch II inhibitor is a compound according to formula (I)

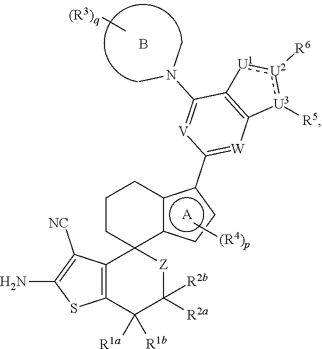

or a pharmaceutically acceptable salt thereof, wherein R^{1a}, R^{1b}, R^{2a}, R^{2b}, R³, R⁴, R⁵, R⁶, Z, U¹, U², U³, V, W, ring A, ring B, p and q are as defined herein below.

In embodiments, the cancer is selected from the group consisting of pancreatic cancer, lung cancer, colorectal cancer, cholangiocarcinoma, uterine cancer, endometrial cancer, thyroid cancer, acute myeloid leukaemia, bladder cancer, urothelial cancer, gastric cancer, cervical cancer, oesophageal cancer, and ovarian cancer.

Another aspect relates to a pharmaceutical composition comprising as active ingredient a KRAS G12D switch II inhibitor.

Another aspect relates to a pharmaceutical composition comprising as active ingredient a KRAS G12D switch II inhibitor, for use in the treatment and/or prevention of cancer, wherein the cancer is resistant to treatment with a tri-complex inhibitor.

Another aspect relates to a method of treating and/or preventing cancer, the method comprising administering to a patient a therapeutically effective amount of a KRAS G12D switch II inhibitor, wherein the cancer is resistant to treatment with a tri-complex inhibitor.

Another aspect relates to a use of a KRAS G12D switch II inhibitor for the manufacture of a medicament for use in the treatment and/or prevention of cancer, wherein the cancer is resistant to treatment a tri-complex inhibitor.

In an embodiment of all aspects disclosed herein, particularly of the pharmaceutical composition, the methods, or the uses, the cancer has become resistant to treatment with a tri-complex inhibitor after earlier treatment with a tri-complex inhibitor.

In an embodiment of all aspects disclosed herein, particularly of the pharmaceutical composition, the methods, or the uses, the KRAS G12D switch II inhibitor is a compound as defined herein or a pharmaceutically acceptable salt thereof.

In an embodiment of all aspects disclosed herein, particularly of the pharmaceutical composition, the methods, or the uses, the cancer is resistant to treatment with Daraxonrasib (RMC-6236) and/or Zoldonrasib (RMC-9805).

In preferred embodiments of all aspects disclosed herein, particularly of the pharmaceutical composition, the methods, or the uses, the cancer has become resistant to treatment with Daraxonrasib (RMC-6236) and/or Zoldonrasib (RMC-9805) after earlier treatment with Daraxonrasib (RMC-6236) and/or Zoldonrasib (RMC-9805).

In an embodiment of all aspects disclosed herein, particularly of the pharmaceutical composition, the methods, or the uses, the cancer cells of the cancer exhibit a primary KRAS G12D mutation and further exhibit a secondary KRAS Y71X mutation, preferably Y71H.

In preferred embodiments of all aspects disclosed herein, particularly of the pharmaceutical composition, the methods, or the uses, the cancer cells of the cancer have been determined to exhibit a primary KRAS G12D mutation and to further exhibit a secondary KRAS Y71X mutation, preferably Y71H.

In embodiments of all aspects disclosed herein, particularly of the pharmaceutical composition, the methods, or the uses, the cancer is selected from the group consisting of pancreatic cancer, lung cancer, colorectal cancer, cholangiocarcinoma, uterine cancer, endometrial cancer, thyroid cancer, acute myeloid leukaemia, bladder cancer, urothelial cancer, gastric cancer, cervical cancer, oesophageal cancer, and ovarian cancer.

Another aspect relates to an in vitro/ex-vivo method for detecting or diagnosing that an individual has acquired resistance to treatment with a tri-complex inhibitors and/or is susceptible to treatment with a KRAS G12D switch II inhibitor, the method comprising the step of:
determining, in a biological sample from an individual, the presence of a KRAS mutation Y71X, preferably Y71H, wherein the individual is classified as being resistant to treatment with a tri-complex inhibitor and being susceptible to treatment with a KRAS G12D switch II inhibitor, based on the detection of the KRAS mutation Y71X, preferably Y71H.

Another aspect relates to a use of a KRAS mutation Y71X, preferably Y71H, as a biomarker for a cancer or cancer cells being resistant to treatment with a tri-complex inhibitor and/or the cancer or cancer cells being susceptible to treatment with a KRAS G12D switch II inhibitor.

### BRIEF DESCRIPTION OF THE FIGURE

FIG. 1:

IC50 ratio of KRAS_G12D-Y71H/KRAS_G12D Ba/F3 and GP2d cells. A ratio of 1 (dotted line) indicates the same sensitivity in both cell lines (“parental”, expressing KRAS_G12D, and “resistant” expressing KRAS_G12D-Y71H). An increased ratio indicates a reduced sensitivity in the KRAS_G12D-Y71H cells (resistant). II-n (open circles): KRAS switch II inhibitors as defined in Table 16, Table 17 and Table 18, “n” being one of the number defined there. TCI-1 (grey triangle) and TCI-2 (grey square): Tri-complex Inhibitor 1 and 2, as defined in table 18.

### DETAILED DESCRIPTION OF THE INVENTION

### Tri-Complex Inhibitors

As used herein, tri-complex inhibitor refers to small molecule inhibitors which form a binary complex with Cyclophilin A, creating a high affinity interface for active (K)RAS, which is in turn recruited, forming a tri-complex.

Preferably, referring to all aspects and embodiments, including methods of treatment, uses, combinations, and compositions, the tri-complex inhibitors are for example described in WO2021091956, WO2022060836 and WO2023060253. Furthermore, referring to all aspects and embodiments, including methods of treatment, uses, combinations, and compositions, the tri-complex inhibitor is Daraxonrasib (RMC-6236) and/or Zoldonrasib (RMC-9805)

### KRAS Switch II Inhibitors

As used herein, KRAS refers to Kirsten rat sarcoma viral oncogene homolog a small GTPase of the Ras family of proteins. As used herein the term “KRAS switch II inhibitor” refers to a compound that inhibits KRAS by binding into its switch II pocket. KRAS switch II inhibitors belonging to different compound classes are known.

KRAS G12D switch II Inhibitors

As used herein, KRAS G12D refers to Kirsten rat sarcoma viral oncogene homolog a small GTPase of the Ras family of proteins mutated in residue Glycine12 to aspartic acid. As used herein the term “KRAS G12D switch II inhibitor” refers to a compound that inhibits KRAS G12D by binding into its switch II pocket. KRAS G12D switch II inhibitors belonging to different compound classes are known.

The term “KRAS G12D switch II inhibitor” as used herein also includes the KRAS G12D switch II inhibitors listed below in the form of a tautomer, of a pharmaceutically acceptable salt, of a hydrate or of a solvate, including a hydrate or solvate of a pharmaceutically acceptable salt. It also includes the KRAS G12D switch II inhibitor in all its solid, preferably crystalline, forms and in all the crystalline forms of its pharmaceutically acceptable salts, hydrates and solvates, including hydrates and solvates of pharmaceutically acceptable salts.

The term “pharmaceutically acceptable salts” as used herein includes both acid and base addition salts. Pharmaceutically acceptable acid addition salts refers to those salts which retain the biological effectiveness and properties of the free bases and which are not biologically or otherwise undesirable, formed with inorganic acids or organic acids. Pharmaceutically acceptable base addition salts include salts derived from inorganic bases or organic nontoxic bases. The term “solvate” as used herein refers to an association or complex of one or more solvent molecules and a compound of the present invention. Examples of solvents include water, isopropanol, ethanol, methanol, DMSO, ethyl acetate, acetic acid and ethanolamine. The term “hydrate” refers to a complex where the solvent molecule is water.

Preferably, referring to all aspects and embodiments, including methods of treatment, uses, combinations, and compositions, the KRAS switch II inhibitor is selected from KRAS inhibitors generically described and/or specifically disclosed in WO2022173678, WO2023099624, WO2023064857, and Wang, X. et al. Journal of Medicinal Chemistry 2022 65 (4), 3123-3133.

Furthermore, referring to all aspects and embodiments, including methods of treatment, uses, combinations, and compositions, the KRAS switch II inhibitor can be selected from the group consisting of
KRAS inhibitors generically described/or specifically disclosed in WO2023064857,
any KRAS inhibitor of example 1 to 73 as disclosed in WO2023064857,
preferably KRAS inhibitor of example 22 and/or 26 as disclosed in WO2023064857 the disclosure being incorporated by reference in its entirety herein, and with the respective synthesis and properties.

Furthermore, referring to all aspects and embodiments, including methods of treatment, uses, combinations, and compositions, the KRAS G12D switch II inhibitor can be selected from the group consisting of
KRAS inhibitors generically described/or specifically disclosed in WO2023064857,
any KRAS inhibitor of example 1 to 73 as disclosed in WO2023064857,
preferably KRAS inhibitor of example 22 and/or 26 as disclosed in WO2023064857
KRAS G12D inhibitors generically described/or specifically disclosed in WO2022173678
any KRAS inhibitor of example 1 to 51 as disclosed in WO2022173678
preferably KRAS inhibitor of example 21 and/or 28 as disclosed in WO2022173678
MRTX1133 (Wang, X. et al. Journal of Medicinal Chemistry 2022 65 (4), 3123-3133) the disclosure being incorporated by reference in its entirety herein, and with the respective synthesis and properties.

Furthermore, referring to all aspects and embodiments, including methods of treatment, uses, combinations, and compositions, the KRAS G12D switch II inhibitor can be selected from WO2023064857, preferably examples 22 and 26, WO2022173678, preferably examples 21 and 28, and MRTX1133 (Wang, X. et al. Journal of Medicinal Chemistry 2022 65 (4), 3123-3133).

In a preferred aspect, the KRAS G12D switch II inhibitor is a compound according to formula (I)

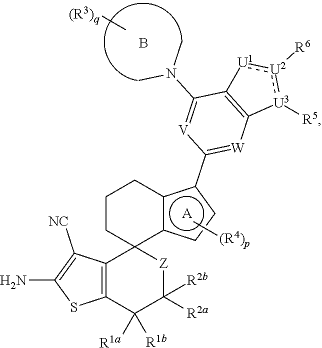

wherein
R^{1a} and R^{1b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
R^{2a} and R^{2b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
and/or, optionally, one of R^{1a} or R^{1b} and one of R^{2a} or R^{2b} together with the carbon atoms they are attached to form a cyclopropane ring;
Z is —(CR^{7a}R^{7b})ₙ—;
each R^{7a} and R^{7b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
or R^{7a} and R^{7b} together with the carbon atom they are attached to form a cyclopropane ring;
n is selected from the group consisting of 0, 1 and 2;
W is nitrogen (—N═) or —CH═;
V is nitrogen (—N═) or —CH═;
ring A is a ring selected from the group consisting of pyrrole, furan, thiophene, imidazole, pyrazole, oxazole, isoxazole, thiazole, isothiazole and triazole;
each R⁴, if present, is independently selected from the group consisting of C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocyclyl;
p is selected from the group consisting of 0, 1, 2 and 3;
U¹ is nitrogen (—N═) or sulphur (—S—);
U² is nitrogen (—N═) or —CR⁶═;
U³ is nitrogen (—NR⁵—) or —CR⁵═;
ring B is a 4-11 membered heterocycle;
each R³, if present, is independently selected from the group consisting of R⁸ and R⁹;
   each R⁸ is independently selected from the group consisting of —OR⁹, —NR⁹R⁹, halogen, and —CN;
   each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
   each R¹⁰ is independently selected from the group consisting of —OR¹¹, —NR¹¹R¹¹ and halogen;
   each R¹¹ is independently selected from the group consisting of C₁₋₆alkyl, C₃₋₁₀cycloalkyl, and 3-11 membered heterocyclyl; wherein the C₁₋₆alkyl, C₃₋₁₀cycloalkyl, and 3-11 membered heterocyclyl are all optionally substituted with one or more identical or different R¹²;
   each R¹² is independently selected from the group consisting of halogen, C₁₋₆alkyl, C₁₋₆alkoxy, C₁₋₆haloalkyl;
q is selected from the group consisting of 0, 1, 2, and 3;
R⁵ is selected from the group consisting of C₁₋₁₀aryl, and 5-13 membered heteroaryl; wherein the C₆₋₁₀aryl, and 5-13 membered heteroaryl are all optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl, wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴
   each R¹⁴ is independently selected from the group consisting of halogen, —CN, C₁₋₆alkyl, C₁₋₆haloalkyl, and C₃₋₁₀cycloalkyl;
R⁶ is a selected from the group consisting of hydrogen, C₁₋₆alkyl, C₁₋₆haloalkyl, C₃₋₁₀cycloalkyl and 3-11 membered heterocyclyl; wherein C₁₋₆alkyl, C₁₋₆haloalkyl, C₃₋₁₀cycloalkyl and 3-11 membered heterocyclyl are all optionally substituted with one or more, identical or different R¹⁵;
   each R¹⁵ is independently selected from the group consisting of halogen, C₁₋₆alkyl, and C₁₋₆haloalkyl;
or a salt thereof.

In another aspect, the KRAS G12D switch II inhibitor relates to the compound of formula (I), or a salt thereof, wherein V and W are nitrogen (—N═).

In another aspect, the KRAS G12D switch II inhibitor relates to the compound of formula (I), or a salt thereof, wherein at least one of V and W is nitrogen (—N═).

In another aspect, the KRAS G12D switch II inhibitor relates to the compound of formula (I), or a salt thereof, wherein
V is —CH═;
W is nitrogen (—N═).

In another aspect, the invention relates to the compound of formula (I), or a salt thereof, wherein
V is nitrogen (—N═),
W is —CH═.

In another aspect, the KRAS G12D switch II inhibitor relates to the compound of formula (I), or a salt thereof, wherein V and W are —CH═.

In another aspect, the KRAS G12D switch II inhibitor relates to the compound of formula (I), or a salt thereof, wherein
U¹ is sulphur (—S—);
U² is —CR⁶═;
U³ is —CR⁵═.

In another aspect, the KRAS G12D switch II inhibitor relates to the compound of formula (I), or a salt thereof, wherein
U¹ is nitrogen (—N═);
U² is nitrogen (—N═); and
U³ is nitrogen (—NR⁵—).

In another aspect, the KRAS G12D switch II inhibitor relates to the compound of formula (I), or a salt thereof, wherein
U¹ is nitrogen (—N═);
U² is —CR⁶═;
U³ is nitrogen (—NR⁵—).

In another aspect, the KRAS G12D switch II inhibitor relates to the compound of formula (II), or a salt thereof,

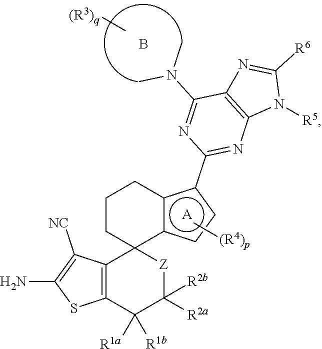

wherein
R^{1a}, R^{1b}, R^{2a}, R^{2b}, R³, R⁴, R⁵, R⁶, Z, ring A, ring B, p and q are as defined herein above or below.

In another aspect, the KRAS G12D switch II inhibitor relates to the compound of formula (II*), or a salt thereof,

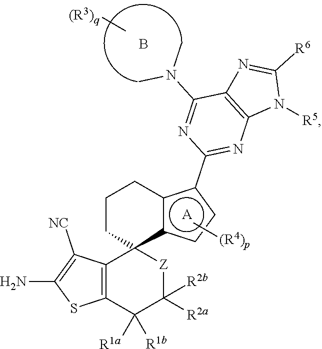

wherein
R^{1a}, R^{1b}, R^{2a}, R^{2b}, R³, R⁴, R⁵, R⁶, Z, ring A, ring B, p and q are as defined herein above or below.

In another aspect, the G12D switch II inhibitor relates to the compound of formula (II), or a salt thereof, wherein R^{1a} and R^{1b} are both independently selected from the group consisting of hydrogen and C₁₋₄alkyl.

In another aspect, the KRAS G12D switch II inhibitor relates to the compound of formula (II), or a salt thereof, wherein R^{2a} and R^{2b} are both independently selected from the group consisting of hydrogen and halogen.

In another aspect, the KRAS G12D switch II inhibitor relates to the compound of formula (II), or a salt thereof, wherein R^{1a} and R^{1b} are both independently selected from the group consisting of hydrogen and methyl.

In another aspect, the KRAS G12D switch II inhibitor relates to the compound of formula (II), or a salt thereof, wherein R^{2a} and R^{2b} are both independently selected from the group consisting of hydrogen and fluorine.

In another aspect, the KRAS G12D switch II inhibitor relates to the compound of formula (II), or a salt thereof, wherein R^{1a}, R^{1b}, R^{2a} and R^{2b} are hydrogen.

In another aspect, the KRAS G12D switch II inhibitor relates to the compound of formula (II), or a salt thereof, wherein n is 0.

In another aspect, the KRAS G12D switch II inhibitor relates to the compound of formula (II), or a salt thereof, wherein n is 1; and each R^{7a} and R^{7b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl.

In another aspect, the KRAS G12D switch II inhibitor relates to the compound of formula (II), or a salt thereof, wherein Z is —CH₂—.

In another aspect, the KRAS G12D switch II inhibitor relates to the compound of formula (II), or a salt thereof, wherein n is 2; and each R^{7a} and R^{7b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl.

In another aspect, the KRAS G12D switch II inhibitor relates to the compound of formula (II), or a salt thereof, wherein p is 0.

In another aspect, the KRAS G12D switch II inhibitor relates to the compound of formula (IIa) or a salt thereof

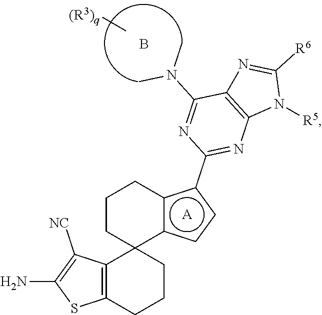

wherein
ring A, ring B, R³, R⁵, R⁶, and q are as defined herein.

In another aspect, the KRAS G12D switch II inhibitor relates to the compound of formula (IIb) or a salt thereof

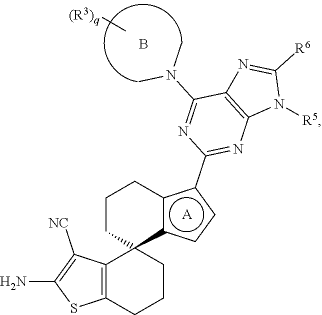

wherein ring A, ring B, R³, R⁵, R⁶, and q are as defined herein.

In another aspect, the KRAS G12D switch II inhibitor relates to the compound of the invention, or a salt thereof, wherein ring A is a ring selected from the group consisting of imidazole, pyrazole, oxazole, isoxazole, thiazole, isothiazole and triazole.

In another aspect, the KRAS G12D switch II inhibitor relates to the compound of the invention, or a salt thereof, wherein ring A is a ring selected from the group consisting of pyrrole, furan, thiophene, imidazole, pyrazole, isoxazole, isothiazole and triazole.

In another aspect, the KRAS G12D switch II inhibitor relates to the compound of the invention, or a salt thereof, wherein ring A is selected from the group consisting of

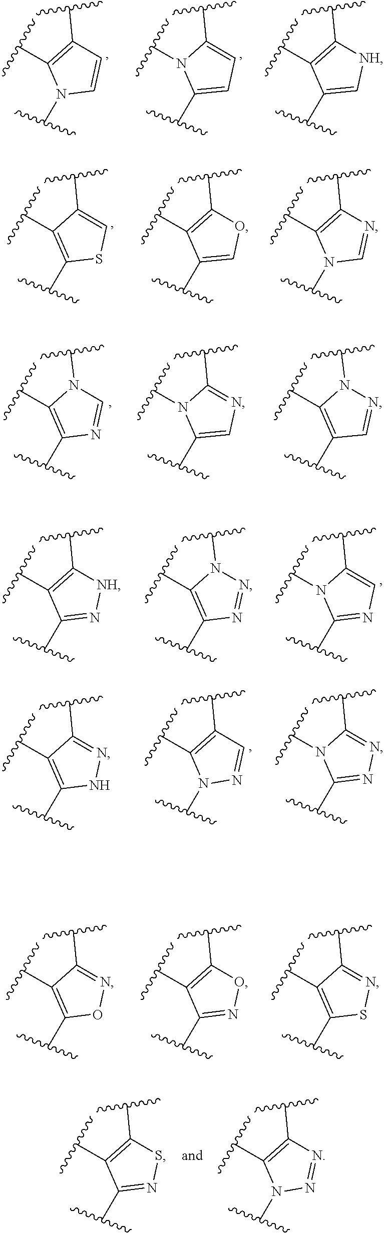

In another aspect, the KRAS G12D switch II inhibitor relates to the compound of the invention, or a salt thereof, wherein ring A is isoxazole or isothiazole.

In another aspect, the KRAS G12D switch II inhibitor relates to the compound of the invention, or a salt thereof, wherein ring A is selected from

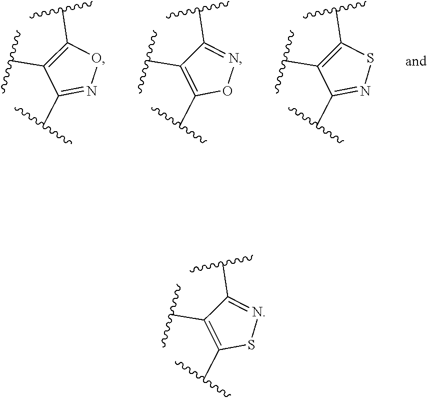 [SMILES: *c1noc(*)c1* ; *c1noc(*)c1* ; *c1nsc(*)c1* ; *c1nsc(*)c1*]

In another aspect, the KRAS G12D switch II inhibitor relates to the compound of the invention, or a salt thereof, wherein ring A is

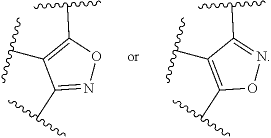 [SMILES: *c1noc(*)c1* ; *c1noc(*)c1*]

In another aspect, the KRAS G12D switch II inhibitor relates to the compound of formula (IIc) or a salt thereof

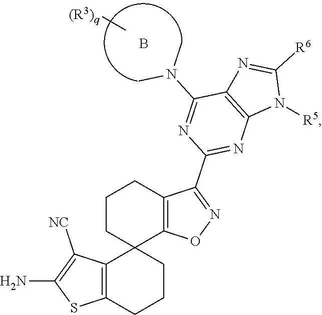

wherein
ring B, R³, R⁵, R⁶, and q are as defined herein.

In another aspect, the KRAS G12D switch II inhibitor relates to the compound of formula (IId) or a salt thereof

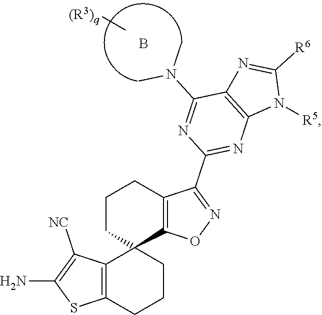

wherein
ring B, R³, R⁵, R⁶, and q are as defined herein.

In another aspect, the KRAS G12D switch II inhibitor relates to the compound of formula (IIe) or a salt thereof

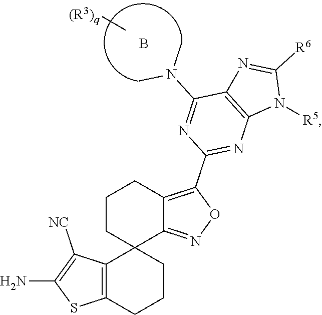

wherein
ring B, R³, R⁵, R⁶, and q are as defined herein.

In another aspect, the KRAS G12D switch II inhibitor relates to the compound of formula (IIf) or a salt thereof

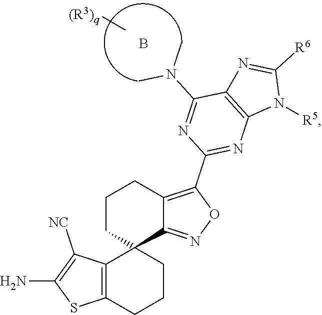

wherein
ring B, R³, R⁵, R⁶, and q are as defined herein.

In another aspect, the KRAS G12D switch II inhibitor relates to the compound of formula (I), (II), (II*), (IIa), (IIb), (IIc), (IId), (IIe) or (IIf), or a salt thereof, wherein ring B is a 4-11 membered heterocycle comprising at least one nitrogen.

In another aspect, the KRAS G12D switch II inhibitor relates to the compound of formula (I), (II), (II*), (IIa), (IIb), (IIc), (IId), (IIe) or (IIf), or a salt thereof, wherein ring B is a 4-8 membered heterocycle.

In another aspect, the KRAS G12D switch II inhibitor relates to the compound of formula (I), (II), (II*), (IIa), (IIb), (IIc), (IId), (IIe) or (IIf), or a salt thereof, wherein ring B is a 4-8 membered heterocycle comprising at least one nitrogen.

In another aspect, the KRAS G12D switch II inhibitor relates to the compound of formula (I), (II), (II*), (IIa), (IIb), (IIc), (IId), (IIe) or (IIf), or a salt thereof, wherein ring B is a ring selected from the group consisting of azetidine, pyrrolidine, imidazolidine, piperidine, piperazine, morpholin, azepane, diazepane, oxazepane, 2-azabicyclo[2.2.1]heptane and 4,7-diazaspiro[2.5]octane.

In another aspect, the KRAS G12D switch II inhibitor relates to the compound of formula (I), (II), (II*), (IIa), (IIb), (IIc), (IId), (IIe) or (IIf), or a salt thereof, wherein ring B is a ring selected from the group consisting of azetidine, pyrrolidine, piperazine, 2-azabicyclo[2.2.1]heptane and 4,7-diazaspiro[2.5]octane

In another aspect, the KRAS G12D switch II inhibitor relates to the compound of formula (I), (II), (II*), (IIa), (IIb), (IIc), (IId), (IIe) or (IIf), or a salt thereof, wherein ring B is a ring selected from the group consisting of azetidine.

In another aspect, the KRAS G12D switch II inhibitor relates to the compound of formula (I), (II), (II*), (IIa), (IIb), (IIc), (IId), (IIe) or (IIf), or a salt thereof, wherein ring B is a ring selected from the group consisting of pyrrolidine.

In another aspect, the invention relates to the compound of the formula (I), (II), (II*), (IIa), (IIb), (IIc), (IId), (IIe) or (IIf), or a salt thereof, wherein ring B is piperazine.

In another aspect, the invention relates to the compound of the formula (I), (II), (II*), (IIa), (IIb), (IIc), (IId), (IIe) or (IIf), or a salt thereof, wherein ring B is 2-azabicyclo[2.2.1]heptane.

In another aspect, the KRAS G12D switch II inhibitor relates to the compound of formula (I), (II), (II*), (IIa), (IIb), (IIc), (IId), (IIe) or (IIf), or a salt thereof, wherein
each R³, if present, is independently selected from the group consisting of R⁸ and R⁹;
   each R⁸ is independently selected from the group consisting of —OR⁹ and —NR⁹R⁹;
   each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
   each R¹⁰ is independently selected from the group consisting of —OR¹¹, —NR¹¹R¹¹ and halogen;
   each R¹¹ is independently selected from the group consisting of C₁₋₆alkyl, C₃₋₁₀cycloalkyl, and 3-11 membered heterocyclyl; wherein the C₁₋₆alkyl, C₃₋₁₀cycloalkyl, and 3-11 membered heterocyclyl are all optionally substituted with one or more identical or different R¹²
   each R¹² is independently selected from the group consisting of halogen, C₁₋₆alkyl, C₁₋₆alkoxy, C₁₋₆haloalkyl.

In another aspect, the KRAS G12D switch II inhibitor relates to the compound of formula (I), (II), (II*), (IIa), (IIb), (IIc), (IId), (IIe) or (IIf), or a salt thereof, wherein
each R³, if present, is independently selected from the group consisting of R⁸ and R⁹;
   each R⁸ is independently selected from the group consisting of —OR⁹ and —NR⁹R⁹;
   each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
   each R¹⁰ is independently selected from the group consisting of —OR¹¹, —NR¹¹R¹¹ and halogen;
   each R¹¹ is independently selected from the group consisting of C₁₋₆alkyl, C₃₋₁₀cycloalkyl, and 3-11 membered heterocyclyl.

In another aspect, the KRAS G12D switch II inhibitor relates to the compound of formula (I), (II), (II*), (IIa), (IIb), (IIc), (IId), (IIe) or (IIf), or a salt thereof, wherein q is selected from the group consisting of 0, 1 and 2.

In another aspect, the KRAS G12D switch II inhibitor relates to the compound of formula (I), (II), (II*), (IIa), (IIb), (IIc), (IId), (IIe) or (IIf), or a salt thereof, wherein q is selected from the group consisting of 1 and 2.

In another aspect, the KRAS G12D switch II inhibitor relates to the compound of formula (I), (II), (II*), (IIa), (IIb), (IIc), (IId), (IIe) or (IIf), or a salt thereof, wherein q is of 0.

In another aspect, the KRAS G12D switch II inhibitor relates to the compound of formula (I), (II), (II*), (IIa), (IIb), (IIc), (IId), (IIe) or (IIf), or a salt thereof, wherein q is 1.

In another aspect, the KRAS G12D switch II inhibitor relates to the compound of formula (I), (II), (II*), (IIa), (IIb), (IIc), (IId), (IIe) or (IIf), or a salt thereof, wherein q is 2.

In another aspect, the KRAS G12D switch II inhibitor relates to the compound of formula (I), (II), (II*), (IIa), (IIb), (IIc), (IId), (IIe) or (IIf), or a salt thereof, wherein ring B—(R³)q is selected from the group consisting of

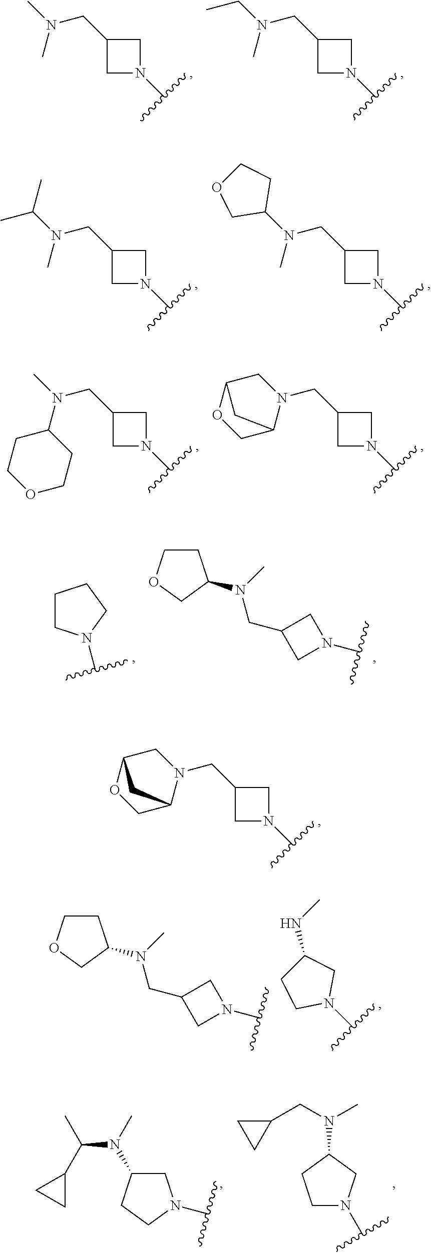

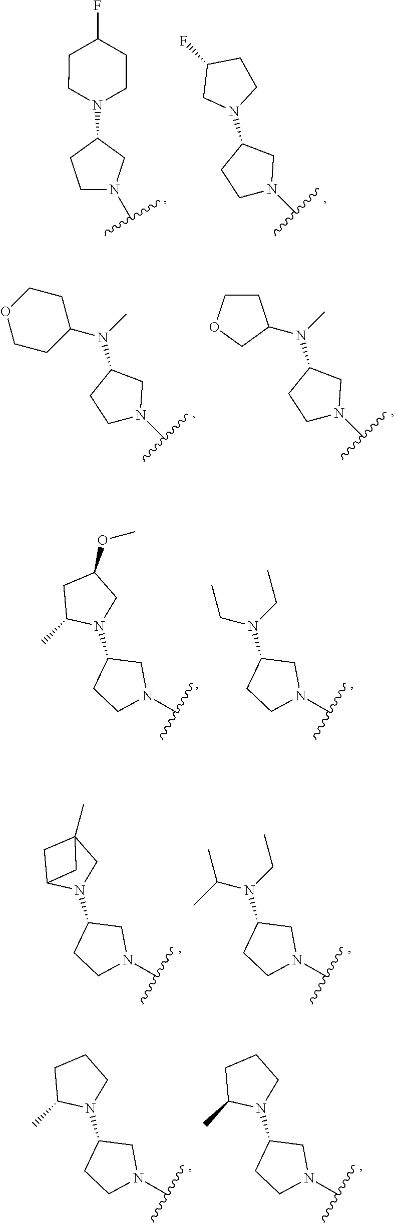

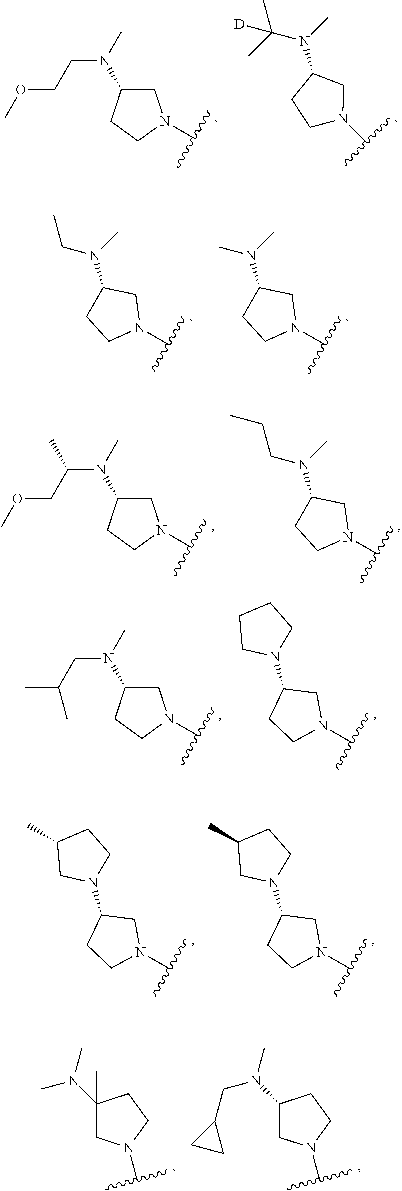

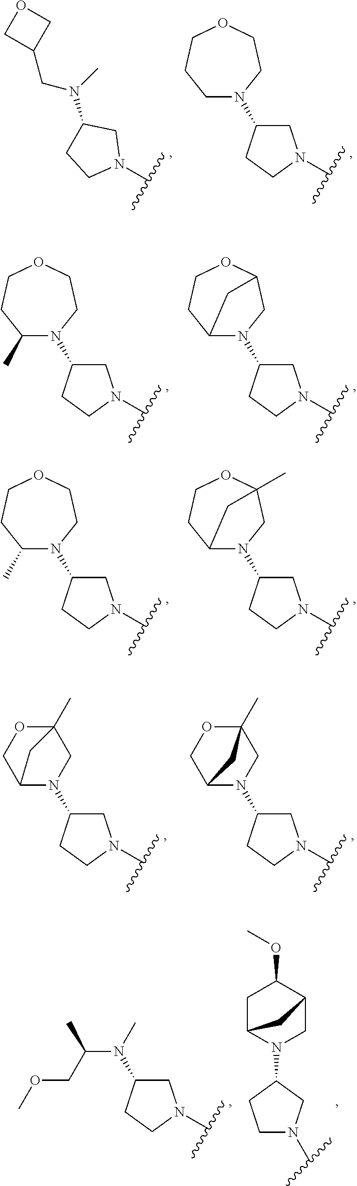

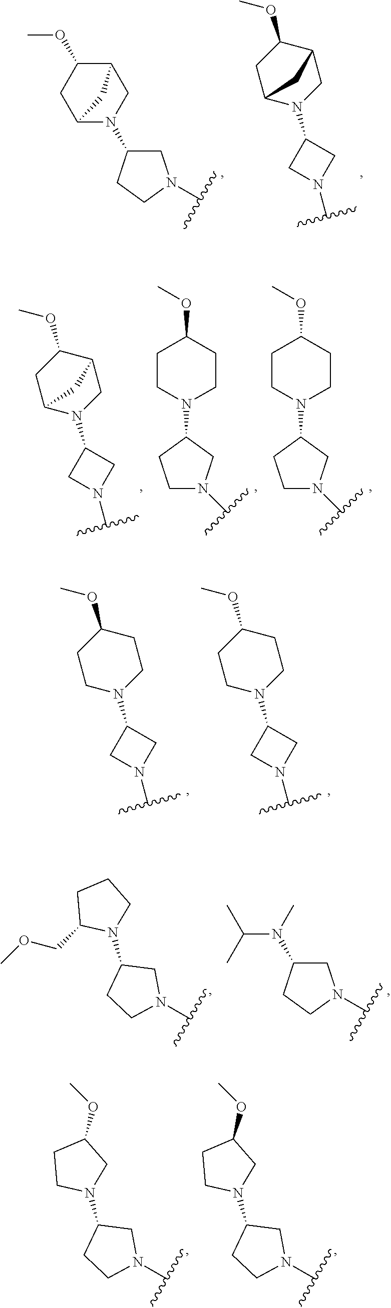

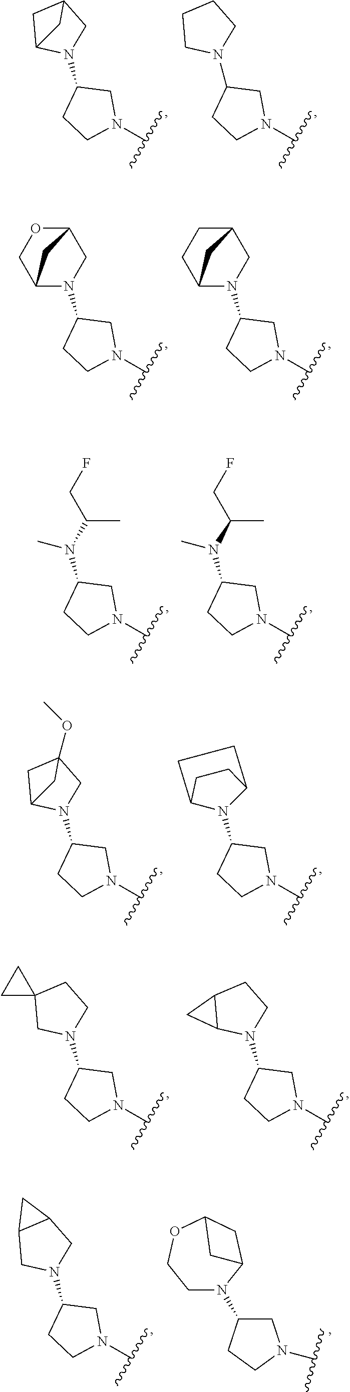

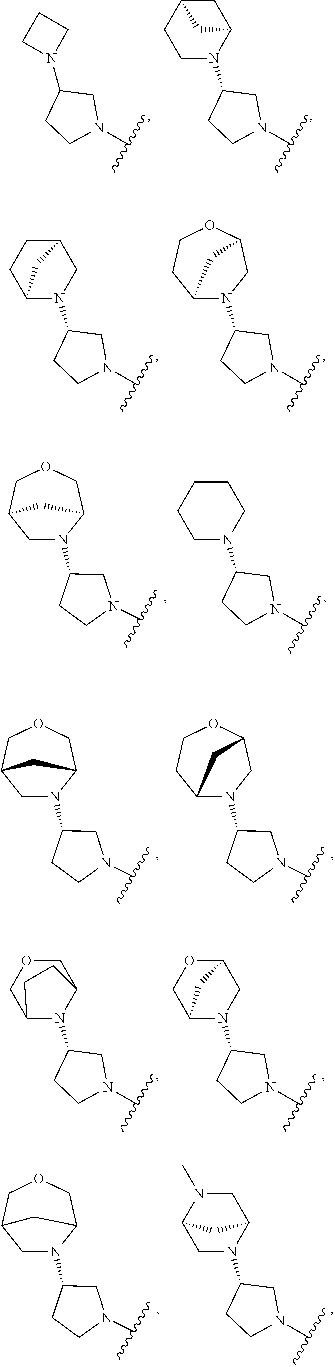

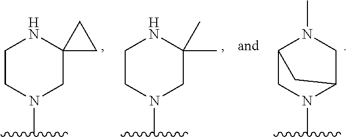

In another aspect, the KRAS G12D switch II inhibitor relates to the compound of formula (I), (II), (II*), (IIa), (IIb), (IIc), (IId), (IIe) or (IIf), or a salt thereof, wherein
R⁵ is C₅₋₁₀aryl, wherein the C₅₋₁₀aryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the KRAS G12D switch II inhibitor relates to the compound of formula (I), (II), (II*), (IIa), (IIb), (IIc), (IId), (IIe) or (IIf), or a salt thereof, wherein
R⁵ is C₆₋₁₀aryl, wherein the C₆₋₁₀aryl is optionally substituted with one or more, identical or different R¹³;
   each R¹³ is independently selected from the group consisting of halogen, —CN, C₁₋₆alkyl, and C₁₋₆haloalkyl; wherein the C₁₋₆alkyl, and C₁₋₆haloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the KRAS G12D switch II inhibitor relates to the compound of formula (I), (II), (II*), (IIa), (IIb), (IIc), (IId), (IIe) or (IIf), or a salt thereof, wherein
R⁵ is phenyl, wherein the phenyl is optionally substituted with one or more, identical or different R¹³;
   each R¹³ is independently selected from the group consisting of halogen, —CN, C₁₋₆alkyl, and C₁₋₆haloalkyl; wherein the C₁₋₆alkyl, and C₁₋₆haloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the KRAS G12D switch II inhibitor relates to the compound of formula (I), (II), (II*), (IIa), (IIb), (IIc), (IId), (IIe) or (IIf), or a salt thereof, wherein
R⁵ is a 5-13 membered heteroaryl; wherein the 5-13 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the KRAS G12D switch II inhibitor relates to the compound of formula (I), (II), (II*), (IIa), (IIb), (IIc), (IId), (IIe) or (IIf), or a salt thereof, wherein
R⁵ is a 5-6 membered heteroaryl; wherein the 5-6 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the KRAS G12D switch II inhibitor relates to the compound of formula (I), (II), (II*), (IIa), (IIb), (IIc), (IId), (IIe) or (IIf), or a salt thereof, wherein
R⁵ is a 7-13 membered heteroaryl; wherein the 7-13 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the KRAS G12D switch II inhibitor relates to the compound of formula (I), (II), (II*), (IIa), (IIb), (IIc), (IId), (IIe) or (IIf), or a salt thereof, wherein
R⁵ is a 7-9 membered heteroaryl; wherein the 7-9 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the KRAS G12D switch II inhibitor relates to the compound of formula (I), (II), (II*), (IIa), (IIb), (IIc), (IId), (IIe) or (IIf), or a salt thereof, wherein R⁵ is selected from the group consisting of

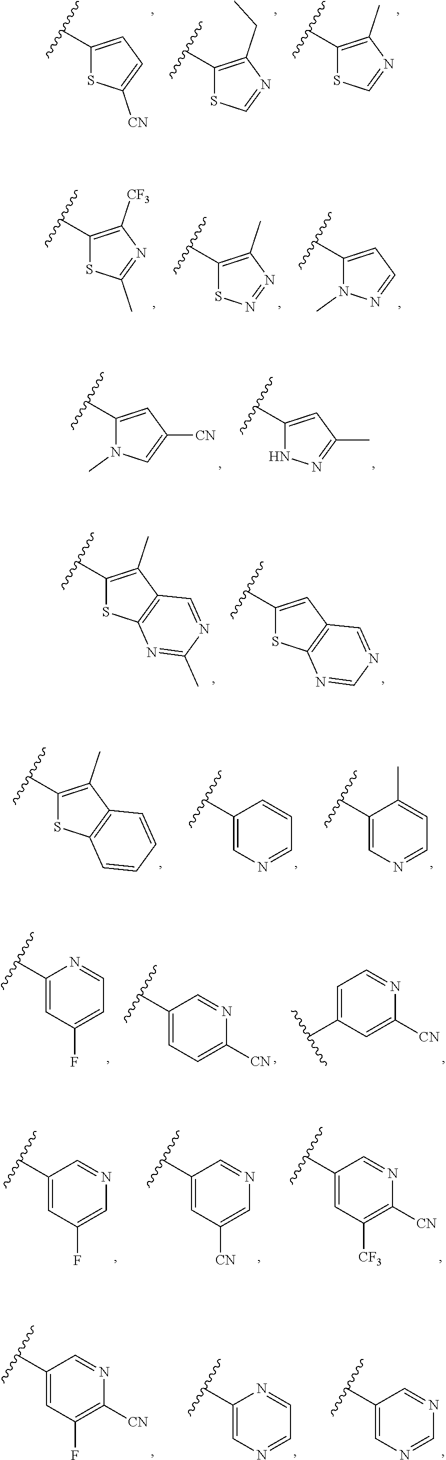

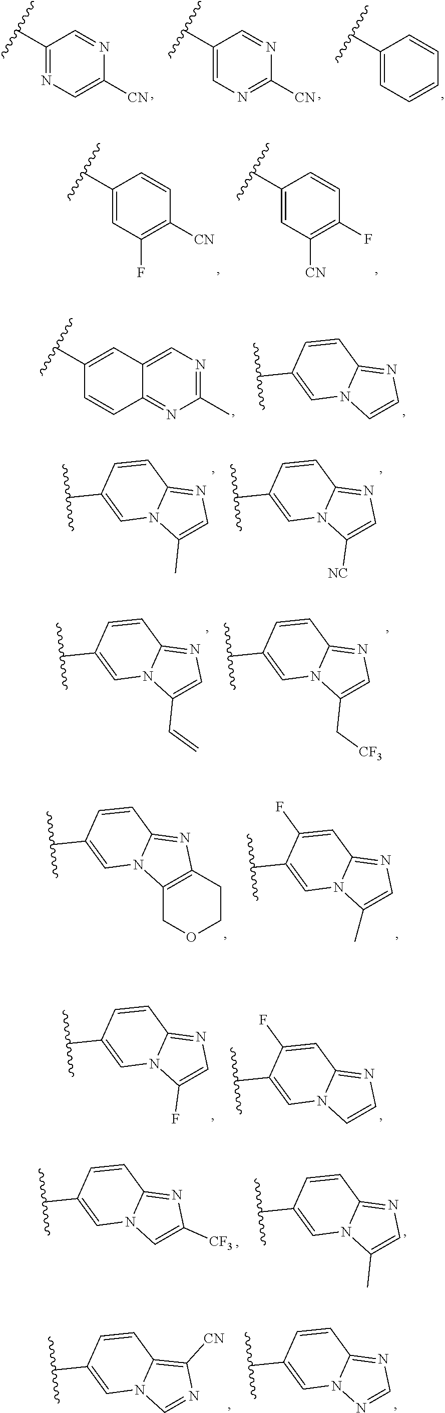

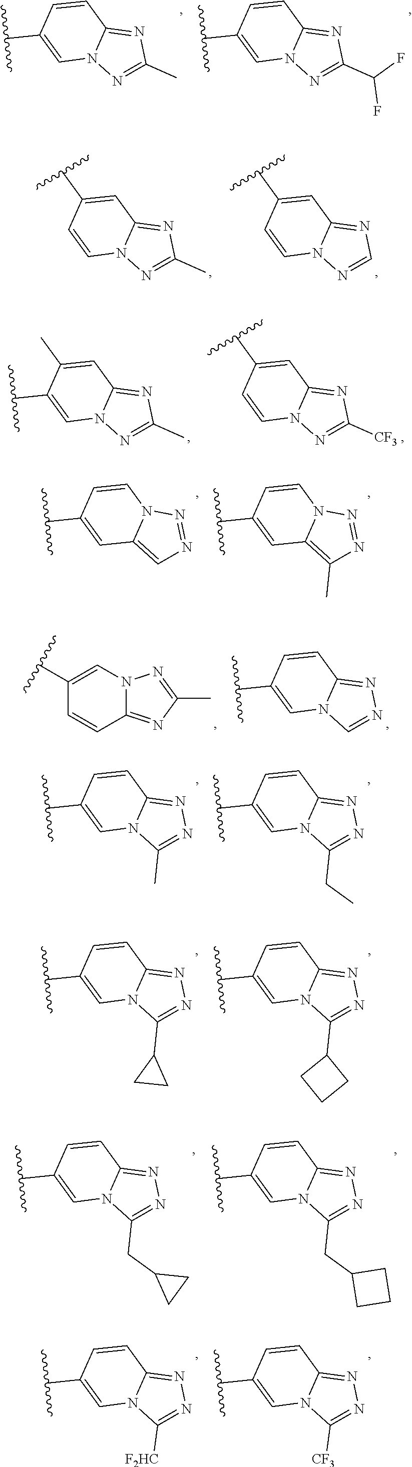

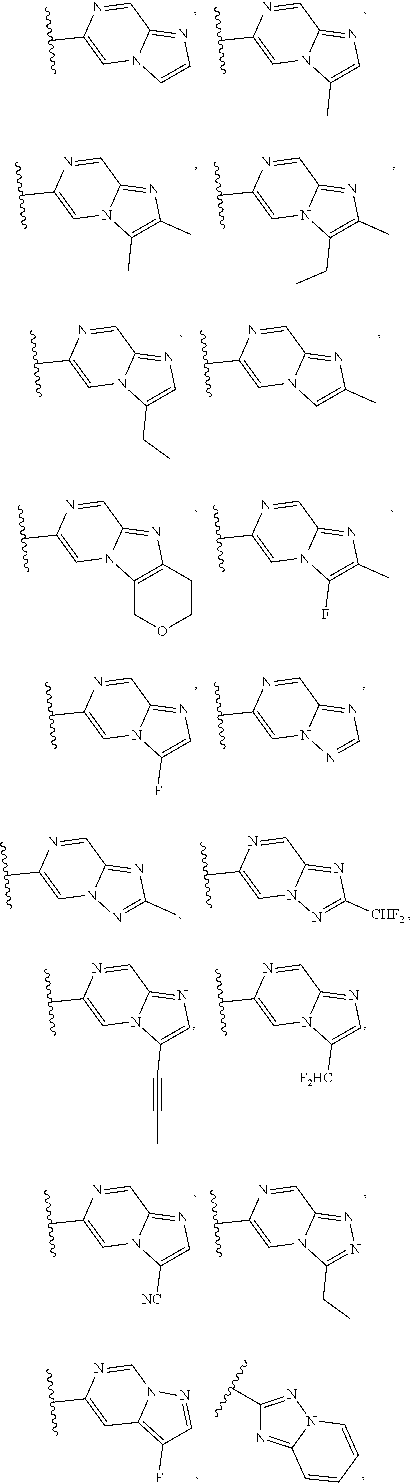

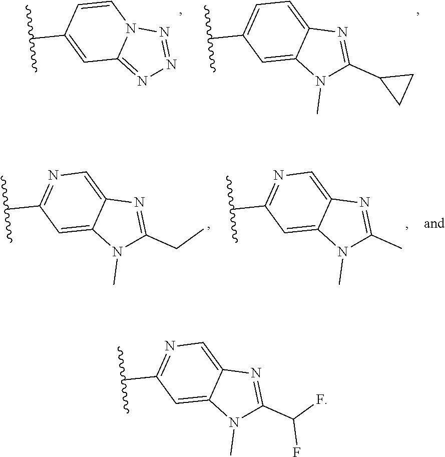

In another aspect, the KRAS G12D switch II inhibitor relates to the compound of formula (I), (II), (II*), (IIa), (IIb), (IIc), (IId), (IIe) or (IIf), or a salt thereof, wherein R⁶ is a selected from the group consisting of C₁₋₆alkyl, C₁₋₆haloalkyl, and C₃₋₁₀cycloalkyl; wherein C₁₋₆alkyl, C₁₋₆haloalkyl, and C₃₋₁₀cycloalkyl are all optionally substituted with one or more, identical or different R¹⁵;
each R¹⁵ is independently selected from the group consisting of halogen, C₁₋₆alkyl, and C₁₋₆haloalkyl.

In another aspect, the KRAS G12D switch II inhibitor relates to the compound of formula (I), (II), (II*), (IIa), (IIb), (IIc), (IId), (IIe) or (IIf), or a salt thereof, wherein R⁶ is selected from the group consisting of

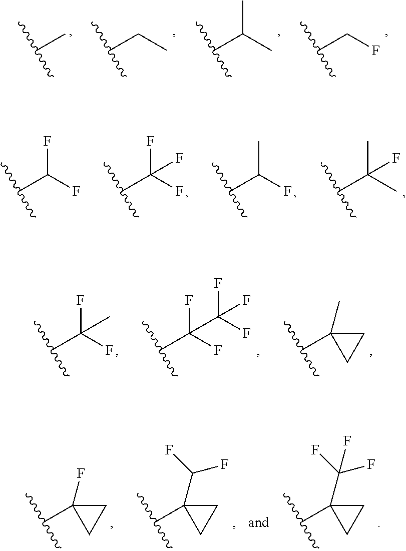

In another aspect, the KRAS G12D switch II inhibitor relates to the compound of formula (IIc), (IId), (IIe) or (IIf), or a salt thereof, wherein
ring B is a 4-8 membered heterocycle;
each R³, if present, is independently selected from the group consisting of R⁸ and R⁹;
   each R⁸ is independently selected from the group consisting of —OR⁹, —NR⁹R⁹, halogen, and —CN;
   each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
   each R¹⁰ is independently selected from the group consisting of —OR¹¹, —NR¹¹R¹¹ and halogen;
   each R¹¹ is independently selected from the group consisting of C₁₋₆alkyl, C₃₋₁₀cycloalkyl, and 3-11 membered heterocyclyl;
q is selected from the group consisting of 1 and 2;
R⁵ is a 5-13 membered heteroaryl; wherein the 5-13 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl, wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl;
R⁶ is a selected from the group consisting of hydrogen, C₁₋₆alkyl, C₁₋₆haloalkyl, C₃₋₁₀cycloalkyl and 3-11 membered heterocyclyl; wherein C₁₋₆alkyl, C₁₋₆haloalkyl, C₃₋₁₀cycloalkyl and 3-11 membered heterocyclyl are all optionally substituted with one or more, identical or different R¹⁵;
   each R¹⁵ is independently selected from the group consisting of halogen, C₁₋₆alkyl, and C₁₋₆haloalkyl;

In another aspect, the KRAS G12D switch II inhibitor relates to the compound of formula (IIc), (IId), (IIe) or (IIf), or a salt thereof, wherein
ring B is a 4-8 membered heterocycle;
each R³, if present, is independently selected from the group consisting of R⁸ and R⁹;
   each R⁸ is independently selected from the group consisting of —OR⁹, and —NR⁹R⁹;
   each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
   each R¹⁰ is independently selected from the group consisting of —OR¹¹, —NR¹¹R¹¹ and halogen;
   each R¹¹ is independently selected from the group consisting of C₁₋₆alkyl, C₃₋₁₀cycloalkyl, and 3-11 membered heterocyclyl;
   q is selected from the group consisting of 1 and 2;
R⁵ is a 5-13 membered heteroaryl; wherein the 5-13 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl, wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is C₃₋₁₀cycloalkyl;
R⁶ is C₁₋₆haloalkyl.

In another aspect, the KRAS G12D switch II inhibitor relates to the compound of formula (IIc), (IId), (IIe) or (IIf), or a salt thereof, wherein
ring B is a 4-8 membered heterocycle;
each R³, if present, is independently selected from the group consisting of R⁸ and R⁹;
   each R⁸ is independently selected from the group consisting of —OR⁹, and —NR⁹R⁹;
   each R⁹ is independently selected from the group consisting of hydrogen, C₁₋₆alkyl, C₁₋₆alkoxy, C₃₋₁₀cycloalkyl, and 3-11 membered heterocyclyl, wherein the C₁₋₆alkyl, C₁₋₆alkoxy, C₃₋₁₀cycloalkyl and 3-11 membered heterocyclyl, are all optionally substituted with one or more, identical or different R¹⁰ and/or R¹¹;
   each R¹⁰ is independently selected from the group consisting of —NR¹¹R¹¹ and halogen;
   each R¹¹ is independently selected from the group consisting of C₁₋₆alkyl, and 3-11 membered heterocyclyl;
q is selected from the group consisting of 1 and 2;
R⁵ is a 5-13 membered heteroaryl; wherein the 5-13 membered heteroaryl is optionally substituted with one or more, identical or different R¹³;
   each R¹³ is independently selected from the group consisting of halogen, —C₁₋₆alkyl, C₁₋₆haloalkyl, and C₃₋₁₀cycloalkyl,
R⁶ is C₁₋₆haloalkyl.

In another aspect, the KRAS G12D switch II inhibitor relates to the compound of formula (IIc), (IId), (IIe) or (IIf), or a salt thereof, wherein
ring B is pyrrolidine;
each R³, if present, is independently selected from the group consisting of R⁸ and R⁹;
   each R⁸ is independently selected from the group consisting of —OR⁹, and —NR⁹R⁹;
   each R⁹ is independently selected from the group consisting of hydrogen, C₁₋₆alkyl, C₁₋₆alkoxy, C₃₋₁₀cycloalkyl, and 3-11 membered heterocyclyl, wherein the C₁₋₆alkyl, C₁₋₆alkoxy, C₃₋₁₀cycloalkyl and 3-11 membered heterocyclyl, are all optionally substituted with one or more, identical or different R¹⁰ and/or R¹¹;
   each R¹⁰ is independently selected from the group consisting of —NR¹¹R¹¹ and halogen; each R¹¹ is independently selected from the group consisting of C₁₋₆alkyl, and 3-11 membered heterocyclyl;
q is 1;
R⁵ is a 5-13 membered heteroaryl; wherein the 5-13 membered heteroaryl is optionally substituted with one or more, identical or different R¹³;
   each R¹³ is independently selected from the group consisting of halogen, —C₁₋₆alkyl, C₁₋₆haloalkyl, and C₃₋₁₀cycloalkyl,
R⁶ is C₁₋₆haloalkyl.

In a preferred embodiment the KRAS G12D switch II inhibitor of formula (II) is selected from

| # | Structure |
|---|---|
| II-1 | 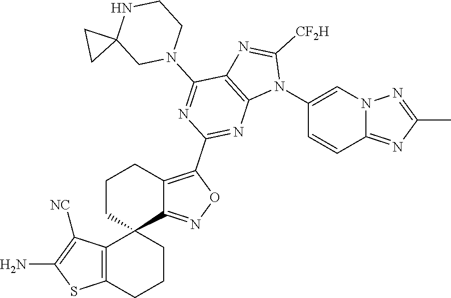 [SMILES: Cc1nc2ccc(-n3c(C(F)F)nc4c(N5CCNC6(CC6)C5)nc(-c5onc6c5CCC[C@@]65CCCc6sc(N)c(C#N)c65)nc43)cn2n1] |
| II-2 | 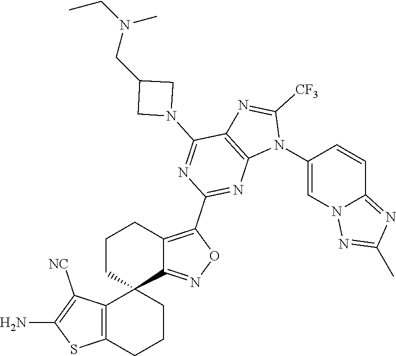 [SMILES: CCN(C)CC1CN(c2nc(-c3onc4c3CCC[C@@]43CCCc4sc(N)c(C#N)c43)nc3c2nc(C(F)(F)F)n3-c2ccc3nc(C)nn3c2)C1] |
| II-3 | 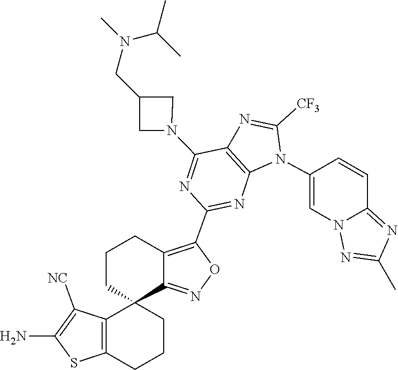 [SMILES: Cc1nc2ccc(-n3c(C(F)(F)F)nc4c(N5CC(CN(C)C(C)C)C5)nc(-c5onc6c5CCC[C@@]65CCCc6sc(N)c(C#N)c65)nc43)cn2n1] |
| II-4 | 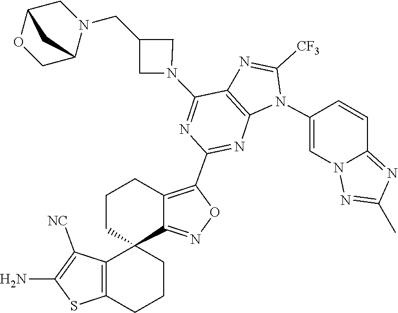 [SMILES: Cc1nc2ccc(-n3c(C(F)(F)F)nc4c(N5CC(CN6C[C@@H]7C[C@H]6CO7)C5)nc(-c5onc6c5CCC[C@@]65CCCc6sc(N)c(C#N)c65)nc43)cn2n1] |
| II-5 | 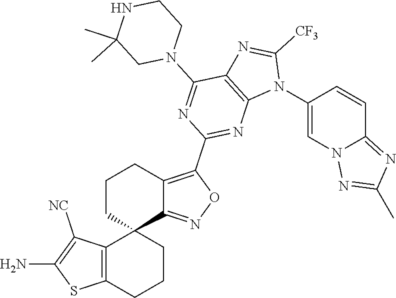 [SMILES: Cc1nc2ccc(-n3c(C(F)(F)F)nc4c(N5CCNC(C)(C)C5)nc(-c5onc6c5CCC[C@@]65CCCc6sc(N)c(C#N)c65)nc43)cn2n1] |
| II-6 | 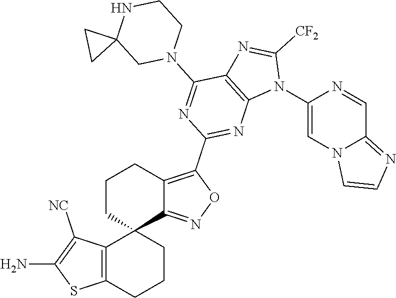 |
| II-7 | 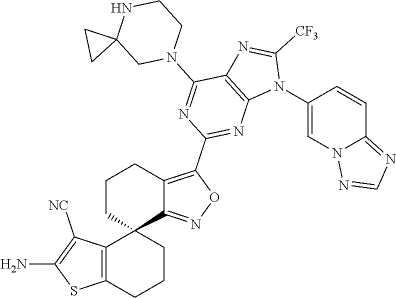 [SMILES: N#Cc1c(N)sc2c1[C@@]1(CCC2)CCCc2c1noc2-c1nc(N2CCNC3(CC3)C2)c2nc(C(F)(F)F)n(-c3ccc4ncnn4c3)c2n1] |
| II-8 | 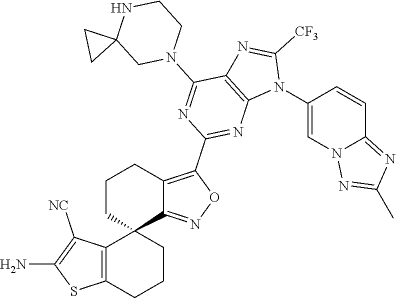 [SMILES: Cc1nc2ccc(-n3c(C(F)(F)F)nc4c(N5CCNC6(CC6)C5)nc(-c5onc6c5CCC[C@@]65CCCc6sc(N)c(C#N)c65)nc43)cn2n1] |
| II-9 | 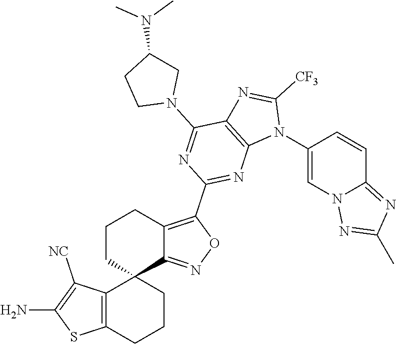 [SMILES: Cc1nc2ccc(-n3c(C(F)(F)F)nc4c(N5CC[C@H](N(C)C)C5)nc(-c5onc6c5CCC[C@@]65CCCc6sc(N)c(C#N)c65)nc43)cn2n1] |
| II-10 | 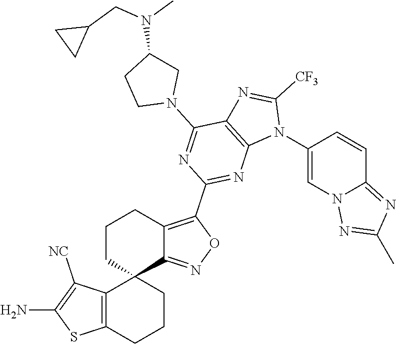 [SMILES: Cc1nc2ccc(-n3c(C(F)(F)F)nc4c(N5CC[C@H](N(C)CC6CC6)C5)nc(-c5onc6c5CCC[C@@]65CCCc6sc(N)c(C#N)c65)nc43)cn2n1] |
| II-11 | 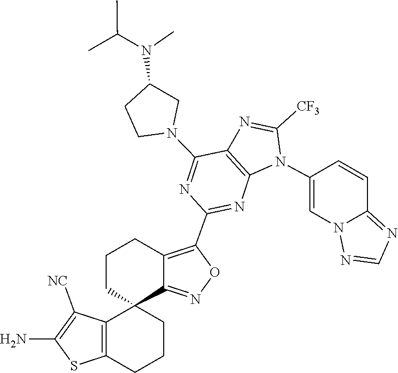 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3ncnn3c2)C1] |
| II-12 | 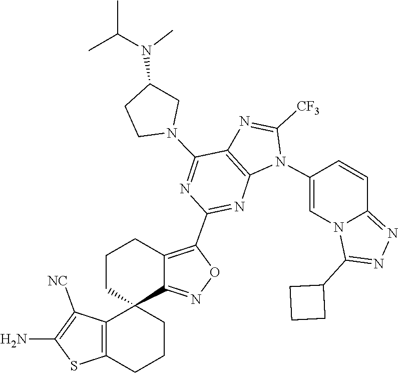 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C4CCC4)n3c2)C1] |
| II-13 | 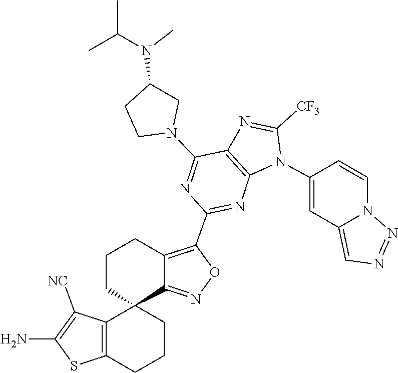 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccn3nncc3c2)C1] |
| II-14 | 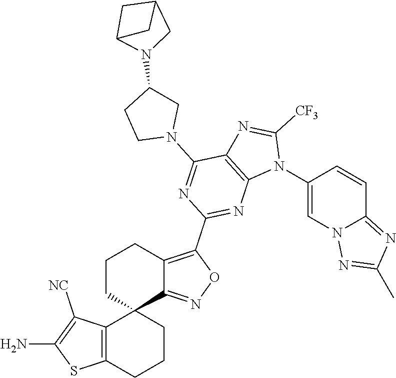 [SMILES: Cc1nc2ccc(-n3c(C(F)(F)F)nc4c(N5CC[C@H](N6CC7CC6C7)C5)nc(-c5onc6c5CCC[C@@]65CCCc6sc(N)c(C#N)c65)nc43)cn2n1] |
| II-15 | 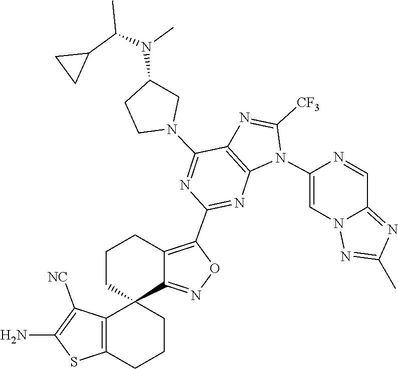 [SMILES: Cc1nc2cnc(-n3c(C(F)(F)F)nc4c(N5CC[C@H](N(C)[C@@H](C)C6CC6)C5)nc(-c5onc6c5CCC[C@@]65CCCc6sc(N)c(C#N)c65)nc43)cn2n1] |
| II-16 | 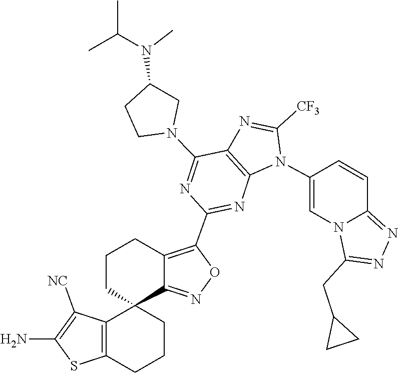 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(CC4CC4)n3c2)C1] |
| II-17 | 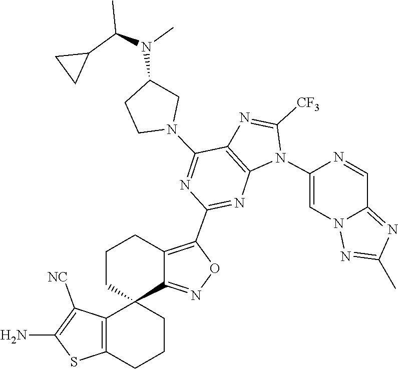 [SMILES: Cc1nc2cnc(-n3c(C(F)(F)F)nc4c(N5CC[C@H](N(C)[C@H](C)C6CC6)C5)nc(-c5onc6c5CCC[C@@]65CCCc6sc(N)c(C#N)c65)nc43)cn2n1] |
| II-18 | 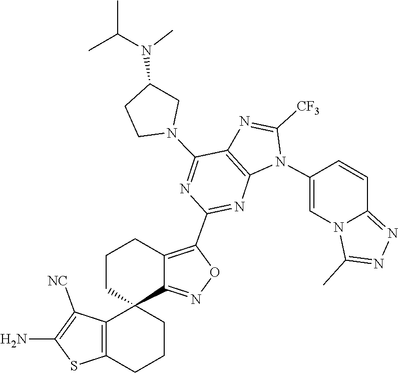 [SMILES: Cc1nnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] |
| II-19 | 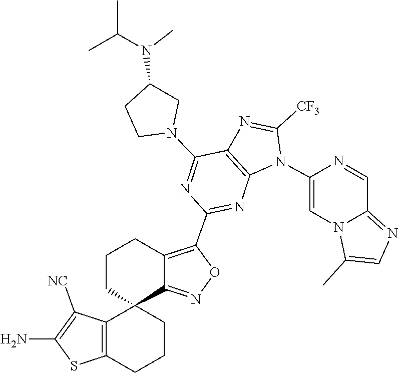 [SMILES: Cc1cnc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] |
| II-20 | 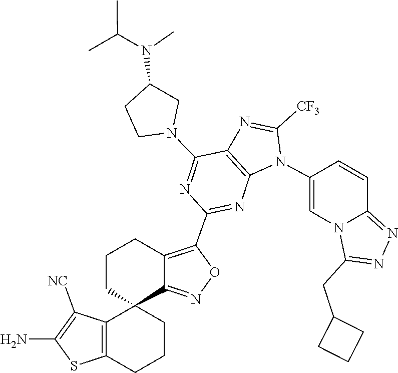 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(CC4CCC4)n3c2)C1] |
| II-21 | 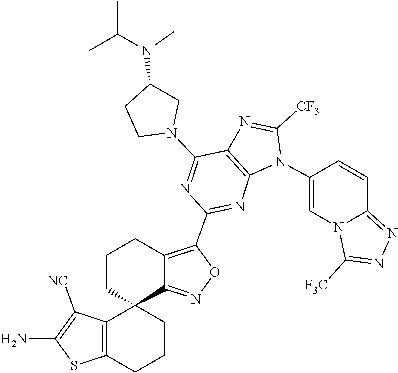 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1] |
| II-22 | 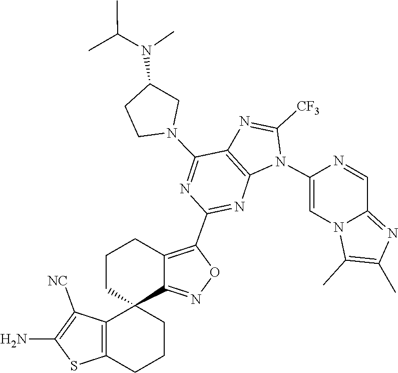 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2c1C] |
| II-23 | 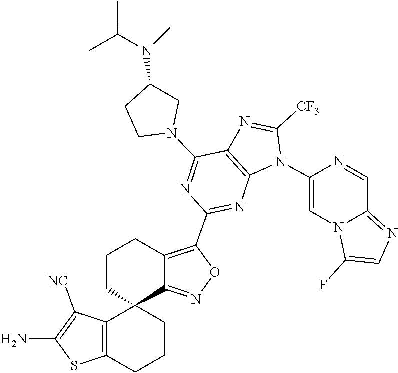 [SMILES: CC(C)N(C)[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1] |
| II-24 | 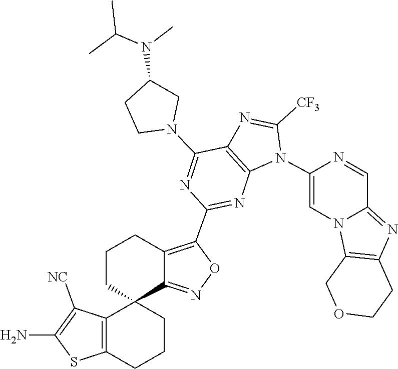 [SMILES: CC(C)N(C)[C@H]1CCN(c2nc(-c3onc4c3CCC[C@@]43CCCc4sc(N)c(C#N)c43)nc3c2nc(C(F)(F)F)n3-c2cn3c4c(nc3cn2)CCOC4)C1] |
| II-25 | 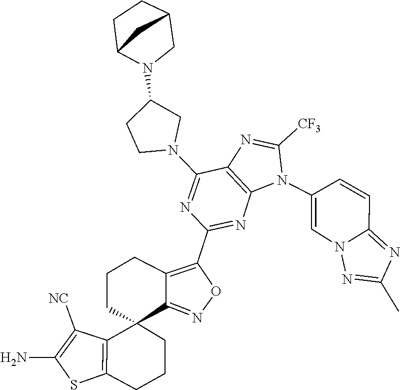 [SMILES: Cc1nc2ccc(-n3c(C(F)(F)F)nc4c(N5CC[C@H](N6C[C@H]7CC[C@@H]6C7)C5)nc(-c5onc6c5CCC[C@@]65CCCc6sc(N)c(C#N)c65)nc43)cn2n1] |
| II-26 | 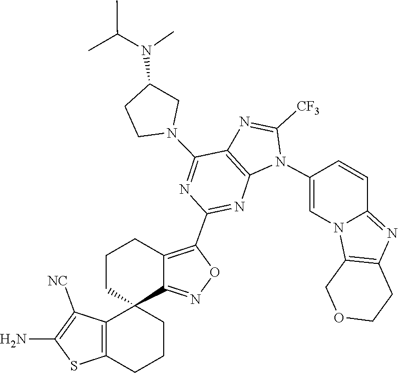 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nc4c(n3c2)COCC4)C1] |
| II-27 | 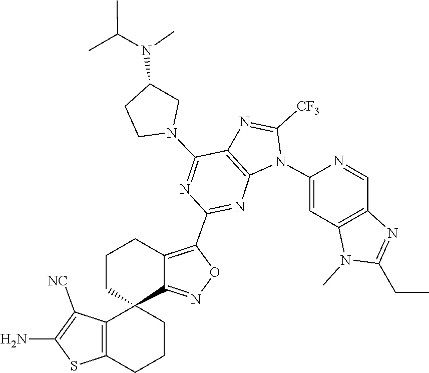 [SMILES: CCc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C] |
| II-28 | 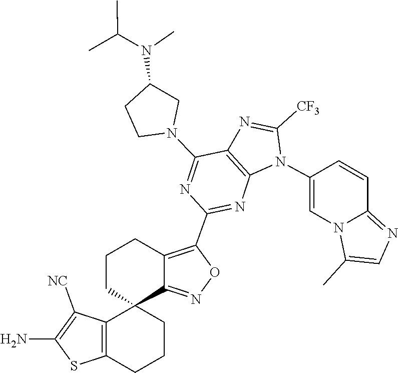 [SMILES: Cc1cnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] |
| II-29 | 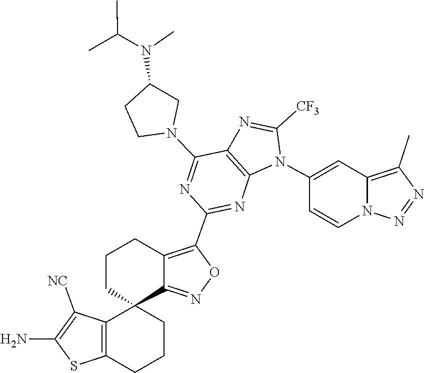 [SMILES: Cc1nnn2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cc12] |
| II-30 | 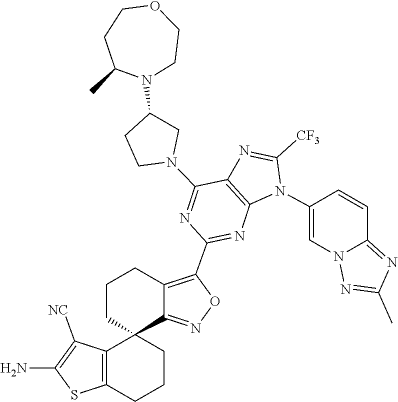 [SMILES: Cc1nc2ccc(-n3c(C(F)(F)F)nc4c(N5CC[C@H](N6CCOCC[C@@H]6C)C5)nc(-c5onc6c5CCC[C@@]65CCCc6sc(N)c(C#N)c65)nc43)cn2n1] |
| II-31 | 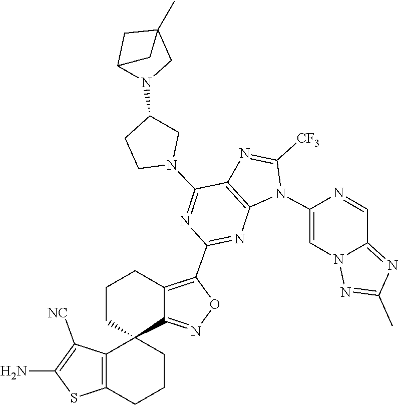 [SMILES: Cc1nc2cnc(-n3c(C(F)(F)F)nc4c(N5CC[C@H](N6CC7(C)CC6C7)C5)nc(-c5onc6c5CCC[C@@]65CCCc6sc(N)c(C#N)c65)nc43)cn2n1] |
| II-32 | 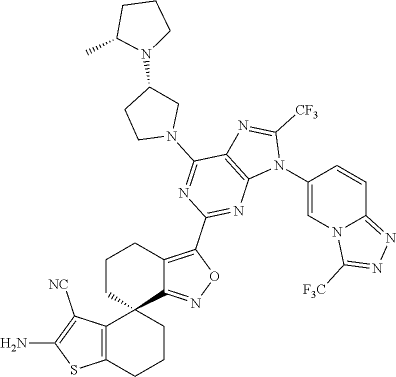 [SMILES: C[C@@H]1CCCN1[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1] |
| II-33 | 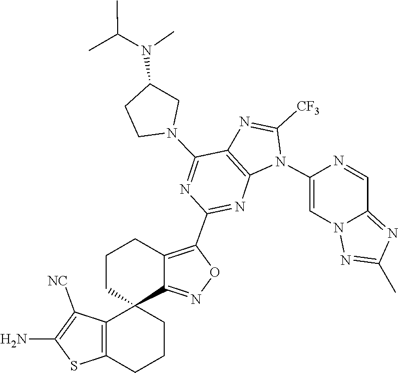 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1] |
| II-34 | 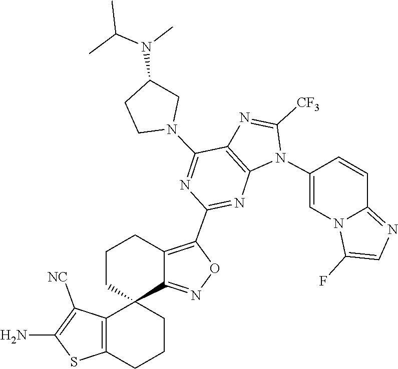 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3ncc(F)n3c2)C1] |
| II-35 | 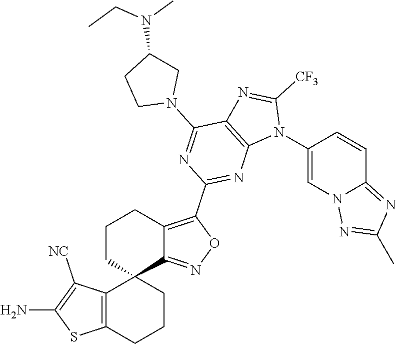 [SMILES: CCN(C)[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1] |
| II-36 | 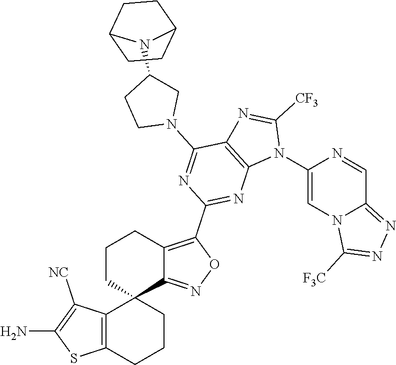 [SMILES: N#Cc1c(N)sc2c1[C@@]1(CCC2)CCCc2c1noc2-c1nc(N2CC[C@H](N3C4CCC3CC4)C2)c2nc(C(F)(F)F)n(-c3cn4c(C(F)(F)F)nnc4cn3)c2n1] |
| II-37 | 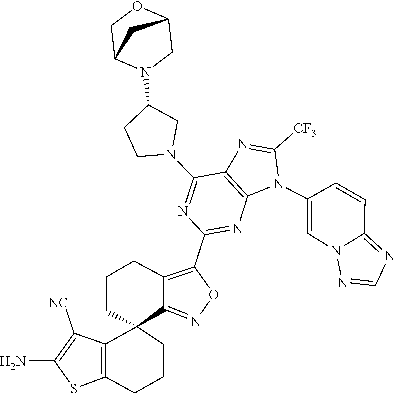 [SMILES: N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3ccc4ncnn4c3)c2n1] |
| II-38 | 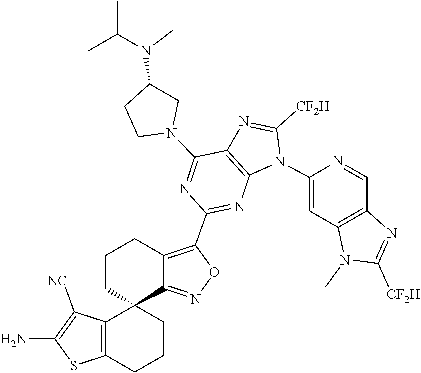 [SMILES: CC(C)N(C)[C@H]1CCN(c2nc(-c3onc4c3CCC[C@@]43CCCc4sc(N)c(C#N)c43)nc3c2nc(C(F)F)n3-c2cc3c(cn2)nc(C(F)F)n3C)C1] |
| II-39 | 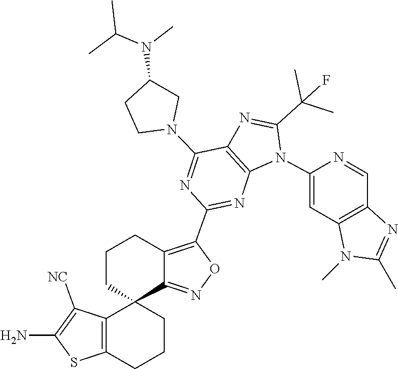 [SMILES: Cc1nc2cnc(-n3c(C(C)(C)F)nc4c(N5CC[C@H](N(C)C(C)C)C5)nc(-c5onc6c5CCC[C@@]65CCCc6sc(N)c(C#N)c65)nc43)cc2n1C] |
| II-40 | 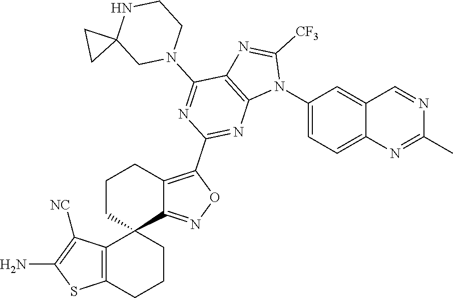 [SMILES: Cc1ncc2cc(-n3c(C(F)(F)F)nc4c(N5CCNC6(CC6)C5)nc(-c5onc6c5CCC[C@@]65CCCc6sc(N)c(C#N)c65)nc43)ccc2n1] |
| II-41 | 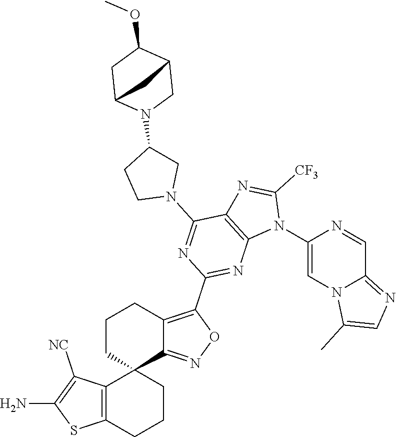 [SMILES: CO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1] |
| II-42 | 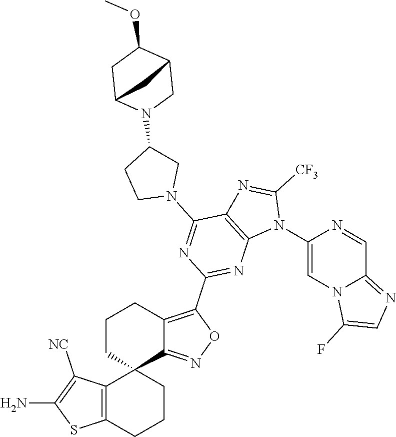 [SMILES: CO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1] |
| II-43 | 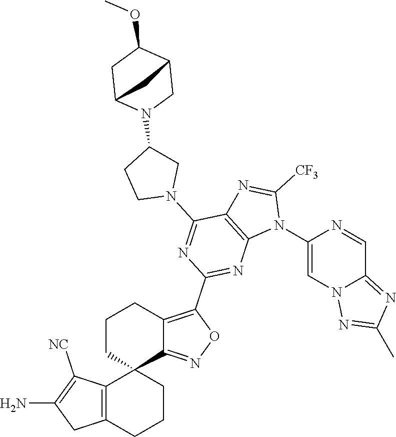 [SMILES: CO[C@@H]1C[C@@H]2C[C@H]1CN2[C@H]1CCN(c2nc(-c3onc4c3CCC[C@@]43CCCC4=C3C(C#N)=C(N)C4)nc3c2nc(C(F)(F)F)n3-c2cn3nc(C)nc3cn2)C1] |
| II-44 | 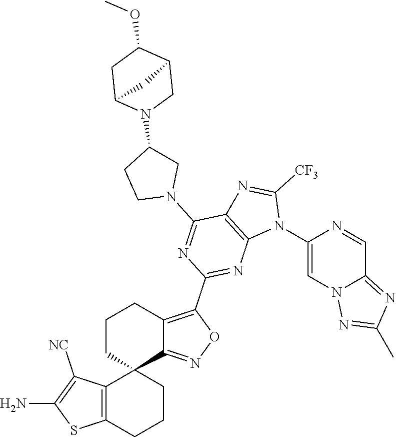 [SMILES: CO[C@H]1C[C@H]2C[C@@H]1CN2[C@H]1CCN(c2nc(-c3onc4c3CCC[C@@]43CCCc4sc(N)c(C#N)c43)nc3c2nc(C(F)(F)F)n3-c2cn3nc(C)nc3cn2)C1] |
| II-47 | 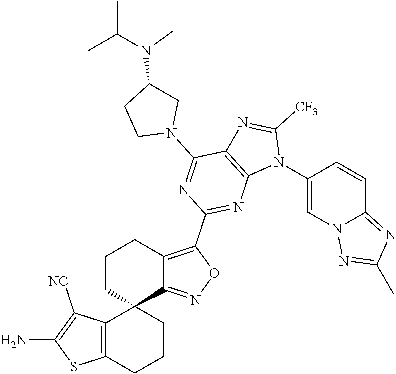 [SMILES: Cc1nc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1] |
| II-48 | 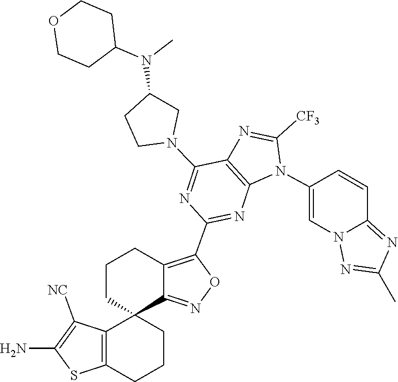 [SMILES: Cc1nc2ccc(-n3c(C(F)(F)F)nc4c(N5CC[C@H](N(C)C6CCOCC6)C5)nc(-c5onc6c5CCC[C@@]65CCCc6sc(N)c(C#N)c65)nc43)cn2n1] |
| II-49 | 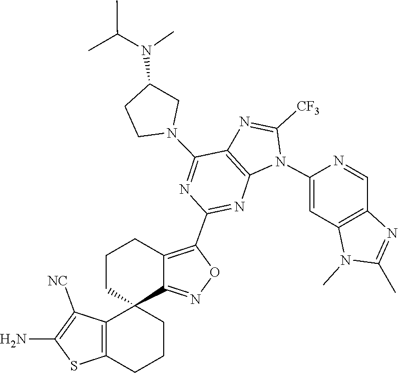 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C] |
| II-50 | 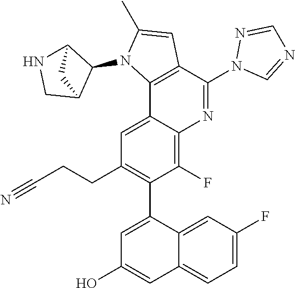 [SMILES: Cc1cc2c(-n3cncn3)nc3c(F)c(-c4cc(O)cc5ccc(F)cc45)c(CCC#N)cc3c2n1[C@H]1[C@H]2CN[C@@H]1C2] |
| II-51 | 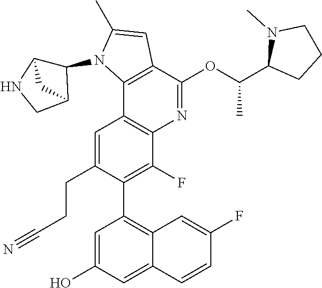 [SMILES: Cc1cc2c(O[C@@H](C)[C@@H]3CCCN3C)nc3c(F)c(-c4cc(O)cc5ccc(F)cc45)c(CCC#N)cc3c2n1[C@H]1[C@H]2CN[C@@H]1C2] |
| II-52 | 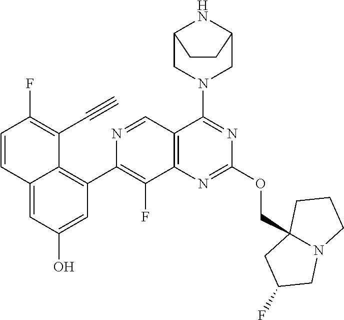 [SMILES: C#Cc1c(F)ccc2cc(O)cc(-c3ncc4c(N5CC6CCC(C5)N6)nc(OC[C@@]56CCCN5C[C@H](F)C6)nc4c3F)c12] |
| II-53 | 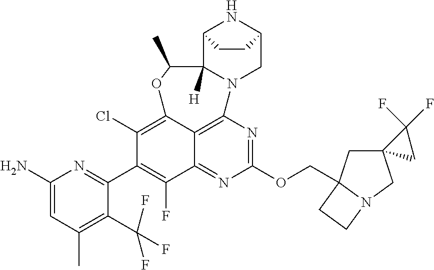 |
| II-54 | 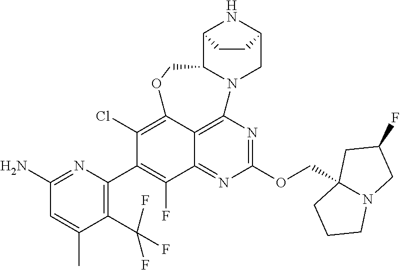 [SMILES: Cc1cc(N)nc(-c2c(Cl)c3c4c(nc(OC[C@@]56CCCN5C[C@H](F)C6)nc4c2F)N2C[C@H]4CC[C@H](N4)[C@H]2CO3)c1C(F)(F)F] |

It is to be understood that any two or more aspects and/or preferred embodiments of formula (I)—or subformulas thereof—may be combined in any way leading to a chemically stable structure to obtain further aspects of formula (I)—or subformulas thereof.

Preferred embodiments of the KRAS switch II inhibitor are example compounds II-1 to II-54.

Most preferred embodiments of the KRAS G12D switch II inhibitor are example compounds II-1 to II-49.

### Single Therapy and Combination Therapy

It is a purpose of the present invention to provide secondary treatment option for cancers with acquired resistance to tri-complex inhibitors.

It was discovered in vitro in cell line models that clones with secondary Y71H mutations were resistant to both tri-complex inhibitors Daraxonrasib (RMC-6236) and/or Zoldonrasib (RMC-9805).

Surprisingly, a KRAS switch II inhibitor, such as described above or below, showed potent activity against cells exhibiting a primary G12D mutation plus a secondary Y71H mutation.

Specifically, treatment with KRAS switch II inhibitors II-1 to II-54 reduced cell proliferation.

Surprisingly, a KRAS G12D switch II inhibitor, such as described above or below, showed potent activity against cells exhibiting a primary G12D mutation plus a secondary Y71H mutation.

Specifically, treatment with KRAS G12D switch II inhibitors II-1 to II-54 reduced cell proliferation.

More specifically, treatment with KRAS G12D switch II inhibitors II-1 to II-49 reduced cell proliferation.

Although cancer treatment with tri-complex inhibitors, such as Daraxonrasib (RMC-6236) and/or Zoldonrasib (RMC-9805), is known in the art, therapeutic concepts for a secondary treatment option for cancers with acquired resistance to tri-complex inhibitors are still lacking.

Thus, the invention relates to KRAS G12D switch II inhibitors, as described herein, for use in anti-cancer therapy when the cancer is resistant to treatment with a tri-complex inhibitor. For single treatment, the KRAS G12D switch II inhibitor can be administered formulated in a pharmaceutical composition or dosage form.

A combined treatment may include that the KRAS G12D switch II inhibitor can be administered together with another pharmacologically active substance formulated either dependently, such as formulated together into one composition, or independently, such as formulated as separate compositions.

### Pharmaceutical Composition

To be used for treatment, the KRAS G12D switch II inhibitor may be included into pharmaceutical compositions appropriate to facilitate administration.

To be used for treatment, the KRAS G12D switch II inhibitor of formula (I), (11), (II*), (IIa), (IIb), (IIc), (IId), (IIe) or (IIf), or a salt thereof, or a pharmaceutically acceptable salt thereof may be included into pharmaceutical compositions appropriate to facilitate administration.

The compound thus may be formulated, in suitable dosage unit formulations containing conventional non-toxic pharmaceutically acceptable carriers, excipients and/or vehicles appropriate for each route of administration. Typical pharmaceutical compositions for administering the KRAS G12D switch II inhibitor, include for example tablets, capsules, suppositories, solutions, e.g. solutions for injection and infusion, elixirs, emulsions or dispersible powders. Dosage forms and formulations of active ingredients are known in the art.

The KRAS G12D switch II inhibitor may be administered by oral routes of administration and may be formulated, in suitable dosage unit formulations containing conventional non-toxic pharmaceutically acceptable carriers, excipients and vehicles appropriate for each route of administration. Although oral administration may be preferred in view of compliance, routes of administration for the KRAS G12D switch II inhibitor described herein, are not limited to oral administration, but the compound may be administered parenterally, e.g. intramuscular, intraperitoneal, intravenous, transdermal or subcutaneous injection or by implant, or enterical, nasal, vaginal, rectal, or topical administration.

The KRAS G12D switch II inhibitor of formula (I), (II), (II*), (IIa), (IIb), (IIc), (IId), (lie) or (IIf), or a salt thereof, or a pharmaceutically acceptable salt thereof may be administered by oral routes of administration and may be formulated, in suitable dosage unit formulations containing conventional non-toxic pharmaceutically acceptable carriers, excipients and vehicles appropriate for each route of administration.

Thus, in a further aspect the invention also relates to a pharmaceutical composition comprising a KRAS G12D switch II inhibitor, or a pharmaceutically acceptable salt thereof—and one or more (preferably one or two, most preferably one) other pharmacologically active substance(s).

Thus, in a further aspect the invention also relates to a pharmaceutical composition comprising a KRAS G12D switch II inhibitor of formula (I), (II), (II*), (IIa), (IIb), (IIc), (IId), (IIe) or (IIf), or a pharmaceutically acceptable salt thereof—and one or more (preferably one or two, most preferably one) other pharmacologically active substance(s).

### Cancer

The compositions, uses, methods and compounds for use according to the present invention are usable for the treatment of cancer that is resistant to treatment with a tri-complex inhibitor.

According to an aspect is provided a KRAS G12D switch II inhibitor for use in the treatment and/or prevention of cancer, where the cancer is resistant to treatment with a tri-complex inhibitor.

According to an aspect is provided a KRAS G12D switch II inhibitor of formula (I), (II), (II*), (IIa), (IIb), (IIc), (IId), (IIe) or (IIf), or a pharmaceutically acceptable salt thereof for use in the treatment and/or prevention of cancer, where the cancer is resistant to treatment with a tri-complex inhibitor.

Another aspect refers to the aforementioned pharmaceutical compositions for use in a method of treating and/or preventing of cancer, wherein the cancer is resistant to treatment with a tri-complex inhibitor as described herein.

Another aspect relates to a method of treating and/or preventing cancer, the method comprising administering to a patient a therapeutically effective amount of a KRAS G12D switch II inhibitor, wherein the cancer is resistant to treatment with a tri-complex inhibitor.

Another aspect relates to a method of treating and/or preventing cancer, the method comprising administering to a patient a therapeutically effective amount of a KRAS G12D switch II inhibitor of formula (I), (II), (II*), (IIa), (IIb), (IIc), (IId), (IIe) or (IIf), or a pharmaceutically acceptable salt thereof, wherein the cancer is resistant to treatment with a tri-complex inhibitor.

Another aspect relates to a use of a KRAS G12D switch II inhibitor for the manufacture of a medicament for use in the treatment and/or prevention of cancer, wherein the cancer is resistant to treatment with a tri-complex inhibitor.

Another aspect relates to a use of a KRAS G12D switch II inhibitor of formula (I), (II), (II*), (IIa), (IIb), (IIc), (IId), (IIe) or (IIf), or a pharmaceutically acceptable salt thereof, for the manufacture of a medicament for use in the treatment and/or prevention of cancer, wherein the cancer is resistant to treatment with a tri-complex inhibitor.

The therapeutic applicability of the KRAS G12D switch II inhibitor may include second line, third line or further lines of treatment of patients. The cancer may be recurrent, relapsed, resistant or refractory to one or more anti-cancer treatments using tri-complex inhibitors.

Thus, the patients may have received previous anti-cancer therapies with one or more tri-complex inhibitor, which have not completely cured the disease.

Therapy using a KRAS G12D switch II inhibitor may be effective at treating subjects whose cancer has relapsed, or whose cancer has become drug resistant to tri-complex inhibitors, or whose cancer has failed one, two or more lines of mono- or combination therapy with one or more tri-complex inhibitors. A cancer which initially responded to a tri-complex inhibitor can relapse and become resistant to the tri-complex inhibitor when the tri-complex inhibitor is no longer effective in treating the subject with the cancer, for example despite the administration of increased dosages of the tri-complex inhibitor.

Accordingly, in an embodiment of the compositions, uses, methods and compounds for use according to the invention, the cancer has become resistant to treatment with a tri-complex inhibitor after earlier treatment with a tri-complex inhibitor.

In another embodiment, the cancer is resistant to treatment with inhibitors Daraxonrasib (RMC-6236) and/or Zoldonrasib (RMC-9805). In preferred embodiments, the cancer has become resistant to treatment with inhibitors Daraxonrasib (RMC-6236) and/or Zoldonrasib (RMC-9805) after earlier treatment with inhibitors Daraxonrasib (RMC-6236) and/or Zoldonrasib (RMC-9805).

As used herein, the term “cancer” refers to a malignant disease condition wherein cell growth is increased over normal levels. Cancers may be classified in several ways: by the type of tissue in which the cancer originates (histological type) and by primary site, or the location in the body, where the cancer first developed, and/or by exhibiting a molecular feature.

In a preferred embodiment of the compositions, uses, methods and compounds for use according to the invention, the cancer is defined as exhibiting a molecular feature, where the cancer cells of the cancer exhibit a primary KRAS G12D mutation and further exhibit a secondary KRAS Y71X mutation, preferably Y71H.

In a preferred embodiment, the cancer cells of the cancer have been determined to exhibit a primary KRAS G12D mutation and to further exhibit a secondary KRAS Y71X mutation, preferably Y71H.

In embodiments, the cancer is one of the following, without being restricted thereto:
pancreatic cancer, lung cancer, colorectal cancer, cholangiocarcinoma, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, chronic lymphocytic leukaemia, hepatocellular cancer, breast cancer, ovarian cancer, prostate cancer, glioblastoma, renal cancer and sarcomas.

### DEFINITIONS

Terms not specifically defined herein should be given the meanings that would be given to them by one of skill in the art in light of the disclosure and the context. As used in the specification, however, unless specified to the contrary, the following terms have the meaning indicated and the following conventions are adhered to:

The use of the prefix C_{x-y}, wherein x and y each represent a positive integer (x<y), indicates that the chain or ring structure or combination of chain and ring structure as a whole, specified and mentioned in direct association, may consist of a maximum of y and a minimum of x carbon atoms.

The indication of the number of members in groups that contain one or more heteroatom(s) (e.g. heteroaryl, heteroarylalkyl, heterocyclyl, heterocycylalkyl) relates to the total number of atoms of all the ring members or the total of all the ring and carbon chain members.

The indication of the number of carbon atoms in groups that consist of a combination of carbon chain and carbon ring structure (e.g. cycloalkylalkyl, arylalkyl) relates to the total number of carbon atoms of all the carbon ring and carbon chain members. Obviously, a ring structure has at least three members.

In general, for groups comprising two or more subgroups (e.g. heteroarylalkyl, heterocycylalkyl, cycloalkylalkyl, arylalkyl) the last named subgroup is the radical attachment point, for example, the substituent aryl-C₁₋₆alkyl means an aryl group which is bound to a C₁₋₆alkyl group, the latter of which is bound to the core or to the group to which the substituent is attached.

In groups like HO, H₂N, (O)S, (O)₂S, NC (cyano), HOOC, F₃C or the like, the skilled artisan can see the radical attachment point(s) to the molecule from the free valences of the group itself.

The expression “compound of the invention” and grammatical variants thereof comprises compounds of formula (I), including all salts, aspects and preferred embodiments thereof as herein defined. Any reference to a compound of the invention or to a compound of formula (I) is intended to include a reference to the respective (sub)aspects and embodiments.

Alkyl denotes monovalent, saturated hydrocarbon chains, which may be present in both straight-chain (unbranched) and branched form. If an alkyl is substituted, the substitution may take place independently of one another, by mono- or polysubstitution in each case, on all the hydrogen-carrying carbon atoms.

The term “C₁₋₅alkyl” includes for example H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

Further examples of alkyl are methyl (Me; —CH₃), ethyl (Et; —CH₂CH₃), 1-propyl (n-propyl; n-Pr; —CH₂CH₂CH₃), 2-propyl (i-Pr; iso-propyl; —CH(CH₃)₂), 1-butyl (n-butyl; n-Bu; —CH₂CH₂CH₂CH₃), 2-methyl-1-propyl (iso-butyl; i-Bu; —CH₂CH(CH₃)₂), 2-butyl (sec-butyl; sec-Bu; —CH(CH₃)CH₂CH₃), 2-methyl-2-propyl (tert-butyl; t-Bu; —C(CH₃)₃), 1-pentyl (n-pentyl; —CH₂CH₂CH₂CH₂CH₃), 2-pentyl (—CH(CH₃)CH₂CH₂CH₃), 3-pentyl (—CH(CH₂CH₃)₂), 3-methyl-1-butyl (iso-pentyl; —CH₂CH₂CH(CH₃)₂), 2-methyl-2-butyl (—C(CH₃)₂CH₂CH₃), 3-methyl-2-butyl (—CH(CH₃)CH(CH₃)₂), 2,2-dimethyl-1-propyl (neo-pentyl; —CH₂C(CH₃)₃), 2-methyl-1-butyl (—CH₂CH(CH₃)CH₂CH₃), 1-hexyl (n-hexyl; —CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), 2,3-dimethyl-1-butyl (—CH₂CH(CH₃)CH(CH₃)CH₃), 2,2-dimethyl-1-butyl (—CH₂C(CH₃)₂CH₂CH₃), 3,3-dimethyl-1-butyl (—CH₂CH₂C(CH₃)₃), 2-methyl-1-pentyl (—CH₂CH(CH₃)CH₂CH₂CH₃), 3-methyl-1-pentyl (—CH₂CH₂CH(CH₃)CH₂CH₃), 1-heptyl (n-heptyl), 2-methyl-1-hexyl, 3-methyl-1-hexyl, 2,2-dimethyl-1-pentyl, 2,3-dimethyl-1-pentyl, 2,4-dimethyl-1-pentyl, 3,3-dimethyl-1-pentyl, 2,2,3-trimethyl-1-butyl, 3-ethyl-1-pentyl, 1-octyl (n-octyl), 1-nonyl (n-nonyl); 1-decyl (n-decyl) etc.

By the terms propyl, butyl, pentyl, hexyl, heptyl, octyl, nonyl, decyl etc. without any further definition are meant saturated hydrocarbon groups with the corresponding number of carbon atoms, wherein all isomeric forms are included.

The above definition for alkyl also applies if alkyl is a part of another (combined) group such as for example C_{x-y}alkylamino or C_{x-y}alkyloxy.

The term alkylene can also be derived from alkyl. Alkylene is bivalent, unlike alkyl, and requires two binding partners. Formally, the second valency is produced by removing a hydrogen atom in an alkyl. Corresponding groups are for example —CH₃ and —CH₂—, —CH₂CH₃ and —CH₂CH₂— or >CHCH₃ etc.

The term “C₁₋₄alkylene” includes for example —(CH₂)—, —(CH₂—CH₂)—, —(CH(CH₃))—, —(CH₂—CH₂—CH₂)—, —(C(CH₃)₂)—, —(CH(CH₂CH₃))—, —(CH(CH₃)—CH₂)—, —(CH₂—CH(CH₃))—, —(CH₂—CH₂—CH₂—CH₂)—, —(CH₂—CH₂—CH(CH₃))—, —(CH(CH₃)—CH₂—CH₂)—, —(CH₂—CH(CH₃)—CH₂)—, —(CH₂—C(CH₃)₂)—, —(C(CH₃)₂—CH₂)—, —(CH(CH₃)—CH(CH₃))—, —(CH₂—CH(CH₂CH₃))—, —(CH(CH₂CH₃)—CH₂)—, —(CH(CH₂CH₂CH₃))—, —(CH(CH(CH₃))₂)— and —C(CH₃)(CH₂CH₃)—.

Other examples of alkylene are methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene, hexylene etc.

By the generic terms propylene, butylene, pentylene, hexylene etc. without any further definition are meant all the conceivable isomeric forms with the corresponding number of carbon atoms, i.e. propylene includes 1-methylethylene and butylene includes 1-methylpropylene, 2-methylpropylene, 1,1-dimethylethylene and 1,2-dimethylethylene. The above definition for alkylene also applies if alkylene is part of another (combined) group such as for example in HO-C_{x-y}alkyleneamino or H₂N—C_{x-y}alkyleneoxy.

Unlike alkyl, alkenyl consists of at least two carbon atoms, wherein at least two adjacent carbon atoms are joined together by a C—C double bond and a carbon atom can only be part of one C—C double bond. If in an alkyl as hereinbefore defined having at least two carbon atoms, two hydrogen atoms on adjacent carbon atoms are formally removed and the free valencies are saturated to form a second bond, the corresponding alkenyl is formed.

Examples of alkenyl are vinyl (ethenyl), prop-1-enyl, allyl (prop-2-enyl), isopropenyl, but-1-enyl, but-2-enyl, but-3-enyl, 2-methyl-prop-2-enyl, 2-methyl-prop-1-enyl, 1-methyl-prop-2-enyl, 1-methyl-prop-1-enyl, 1-methylidenepropyl, pent-1-enyl, pent-2-enyl, pent-3-enyl, pent-4-enyl, 3-methyl-but-3-enyl, 3-methyl-but-2-enyl, 3-methyl-but-1-enyl, hex-1-enyl, hex-2-enyl, hex-3-enyl, hex-4-enyl, hex-5-enyl, 2,3-dimethyl-but-3-enyl, 2,3-dimethyl-but-2-enyl, 2-methylidene-3-methylbutyl, 2,3-dimethyl-but-1-enyl, hexa-1,3-dienyl, hexa-1,4-dienyl, penta-1,4-dienyl, penta-1,3-dienyl, buta-1,3-dienyl, 2,3-dimethylbuta-1,3-diene etc.

By the generic terms propenyl, butenyl, pentenyl, hexenyl, butadienyl, pentadienyl, hexadienyl, heptadienyl, octadienyl, nonadienyl, decadienyl etc. without any further definition are meant all the conceivable isomeric forms with the corresponding number of carbon atoms, i.e. propenyl includes prop-1-enyl and prop-2-enyl, butenyl includes but-1-enyl, but-2-enyl, but-3-enyl, 1-methyl-prop-1-enyl, 1-methyl-prop-2-enyl etc.

Alkenyl may optionally be present in the cis or trans or E or Z orientation with regard to the double bond(s).

The above definition for alkenyl also applies when alkenyl is part of another (combined) group such as for example in C_{x-y}alkenylamino or C_{x-y}alkenyloxy.

Unlike alkyl, alkvnyl consists of at least two carbon atoms, wherein at least two adjacent carbon atoms are joined together by a C—C triple bond. If in an alkyl as hereinbefore defined having at least two carbon atoms, two hydrogen atoms in each case at adjacent carbon atoms are formally removed and the free valencies are saturated to form two further bonds, the corresponding alkynyl is formed.

Examples of alkynyl are ethynyl, prop-1-ynyl, prop-2-ynyl, but-1-ynyl, but-2-ynyl, but-3-ynyl, 1-methyl-prop-2-ynyl, pent-1-ynyl, pent-2-ynyl, pent-3-ynyl, pent-4-ynyl, 3-methyl-but-1-ynyl, hex-1-ynyl, hex-2-ynyl, hex-3-ynyl, hex-4-ynyl, hex-5-ynyl etc.

By the generic terms propynyl, butynyl, pentynyl, hexynyl, heptynyl, octynyl, nonynyl, decynyl etc. without any further definition are meant all the conceivable isomeric forms with the corresponding number of carbon atoms, i.e. propynyl includes prop-1-ynyl and prop-2-ynyl, butynyl includes but-1-ynyl, but-2-ynyl, but-3-ynyl, 1-methyl-prop-1-ynyl,1-methyl-prop-2-ynyl, etc.

If a hydrocarbon chain carries both at least one double bond and also at least one triple bond, by definition it belongs to the alkynyl subgroup.

The above definition for alkynyl also applies if alkynyl is part of another (combined) group, as for example in C_{x-y}alkynylamino or C_{x-y}alkynyloxy.

By heteroatoms are meant oxygen, nitrogen and sulphur atoms.

Haloalkyl (haloalkenyl, haloalkynyl) is derived from the previously defined alkyl (alkenyl, alkynyl) by replacing one or more hydrogen atoms of the hydrocarbon chain independently of one another by halogen atoms, which may be identical or different. If a haloalkyl (haloalkenyl, haloalkynyl) is to be further substituted, the substitutions may take place independently of one another, in the form of mono- or polysubstitutions in each case, on all the hydrogen-carrying carbon atoms.

Examples of haloalkyl (haloalkenyl, haloalkynyl) are —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCl═CH₂, —CBr═CH₂, —C≡C—CF₃, —CHFCH₂CH₃, —CHFCH₂CF₃ etc.

From the previously defined haloalkyl (haloalkenyl, haloalkynyl) are also derived the terms haloalkylene (haloalkenylene, haloalkynylene). Haloalkylene (haloalkenylene, haloalkynylene), unlike haloalkyl (haloalkenyl, haloalkynyl), is bivalent and requires two binding partners. Formally, the second valency is formed by removing a hydrogen atom from a haloalkyl (haloalkenyl, haloalkynyl).

Corresponding groups are for example —CH₂F and —CHF—, —CHFCH₂F and —CHFCHF— or >CFCH₂F etc.

The above definitions also apply if the corresponding halogen-containing groups are part of another (combined) group.

Halogen denotes fluorine, chlorine, bromine and/or iodine atoms.

Cycloalkyl is made up of the subgroups monocyclic cycloalkyl, bicyclic cycloalkyl and spiro-cycloalkyl. The ring systems are saturated and formed by linked carbon atoms. In bicyclic cycloalkyl two rings are joined together so that they have at least two carbon atoms in common Forming either fused or bridged ring systems. In spiro-cycloalkyl one carbon atom (spiroatom) belongs to two rings together.

If a cycloalkyl is to be substituted, the substitutions may take place independently of one another, in the form of mono- or polysubstitutions in each case, on all the hydrogen-carrying carbon atoms. Cycloalkyl itself may be linked as a substituent to the molecule via every suitable position of the ring system.

Examples of cycloalkyl are cyclopropyl, cyclobutyl, cyclopentyl, cyclohexyl, cycloheptyl, bicyclo[2.2.0]hexyl, bicyclo[3.2.0]heptyl, bicyclo[3.2.1]octyl, bicyclo[2.2.2]octyl, bicyclo[4.3.0]nonyl (octahydroindenyl), bicyclo[4.4.0]decyl (decahydronaphthyl), bicyclo[2.2.1]heptyl (norbornyl), bicyclo[4.1.0]heptyl (norcaranyl), bicyclo[3.1.1]heptyl (pinanyl), spiro[2.5]octyl, spiro[3.3]heptyl etc.

The above definition for cycloalkyl also applies if cycloalkyl is part of another (combined) group as for example in C_{x-y}cycloalkylamino, C_{x-y}cycloalkyloxy or C_{x-y}cycloalkylalkyl. If the free valency of a cycloalkyl is saturated, then an alicycle is obtained.

Aryl denotes mono-, bi- or tricyclic carbocycles with at least one aromatic carbocycle. Preferably, it denotes a monocyclic group with six carbon atoms (phenyl) or a bicyclic group with nine or ten carbon atoms (two six-membered rings or one six-membered ring with a five-membered ring), wherein the second ring may also be aromatic or, however, may also be partially saturated.

If an aryl is to be substituted, the substitutions may take place independently of one another, in the form of mono- or polysubstitutions in each case, on all the hydrogen-carrying carbon atoms. Aryl itself may be linked as a substituent to the molecule via every suitable position of the ring system.

Examples of aryl are phenyl, naphthyl, indanyl (2,3-dihydroindenyl), indenyl, anthracenyl, phenanthrenyl, tetrahydronaphthyl (1,2,3,4-tetrahydronaphthyl, tetralinyl), dihydronaphthyl (1,2-dihydronaphthyl), fluorenyl etc. Most preferred is phenyl.

The above definition of aryl also applies if aryl is part of another (combined) group as for example in arylamino, aryloxy or arylalkyl.

If the free valency of an aryl is saturated, then an arene is obtained.

Heterocyclyl denotes ring systems, which are derived from the previously defined cycloalkyl, cycloalkenyl and aryl by replacing one or more of the groups —CH₂— independently of one another in the hydrocarbon rings by the groups —O—, —S— or —NH— or by replacing one or more of the groups ═CH— by the group ═N—, wherein a total of not more than five heteroatoms may be present, at least one carbon atom must be present between two oxygen atoms and between two sulphur atoms or between an oxygen and a sulphur atom and the ring as a whole must have chemical stability. Heteroatoms may optionally be present in all the possible oxidation stages (sulphur→sulfoxide —SO—, sulphone —SO₂—; nitrogen→N-oxide). In a heterocyclyl there is no heteroaromatic ring, i.e. no heteroatom is part of an aromatic system.

A direct result of the derivation from cycloalkyl, cycloalkenyl and aryl is that heterocyclyl is made up of the subgroups monocyclic heterocyclyl, bicyclic heterocyclyl, tricyclic heterocyclyl and spiro-heterocyclyl, which may be present in saturated or unsaturated form.

By unsaturated is meant that there is at least one double bond in the ring system in question, but no heteroaromatic system is formed. In bicyclic heterocyclyl two rings are linked together so that they have at least two (hetero)atoms in common forming either fused or bridged ring systems. In spiro-heterocyclyl one carbon atom (spiroatom) belongs to two rings together.

If a heterocyclyl is substituted, the substitutions may take place independently of one another, in the form of mono- or polysubstitutions in each case, on all the hydrogen-carrying carbon and/or nitrogen atoms. Heterocyclyl itself may be linked as a substituent to the molecule via every suitable position of the ring system. Substituents on heterocyclyl do not count for the number of members of a heterocyclyl.

Examples of heterocyclyl are tetrahydrofuryl, pyrrolidinyl, pyrrolinyl, imidazolidinyl, thiazolidinyl, imidazolinyl, pyrazolidinyl, pyrazolinyl, piperidinyl, piperazinyl, oxiranyl, aziridinyl, azetidinyl, 1,4-dioxanyl, azepanyl, diazepanyl, morpholinyl, thiomorpholinyl, homomorpholinyl, homopiperidinyl, homopiperazinyl, homothiomorpholinyl, thiomorpholinyl-S-oxide, thiomorpholinyl-S,S-dioxide, 1,3-dioxolanyl, tetrahydropyranyl, tetrahydrothiopyranyl, [1,4]-oxazepanyl, tetrahydrothienyl, homothiomorpholinyl-S,S-dioxide, oxazolidinonyl, dihydropyrazolyl, dihydropyrrolyl, dihydropyrazinyl, dihydropyridyl, dihydro-pyrimidinyl, dihydrofuryl, dihydropyranyl, tetrahydrothienyl-S-oxide, tetrahydrothienyl-S,S-dioxide, homothiomorpholinyl-S-oxide, 2,3-dihydroazet, 2H-pyrrolyl, 4H-pyranyl, 1,4-dihydropyridinyl, 8-aza-bicyclo[3.2.1]octyl, 8-aza-bicyclo[5.1.0]octyl, 2-oxa-5-azabicyclo[2.2.1]heptyl, 8-oxa-3-aza-bicyclo[3.2.1]octyl, 3,8-diaza-bicyclo[3.2.1]octyl, 2,5-diaza-bicyclo[2.2.1]heptyl, 1-aza-bicyclo[2.2.2]octyl, 3,8-diaza-bicyclo[3.2.1]octyl, 3,9-diaza-bicyclo[4.2.1]nonyl, 2,6-diaza-bicyclo[3.2.2]nonyl, 1,4-dioxa-spiro[4.5]decyl, 1-oxa-3,8-diaza-spiro[4.5]decyl, 2,6-diaza-spiro[3.3]heptyl, 2,7-diaza-spiro[4.4]nonyl, 2,6-diaza-spiro[3.4]octyl, 3,9-diaza-spiro[5.5]undecyl, 2.8-diaza-spiro[4,5]decyl etc.

Further examples are the structures illustrated below, which may be attached via each hydrogen-carrying atom (exchanged for hydrogen):

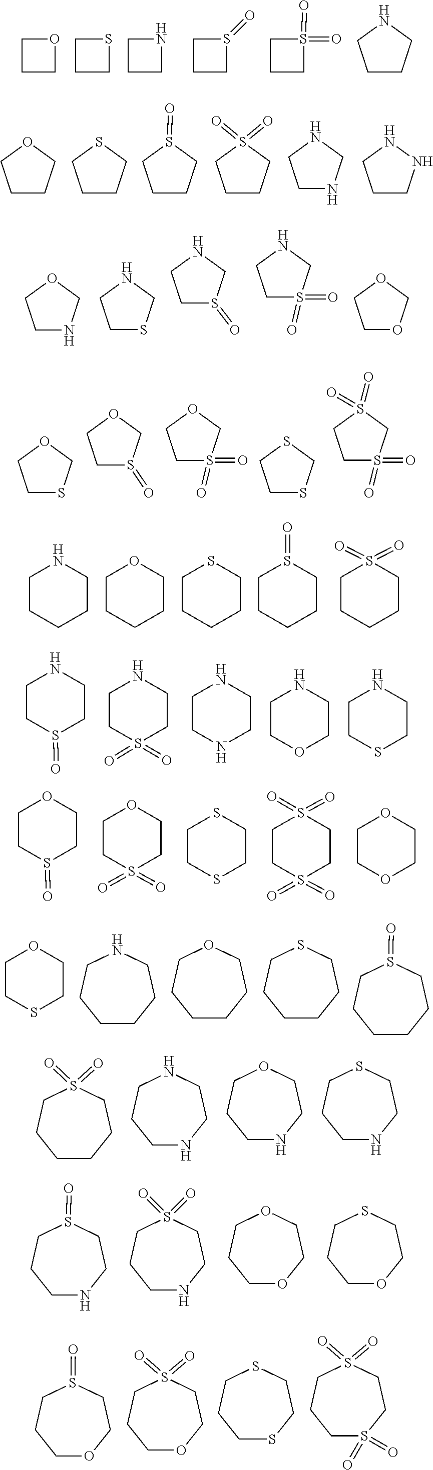

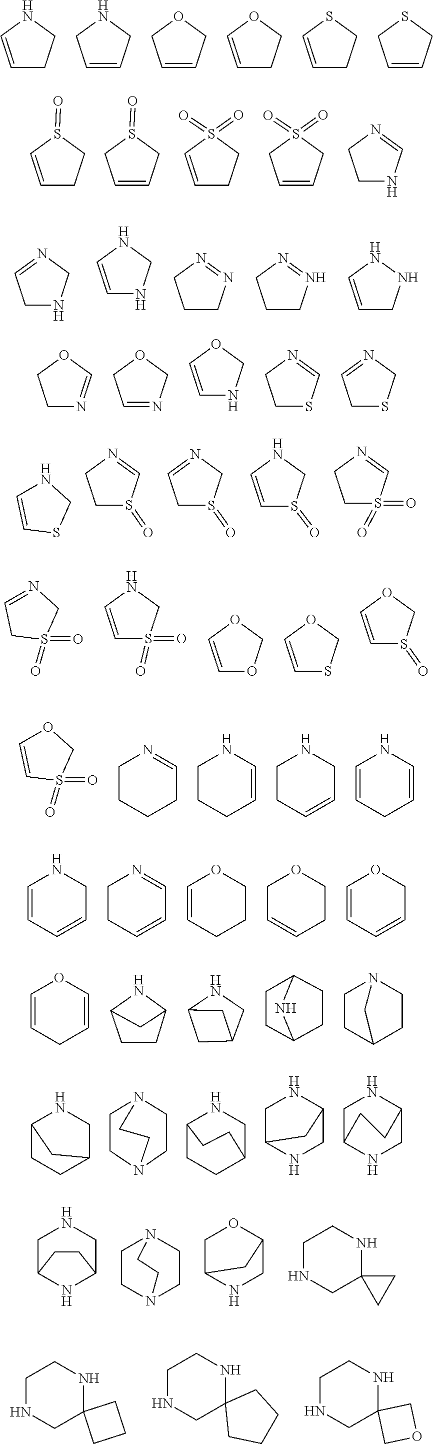

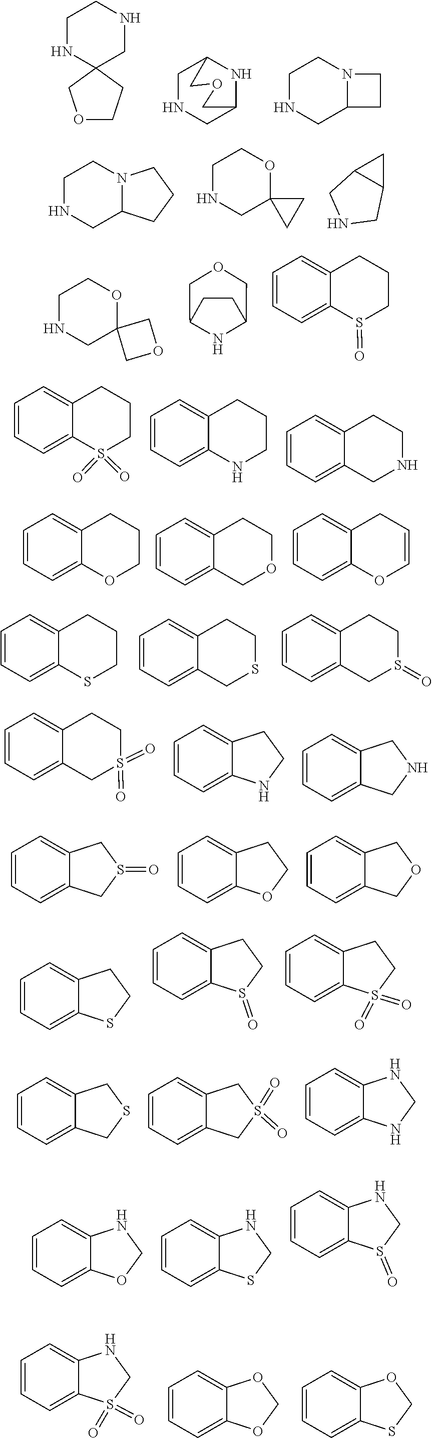

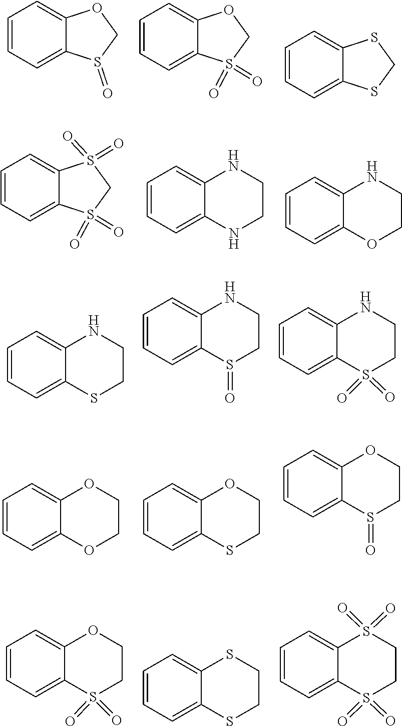

Preferred monocyclic heterocyclyl is 4 to 7 membered and has one or two heteroatoms independently selected from oxygen, nitrogen and sulfur.

Preferred monocyclic heterocyclyls are: piperazinyl, piperidinyl, morpholinyl, pyrrolidinyl, and azetidinyl.

Preferred bicyclic heterocyclyl is 6 to 10 membered and has one or two heteroatoms independently selected from oxygen, nitrogen and sulfur.

Preferred tricyclic heterocyclyl is 9 membered and has one or two heteroatoms independently selected from oxygen, nitrogen and sulfur.

Preferred spiro-heterocyclyl is 7 to 11 membered and has one or two heteroatoms independently selected from oxygen, nitrogen and sulfur.

The above definition of heterocyclyl also applies if heterocyclyl is part of another (combined) group as for example in heterocyclylamino, heterocyclyloxy or heterocyclylalkyl.

If the free valency of a heterocyclyl is saturated, then a heterocycle is obtained.

Heteroaryl denotes monocyclic heteroaromatic rings or polycyclic rings with at least one heteroaromatic ring, which compared with the corresponding aryl or cycloalkyl (cycloalkenyl) contain, instead of one or more carbon atoms, one or more identical or different heteroatoms, selected independently of one another from among nitrogen, sulphur and oxygen, wherein the resulting group must be chemically stable. The prerequisite for the presence of heteroaryl is a heteroatom and a heteroaromatic system.

If a heteroaryl is to be substituted, the substitutions may take place independently of one another, in the form of mono- or polysubstitutions in each case, on all the hydrogen-carrying carbon and/or nitrogen atoms. Heteroaryl itself may be linked as a substituent to the molecule via every suitable position of the ring system, both carbon and nitrogen. Substituents on heteroaryl do not count for the number of members of a heteroaryl.

Examples of heteroaryl are furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidyl, pyridazinyl, pyrazinyl, triazinyl, pyridyl-N-oxide, pyrrolyl-N-oxide, pyrimidinyl-N-oxide, pyridazinyl-N-oxide, pyrazinyl-N-oxide, imidazolyl-N-oxide, isoxazolyl-N-oxide, oxazolyl-N-oxide, thiazolyl-N-oxide, oxadiazolyl-N-oxide, thiadiazolyl-N-oxide, triazolyl-N-oxide, tetrazolyl-N-oxide, indolyl, isoindolyl, benzofuryl, benzothienyl, benzoxazolyl, benzothiazolyl, benzisoxazolyl, benzisothiazolyl, benzimidazolyl, indazolyl, isoquinolinyl, quinolinyl, quinoxalinyl, cinnolinyl, phthalazinyl, quinazolinyl, benzotriazinyl, indolizinyl, oxazolopyridyl, imidazopyridyl, naphthyridinyl, benzoxazolyl, pyridopyridyl, pyrimidopyridyl, purinyl, pteridinyl, benzothiazolyl, imidazopyridyl, imidazothiazolyl, quinolinyl-N-oxide, indolyl-N-oxide, isoquinolyl-N-oxide, quinazolinyl-N-oxide, quinoxalinyl-N-oxide, phthalazinyl-N-oxide, indolizinyl-N-oxide, indazolyl-N-oxide, benzothiazolyl-N-oxide, benzimidazolyl-N-oxide etc.

Further examples are the structures illustrated below, which may be attached via each hydrogen-carrying atom (exchanged for hydrogen):

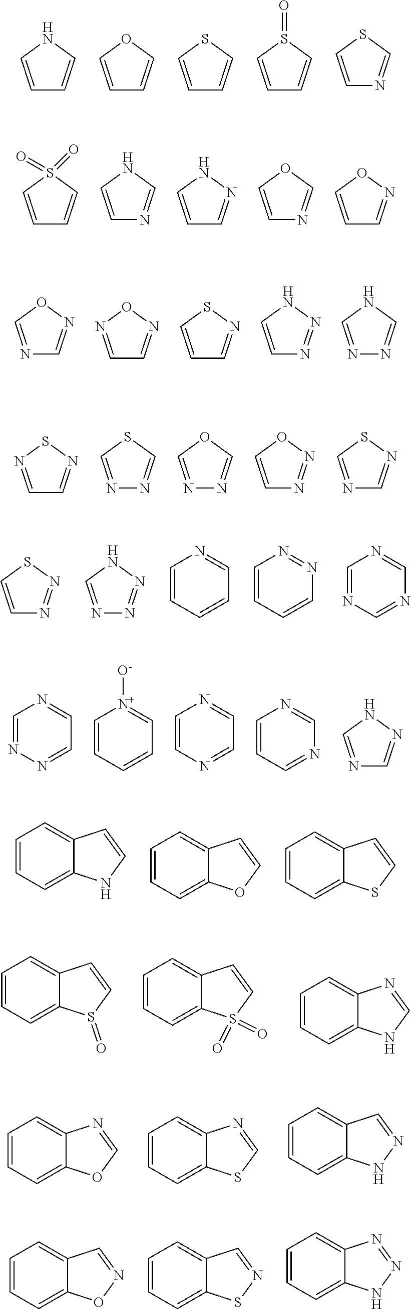

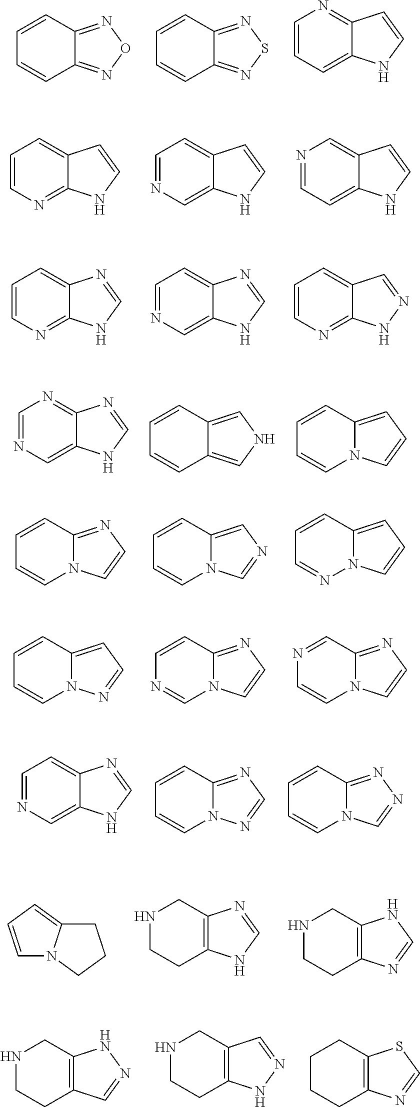

Preferably, heteroaryls are 5-6 membered monocyclic or 9-10 membered bicyclic, each with 1 to 4 heteroatoms independently selected from oxygen, nitrogen and sulfur.

The above definition of heteroaryl also applies if heteroaryl is part of another (combined) group as for example in heteroarylamino, heteroaryloxy or heteroarylalkyl.

If the free valency of a heteroaryl is saturated, a heteroarene is obtained.

By substituted is meant that a hydrogen atom which is bound directly to the atom under consideration, is replaced by another atom or another group of atoms (substituent). Depending on the starting conditions (number of hydrogen atoms) mono- or polysubstitution may take place on one atom. Substitution with a particular substituent is only possible if the permitted valencies of the substituent and of the atom that is to be substituted correspond to one another and the substitution leads to a stable compound (i.e. to a compound which is not converted spontaneously, e.g. by rearrangement, cyclisation or elimination).

Bivalent substituents such as ═S, ═NR, ═NOR, ═NNRR, ═NN(R)C(O)NRR, ═N₂ or the like, may only be substituents on carbon atoms, whereas the bivalent substituents ═O and ═NR may also be a substituent on sulphur. Generally, substitution may be carried out by a bivalent substituent only at ring systems and requires replacement of two geminal hydrogen atoms, i.e. hydrogen atoms that are bound to the same carbon atom that is saturated prior to the substitution. Substitution by a bivalent substituent is therefore only possible at the group —CH₂— or sulphur atoms (═O group or ═NR group only, one or two ═O groups possible or, e.g., one ═O group and one ═NR group, each group replacing a free electron pair) of a ring system.

Isotopes: It is to be understood that all disclosures of an atom or compound of the invention include all suitable isotopic variations. In particular, a reference to hydrogen also includes deuterium.

Stereochemistry/solvates/hydrates: Unless specifically indicated, throughout the specification and appended claims, a given chemical formula or name shall encompass tautomers and all stereo, optical and geometrical isomers (e.g. enantiomers, diastereomers, E/Z isomers, etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates and hydrates of the free compound or solvates and hydrates of a salt of the compound.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases, or by resolution of a racemic mixture using an appropriate resolving agent, e.g. by means of diastereomeric salt formation of the racemic compound with optically active acids or bases, subsequent resolution of the salts and release of the desired compound from the salt, or by derivatization of the corresponding racemic compounds with optically active chiral auxiliary reagents, subsequent diastereomer separation and removal of the chiral auxiliary group, or by kinetic resolution of a racemate (e.g. by enzymatic resolution); by enantioselective crystallization from a conglomerate of enantiomorphous crystals under suitable conditions, or by (fractional) crystallization from a suitable solvent in the presence of an optically active chiral auxiliary.

Salts: The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication and commensurate with a reasonable benefit/risk ratio.

As used herein “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base form of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts), also comprise a part of the invention.

In a representation such as for example

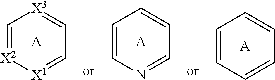

the letter A has the function of a ring designation in order to make it easier, for example, to indicate the attachment of the ring in question to other rings.

For bivalent groups in which it is crucial to determine which adjacent groups they bind and with which valency, the corresponding binding partners are indicated in brackets where necessary for clarification purposes, as in the following representations:

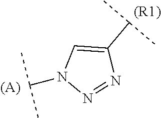

or (R²) —C(═O)NH— or (R²) —NHC(═O)—.

If such a clarification is missing then the bivalent group can bind in both directions, i.e., e.g., —C(═O)NH— also includes —NHC(═O)— (and vice versa).

Groups or substituents are frequently selected from among a number of alternative groups/substituents with a corresponding group designation (e.g. R^{a}, R^{b} etc). If such a group is used repeatedly to define a compound according to the invention in different parts of the molecule, it is pointed out that the various uses are to be regarded as totally independent of one another.

By a therapeutically effective amount for the purposes of this invention is meant a quantity of substance that is capable of obviating symptoms of illness or of preventing or alleviating these symptoms, or which prolong the survival of a treated patient.

### Experimental Procedures

List of abbreviations

| | |
|---|---|
| Ac | acetyl |
| ACN | acetonitrile |
| aq. | aquatic, aqueous |
| ATP | adenosine triphosphate |
| Bn | benzyl |
| Boc | tert-butyloxycarbonyl |
| Bu | butyl |
| c | concentration |
| CDI | 1,1′-carbonyldiimidazole |
| d | day(s) |
| TLC | thin layer chromatography |
| DBU | Diazabicycloundecen |
| DCM | dichloromethane |
| DEA | Diethylamine |
| DIPEA | N-ethyl-N,N-diisopropylamine (Hünig's base) |
| DMAP | 4-N,N-dimethylaminopyridine |
| DME | 1,2-dimethoxyethane |
| DMF | N,N-dimethylformamide |
| DMSO | Dimethyl sulfoxide |
| dppf | 1.1′-bis(diphenylphosphino)ferrocene |
| equiv. | equivalent(s) |
| ESI | electron spray ionization |
| Et | ethyl |
| Et₂O | diethyl ether |
| EtOAc | ethyl acetate |
| EtOH | ethanol |
| h | hour(s) |
| HATU | O-(7-azabenzotriazol-1-yl)-N,N,N′,N′-tetramethyl-uronium |
| | hexafluorophosphate |
| HPLC | high performance liquid chromatography |
| i | iso |
| conc. | concentrated |
| LC | liquid chromatography |
| LiHMDS | lithium bis(trimethylsilyl)amide |
| m-CPBA | meta-chloroperoxybenzoic acid |
| Me | methyl |
| MeOH | methanol |
| min | minute(s) |
| MS | mass spectrometry |
| NP | normal phase |
| n.a. | not available |
| PBS | phosphate-buffered saline |
| Ph | phenyl |
| Pr | propyl |
| Py | pyridine |
| rac | racemic |
| red. | reduction |
| Rf (R_{f}) | retention factor |
| RP | reversed phase |
| rt | ambient temperature |
| s | second(s) |
| sat. | saturated |
| SFC | supercritical fluid chromatography |
| S_{N} | nucleophilic substitution |
| tBu | tert-butyl |
| TEA | triethyl amine |
| temp. | temperature |
| tert | tertiary |
| Tf | triflate |
| TFA | trifluoroacetic acid |
| THF | tetrahydrofuran |
| TLC | thin layer chromatography |
| t_{Ret.} | retention time (HPLC) |
| Ts | tosylate |
| UPLC | ultra performance liquid chromatography |
| UV | ultraviolet |

Features and advantages of the present invention will become apparent from the following detailed examples which illustrate the principles of the invention by way of example without restricting its scope:

### Preparation of the Compounds According to the Invention

### General

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon).

If a compound is to be represented both by a structural formula and by its nomenclature, in the event of a conflict the structural formula is decisive.

Microwave reactions are carried out in an initiator/reactor made by Biotage or in an Explorer made by CEM or in Synthos 3000 or Monowave 3000 made by Anton Paar in sealed containers (preferably 2, 5 or 20 mL), preferably with stirring.

### Chromatography

The thin layer chromatography is carried out on ready-made silica gel 60 TLC plates on glass (with fluorescence indicator F-254) made by Merck.

The preparative high pressure chromatography (RP HPLC) of the example compounds according to the invention is carried out on Agilent or Gilson systems with columns made by Waters (names: SunFire™ Prep C18, OBD™ 10 μm, 50×150 mm or SunFire™ Prep C18 OBD™ 5 μm, 30×50 mm or XBridge™ Prep C18, OBD™ 10 μm, 50×150 mm or XBridge™ Prep C18, OBD™ 5 μm, 30×150 mm or XBridge™ Prep C18, OBD™ 5 μm, 30×50 mm) and YMC (names: Actus-Triart Prep C18, 5 μm, 30×50 mm).

Different gradients of H₂O/ACN are used to elute the compounds, while for Agilent systems 5% acidic modifier (20 mL HCOOH to 1 L H₂O/ACN (1/1)) is added to the water (acidic conditions). For Gilson systems to water is added 0.1% HCOOH.

For the chromatography under basic conditions for Agilent systems H₂O/ACN gradients are used as well, while the water is made alkaline by addition of 5% basic modifier (50 g NH₄HCO₃+50 mL NH₃ (25% in H₂O) to 1 L with H₂O). For Gilson systems the water is made alkaline as follows: 5 mL NH₄HCO₃ solution (158 g in 1 L H₂O) and 2 mL NH₃ (28% in H₂O) are replenished to 1 L with H₂O.

The supercritical fluid chromatography (SFC) of the intermediates and example compounds according to the invention is carried out on a JASCO SFC-system with the following columns: Chiralcel OJ (250×20 mm, 5 μm), Chiralpak AD (250×20 mm, 5 μm), Chiralpak AS (250×20 mm, 5 μm), Chiralpak IC (250×20 mm, 5 μm), Chiralpak IA (250×20 mm, 5 μm), Chiralcel OJ (250×20 mm, 5 μm), Chiralcel OD (250×20 mm, 5 μm), Phenomenex Lux C2 (250×20 mm, 5 μm).

The analytical HPLC (reaction control) of intermediate and final compounds is carried out using columns made by Waters (names: XBridge™ C18, 2.5 μm, 2.1×20 mm or XBridge™ C18, 2.5 μm, 2.1×30 mm or Aquity UPLC BEH C18, 1.7 μm, 2.1×50 mm) and YMC (names: Triart C18, 3.0 μm, 2.0×30 mm) and Phenomenex (names: Luna C18, 5.0 μm, 2.0×30 mm). The analytical equipment is also equipped with a mass detector in each case.

### HPLC-Mass Spectroscopy/UV-Spectrometry

The retention times/MS-ESI⁺ for characterizing the example compounds according to the invention are produced using an HPLC-MS apparatus (high performance liquid chromatography with mass detector). Compounds that elute at the injection peak are given the retention time t_{Ret.}=0.00.

Method A

| HPLC | Agilent 1200 system | |
|---|---|---|
| MS | 1200Series LC/MSD(API-ES +/− | |
| | 3000 V, Quadrupol, G6140) | |
| MSD signal settings | Scan pos/neg 120-900 m/z | |
| Detection signal | 315 nm (bandwidth 170 nm, reference off) | |
| Spectrum range | 230-400 nm | |
| Peak width | <0.01 min | |
| Column | Waters, Xbridge C18, 2.5 μm, | |
| | 2.1 × 20 mm column | |
| Column temperature | 60 °C | |
| Solvent | A: 20 mM aq. NH₄HCO₃/NH₃ pH 9 | |
| | B: ACN HPLC grade | |
| Flow | 1.00 mL/min | |
| Gradient | 0.00-1.50 min | 10% to 95% B |
| | 1.50-2.00 min | 95% B |
| | 2.00-2.10 min | 95% to 10% B |

Method B

| HPLC | Agilent 1100/1200/1260 system | |
|---|---|---|
| MS | 1200 Series LC/MSD (MM-ES + | |
| | APCI +/− 3000 V, | |
| | Quadrupol, G6130B) | |
| MSD signal | Scan pos/neg | |
| settings | 50-500 | |
| Column | Waters, Part. No. 186003389, | |
| | XBridge BEH C18, | |
| | 2.5 μm, 2.1 × 30 mm) column | |
| Solvent | A: 5 mM NH₄HCO₃/ | |
| | 18 mM NH₃ (pH = 9.2) | |
| | B: acetonitrile (HPLC grade) | |
| Detection signal | UV 254 nm, 230 nm, 214 nm | |
| | (bandwidth 8, reference off) | |
| Spectrum | range: 190-400 nm; slit: 4 nm | |
| Peak width | >0.0031 min (0.063 s response time, 80 Hz) | |
| Injection | 0.5 μL standard injection | |
| Flow | 1.4 mL/min | |
| Column temperature | 45 °C | |
| Gradient | 0.0-1.0 min | 15% → 95% B |
| | 1.0-1.1 min | 95% B |

Stop time: 1.3 min

Method C

| | |
|---|---|
| HPLC | Agilent 1260 system |
| MS | 1200 Series LC/MSD (MM-ES + APCI +/− |
| | 3000 V, Quadrupol, G6130) |
| Detection | UV: 254 nm (bandwidth 8, reference off) |
| | UV: 230 nm (bandwidth 8, reference off) |
| | UV spectrum range: 190-400 nm; step: 4 nm |
| | MS: positive and negative mode |
| Mass range | 100-800 m/z |
| Column | Waters; Part. No. 186003389; |
| | XBridge BEH C18, 2.5 μm, |
| | 30 × 2.1 mm |
| Column temperature | 45 °C |
| Solvent | A: 5 mM NH4HCO3/19 mM NH3 in |
| | H2O; B: ACN (HPLC grade) |
| Flow | 1.40 mL/min |
| Gradient | 0.00-1.00 min: 5% B to 100% B |
| | 1.00-1.37 min: 100% B |
| | 1.37-1.40 min: 100% B to 5% B |

Method D

| LC-MS | Waters Arc-HPLC-SQ Detector-2 | |
|---|---|---|
| MSD signal settings | ESI Scan pos & neg | |
| | Capillary Voltage 3.50 Kv cone | |
| | voltage 30 V Disolvation gas | |
| | 750 L/hr Disolvation Temp 350 °c | |
| Column | X-Bridge C18, 4.6 × 75 mm, 3.5 μm | |
| Column temperature | 40 °C | |
| Solvent | A: 10 mM Ammonium Acetate in water | |
| | B: ACN | |
| Flow | 1.2 mL/min | |
| Gradient | 0.0-0.3 min | 2% B |
| | 0.3-1.3 min | 2% to 98% B |
| | 1.3-4.5 min | 98% B |
| | 3.0-4.8 min | 100% B |

Method F

| HPLC | Agilent 1100/1200 system | |
|---|---|---|
| MS | 1200 Series LC/MSD (API-ES +/− | |
| | 3000/3500 V, Quadrupol, G6140A) | |
| MSD signal settings | Scan pos/neg 150-750 | |
| Detection signal | UV 254 nm, 230 nm, 214 nm | |
| | (bandwidth 10, reference off) | |
| Spectrum | range: 190-400 nm; slit: 4 nm | |
| Peak width | >0.0031 min (0.063 s response time, 80 Hz) | |
| Column | YMC; Part. No. TA12S03-0302WT; Triart | |
| | C18, 3 μm, 12 nm; 30 × 2.0 mm column | |
| Column temperature | 45 °C | |
| Solvent | A: H2O + 0.11% formic acid | |
| | B: ACN + 0.1% formic acid (HPLC grade) | |
| Flow | 1.4 mL/min | |
| Gradient | 0.0-1.0 min | 15% to 95% B |
| | 1.0-1.1 min | 95% B |

| | |
|---|---|
| Stop time: | 1.23 min |

SFC
Method SFC-1

| Make | Waters UPC²-MS | | |
|---|---|---|---|
| Soft | Empower3 | | |
| MS | QDa | | |
| Column | CHIRALCEL OX-3(4.6*150 MM) 3 μm | | |
| A-Solvent | CO₂ | | |
| B-solvent | ACN | | |
| Total Flow | 3 g/min | | |
| % of Co-Solvent | 15 | | |
| ABPR | 1500 psi | | |
| Colum temp | 30 °C | | |
| PDA range | 200 nm to 400 nm | | |
| Resolution | 1.2 nm | | |
| MS Parameters | — | | |
| QDa MS scan range | 100 Da to 1000 Da | | |
| | Cone voltage | | |
| Positive scan | 20 V | | |
| Negative Scan | 15 V | | |
| | 12 min | 1% B | 2 mL/min |

| | |
|---|---|
| Pressure limit | 20-550 bar |

Method SFC-2

| | |
|---|---|
| Make | Waters UPC²-MS |
| Column | CHIRALPAK AD H(4.6*250 MM) 5 μm |
| A-Solvent | CO₂ |
| B-solvent | 0.5% Methanolic Ammonia in Methanol |
| Total Flow | 3 mL/min |
| % of Co-Solvent | 10 |
| ABPR | 1500 psi |
| Colum temp | 30 °C |
| PDA range | 200 nm to 400 nm |
| MS Parameters | — |

Method X

| UPLC-MS | Waters Acquity-Binary Solvent | |
|---|---|---|
| | Manager-UPLC-SQ Detector-2 | |
| MSD signal settings | Scan pos & neg 100-1500, | |
| | Source Voltage: Capillary | |
| | Vol(kV)- 3.50, Cone(V): 50 | |
| | Source Temp: Desolvation Temp(°C): 350 | |
| | Source Gas Flow: Desolvation(L/Hr): | |
| | 750, Cone (L/hr): 50 | |
| Column | XBridge BEH C18 2.5 μm, 2.1 × 50 mm | |
| Eluent | A: 0.07% formic acid in Acetonitrile | |
| | B: 0.07% formic acid in water | |
| Detection signal | Diode Array | |
| Spectrum | Range: 200-400 nm; Resolution: 1.2 nm | |
| Sampling rate | 20 point/sec | |
| Injection | 0.5 μL standard injection | |
| Flow | 0.7 mL/min | |
| Column temperature | 35 °C | |
| Gradient | 0.0-0.40 min | 95% B |
| | 0.40-1.0 min | 95% → 5% B |
| | 1.00-2.00 min | 5% B |
| | 2.00-2.10 min | 5% → 95% B |
| | 2.10-2.50 min | 95% B |

Method Y

| UPLC-MS | Waters Acquity-UPLC-SQ Detector-2 | |
|---|---|---|
| MSD signal settings | Scan pos & neg 100-1500, | |
| | Source Voltage: Capillary Vol(kV)- | |
| | 3.50, Cone(V): 50 | |
| | Source Temp: Desolvation Temp(°C): 350 | |
| | Source Gas Flow: Desolvation(L/Hr): | |
| | 700, Cone (L/hr): 50 | |
| Column | AQUITY UPLC BEH C18 1.7 μm, 2.1 × 50 mm | |
| Eluent | A: 0.07% formic acid in Acetonitrile | |
| | B: 0.07% formic acid in water | |
| Detection signal | Diode Array | |
| Spectrum | Range: 200-400 nm; Resolution: 1.2 nm | |
| Sampling rate | 20 point/sec | |
| Injection | 0.5 μL standard injection | |
| Flow | 0.6 mL/min | |
| Column temperature | 35 °C | |
| Gradient | 0.0-0.40 min | 97% B |
| | 0.40-2.50 min | 97% → 2% B |
| | 2.50-3.40 min | 2% B |
| | 3.40-3.50 min | 2% → 97% B |
| | 3.50-4.00 min | 97% B |

| | |
|---|---|
| ELSD Parameters: - | GAS: 40 psi, Gain: 500, Drift Temp: 45 °C |

Method AB

| UPLC-MS | SHIMADZU LCMS-2020, LabSolution | |
|---|---|---|
| | Version 5.97SP1 | |
| MSD signal settings | ESI Scan pos 120-1000, Drying Gas: N₂ | |
| | Drying Gas Flow: 15(L/min) | |
| | DL Voltage: 120(v) | |
| | Qarray DC Voltage: 20(V) | |
| Column | Kinetex ® EVO C18 2.1 × 30 mm 5 um | |
| Eluent | A: 0.0375% TFA in water (v/v) | |
| | B: 0.01875% TFA in Acetonitrile (v/v) | |
| Detection signal | PDA Detector | |
| Spectrum | Range: 120-1000 nm | |
| Flow | 2.0 mL/min | |
| Column temperature | 50 °C | |
| Gradient | 0.0-0.6 min | 0% → 60% B |
| | 0.6-0.78 min | 60% |
| | 0.78-0.79 min | 60% → 0% B |
| | 0.79-0.80 min | 0% B |

Method AC

| HPLC-MS | Waters - Alliance 2695, SQ Detector-2 | |
|---|---|---|
| MSD signal settings | Scan pos & neg 100-1500, | |
| | Source Voltage: Capillary Vol(kV)- | |
| | 3.50, Cone(V): 35 | |
| | Source Temp: Desolvation Temp(°C): 350 | |
| | Source Gas Flow: Desolvation(L/Hr): 650, | |
| Column | XBridge C18 (4.6 × 75) mm, 3.5 μm | |
| Eluent | A: 10 mM Ammonium Bicarbonate in Water | |
| | B: Acetonitrile | |
| Flow | 1.30 mL/min | |
| Column temperature | 35 °C | |
| Gradient | 0.0-0.50 min | 5% B |
| | 0.50-1.0 min | 5% → 15% B |
| | 1.00-4.00 min | 15% → 98% B |
| | 4.00-7.0 min | 98% B |
| | 7.0-7.50 min | 98% à5% B |
| | 7.50-8.0 min | 5% B |

Method AD

| UPLC-MS | Waters Acquity-UPLC-SQ Detector-2 | |
|---|---|---|
| MSD signal settings | Scan pos & neg 100-1500, | |
| | Source Voltage: Capillary Vol(Kv)- | |
| | 3.50, Cone(V): 30 | |
| | Source Temp: Desolvation Temp(°C): 350 | |
| | Source Gas Flow: Desolvation (L/Hr): 700, | |
| Column | Acquity UPLC BEH C18 (3.0 × 30)mm 1.7 μm | |
| Eluent | A: 0.05% formic acid in Water | |
| | B: 0.05% formic acid in ACN | |
| Flow | 0.85 mL/min | |
| Column temperature | 50 °C | |
| Gradient | 0.0-0.05 min | 3% B |
| | 0.05-1.20 min | 3% → 98% B |
| | 1.20-1.75 min | 98% B |
| | 1.75-1.80 min | 98% → 3% B |
| | 1.80-2.10 min | 3% B |

| | |
|---|---|
| ELSD Parameters: | Gas −50 PSI; GAIN-500, DRIFT TEMP-50 °C |

Method AE

| | |
|---|---|
| Instrument | Waters - UPLC Aquity |
| | Mobile Phase-A: 0.05% Trifluoroacetic |
| | acid in Water |
| | Mobile Phase-B: 0.05% Trifluoroacetic |
| | acid Acetonitrile |
| Gradient Programme: | [Time in min/% of B]: 0/5, 2.0/5, |
| | 6.0/90, 8.5/90, 9.0/5, 10/5 |
| Column: | Aquity-UPLC HSS C18 (3.0 × 150 mm) 1.8 μm |
| Column Temp: | 50 °C |
| Flow rate: | 0.3 mL/min |
| Detector: | PDA Detector |
| Spectrum Range: | 210-400 nm |
| ELSD Parameters: | Nebuliser temp-40° |
| | Evaporator temp-45° |
| | Gas Flow Rate-3.0 |
| ELSD Parameters: | Gas −50 PSI; GAIN-500, DRIFT TEMP-50 °C |

Method AF

| | |
|---|---|
| Instrument | Waters - Arc-HPLC-SQ Detector-2 |
| | Mobile Phase-A: 10 mM Ammonium Acetate |
| | in water |
| | Mobile Phase-B: Acetonitrile |
| Gradient Programme: | [Time in min/% of B]: 0/2, 0.3/2, |
| | 1.3/98, 4.5/98, 5.0/2, 5.01/2 |
| Column: | X-Bridge C18, 4.6 × 75 mm, 3.5 μm |
| Column Temp: | 40 °C |
| Flow rate: | 1.2 mL/min |
| Detector: | ELSD Detector |
| Spectrum Range: | 210-400 nm |
| ELSD Parameters: | Nebuliser temp-40° |
| | Evaporator temp-45° |
| | Gas Flow Rate-3.0 |

The compounds according to the invention and intermediates are prepared by the methods of synthesis described hereinafter in which the substituents of the general formulae have the meanings given hereinbefore. These methods are intended as an illustration of the invention without restricting its subject matter and the scope of the compounds claimed to these examples. Where the preparation of starting compounds is not described, they are commercially obtainable or their synthesis is described in the prior art or they may be prepared analogously to known prior art compounds or methods described herein, i.e. it is within the skills of an organic chemist to synthesize these compounds. Substances described in the literature can be prepared according to the published methods of synthesis. If a chemical structure in the following is depicted without exact configuration of a stereo center, e.g. of an asymmetrically substituted carbon atom, then both configurations shall be deemed to be included and disclosed in such a representation. The representation of a stereo center in racemic form shall always deem to include and disclose both enantiomers (if no other defined stereo center exists) or all other potential diastereomers and enantiomers (if additional, defined or undefined, stereo centers exist).

### Synthesis of N,N-bis-substituted 3-amino pyrrolidines

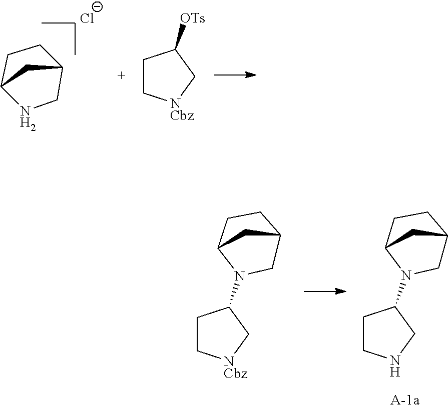

A solution of benzyl (3R)-3-[(4-methylbenzenesulfonyl)oxy]pyrrolidine-1-carboxylate (750 mg, 2.00 mmol, 1.0 equiv.) in acetonitrile (ACN) is added to a mixture of (1R,4S)-2-azabicyclo[2.2.1]heptane hydrochloride (309.1 mg, 2.2 mmol, 1.1 equiv.) and Caesium carbonate (1953 mg, 5.99 mmol, 3.0 equiv.). The reaction mixture is heated to 75 °C and stirred for 3 h.

After cooling to room temperature, the reaction mixture is diluted with CH₂Cl₂ and extracted with water. The aqueous phase is further extracted with DCM, the combined organic phases are dried over magnesium sulfate (MgSO4), filtered and the solvent is removed under reduced pressure. The residue is redissolved in MeOH (5 mL) and subsequently added to a slurry of Pd/C in CH₂Cl₂ (5 mL). The reaction mixture is pressurized with hydrogen gas (5 bar) and stirred at room temperature for 4 h. CH₂Cl₂ is added and the suspension is filtered over celite. The filtrate is evaporated to dryness and the residue is purified by chromatography to yield product A-1a.

The following intermediate compounds (Table 1) are obtained analogously.

TABLE 1

| | | | | HPLC |
|---|---|---|---|---|
| # | Structure | tᵣₑₜ [min] | [M + H]⁺ | method |
| A-1a | 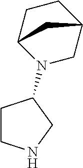 [SMILES: C1C[C@H](N2C[C@H]3CC[C@@H]2C3)CN1] | 0.75 | 167 | A |
| A-1b | 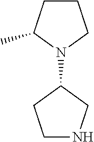 | 0.75 | 191 | A |
| A-1c | 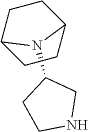 [SMILES: C1C[C@H](N2C3CCC2CC3)CN1] | 0.84 | 203 | A |
| A-1d | 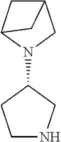 [SMILES: C1C[C@H](N2CC3CC2C3)CN1] | 0.84 | 153 | A |
| A-1e | 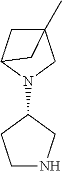 | 0.87 | 167 | A |
| A-1f | 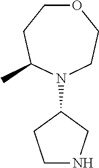 | 0.89 | 184 | A |
| A-1g | 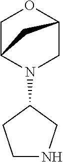 [SMILES: C1C[C@H](N2C[C@@H]3C[C@H]2CO3)CN1] | 0.82 | 169 | A |

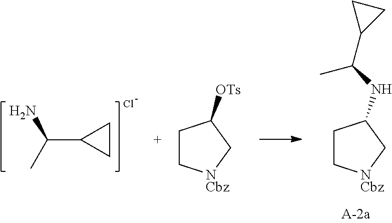

(3R)-3-[(4-methylbenzenesulfonyl)oxy]pyrrolidine-1-carboxylate (500 mg, 1.67 mmol, 1.0 equiv.), (R)-1-cyclopropylethanamine hydrochloride (428 mg 3.34 mmol, 2.0 equiv) and potassium carbonate (923 mg, 6.68 mg, 4.0 equiv.) are dissolved in ACN (5.0 mL). The reaction is stirred for 5 min at room temperature before heating up to 90 °C for 2 days. Upon completion the reaction is extracted with DCM and water. The organic layer is evaporated and purified by NP chromatography to afford A-2a.

The following intermediate compounds ( ) are obtained analogously.

TABLE 2

| | | | | HPLC |
|---|---|---|---|---|
| # | Structure | tᵣₑₜ [min] | [M + H]⁺ | method |
| A-2a | 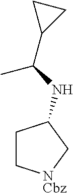 [SMILES: C[C@H](N[C@H]1CCN(C(=O)OCc2ccccc2)C1)C1CC1] | 0.80 | 289 | F |
| A-2b | 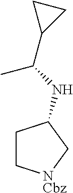 [SMILES: C[C@@H](N[C@H]1CCN(C(=O)OCc2ccccc2)C1)C1CC1] | 0.84 | 289 | F |

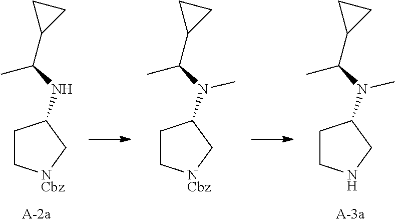 [SMILES: C[C@H](N[C@H]1CCN(C(=O)OCc2ccccc2)C1)C1CC1 ; C[C@@H](C1CC1)N(C)[C@H]1CCN(C(=O)OCc2ccccc2)C1 ; C[C@@H](C1CC1)N(C)[C@H]1CCNC1]

To an ice-cold solution of A-2a (218 mg, 0.76 mmol, 1.0 equiv.) in DMF (2 mL), NaH (51.0 mg, 1.28 mmol, 1.7 equiv.) is added and stirred at room temperature for 10 min. Then, methyl iodide (0.14 mL, 2.24 mmol, 2.96 equiv.) is added and the reaction is stirred at room temperature for 1 h. Upon completion the reaction is quenched with water and extracted with CH₂Cl₂ and sat. NaHCO₃ solution. The organic layer is dried under reduced pressure. The residue is redissolved in CH₂Cl₂:MeOH (1:2) (15 mL) and Pd/C (20 mg) is added. The reaction mixture is pressurized with hydrogen gas (6 bar) and stirred at room temperature for 2.5 h. The reaction mixture is filtered over celite and reduced to dryness under reduced pressure at 50 °C to afford A-3a.

The following intermediate compounds ( ) are obtained analogously.

TABLE 3

| | | | | HPLC |
|---|---|---|---|---|
| # | Structure | tᵣₑₜ [min] | [M + H]⁺ | method |
| A-3a | 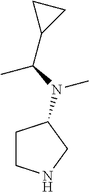 | 0.27 | 169 | F |
| A-3b | 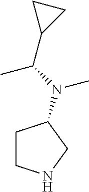 | 0.29 | 169 | F |

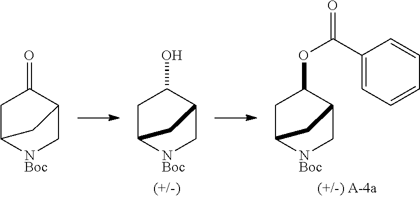 [SMILES: CC(C)(C)OC(=O)N1CC2CC1CC2=O ; CC(C)(C)OC(=O)N1C[C@@H]2C[C@H]1C[C@@H]2O ; CC(C)(C)OC(=O)N1C[C@@H]2C[C@H]1C[C@H]2OC(=O)c1ccccc1]

To a stirred solution of racemic tert-butyl 5-oxo-2-azabicyclo[2.2.1]heptane-2-carboxylate (100 g, 473.4 mmol, 1.0 equiv) in MeOH (1000 mL) NaBH₄ (17.38 g, 473.4 mmol, 1.0 equiv) is added portion wise at 0 °C. Then, the reaction mixture is allowed to RT and stirred for 3 hours. The reaction mixture is quenched with ice-cold water and extracted with DCM. The combined organic layers are dried over sodium sulphate, filtered and concentrated under reduced pressure to get a crude racemic mixture of tert-butyl (1S,4S,5S)-5-hydroxy-2-azabicyclo[2.2.1]heptane-2-carboxylate and tert-butyl (1R,4R,5R)-5-hydroxy-2-azabicyclo[2.2.1]heptane-2-carboxylate, which is used without further purification.

To a stirred solution of the intermediate racemic mixture of tert-butyl (1S,4S,5S)-5-hydroxy-2-azabicyclo[2.2.1]heptane-2-carboxylate and tert-butyl (1R,4R,5R)-5-hydroxy-2-azabicyclo[2.2.1]heptane-2-carboxylate (190.00 g, 891 mmol, 1.0 equiv) in THF (3800 mL) benzoic acid (163.2 g, 1336.3 mmol, 1.5 equiv) and PPh₃ (350.37 g, 1336.3 mmol, 1.5 equiv) are added at 0 °C. The resulting reaction mixture is stirred for 10 min at 0 °C and DIAD (265 mL, 1336.3 mmol, 1.5 equiv.) is added dropwise at same temperature. The reaction mixture is stirred for 16 h at RT. The reaction mixture is quenched with NaHCO₃ solution and extracted with ethyl acetate. The combined organic layers are dried over sodium sulphate, filtered and concentrated under reduced pressure The crude compound is purified by NP chromatography to afford racemic A-4a. (HPLC method Y, tᵣₑₜ=2.40 min; [M+H-^{t}butyl]=262).

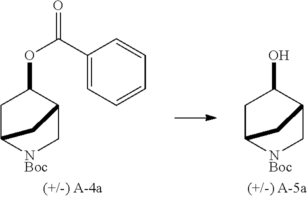 [SMILES: CC(C)(C)OC(=O)N1C[C@@H]2C[C@H]1C[C@H]2OC(=O)c1ccccc1 ; CC(C)(C)OC(=O)N1C[C@@H]2C[C@H]1C[C@H]2O]

To a stirred solution of racemic A-4a (210 g, 661.7 mmol, 1.0 equiv.) in MeOH (2100 mLI) 1M KOH in MeOH (992.5 mL, 992.5 mmol, 1.5 equiv.) is added at 0 °C and stirred for 4 h at RT. The reaction mixture is quenched with ice-cold water and extracted with ethyl acetate. The combined organic layers are dried over sodium sulphate, filtered and concentrated under reduced pressure to get crude compound, which is purified by NP chromatography to afford racemic A-5a. (H PLC method Y, tᵣₑₜ=1.5 min; [M+H-^{t}butyl]=158)

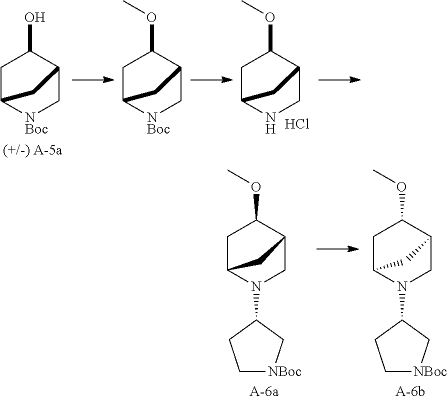

To a stirred solution of racemic A-5a (150 g, 703.3 mmol, 1 equiv.) in THF (1500 mL) 60% NaH (50.64 g, 2110 mmol, 3 equiv.) is added portion wise at 0 °C and stirred for 30 min at RT. Then, Mel (57 mL, 914 mmol, 1.3 equiv.) in THF (15 mL) is added dropwise at 0 °C and stirred for 4 h at RT. The reaction mixture is quenched with ice-cold water and extracted with ethyl acetate. The combined organic layers are dried over sodium sulphate, filtered and concentrated under reduced pressure to get a crude racemic mixture of tert-butyl (1S,4S,5R)-5-methoxy-2-azabicyclo[2.2.1]heptane-2-carboxylate and tert-butyl (1R,4R,5S)-5-methoxy-2-azabicyclo[2.2.1]heptane-2-carboxylate.

The intermediate racemic mixture of tert-butyl (1S,4S,5R)-5-methoxy-2-azabicyclo[2.2.1]heptane-2-carboxylate and tert-butyl (1R,4R,5S)-5-methoxy-2-azabicyclo[2.2.1]heptane-2-carboxylate (180.00 g, 71.92 mmol, 1 equiv.) is redissolved in DCM (1800 mL). 4M HCl in dioxane (360 mL, 1440 mmol, 1.82 equiv.) is added at 0 °C and stirred for 16 h at RT. The solvent is removed under reduced pressure to get a crude racemic mixture of (1S,4S,5R)-5-methoxy-2-azabicyclo[2.2.1]heptane hydrochloride and (1R,4R,5S)-5-methoxy-2-azabicyclo[2.2.1]heptane hydrochloride.

To a stirred solution of (R)-tert-butyl 3-(tosyloxy)pyrrolidine-1-carboxylate (150.00 g, 439 mmol, 1 equiv) and a racemic mixture of (1S,4S,5R)-5-methoxy-2-azabicyclo[2.2.1]heptane hydrochloride and (1R,4R,5S)-5-methoxy-2-azabicyclo[2.2.1]heptane hydrochloride (107.84 g, 659 mmol, 1.5 equiv) in acetonitrile (1500 mL) is added K₂CO₃ (242.87 g, 1757.3 mmol, 4 equiv) at RT and stirred for 16 h at 90 °C. The solvent is removed under reduced pressure and the obtained residue is diluted ethyl acetate washed with water. The organic layer is dried over Na₂SO₄ and concentrated under reduced pressure to get crude mixture, which is purified by NP chromatography. Then, the mixture is chirally separated with Method SFC 2 to afford A-6a and A-6b

| | | | | HPLC |
|---|---|---|---|---|
| # | Structure | tᵣₑₜ [min] | [M + H]⁺ | method |
| A-6a | 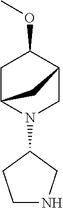 | 1.24 | 297 | Y |
| A-6b | 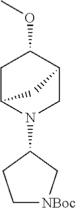 [SMILES: CO[C@H]1C[C@H]2C[C@@H]1CN2[C@H]1CCN(C(=O)OC(C)(C)C)C1] | 1.23 | 297 | Y |

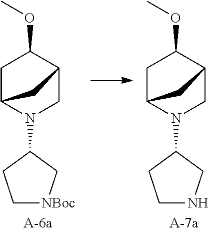 [SMILES: CO[C@@H]1C[C@@H]2C[C@H]1CN2[C@H]1CCN(C(=O)OC(C)(C)C)C1 ; CO[C@@H]1C[C@@H]2C[C@H]1CN2[C@H]1CCNC1]

To a stirred solution of A-6a (13.00 g, 43.8 mmol) in dichloromethane (130 mL) is added 4M HCl in dioxane (66 mL, 104 mmol, 2.37 mmol) at 0 °C and stirred for 16 h at RT. The solvent is removed under reduced pressure and then triturated with n-pentane to get of A-7a (H PLC method Y, tᵣₑₜ=2.40 min; [M+H-^{t}butyl]=262) as brown gummy liquid.

The following intermediate compounds (Table 4) are obtained analogously.

TABLE 4

| | | | | HPLC |
|---|---|---|---|---|
| # | Structure | tᵣₑₜ [min] | [M + H]⁺ | method |
| A-7a | 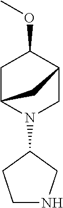 | 1.20 | 197 | AC |
| A-7b | 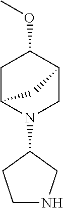 | 1.19 | 197 | AC |

### Synthesis of N,N-bis-substituted 2-amino azetidines

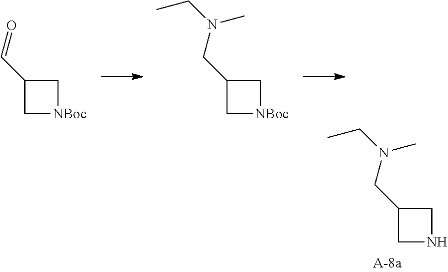

A solution of tert-butyl 3-formylazetidine-1-carboxylate (500 mg, 2.7 mmol, 1.0 equiv.) in dry methanol (7.5 mL) is prepared. To this, acetic acid (0.081 g, 1.36 mmol, 0.50 equiv.) and N-ethylmethylamine (329 mg, 5.4 mmol, 2.0 equiv.) are added. This mixture is stirred at room temperature for 10 minutes. Sodium triacetoxyborohydride (1.18 g, 5.4 mmol, 2.0 equiv.) is then added to the reaction mixture, which is stirred at room temperature for 1 h. The reaction mixture was filtered and evaporated to dryness. The residue was redissolved in dioxane (4 mL) and 6 M HCl (2.5 mL, 14.5 mmol, 10 equiv) was added. The reaction mixture is directly loaded on Isolute and purified by chromatography to give the product A-8a (H PLC method A, tᵣₑₜ=0.71 min; [M+H]=202).

The following intermediate compounds ( ) are obtained analogously.

TABLE 5

| | | | | HPLC |
|---|---|---|---|---|
| # | Structure | tᵣₑₜ [min] | [M + H]⁺ | method |
| A-8a | 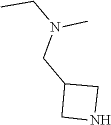 | 0.71 | 202 | A |
| A-8b | 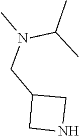 | 0.73 | 142 | A |
| A-8c | 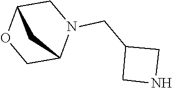 [SMILES: C1NCC1CN1C[C@@H]2C[C@H]1CO2] | 0.74 | 168 | A |

### Synthesis of Substituted Heterocyclic Anilines

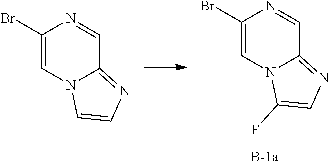 [SMILES: Brc1cn2ccnc2cn1 ; Fc1cnc2cnc(Br)cn12]

A solution of 6-bromoimidazo[1,2-a]pyrazine (24 g, 121 mmol, 1.0 equiv.) in acetonitrile (ACN, 240 mL) is cooled to 0 °C. Sodium hydride (NaH, 7.27 g, 182 mmol, 1.5 equiv.) is added to the solution. The reaction mixture is stirred at room temperature for 30 minutes. Following this, Selectfluor (85.87 g, 242 mmol, 2.0 equiv.) is added in acetonitrile (480 mL). The reaction mixture is stirred at room temperature for 16 hours.

The reaction mixture is quenched with water and extracted with ethyl acetate. The organic layer is dried over sodium sulfate (Na₂SO₄) and the solvent is evaporated under reduced pressure. The crude product is purified using neutral alumina and eluted in 4% ethyl acetate/hexane to afford the product, 6-bromo-3-fluoro-imidazo[1,2-a]pyrazine B-1a (H PLC method AF, tᵣₑₜ=1.17 min; [M+H]=216).

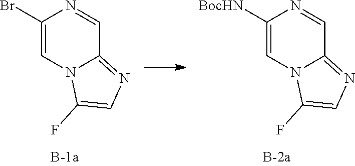 [SMILES: Fc1cnc2cnc(Br)cn12 ; CC(C)(C)OC(=O)Nc1cn2c(F)cnc2cn1]

A solution of B-1a (4.75 g, 22 mmol, 1.0 equiv.) in 1,4-dioxane (90.5 mL) is stirred. To this, cesium carbonate (Cs₂CO₃, 0.79 g, 33 mmol, 1.5 equiv.), tert-butyl carbonate (5.87 g, 44 mmol, 2.0 equiv.), and Xantphos (12.72 g, 22 mmol, 1.0 equiv.) are added. The mixture is degassed for 5 minutes. Following this, palladium(II) acetate (Pd(OAc)₂, 2.3 mL, 22 mmol, 1.0 equiv.) is added and the mixture is degassed for another 5 minutes. The reaction mixture is then stirred at 100 °C for 3 hours.

The reaction mixture is filtered through a celite bed and the solvent is evaporated under reduced pressure. The crude product is purified using basic silica and eluted in 27% ethyl acetate/hexane to afford the product, tert-butyl N-(3-fluoroimidazo[1,2-a]pyrazin-6-yl)carbamate B-2a (H PLC method X, tᵣₑₜ=2.62 min; [M+H]=253).

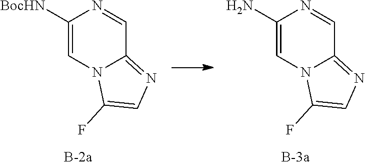 [SMILES: CC(C)(C)OC(=O)Nc1cn2c(F)cnc2cn1 ; Nc1cn2c(F)cnc2cn1]

A solution of B-2a (4.00 g, 15.9 mmol, 1.0 equiv.) in dichloromethane (DCM, 40 mL) is stirred. To this, trifluoroacetic acid (12 mL) is added. The reaction mixture is stirred at room temperature for 4 hours.

The reaction mixture is then evaporated to afford the crude product. This is diluted with tetrahydrofuran (THF, 10 mL) and a 20% sodium hydroxide (NaOH) solution (30 mL) is added. The mixture is stirred for 1 hour and then extracted with DCM (3×15 mL). The organic layer is washed with water (2×10 mL), then a brine solution (10 mL), and dried over sodium sulfate (Na₂SO₄). The solvent is evaporated under reduced pressure to afford a pale brown solid. This is washed with diethyl ether (3×6 mL) and dried to afford the product, 3-fluoroimidazo[1,2-a]pyrazin-6-amine B-3a (HPLC method X, tᵣₑₜ=2.14 min; [M+H]=167).

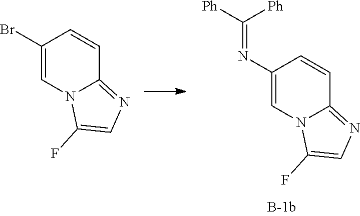 [SMILES: Fc1cnc2ccc(Br)cn12 ; Fc1cnc2ccc(N=C(c3ccccc3)c3ccccc3)cn12]

A solution of 6-bromo-3-fluoro-imidazo[1,2-a]pyridine (21.00 g, 97.7 mmol, 1.0 equiv.) in dioxane (420 mL) is stirred at room temperature and degassed with nitrogen. To this, sodium tert-butoxide (NaOtBu, 14.92 g, 155 mmol, 1.59 equiv.), benzophenone imine (27.08 g, 149 mmol, 1.53 equiv.), BINAP (15.94 g, 19.5 mmol, 0.20 equiv.), and Pd₂(dba)₃ (8.94 g, 9.77 mmol, 0.10 equiv.) are added. The reaction mixture is heated to 80 °C for 2 hours.

After the starting material is consumed (as monitored by TLC and LCMS), the reaction mixture is quenched with water and extracted with ethyl acetate (2×30 mL). The organic layer is separated, dried over anhydrous sodium sulfate (Na₂SO₄), and the solvent is evaporated under reduced pressure to afford the crude product. This is purified by column chromatography on basic silica gel, eluting with 45% ethyl acetate in petroleum ether to afford the product, N-(3-fluoroimidazo[1,2-a]pyridin-6-yl)-1,1-diphenyl-methanimine B-1b (HPLC method X, tᵣₑₜ=1.00 min; [M+H]=316).

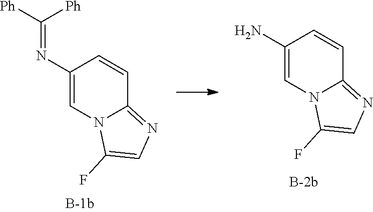 [SMILES: Fc1cnc2ccc(N=C(c3ccccc3)c3ccccc3)cn12 ; Nc1ccc2ncc(F)n2c1]

A solution of B-1b (0.10 g, 0.317 mmol, 1.0 equiv.) in tetrahydrofuran (THF, 2 mL) is stirred at 0 °C. To this, 1M hydrochloric acid (HCl, 4.0 mL, 0.317 mmol, 1.0 equiv.) is added. The reaction mixture is then stirred at room temperature for 1 hour.

The reaction mixture is dissolved in dichloromethane (50 mL) and the organic layer is separated. The aqueous layer is basified with potassium carbonate (K₂CO₃) and extracted with a 20% methanol (MeOH) in DCM solution (2×100 mL). The organic layers are combined, concentrated under reduced pressure, and dried. The solid is washed with diethyl ether and dried to afford the product, 3-fluoroimidazo[1,2-a]pyridin-6-amine B-2b (HPLC method AD, tᵣₑₜ=2.21 min; [M+H]=152).

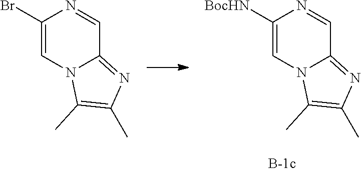 [SMILES: Cc1nc2cnc(Br)cn2c1C ; Cc1nc2cnc(NC(=O)OC(C)(C)C)cn2c1C]

A solution of 6-bromo-2,3-dimethyl-imidazo[1,2-a]pyrazine (18.00 g, 79.6 mmol, 1.0 equiv.) in 1,4-dioxane (180 mL) is stirred. To this, tert-butyl carbamate (18.65 g, 159 mmol, 2.0 equiv.) and cesium carbonate (77.82 g, 239 mmol, 3.0 equiv.) are added. The reaction mixture is degassed with argon for 5 minutes. Then, palladium(II) acetate (1.79 g, 7.96 mmol, 0.1 equiv.) and xanthphos (9.20 g, 15.9 mmol, 0.2 equiv.) are added. The reaction mixture is stirred at 100 °C for 16 hours and the progress is monitored by LCMS.

After completion, the reaction mixture is filtered through a celite bed and washed with ethyl acetate. The filtrate is concentrated under reduced pressure to afford the crude product. This is purified by column chromatography on neutral alumina, eluting with a 60-70% ethyl acetate in hexane solution to afford the product, tert-butyl N-(2,3-dimethylimidazo[1,2-a]pyrazin-6-yl)carbamate, as a brown solid (7.00 g, 24.0 mmol, 30.17% yield) B-1c (HPLC method AD, tᵣₑₜ=2.56 min; [M+H]=249).

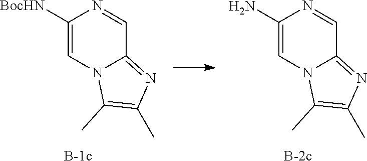

A solution of B-1c (100 mg, 0.381 mmol, 1.0 equiv.) in 4M HCl in dioxane (3 mL) is prepared at 0 °C. The reaction mixture is stirred at room temperature for 12 hours and the progress is monitored by LCMS.

After completion, the reaction mixture is concentrated to dryness. The residue is dissolved in water and the pH is adjusted to 8-9 with a saturated sodium carbonate solution. The product is extracted with dichloromethane (DCM). The organic layer is washed with water and then with a saturated brine solution. It is then dried over sodium sulfate (Na₂SO₄) and the solvent is evaporated to afford the product, 2,3-dimethylimidazo[1,2-a]pyrazin-6-amine, as a solid B-2c (HPLC method AD, tᵣₑₜ=2.15 min; [M+H]=163).

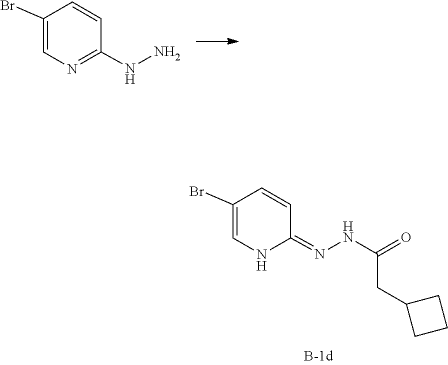 [SMILES: NNc1ccc(Br)cn1 ; O=C(CC1CCC1)N/N=c1\ccc(Br)c[nH]1]

5-bromo-2-hydrazineylpyridine (10 g, 53 mmol, 1 equiv.) is dissolved in toluene (100 mL) and 2-cylcobutylacetic acid (6.1 g, 53 mmol, 1 equiv.) is added at 0 °C. Next, reaction mixture is heated to 120 °C and stirred for 3 days. Upon complete conversion the reaction mixture is concentrated in vacuo and the crude material B-1d is used without further purification (4.5 g, 4.6 mmol; TLC: 5% MeOH in DCM, R_{f}: 0.3; HPLC method: AC; tᵣₑₜ=1.25)

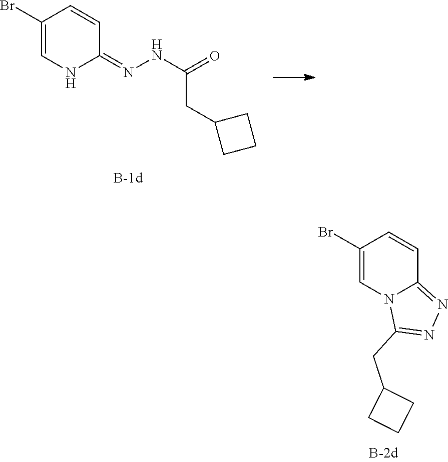 [SMILES: O=C(CC1CCC1)N/N=c1\ccc(Br)c[nH]1 ; Brc1ccc2nnc(CC3CCC3)n2c1]

B-1d (14 g, 74 mmol, 1 equiv.) is dissolved in toluene (140 mL) at 0 °C, next pTSA (1.7 g, 9.8 mmol, 0.2 equiv.) is added and the reaction is heated to 120 °C and stirred for 72 h. Upon complete conversion, the reaction is basified with sat. aq. NaHCO₃ and extracted three times with EtOAc. The combined organic layers are dried over Na₂SO₄, filtered, and concentrated in vacuo. The crude material is purified by NP chromatography to give B-2d (8 g, 27 mmol; TLC: 50% EtOAc in Hexane, R_{f}: 0.5; HPLC method AC; tᵣₑₜ=1.4 min; [M+H]⁺=266).

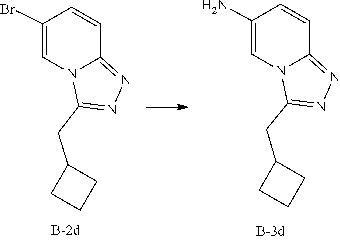 [SMILES: Brc1ccc2nnc(CC3CCC3)n2c1 ; Nc1ccc2nnc(CC3CCC3)n2c1]

B-2d (6 g, 22.5 mmol, 1 eqiuv.), diphenylmethanimine (6.13 g, 33.8 mmol, 1.5 equiv.), sodium t-butoxide (4.3 g, 45 mmol, 2 equiv.) and Xanthphos (1.3 g, 2.2 mmol, 0.1 equiv.) in dioxane (60 mL) is degassed for 5 minutes with nitrogen. Next, Pd(dba)₃ (1 g, 1.1 mmol, 0.05 equiv.) is added and the reaction mixture is stirred at 100 °C for 12 h. Upon complete conversion, the reaction mixture is filtered over a pad of celite and washed with 5% MeOH in DCM (50 mL). The filtrate is concentrated in vacuo and the crude material is purified by NP chromatography to give B-3d (TLC: 10% MeOH in DCM, R_{f}: 0.2; HPLC method AC; tᵣₑₜ=1.2 min, [M+H]⁺=203).

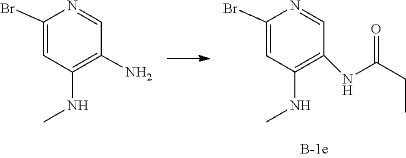

To a stirred solution 6-bromo-N4-methyl-pyridine-3,4-diamine (4.00 g, 19.8 mmol. 1.00 equiv.) and propionic acid (1.5 mL, 19.8 mmol, 1.00 equiv.) in DCM (40 mL), EDC-HCl (5.69 g, 29.7 mmol, 1.50 equiv) and DIPEA (5.2 mL, 29.7 mmol, 1.50 equiv.) were added. The reaction was stirred at room temperature for 16 h. Upono completion the reaction was concentrated and purified by NP chromatography to afford B-1e. (TLC: 10% MeOH in DCM, UV active, Rf=0.4, HPLC method X; tᵣₑₜ=0.47 min; [M+H]⁺=258)

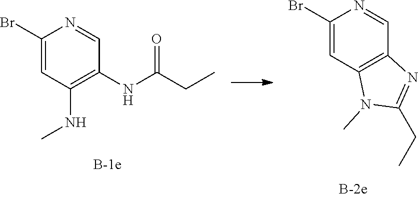

B-1e (30 g, 116 mmol, 1 equiv.) is dissolved in AcOH (150 mL) and heated to 100 °C and stirred for 16 h. Upon complete conversion, the reaction mixture is concentrated, then basified with sodium carbonate to pH 8.5. The aqueous mixture is extracted three times with DCM and the combined organic layers are washed sequentially with ice-cold water and brine. The combined organic layers are dried over Na₂SO₄, filtered, and concentrated in vacuo. The crude material is purified by NP chromatography to give B-2e (20 g, 59 mmol; TLC: 10% MeOH in DCM, R_{f}: 0.5; HPLC method AD; tᵣₑₜ=1.1 min; [M+H]⁺=241).

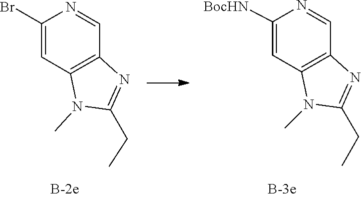 [SMILES: CCc1nc2cnc(Br)cc2n1C ; CCc1nc2cnc(NC(=O)OC(C)(C)C)cc2n1C]

B-2e (2.40 g, 10.0 mmol, 1.0 equiv.) is dissolved in 1,4-dioxane (48 mL), then t-butyl carbamate (3.51 g, 30.0 mmol, 3.0 equiv.) and cesium carbonate (13.03 g, 40 mmol, 4.0 equiv.) are added. The reaction mixture is purged with nitrogen gas for 5-10 minutes, and subsequently BrettPhos Pd G3 (0.54 g, 0.54 mmol, 0.60 equiv.) is added. The reaction mixture is stirred at 100 °C for 6 h. Upon complete conversion, the reaction mixture is concentrated, quenched with water and extracted with DCM (3×10 mL). The organic layers are washed with ice water and brine, then dried over Na₂SO₄, filtered, and concentrated in vacuo to give the crude solid. The material is purified by NP chromatography to give product B-3e (HPLC method AD; tᵣₑₜ=1.1 min, [M+H]+=277).

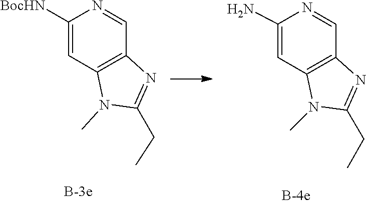 [SMILES: CCc1nc2cnc(NC(=O)OC(C)(C)C)cc2n1C ; CCc1nc2cnc(N)cc2n1C]

B-3e (13 g, 47 mmol, 1 equiv.) is dissolved in 6 N HCl (130 mL) and the reaction mixture is stirred for 16 h. Upon complete conversion, the reaction mixture is basified with sodium carbonate and extracted three times with DCM. The combined organic layers are washed ice cold water and brine, dried over Na₂SO₄, filtered, and concentrated in vacuo. The crude material is purified by NP chromatography to give B-4e (4.7 g, 24.9 mmol; TLC: 10% MeOH in DCM, R_{f}: 0.2; HPLC method AD; tᵣₑₜ=2.1 min; [M+H]⁺=186).

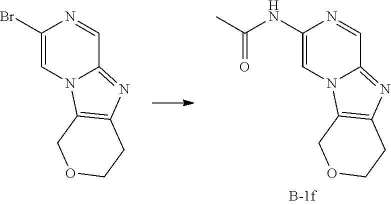 [SMILES: Brc1cn2c3c(nc2cn1)CCOC3 ; CC(=O)Nc1cn2c3c(nc2cn1)CCOC3]

12-bromo-4-oxa-1,8,11-triazatricyclo[7.4.0.02,7]trideca-2(7),8,10,12-tetraene (2.5 g, 9.9 mmol, 1 equiv.) is dissolved in toluene (25 mL) and degassed with nitrogen for 15 minutes. Next, K₂CO₃ (4.1 g, 29 mmol, 3 equiv.) is added and the reaction mixture stirred for 15 minutes continuing to purge with nitrogen. Copper iodide (0.19 g, 0.9 mmol, 0.1 equiv.) and 1,2-dimethylethylenediamine (0.87 g, 9.8 mmol, 1 equiv.) are added and the reaction mixture stirred at 120 °C for 2 h. Upon complete conversion, the reaction mixture is filtered over a pad of celite using EtOAc. The filtrate is washed with water, and the combined organic layer dried over Na2SO4, filtered and concentrated in vacuo. The crude material is purified by NP chromatography to give B-1f. (HPLC method AC; tᵣₑₜ=2.14 min, [M+H]⁺=233)

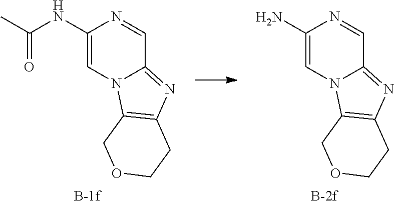 [SMILES: CC(=O)Nc1cn2c3c(nc2cn1)CCOC3 ; Nc1cn2c3c(nc2cn1)CCOC3]

B-1f (2.3 g, 9.9 mmol, 1 equiv.) is dissolved in methanol (25 mL) at 0 °C. KOH (1.11 g, 19.8 mmol, 2 equiv.) is added and the reaction mixture stirred at 70 °C for 16 h. Upon complete conversion, the reaction mixture is diluted with water and extracted twice with 10% MeOH/DCM. The combined organic layers are dried over Na₂SO₄, filtered, and concentrated in vacuo. The crude material is washed three times with Et₂O to give product B-2f (0.77 g, 3.6 mmol; TLC: 10% MeOH in DCM, R_{f}: 0.2; HPLC method AD; tᵣₑₜ=4.9 min, [M+H]⁺=191).

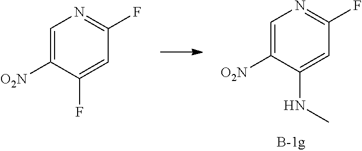

To a solution of methylamine (6.55 g, 15.2 mmol, 1.2 equiv.) and triethylamine (1.41 g, 13.9 mmol, 1.1 equiv.) in THF (5 mL), a solution of 2,4-difluoro-5-nitropyridine (2.11 g, 12.7 mmol, 1.0 equiv.) in THF (5 mL) is added dropwise. The reaction is stirred at room temperature for 40 min. The reaction is extracted with ethylacetate and water. The organic layer is dried over MgSO₄ and evaporated to dryness to afford B-1g (HPLC method C; tᵣₑₜ=0.66; [M+H]⁺=170), which is used without further purification.

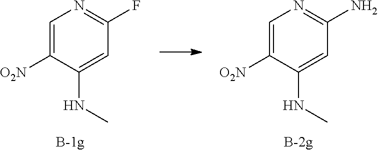

B-1g (2.2 g, 11.6 mmol, 1.0 equiv.) is dissolved in NH₃ (28% in water, 30 mL) and stirred at 60 °C for 2 h. The reaction is dissolved in ethyl acetate/2-propanol and extracted with water. The organic layer is dried over MgSO₄, filtered and evaporated under reduced pressure to afford B-2g (HPLC method C; tᵣₑₜ=0.50-0.54; [M+H]+=169), which is used without further purification.

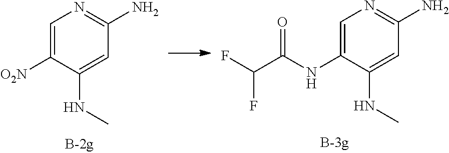

To a solution of B-2g (2.05 g, 12.2 mmol, 1.0 equiv.) in difluoroacetic acid (15 mL) Fe (3.5 g, 5.12 mmol, 5.1 equiv.) is added. The reaction is stirred at 60 °C for 2 h. The reaction is diluted with ethyl acetate and sat. NaHCO₃ is added slowly. The mixture is filtered over celite and then extracted. The organic layer is dried over MgSO₄ and evaporated to dryness to afford B-3g (H PLC method C; tᵣₑₜ=0.14; [M+H]+=217), which is used without further purification.

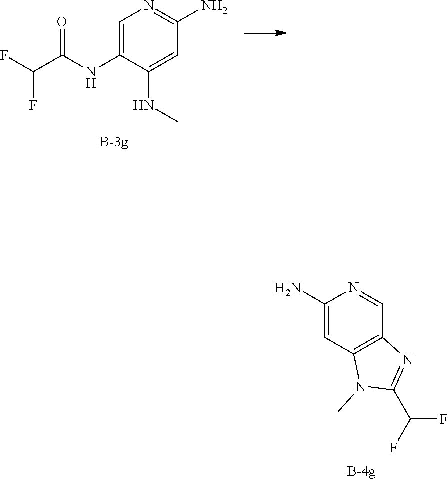

B-3g (3.21 g, 11.9 mmol, 1.0 equiv.) is dissolved in DCE (20 mL) and N,O-bis(trimethylsilyl)acetamide (10.0 mL, 40.8 mmol, 3.4 equiv) is added. The reaction mixture is stirred at 90 °C for 24 h. Upon complete conversion, the reaction mixture is extracted with ethylacetate and sat. NaHCO₃ solution. The organic layer is dried over MgSO₄ and concentrated under reduced pressure, then purified by basic RP chromatography and concentrated in vacuo to give B-4g (HPLC method C; tᵣₑₜ=0.52; [M+H]+=199).

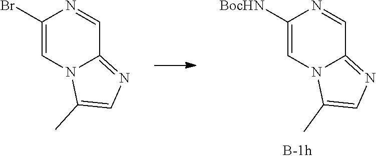 [SMILES: Cc1cnc2cnc(Br)cn12 ; Cc1cnc2cnc(NC(=O)OC(C)(C)C)cn12]

To a stirred solution of 6-bromo-3-methyl-imidazo[1,2-a]pyrazine (22.00 g, 104 mmol, 1.00 equiv) in 1,4-dioxane (330 mL) tert-butyl carbamate (36.42 g, 311 mmol, 3.00 equiv) and cesium carbonate (134.87 g, 415 mmol, 4.00 equiv) are added. Then, nitrogen gas is purged into the reaction for 5-10 min, then finally added Brettphos Pd G3 (5.64 g, 6.22 mmol, 0.06 equiv). The reaction is stirred at 100 °C for 16 hours. Upon completion the reaction is cooled to 0 °C and diluted with ethyl acetate, filtered over celite and washed with water and brine. The combined organic layers is dried with Na₂SO₄, concentrated under reduced pressure and purified with NP chromatography to afford B-1h. (HPLC method D; tᵣₑₜ=2.52 min; [M+H]⁺=249)

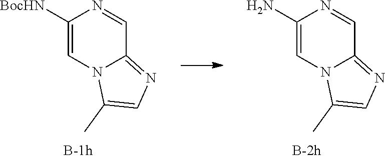 [SMILES: Cc1cnc2cnc(NC(=O)OC(C)(C)C)cn12 ; Cc1cnc2cnc(N)cn12]

To a stirred solution of B-1h (50 mg, 0.20 mmol, 1.00 equiv.) in DCM (3 mL) TFA (0.15 mL, 1.84 mmol, 9.15 equiv.) is added at 0 °C. The reaction is stirred at room temperature for 7 h. Upon completion the reaction is concentrated under reduced pressure and diluted with water and ethyl acetate. The aq. layer is basified with sat. NaHCO₃ and extracted with ethyl acetate and dried over Na₂SO₄ to afford B-2h. (HPLC method AC; tᵣₑₜ=2.12 min: [M+H]=149)

### Synthesis of C-3a

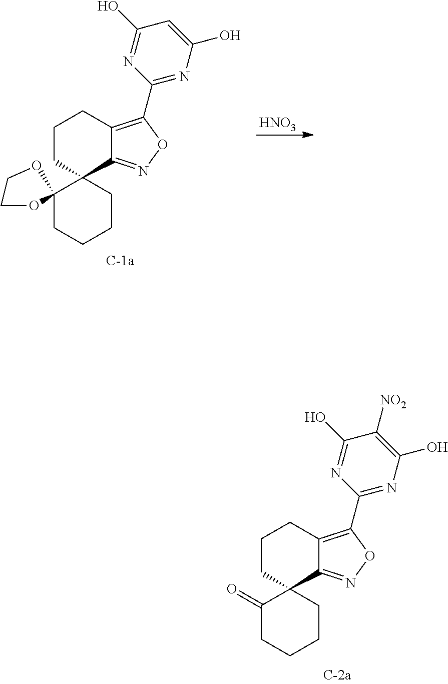

A solution of C-1a (WO2024115529,130 g, 362 mmol, 1.0 equiv.) in glacial acetic acid (650 mL) is stirred at 0 °C. To this, fuming nitric acid (65 mL, 362 mmol, 1.0 equiv.) is added. The resulting mixture is allowed to warm to room temperature and stirred for 2 hours. The progress of the reaction is monitored by TLC. Upon completion, the reaction is quenched with ice water and stirred for 30 minutes. The solid product is collected by filtration, then slurried in methyl tert-butyl ether, stirred for 30 minutes, filtered and dried. The solid is then co-evaporated with toluene under reduced pressure to give the product, C-2a, as a pale yellow solid (HPLC method X, tᵣₑₜ=1.22 min; [M+H]=361).

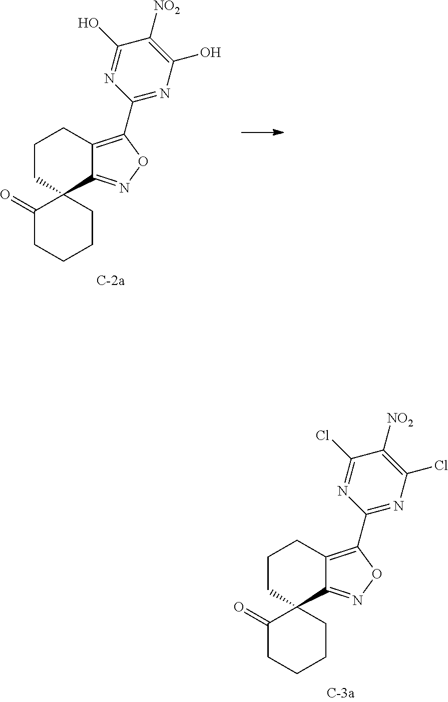

A solution of C-2a (120 g, 333 mmol, 1.0 equiv.) in toluene (1200 mL) is stirred at room temperature. To this, N,N-Diisopropylethylamine (290 mL, 1665 mmol, 5.0 equiv.) is added, followed by a dropwise addition of phosphoryl chloride (155 mL, 1665 mmol, 5.0 equiv.) at 0 °C. The resulting reaction mixture is stirred at 0 °C to room temperature for 3 hours. The progress of the reaction is monitored by TLC. Upon completion, the reaction is quenched with ice water and stirred for 10 minutes. The mixture is then extracted with methyl tert-butyl ether. The organic layer is washed with aqueous sodium bicarbonate and brine, dried over anhydrous sodium sulfate, filtered and concentrated under reduced pressure to give the crude product as a brown solid. The crude product is purified by flash column chromatography, eluting with 15% ethyl acetate in hexane. The desired fractions are concentrated in vacuo to give the product, C-3a, as a solid (HPLC method AD, tᵣₑₜ=01.24 min; [M+H]=397).

### Coupling of Amines A-1 and Anilines B-1 to Building Block C-4a

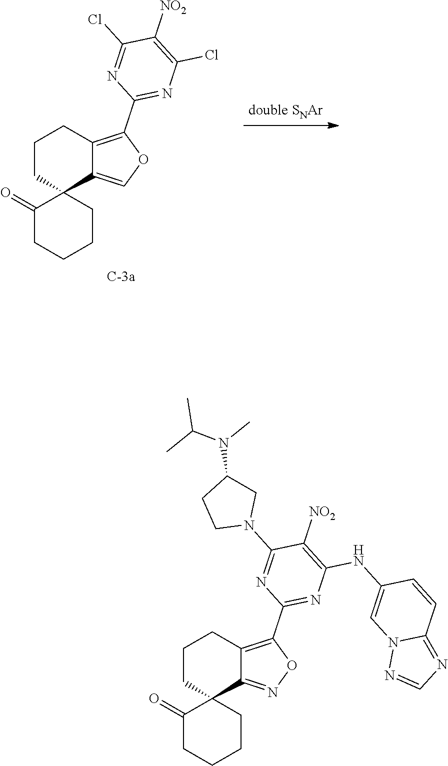 [SMILES: O=C1CCCC[C@@]12CCCc1c2coc1-c1nc(Cl)c([N+](=O)[O-])c(Cl)n1 ; CC(C)N(C)[C@H]1CCN(c2nc(-c3onc4c3CCC[C@@]43CCCCC3=O)nc(Nc3ccc4ncnn4c3)c2[N+](=O)[O-])C1]

C-3a (250 mg, 0.59 mmol, 1 equiv) is dissolved in CH₂Cl₂ (1 mL) and the solution was cooled to −10 °C. A solution of the amine coupling partner (S)—N-isopropyl-N-methylpyrrolidin-3-amine dihydrochloride (152 mg, 0.59 mmol, 1 equiv) in CH₂Cl₂ (1 mL) and DIPEA (0.35 mL, 2.09 mmol, 3.5 equiv) is added dropwise to this solution. After stirring for 10 min at −10 °C, the mixture is evaporated to dryness and the crude was redissolved in THF (5 mL) and cooled to −10 °C. Aniline coupling partner [1,2,4]triazolo[1,5-a]pyridin-6-amine (113 mg, 0.71 mmol, 1.2 equiv), followed by LiHMDS solution (1 M, 2.92 mL, 2.92 mmol, 4.9 equiv) were added and the solution was stirred for 30 min. The reaction mixture was diluted with CH₂Cl₂ and aqeuous NaHCO₃ solution and extracted with CH₂Cl₂ three times. The combined extracts are dried with Na₂SO₄ and evaporated to dryness. The crude product is purified by RP-chromatography to give the desired product product C-4a.

The following intermediate compounds C-4 (Table 6) are obtained analogously using the corresponding amine and aniline as coupling partners.

TABLE 6

| | | tᵣₑₜ | | HPLC |
|---|---|---|---|---|
| # | structure | [min] | [M + H]⁺ | method |
| C-4a | 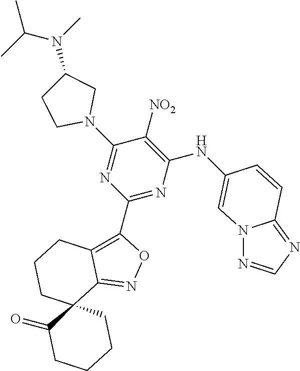 [SMILES: CC(C)N(C)[C@H]1CCN(c2nc(-c3onc4c3CCC[C@@]43CCCCC3=O)nc(Nc3ccc4ncnn4c3)c2[N+](=O)[O-])C1] | 1.16 | 601 | A |
| C-4b | 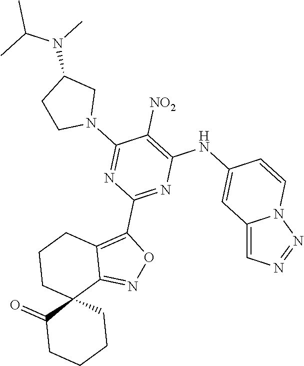 [SMILES: CC(C)N(C)[C@H]1CCN(c2nc(-c3onc4c3CCC[C@@]43CCCCC3=O)nc(Nc3ccn4nncc4c3)c2[N+](=O)[O-])C1] | 1.06 | 601 | A |
| C-4c | 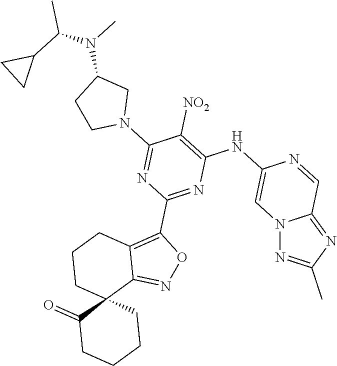 [SMILES: Cc1nc2cnc(Nc3nc(-c4onc5c4CCC[C@@]54CCCCC4=O)nc(N4CC[C@H](N(C)[C@@H](C)C5CC5)C4)c3[N+](=O)[O-])cn2n1] | 1.18 | 642 | A |
| C-4d | 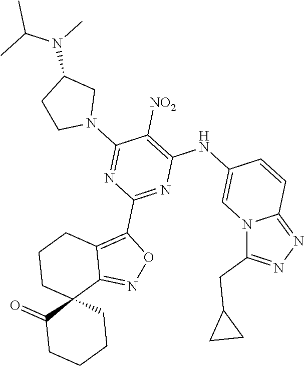 [SMILES: CC(C)N(C)[C@H]1CCN(c2nc(-c3onc4c3CCC[C@@]43CCCCC3=O)nc(Nc3ccc4nnc(CC5CC5)n4c3)c2[N+](=O)[O-])C1] | 1.11 | 655 | A |
| C-4e | 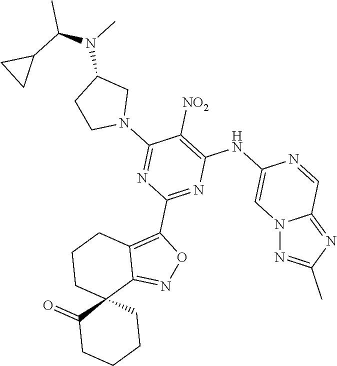 [SMILES: Cc1nc2cnc(Nc3nc(-c4onc5c4CCC[C@@]54CCCCC4=O)nc(N4CC[C@H](N(C)[C@H](C)C5CC5)C4)c3[N+](=O)[O-])cn2n1] | 1.17 | 642 | A |
| C-4f | 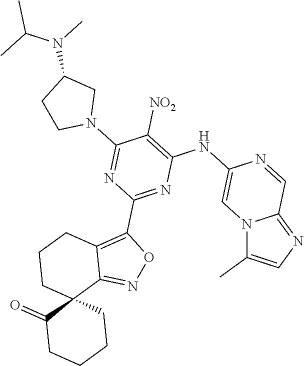 [SMILES: Cc1cnc2cnc(Nc3nc(-c4onc5c4CCC[C@@]54CCCCC4=O)nc(N4CC[C@H](N(C)C(C)C)C4)c3[N+](=O)[O-])cn12] | 1.10 | 615 | A |
| C-4g | 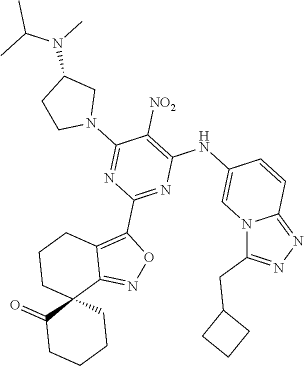 [SMILES: CC(C)N(C)[C@H]1CCN(c2nc(-c3onc4c3CCC[C@@]43CCCCC3=O)nc(Nc3ccc4nnc(CC5CCC5)n4c3)c2[N+](=O)[O-])C1] | 1.14 | 669 | A |
| C-4h | 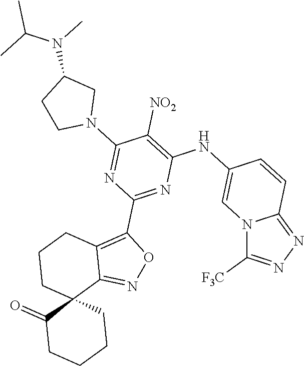 [SMILES: CC(C)N(C)[C@H]1CCN(c2nc(-c3onc4c3CCC[C@@]43CCCCC3=O)nc(Nc3ccc4nnc(C(F)(F)F)n4c3)c2[N+](=O)[O-])C1] | 1.10 | 669 | A |
| C-4i | 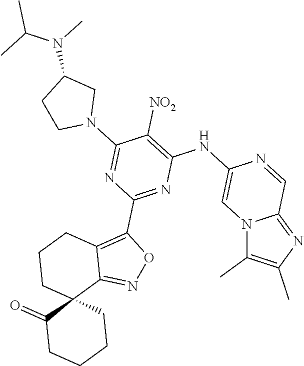 [SMILES: Cc1nc2cnc(Nc3nc(-c4onc5c4CCC[C@@]54CCCCC4=O)nc(N4CC[C@H](N(C)C(C)C)C4)c3[N+](=O)[O-])cn2c1C] | 1.14 | 629 | A |
| C-4j | 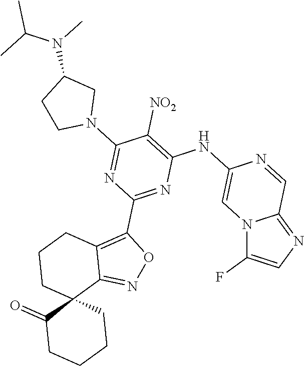 [SMILES: CC(C)N(C)[C@H]1CCN(c2nc(-c3onc4c3CCC[C@@]43CCCCC3=O)nc(Nc3cn4c(F)cnc4cn3)c2[N+](=O)[O-])C1] | 1.11 | 619 | A |
| C-4k | 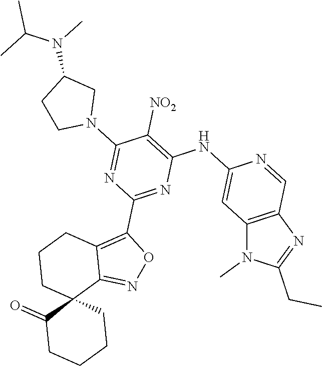 [SMILES: CCc1nc2cnc(Nc3nc(-c4onc5c4CCC[C@@]54CCCCC4=O)nc(N4CC[C@H](N(C)C(C)C)C4)c3[N+](=O)[O-])cc2n1C] | 1.14 | 633 | A |
| C-4l | 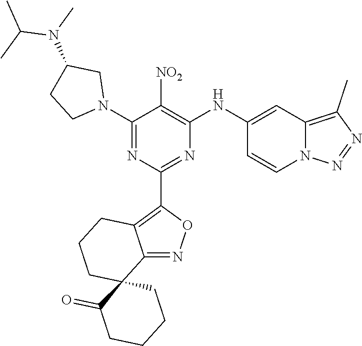 [SMILES: Cc1nnn2ccc(Nc3nc(-c4onc5c4CCC[C@@]54CCCCC4=O)nc(N4CC[C@H](N(C)C(C)C)C4)c3[N+](=O)[O-])cc12] | 1.07 | 615 | A |
| C-4m | 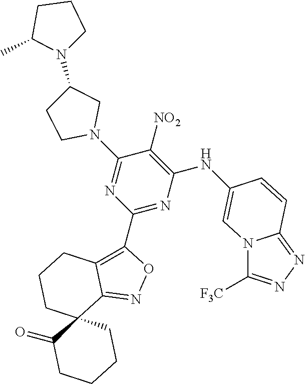 [SMILES: C[C@@H]1CCCN1[C@H]1CCN(c2nc(-c3onc4c3CCC[C@@]43CCCCC3=O)nc(Nc3ccc4nnc(C(F)(F)F)n4c3)c2[N+](=O)[O-])C1] | 1.12 | 681 | A |
| C-4n | 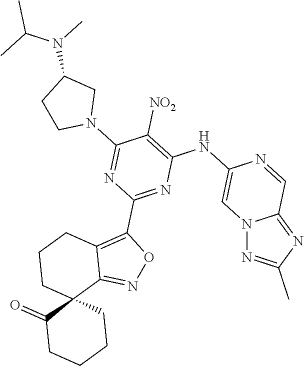 [SMILES: Cc1nc2cnc(Nc3nc(-c4onc5c4CCC[C@@]54CCCCC4=O)nc(N4CC[C@H](N(C)C(C)C)C4)c3[N+](=O)[O-])cn2n1] | 1.12 | 616 | A |
| C-4o | 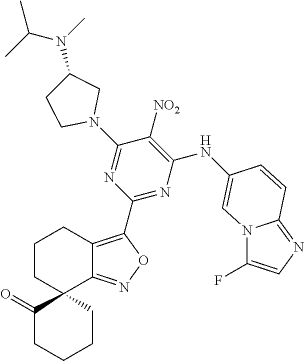 [SMILES: CC(C)N(C)[C@H]1CCN(c2nc(-c3onc4c3CCC[C@@]43CCCCC3=O)nc(Nc3ccc4ncc(F)n4c3)c2[N+](=O)[O-])C1] | 1.12 | 618 | A |
| C-4p | 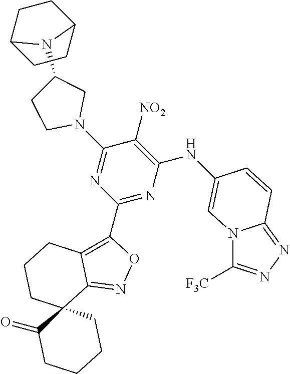 [SMILES: O=C1CCCC[C@@]12CCCc1c2noc1-c1nc(Nc2ccc3nnc(C(F)(F)F)n3c2)c([N+](=O)[O-])c(N2CC[C@H](N3C4CCC3CC4)C2)n1] | 1.17 | 693 | A |
| C-4q | 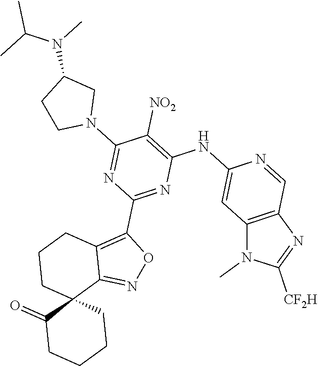 [SMILES: CC(C)N(C)[C@H]1CCN(c2nc(-c3onc4c3CCC[C@@]43CCCCC3=O)nc(Nc3cc4c(cn3)nc(C(F)F)n4C)c2[N+](=O)[O-])C1] | 1.14 | 666 | A |
| C-4r | 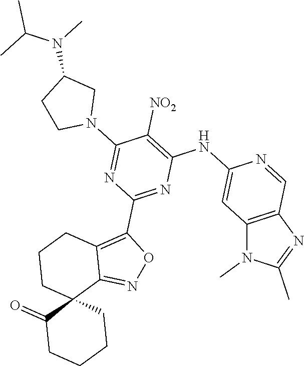 [SMILES: Cc1nc2cnc(Nc3nc(-c4onc5c4CCC[C@@]54CCCCC4=O)nc(N4CC[C@H](N(C)C(C)C)C4)c3[N+](=O)[O-])cc2n1C] | 1.05 | 629 | A |
| C-4s | 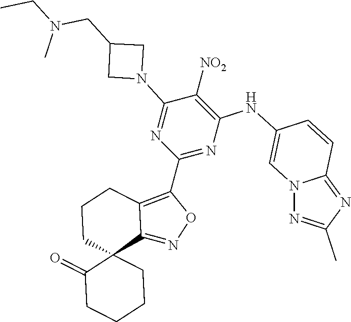 [SMILES: CCN(C)CC1CN(c2nc(-c3onc4c3CCC[C@@]43CCCCC3=O)nc(Nc3ccc4nc(C)nn4c3)c2[N+](=O)[O-])C1] | 1.03 | 601 | C |
| C-4t | 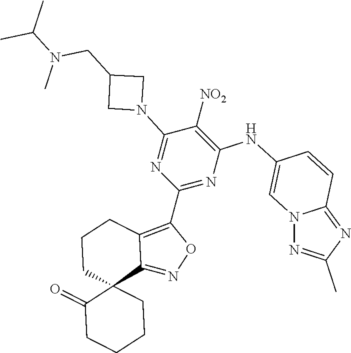 [SMILES: Cc1nc2ccc(Nc3nc(-c4onc5c4CCC[C@@]54CCCCC4=O)nc(N4CC(CN(C)C(C)C)C4)c3[N+](=O)[O-])cn2n1] | 1.04 | 615 | C |
| C-4u | 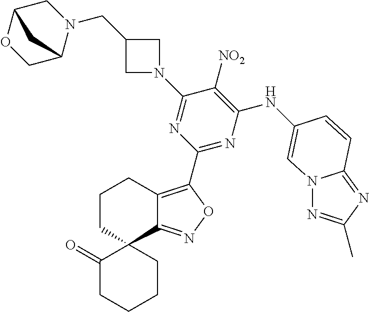 [SMILES: Cc1nc2ccc(Nc3nc(-c4onc5c4CCC[C@@]54CCCCC4=O)nc(N4CC(CN5C[C@@H]6C[C@H]5CO6)C4)c3[N+](=O)[O-])cn2n1] | 0.92 | 641 | C |
| C-4v | 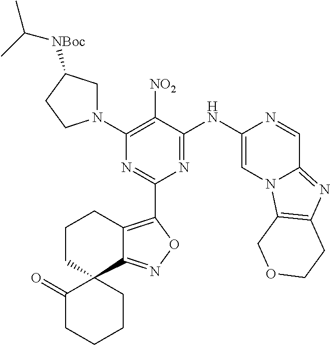 [SMILES: CC(C)N(C(=O)OC(C)(C)C)[C@H]1CCN(c2nc(-c3onc4c3CCC[C@@]43CCCCC3=O)nc(Nc3cn4c5c(nc4cn3)CCOC5)c2[N+](=O)[O-])C1] | 1.19 | 743 | C |
| C-4w | 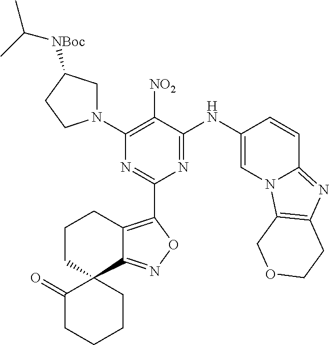 [SMILES: CC(C)N(C(=O)OC(C)(C)C)[C@H]1CCN(c2nc(-c3onc4c3CCC[C@@]43CCCCC3=O)nc(Nc3ccc4nc5c(n4c3)COCC5)c2[N+](=O)[O-])C1] | 1.10 | 714 | C |
| C-4x | 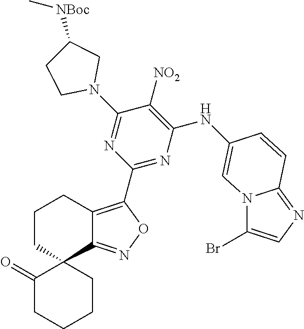 [SMILES: CN(C(=O)OC(C)(C)C)[C@H]1CCN(c2nc(-c3onc4c3CCC[C@@]43CCCCC3=O)nc(Nc3ccc4ncc(Br)n4c3)c2[N+](=O)[O-])C1] | 1.16 | 736 | C |
| C-4y | 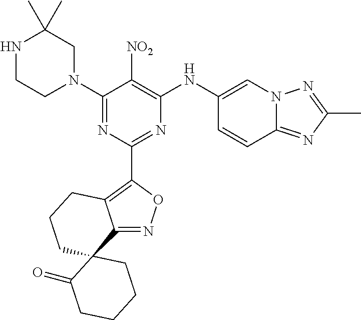 [SMILES: Cc1nc2ccc(Nc3nc(-c4onc5c4CCC[C@@]54CCCCC4=O)nc(N4CCNC(C)(C)C4)c3[N+](=O)[O-])cn2n1] | 0.94 | 587 | C |
| C-4z | 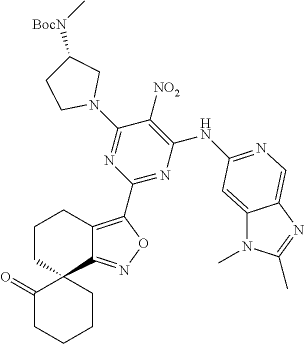 [SMILES: Cc1nc2cnc(Nc3nc(-c4onc5c4CCC[C@@]54CCCCC4=O)nc(N4CC[C@H](N(C)C(=O)OC(C)(C)C)C4)c3[N+](=O)[O-])cc2n1C] | 1.13 | 687 | C |
| C-4aa | 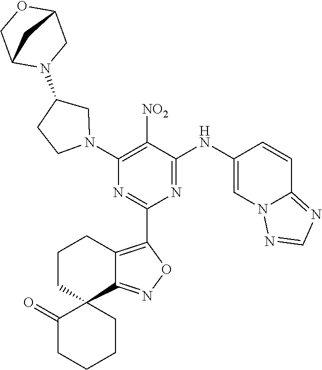 [SMILES: O=C1CCCC[C@@]12CCCc1c2noc1-c1nc(Nc2ccc3ncnn3c2)c([N+](=O)[O-])c(N2CC[C@H](N3C[C@@H]4C[C@H]3CO4)C2)n1] | 0.91 | 627 | C |
| C-4ab | 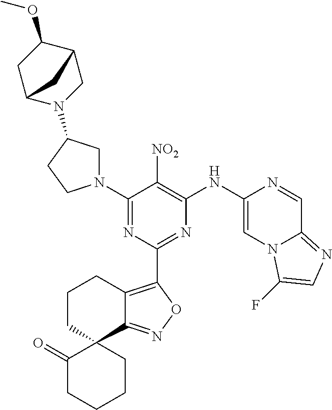 [SMILES: CO[C@@H]1C[C@@H]2C[C@H]1CN2[C@H]1CCN(c2nc(-c3onc4c3CCC[C@@]43CCCCC3=O)nc(Nc3cn4c(F)cnc4cn3)c2[N+](=O)[O-])C1] | 673 | 1.11 | C |
| C-4ac | 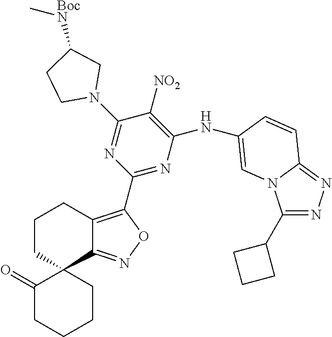 [SMILES: CN(C(=O)OC(C)(C)C)[C@H]1CCN(c2nc(-c3onc4c3CCC[C@@]43CCCCC3=O)nc(Nc3ccc4nnc(C5CCC5)n4c3)c2[N+](=O)[O-])C1] | 1.24 | 713 | C |
| C-4ad | 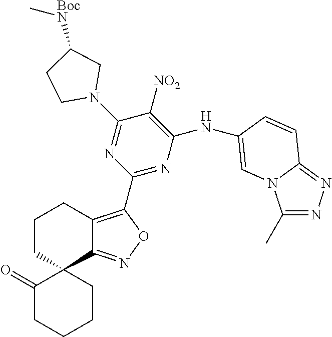 [SMILES: Cc1nnc2ccc(Nc3nc(-c4onc5c4CCC[C@@]54CCCCC4=O)nc(N4CC[C@H](N(C)C(=O)OC(C)(C)C)C4)c3[N+](=O)[O-])cn12] | 0.78 | 673 | C |

### Reductive Purine Formation

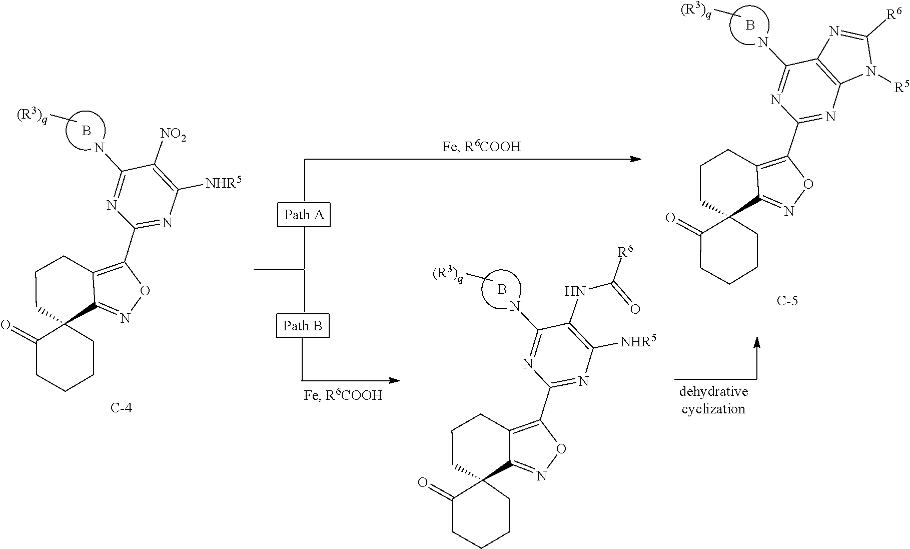

### General Procedure Path A

The C-4 was dissolved in trifluoroacetic acid (0.13 M), followed by addition of iron powder (5.0 equiv). The reaction mixture was stirred for 16 h at 60 °C. If reaction monitoring with HPLC-MS indicates full conversion, the reaction mixture was diluted with CH₂Cl₂ and aqeuous NaHCO₃ solution was added carefully until gas evolution ceased. The suspension was filtered over celite and extracted with CH₂Cl₂ three times. The combined extracts were dried with Na₂SO₄ and evaporated to dryness. The crude product was purified by RP-chromatography to give the desired product C-5.

### General Procedure Path B

The C-4 was dissolved in trifluoroacetic acid (0.13 M), followed by addition of iron powder (5.0 equiv). The reaction mixture was stirred for 16 h at 60 °C. If reaction monitoring with HPLC-MS indicates full conversion, the reaction mixture was diluted with CH₂Cl₂ and aqeuous NaHCO₃ solution was added carefully until gas evolution ceased. The suspension was filtered over celite and extracted with CH₂Cl₂ three times. The crude product was dissolved in dichloro ethane (0.2 M) and N,O-bis(trimethylsilyl)acetamide (12 equiv) was added. The reaction mixture was stirred for 1 h at 90 °C. If reaction monitoring with HPLC-MS indicates full conversion, the reaction mixture was diluted with CH₂Cl₂ and aqeuous NaHCO₃ solution was added carefully until gas evolution ceased. The suspension was filtered over celite and extracted with CH₂Cl₂ three times. The combined extracts were dried with Na₂SO₄ and evaporated to dryness. The crude product was purified by RP-chromatography to give the desired product C-5.

The following intermediate products C-5 ( ) are obtained by applying the indicated general procedures. For C-5q Difluoroacetic acid and for C-5r 2-Fluoro-2-methyl propanal is used.

TABLE 7

| | | | | HPLC | General |
|---|---|---|---|---|---|
| # | structure | tᵣₑₜ [min] | [M + H]⁺ | method | Procedure |
| C-5a | 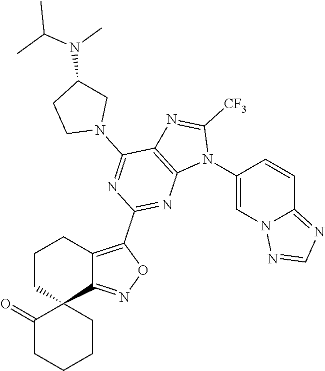 [SMILES: CC(C)N(C)[C@H]1CCN(c2nc(-c3onc4c3CCC[C@@]43CCCCC3=O)nc3c2nc(C(F)(F)F)n3-c2ccc3ncnn3c2)C1] | 1.11 | 649 | A | A |
| C-5b | 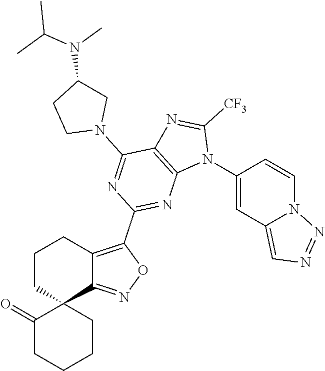 [SMILES: CC(C)N(C)[C@H]1CCN(c2nc(-c3onc4c3CCC[C@@]43CCCCC3=O)nc3c2nc(C(F)(F)F)n3-c2ccn3nncc3c2)C1] | 1.11 | 649 | A | A |
| C-5c | 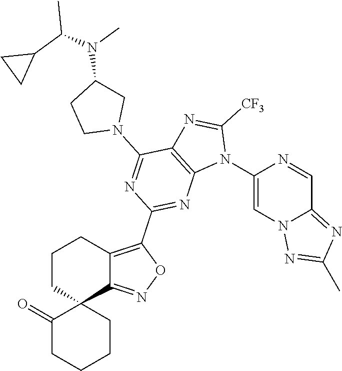 [SMILES: Cc1nc2cnc(-n3c(C(F)(F)F)nc4c(N5CC[C@H](N(C)[C@@H](C)C6CC6)C5)nc(-c5onc6c5CCC[C@@]65CCCCC5=O)nc43)cn2n1] | 1.15 | 690 | A | B |
| C-5d | 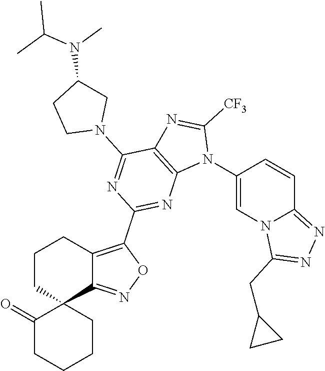 [SMILES: CC(C)N(C)[C@H]1CCN(c2nc(-c3onc4c3CCC[C@@]43CCCCC3=O)nc3c2nc(C(F)(F)F)n3-c2ccc3nnc(CC4CC4)n3c2)C1] | 1.09 | 702 | A | A |
| C-5e | 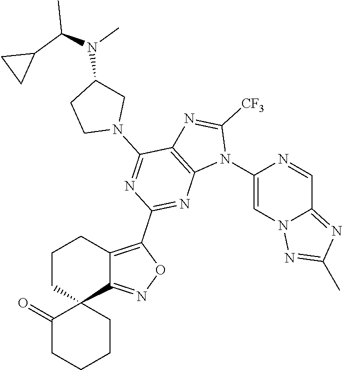 [SMILES: Cc1nc2cnc(-n3c(C(F)(F)F)nc4c(N5CC[C@H](N(C)[C@H](C)C6CC6)C5)nc(-c5onc6c5CCC[C@@]65CCCCC5=O)nc43)cn2n1] | 0.89 | 708 | A | B |
| C-5f | 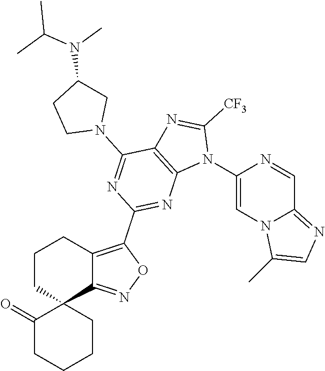 [SMILES: Cc1cnc2cnc(-n3c(C(F)(F)F)nc4c(N5CC[C@H](N(C)C(C)C)C5)nc(-c5onc6c5CCC[C@@]65CCCCC5=O)nc43)cn12] | 1.11 | 663 | A | B |
| C-5g | 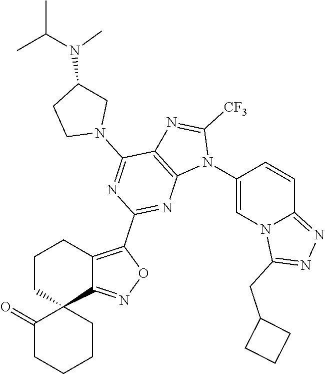 [SMILES: CC(C)N(C)[C@H]1CCN(c2nc(-c3onc4c3CCC[C@@]43CCCCC3=O)nc3c2nc(C(F)(F)F)n3-c2ccc3nnc(CC4CCC4)n3c2)C1] | 1.11 | 717 | A | A |
| C-5h | 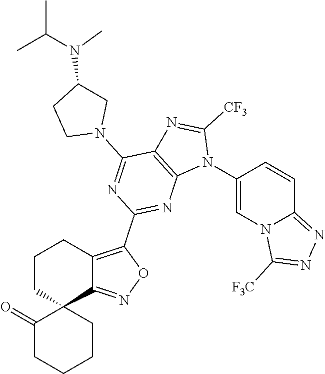 [SMILES: CC(C)N(C)[C@H]1CCN(c2nc(-c3onc4c3CCC[C@@]43CCCCC3=O)nc3c2nc(C(F)(F)F)n3-c2ccc3nnc(C(F)(F)F)n3c2)C1] | 1.13 | 717 | A | A |
| C-5i | 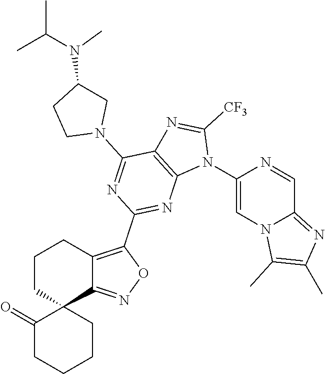 [SMILES: Cc1nc2cnc(-n3c(C(F)(F)F)nc4c(N5CC[C@H](N(C)C(C)C)C5)nc(-c5onc6c5CCC[C@@]65CCCCC5=O)nc43)cn2c1C] | 1.14 | 677 | A | B |
| C-5j | 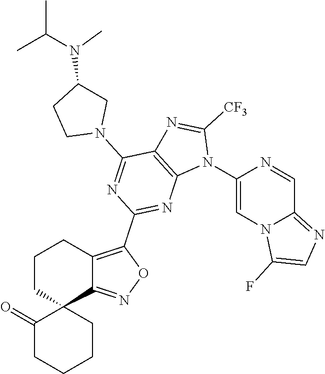 [SMILES: CC(C)N(C)[C@H]1CCN(c2nc(-c3onc4c3CCC[C@@]43CCCCC3=O)nc3c2nc(C(F)(F)F)n3-c2cn3c(F)cnc3cn2)C1] | 1.09 | 667 | A | B |
| C-5k | 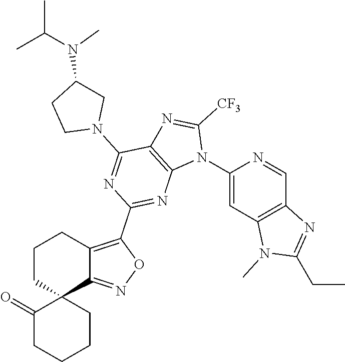 [SMILES: CCc1nc2cnc(-n3c(C(F)(F)F)nc4c(N5CC[C@H](N(C)C(C)C)C5)nc(-c5onc6c5CCC[C@@]65CCCCC5=O)nc43)cc2n1C] | 1.11 | 691 | A | B |
| C-5l | 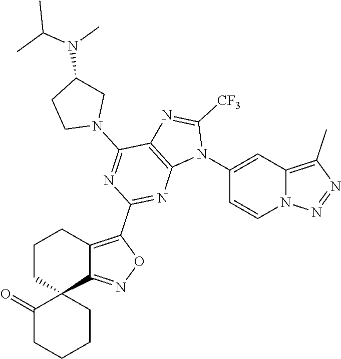 [SMILES: Cc1nnn2ccc(-n3c(C(F)(F)F)nc4c(N5CC[C@H](N(C)C(C)C)C5)nc(-c5onc6c5CCC[C@@]65CCCCC5=O)nc43)cc12] | 1.12 | 663 | A | A |
| C-5m | 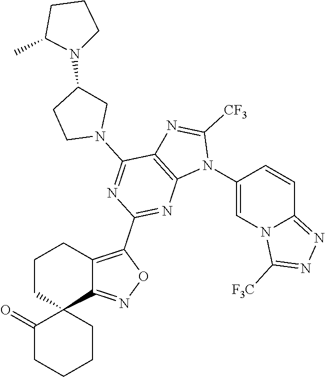 [SMILES: C[C@@H]1CCCN1[C@H]1CCN(c2nc(-c3onc4c3CCC[C@@]43CCCCC3=O)nc3c2nc(C(F)(F)F)n3-c2ccc3nnc(C(F)(F)F)n3c2)C1] | 1.11 | 729 | A | A |
| C-5n | 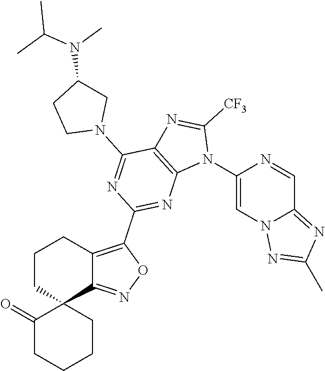 [SMILES: Cc1nc2cnc(-n3c(C(F)(F)F)nc4c(N5CC[C@H](N(C)C(C)C)C5)nc(-c5onc6c5CCC[C@@]65CCCCC5=O)nc43)cn2n1] | 0.93 | 682 | A | A |
| C-5o | 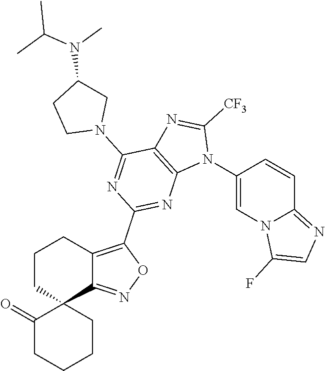 [SMILES: CC(C)N(C)[C@H]1CCN(c2nc(-c3onc4c3CCC[C@@]43CCCCC3=O)nc3c2nc(C(F)(F)F)n3-c2ccc3ncc(F)n3c2)C1] | 1.11 | 666 | A | A |
| C-5p | 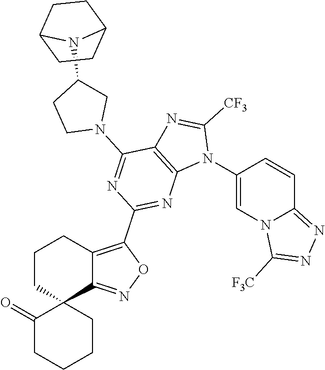 [SMILES: O=C1CCCC[C@@]12CCCc1c2noc1-c1nc(N2CC[C@H](N3C4CCC3CC4)C2)c2nc(C(F)(F)F)n(-c3ccc4nnc(C(F)(F)F)n4c3)c2n1] | 1.15 | 741 | A | A |
| C-5q | 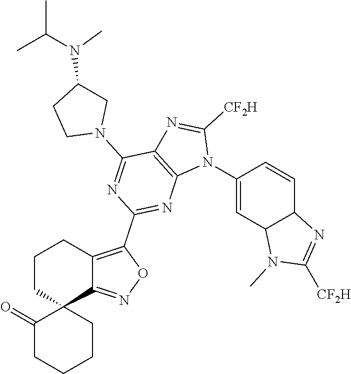 [SMILES: CC(C)N(C)[C@H]1CCN(c2nc(-c3onc4c3CCC[C@@]43CCCCC3=O)nc3c2nc(C(F)F)n3C2=CC3C(C=C2)N=C(C(F)F)N3C)C1] | 1.13 | 695 | A | B |
| C-5r | 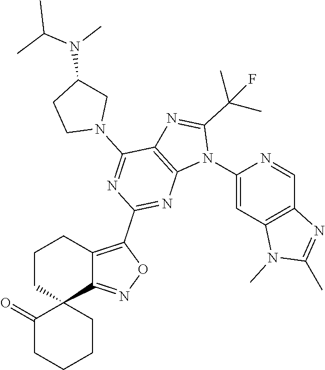 [SMILES: Cc1nc2cnc(-n3c(C(C)(C)F)nc4c(N5CC[C@H](N(C)C(C)C)C5)nc(-c5onc6c5CCC[C@@]65CCCCC5=O)nc43)cc2n1C] | 1.03 | 669 | A | B |
| C-5s | 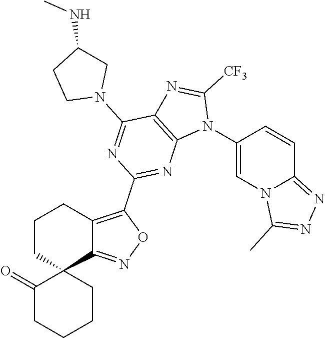 [SMILES: CN[C@H]1CCN(c2nc(-c3onc4c3CCC[C@@]43CCCCC3=O)nc3c2nc(C(F)(F)F)n3-c2ccc3nnc(C)n3c2)C1] | 0.90 | 621 | C | A |
| C-5t | 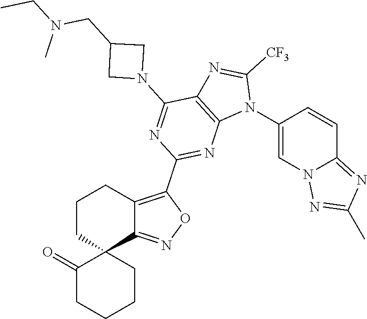 [SMILES: CCN(C)CC1CN(c2nc(-c3onc4c3CCC[C@@]43CCCCC3=O)nc3c2nc(C(F)(F)F)n3-c2ccc3nc(C)nn3c2)C1] | 0.85 | 667 | C | B |
| C-5u | 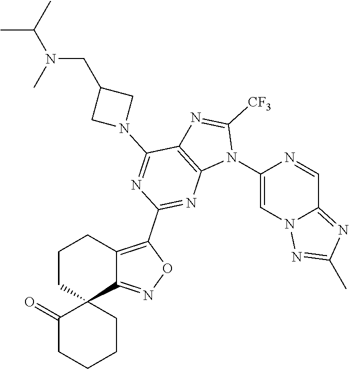 [SMILES: Cc1nc2cnc(-n3c(C(F)(F)F)nc4c(N5CC(CN(C)C(C)C)C5)nc(-c5onc6c5CCC[C@@]65CCCCC5=O)nc43)cn2n1] | 0.87 | 681 | C | B |
| C-5v | 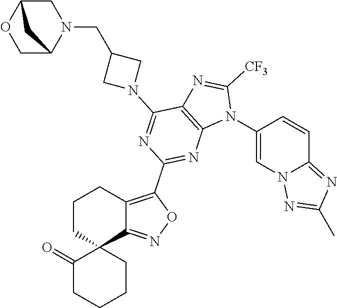 [SMILES: Cc1nc2ccc(-n3c(C(F)(F)F)nc4c(N5CC(CN6C[C@@H]7C[C@H]6CO7)C5)nc(-c5onc6c5CCC[C@@]65CCCCC5=O)nc43)cn2n1] | 0.77 | 770 | C | B |
| C-5w | 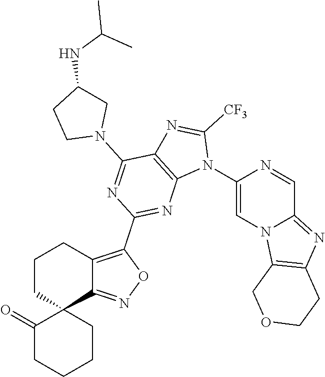 [SMILES: CC(C)N[C@H]1CCN(c2nc(-c3onc4c3CCC[C@@]43CCCCC3=O)nc3c2nc(C(F)(F)F)n3-c2cn3c4c(nc3cn2)CCOC4)C1] | 1.03 | 691 | C | B |
| C-5x | 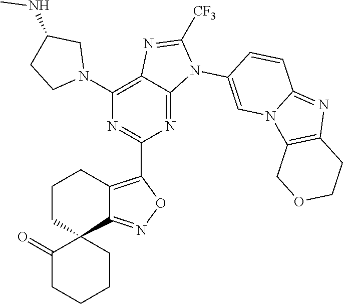 [SMILES: CN[C@H]1CCN(c2nc(-c3onc4c3CCC[C@@]43CCCCC3=O)nc3c2nc(C(F)(F)F)n3-c2ccc3nc4c(n3c2)COCC4)C1] | 0.94 | 662 | C | A |
| C-5y | 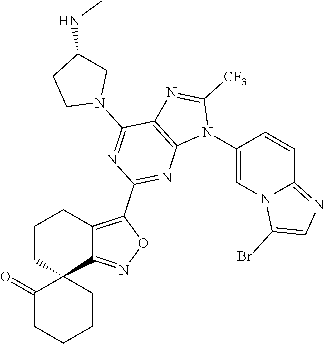 [SMILES: CN[C@H]1CCN(c2nc(-c3onc4c3CCC[C@@]43CCCCC3=O)nc3c2nc(C(F)(F)F)n3-c2ccc3ncc(Br)n3c2)C1] | 1.02 | 684 | C | A |
| C-5z | 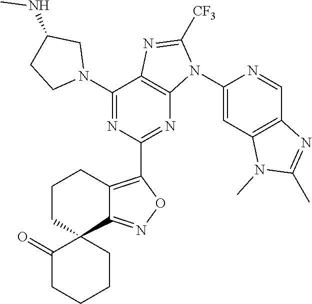 | 0.97 | 635 | C | B |
| C-5aa | 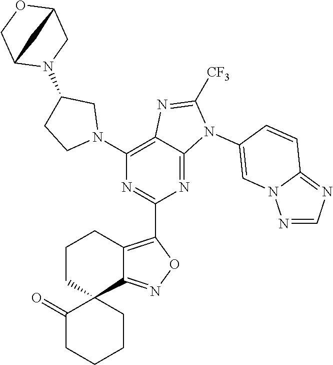 [SMILES: O=C1CCCC[C@@]12CCCc1c2noc1-c1nc(N2CC[C@H](N3C[C@@H]4C[C@H]3CO4)C2)c2nc(C(F)(F)F)n(-c3ccc4ncnn4c3)c2n1] | 0.95 | 675 | C | A |
| C-5ab | 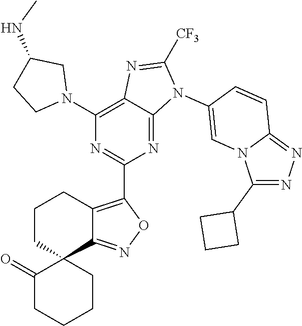 [SMILES: CN[C@H]1CCN(c2nc(-c3onc4c3CCC[C@@]43CCCCC3=O)nc3c2nc(C(F)(F)F)n3-c2ccc3nnc(C4CCC4)n3c2)C1] | 0.98 | 661 | C | A |
| C-5ac | 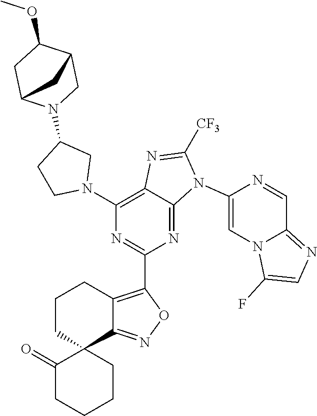 [SMILES: CO[C@@H]1C[C@@H]2C[C@H]1CN2[C@H]1CCN(c2nc(-c3onc4c3CCC[C@@]43CCCCC3=O)nc3c2nc(C(F)(F)F)n3-c2cn3c(F)cnc3cn2)C1] | 1.05 | 721 | C | B |

### Overview Scheme to F-1a and F-1b

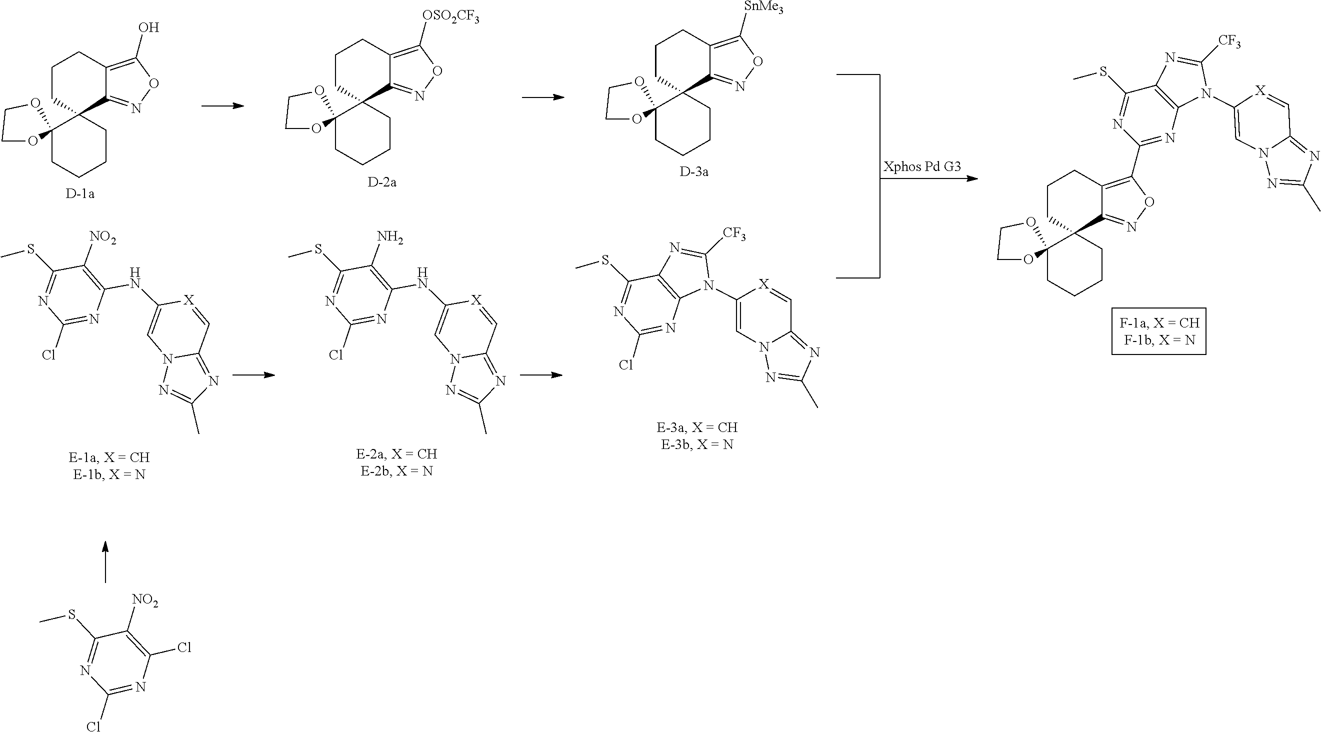

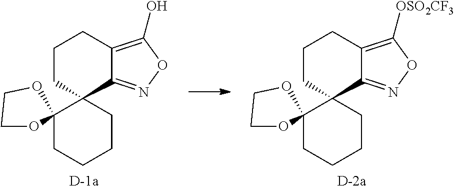

A solution of D-1a (10 g, 37.7 mmol, 1.0 equiv.) in dichloromethane (200 mL) is stirred at room temperature. To this, triethylamine (2.0 equiv., 7.61 g, 75.4 mmol) is added. The reaction mixture is then cooled to −78 °C. Triflic anhydride (1.5 equiv., 15.94 g, 56.5 mmol) is added dropwise to the cooled mixture. The resulting mixture is allowed to stir at −78 °C for 4 hours. The reaction is monitored by LCMS. Upon completion, ice-cold water is added to the reaction mixture. The organic layer is separated and washed with water. The organics are then dried with sodium sulfate (Na₂SO₄) and concentrated to dryness. The crude product is purified by flash column chromatography, eluting with 10% ethyl acetate in hexane. The desired fractions are concentrated in vacuo to afford the product D-2a.

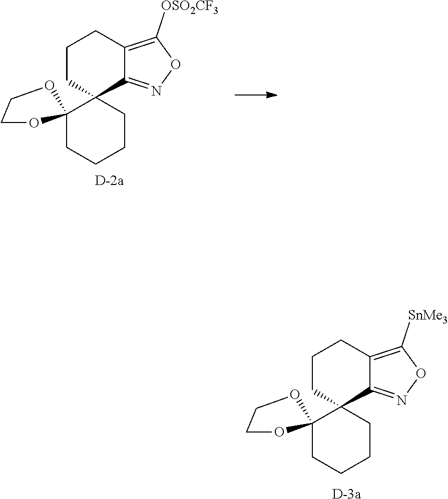

A solution of D-2a (40 g, 101 mmol, 1.0 equiv.) in 1,4-dioxane (400 mL) is degassed with nitrogen for 15-20 minutes. To this, lithium chloride (3.0 equiv., 12.68 g, 302 mmol) and hexamethylditin (1.3 equiv., 42.86 g, 131 mmol) are added. The reaction mixture is stirred at room temperature under a nitrogen atmosphere for 15 minutes. Tetrakis(triphenylphosphine)palladium(0) (0.08 equiv., 9.305 g, 8.05 mmol) is then added. The reaction mixture is heated to 100 °C and maintained at this temperature for 3 hours. Upon completion, the reaction mixture is filtered through a celite pad and washed with a 10% solution of ethyl acetate in petroleum ether. The filtrate is then concentrated under reduced pressure to afford the crude product. The crude product is purified by reverse phase column chromatography to give the desired product D-3a (HPLC method AF, tᵣₑₜ=5.18 min: [M+H]=414).

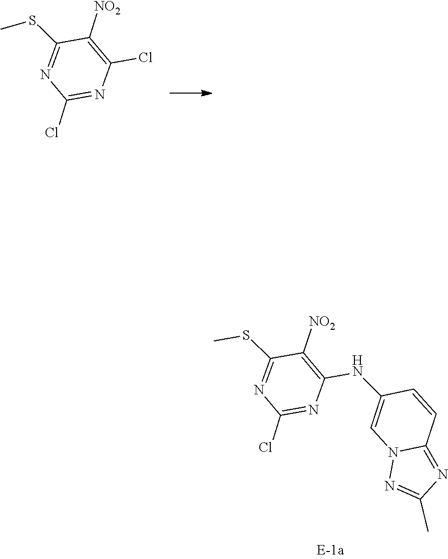 [SMILES: CSc1nc(Cl)nc(Cl)c1[N+](=O)[O-] ; CSc1nc(Cl)nc(Nc2ccc3nc(C)nn3c2)c1[N+](=O)[O-]]

A solution of 2-methyl-[1,2,4]triazolo[1,5-a]pyridin-6-amine (50 g, 337 mmol, 1.0 equiv.) in acetonitrile (250 mL) is stirred at room temperature. To this, DIPEA (2.0 equiv., 87.22 g, 675 mmol) is added and the mixture is stirred for 1 h. 2,4-dichloro-6-methylsulfanyl-5-nitro-pyrimidine (1.1 equiv., 89.11 g, 371 mmol) is then added and the reaction is stirred for an additional 4 h. Upon completion, the reaction mixture is diluted with ice-cold water and filtered. The solid is washed with ice-cold water and dried under vacuum to afford the product, E-1a, as a light yellow solid (HPLC method AD, tᵣₑₜ=1.07 min; [M+H]=352). The following products E-1 (Table 8) are available in an analogous manner.

TABLE 8

| | | tᵣₑₜ | | HPLC |
|---|---|---|---|---|
| # | structure | [min] | [M + H]⁺ | method |
| E- 1a | 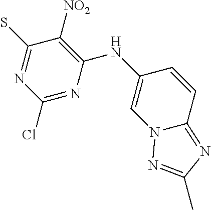 [SMILES: Cc1nc2ccc(Nc3nc(Cl)nc(S)c3[N+](=O)[O-])cn2n1] | 1.07 | 352 | AD |
| E- 1b | 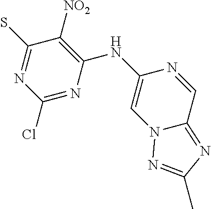 [SMILES: Cc1nc2cnc(Nc3nc(Cl)nc(S)c3[N+](=O)[O-])cn2n1] | 4.65 | 353 | AF |

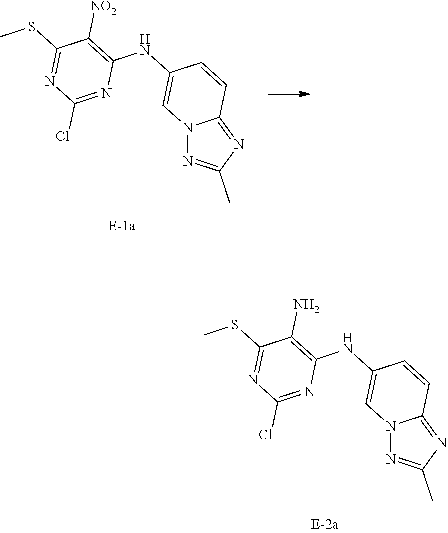 [SMILES: CSc1nc(Cl)nc(Nc2ccc3nc(C)nn3c2)c1[N+](=O)[O-] ; CSc1nc(Cl)nc(Nc2ccc3nc(C)nn3c2)c1N]

A solution of E-1a (350 g, 995 mmol, 1.0 equiv.) in ethanol (2800 mL) and water (700 mL) is stirred. To this, iron powder (3.0 equiv., 166.71 g, 2985 mmol) and ammonium chloride (3.0 equiv., 92.53 g, 2985 mmol) are added. The mixture is then stirred at 90 °C for 16 hours. The progress of the reaction is monitored by TLC and LCMS. Upon completion, the reaction mixture is concentrated under vacuum and passed through a celite bed, washing with ethyl acetate (500 mL). The residue is diluted with water (300 mL) and extracted with ethyl acetate (2×250 mL). The combined organic layers are washed with water (200 mL) and brine (200 mL), dried over Na₂SO₄, and the solvent is evaporated to give the crude product. The crude product is passed through basic silica gel (60-100 mesh) and eluted with a 5% methanol/DCM mixture to give the product E-2a (HPLC method AD, tᵣₑₜ=2.53 min; [M+H]=322).

The following products E-2 (Table 9) are available in an analogous manner.

TABLE 9

| | | tᵣₑₜ | | HPLC |
|---|---|---|---|---|
| # | structure | [min] | [M + H]⁺ | method |
| E- 2a | 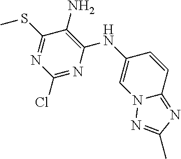 [SMILES: CSc1nc(Cl)nc(Nc2ccc3nc(C)nn3c2)c1N] | 2.53 | 322 | AD |
| E- 2b | 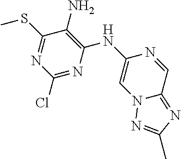 [SMILES: CSc1nc(Cl)nc(Nc2cn3nc(C)nc3cn2)c1N] | 4.27 | 323 | AF |

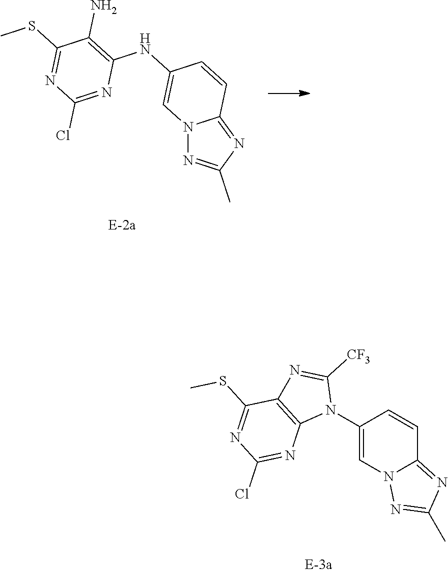

A solution of E-2a (283 g, 879 mmol, 1.0 equiv.) in 1,2-dichloroethane (2264 mL) is stirred at room temperature. To this, triethylamine (3.0 equiv., 267 g, 2640 mmol) is added, followed by the addition of trifluoroacetic anhydride (2.0 equiv., 369.4 g, 1760 mmol) in 1,2-dichloroethane (476 mL). The reaction mixture is stirred at room temperature for 2 hours. The progress of the reaction is monitored by TLC. Upon completion, the reaction mixture is concentrated under vacuum. The residue is diluted with cold water (500 mL) and extracted with ethyl acetate (2×500 mL). The combined organic layers are washed with water (500 mL) and brine (500 mL), dried over sodium sulfate, and the solvent is evaporated to give the crude product. The crude product is purified by flash column chromatography using 100-200 mesh silica gel and 30% ethyl acetate in petroleum ether as the eluent to give the product E-3a (HPLC method AD, tᵣₑₜ=2.72 min; [M+H]=400).

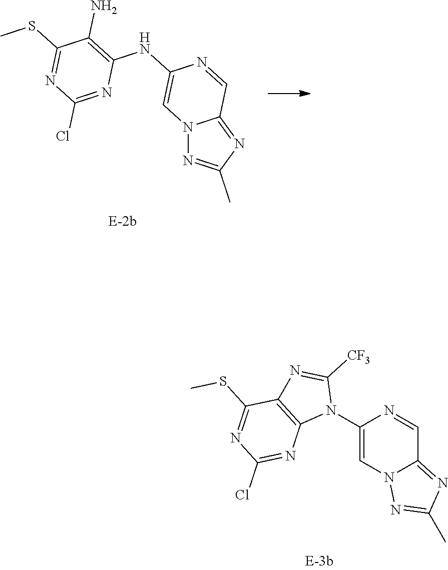

A solution of E-2b (4 g, 12.4 mmol, 1.0 equiv.) in toluene (60 mL) is stirred at 0 °C. To this, p-toluenesulfonic acid (1.18 g, 6.2 mmol, 0.5 equiv.) and trifluoroacetic anhydride (8 mL, 57.1 mmol, 4.61 equiv.) are added. The reaction mixture is stirred at 120 °C for 36 hours. The progress of the reaction is monitored by TLC. Upon completion, the reaction mixture is concentrated under vacuum to give a residue. The residue is basified with aqueous sodium bicarbonate solution and extracted with ethyl acetate. The organic layer is dried over sodium sulfate and concentrated under vacuum to give the crude product. The crude product is purified by flash column chromatography, eluting with 30% ethyl acetate in petroleum ether, to give the pure product E-3b as a solid (H PLC method AF, tᵣₑₜ=4.64 min; [M+H]=401).

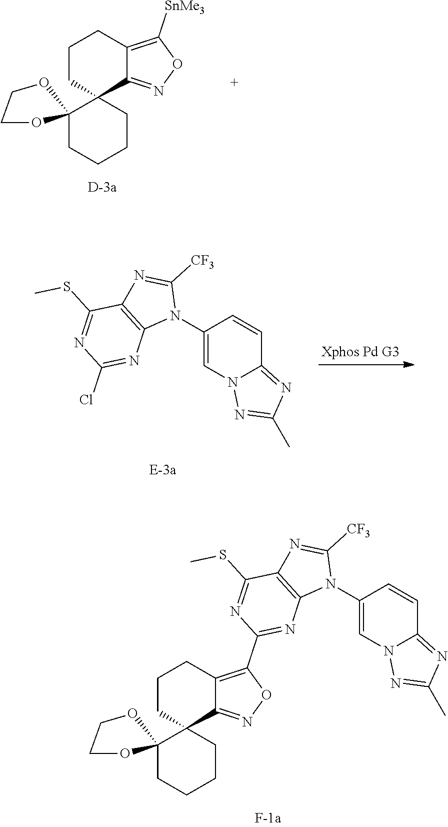

A solution of E-1a (32 g, 80 mmol, 1.0 equiv.) and D-3a (49.48 g, 120 mmol, 1.5 equiv.) in 1,4-dioxane (350 mL) is degassed under a nitrogen atmosphere for 10 minutes. To this, Xphos Pd G3 (6.78 g, 8 mmol, 0.1 equiv.) is added at room temperature. The mixture is then degassed under a nitrogen atmosphere for another 10 minutes and stirred at 120 °C in a microwave for 2 h. The progress of the reaction is monitored using TLC (70% ethyl acetate/hexane, Rf=0.5). Upon completion, the reaction mixture is filtered through celite and the solvent is evaporated under vacuum to give the crude product. The crude product is purified by flash column chromatography (70% ethyl acetate/hexane) to give the product F-1a, as an off-white solid.

The following products F-1 ( ) are available in an analogous manner.

TABLE 10

| | | tᵣₑₜ | | HPLC |
|---|---|---|---|---|
| # | structure | [min] | [M + H]⁺ | method |
| F-1a | 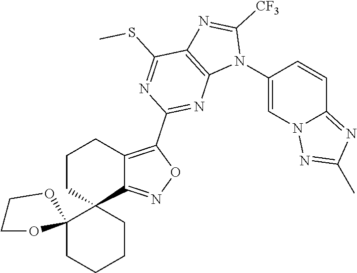 | 2.32 | 613 | AD |
| F-1b | 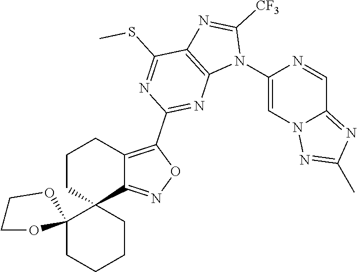 | 4.27 | 614 | AF |

### Coupling of Amines to Building Blocks F-1a/F-1b

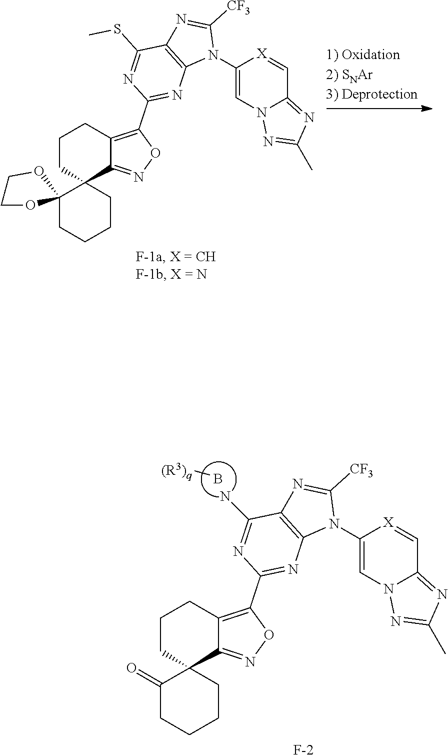

[6-methylsulfanyl-9-(2-methyl-[1,2,4]triazolo[1,5-a]pyridin-6-yl)-8-(trifluoromethyl)purin-2-yl]dispiro (X═CH) F-1a (539.1 mg, 0.880 mmol, 1.0 equiv) is dissolved in CH₂Cl₂ (5 mL), 3-chloroperbenzoic acid (334.1 mg, 1.94 mmol, 2.2 equiv) was added and the reaction mixture was stirred at room temperature for 5 h. After addition of water (20 mL), the phases were separated and the aqueous phase was extracted with CH₂Cl₂ three times. The combined extracts were dried with Na₂SO₄ and evaporated to dryness. The crude product was used without further purification in the S_{N}Ar step.

The corresponding amine (1.5 equiv) and the crude product from above were dissolved in acetonitrile (0.1 M), cesium carbonate (2.0 equiv) was added and the reaction mixture was stirred at 90 °C for 16 h. After addition of water, the phases were separated, and the aqueous phase was extracted with CH₂Cl₂ three times. The combined extracts were dried with Na₂SO₄ and evaporated to dryness. The crude product was used without further purification in the deprotection step.

The crude product from above was dissolved in dioxane (0.2 M) and 6 M hydrochloric acid (16 equiv) was added. The reaction mixture was stirred at 50 °C for 1 hour. After addition of water, the phases were separated, and the aqueous phase was extracted with CH₂Cl₂ three times. The combined extracts were dried with Na₂SO₄ and evaporated to dryness. The crude product was purified by RP-chromatography to give the desired products F-2.

The following products F-2 (Table 11) are available in an analogous manner.

TABLE 11

| | | tᵣₑₜ | | |
|---|---|---|---|---|
| # | structure | [min] | [M + H]⁺ | method |

| | | | | |
|---|---|---|---|---|
| F-2a | 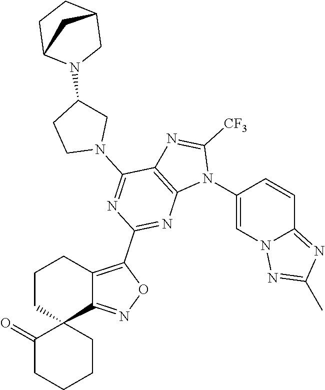 [SMILES: Cc1nc2ccc(-n3c(C(F)(F)F)nc4c(N5CC[C@H](N6C[C@H]7CC[C@@H]6C7)C5)nc(-c5onc6c5CCC[C@@]65CCCCC5=O)nc43)cn2n1] | 1.13 | 687 | A |

| | | | | |
|---|---|---|---|---|
| F-2b | 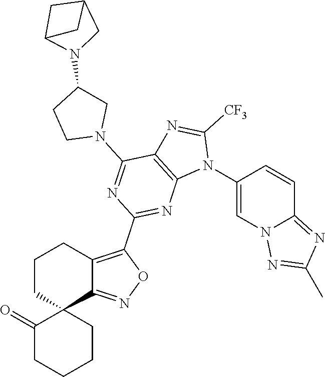 [SMILES: Cc1nc2ccc(-n3c(C(F)(F)F)nc4c(N5CC[C@H](N6CC7CC6C7)C5)nc(-c5onc6c5CCC[C@@]65CCCCC5=O)nc43)cn2n1] | 1.09 | 673 | A |
| F-2c | 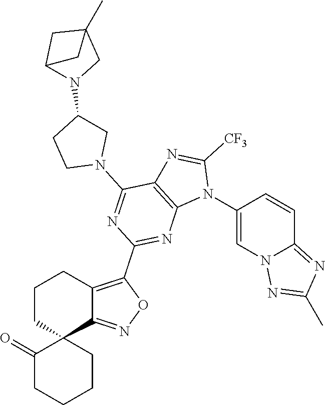 [SMILES: Cc1nc2ccc(-n3c(C(F)(F)F)nc4c(N5CC[C@H](N6CC7(C)CC6C7)C5)nc(-c5onc6c5CCC[C@@]65CCCCC5=O)nc43)cn2n1] | 1.05 | 687 | A |
| F-2d | 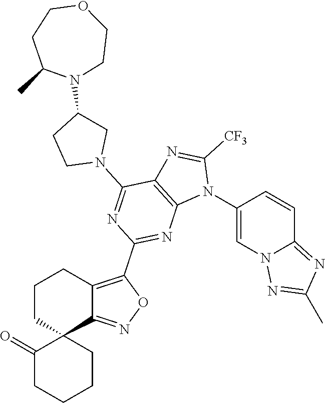 [SMILES: Cc1nc2ccc(-n3c(C(F)(F)F)nc4c(N5CC[C@H](N6CCOCC[C@@H]6C)C5)nc(-c5onc6c5CCC[C@@]65CCCCC5=O)nc43)cn2n1] | 1.09 | 705 | A |
| F-2e | 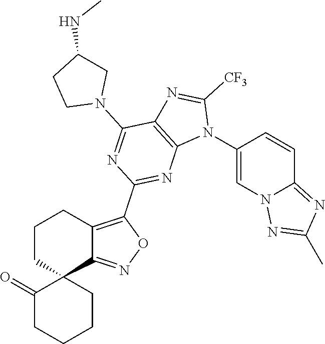 [SMILES: CN[C@H]1CCN(c2nc(-c3onc4c3CCC[C@@]43CCCCC3=O)nc3c2nc(C(F)(F)F)n3-c2ccc3nc(C)nn3c2)C1] | 1.35 | 621 | A |
| F-2f | 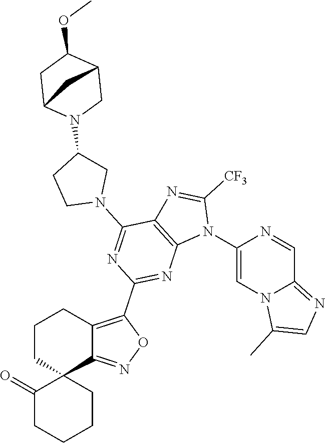 [SMILES: CO[C@@H]1C[C@@H]2C[C@H]1CN2[C@H]1CCN(c2nc(-c3onc4c3CCC[C@@]43CCCCC3=O)nc3c2nc(C(F)(F)F)n3-c2cn3c(C)cnc3cn2)C1] | 1.03 | 717 | C |
| F-2g | 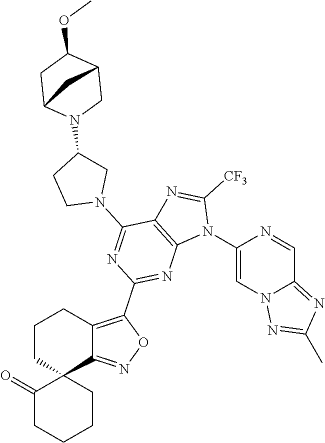 [SMILES: CO[C@@H]1C[C@@H]2C[C@H]1CN2[C@H]1CCN(c2nc(-c3onc4c3CCC[C@@]43CCCCC3=O)nc3c2nc(C(F)(F)F)n3-c2cn3nc(C)nc3cn2)C1] | 1.11 | 718 | C |
| F-2h | 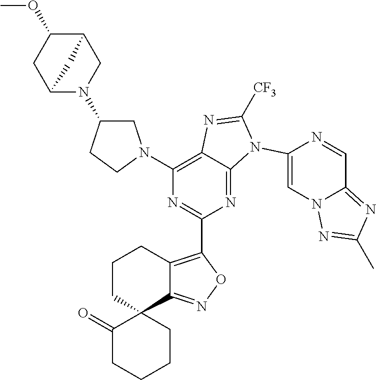 | 1.11 | 718 | C |

### Synthesis of Spiropiperidine Intermediates G-5a and H-1a

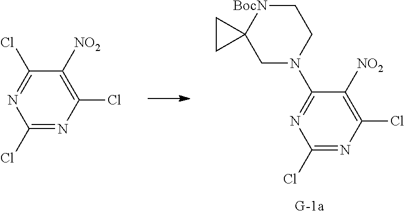 [SMILES: O=[N+]([O-])c1c(Cl)nc(Cl)nc1Cl ; CC(C)(C)OC(=O)N1CCN(c2nc(Cl)nc(Cl)c2[N+](=O)[O-])CC12CC2]

A solution of 2,4,6-trichloro-5-nitro-pyrimidine 2,4,6-Trichloro-5-nitropyrimidine (10 g, 43.8 mmol, 1.0 equiv.) in DCM (100 mL) is stirred at room temperature. To this, N-Ethyldiisopropylamine (48.11 g, 52.5 mmol, 1.20 equiv.) and tert-butyl 4,7-diazaspiro[2.5]octane-4-carboxylate (9.29 g, 43.8 mmol, 1.0 equiv.) are added. The reaction mixture is stirred at room temperature for 1 hour.

After the reaction is complete, the reaction mixture is extracted with DCM (2×100 mL), dried over sodium sulfate, and concentrated under vacuum to afford the crude product. The crude compound is purified by column chromatography on silica gel, eluting the desired compound G-1a (HPLC method A, tᵣₑₜ=1.60 min: [M+H]=404).

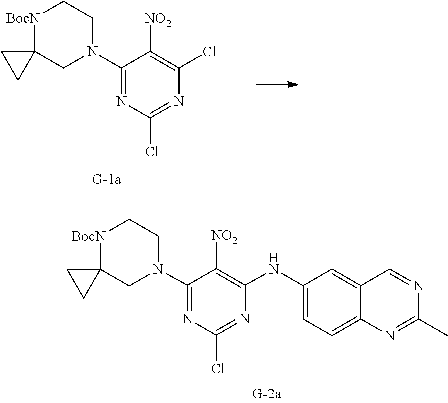 [SMILES: CC(C)(C)OC(=O)N1CCN(c2nc(Cl)nc(Cl)c2[N+](=O)[O-])CC12CC2 ; Cc1ncc2cc(Nc3nc(Cl)nc(N4CCN(C(=O)OC(C)(C)C)C5(CC5)C4)c3[N+](=O)[O-])ccc2n1]

A solution of G-1a (1.00 g, 2.29 mmol, 1.0 equiv.) and 2-methylquinazolin-6-amine (384 mg, 2.29 mmol, 1.0 equiv.) in THF (5 mL) is treated with DIPEA (0.50 ml, 2.86 mmol, 1.3 equiv.) and shaked until it is a clear solution. Then it is put into the fridge at 4 °C overnight.

The reaction mixture is then diluted with DCM and extracted with an ammonium chloride solution. The organic phase is dried with magnesium sulfate, filtered, and the solvents are removed under reduced pressure to afford the product G-2a (HPLC method A, tᵣₑₜ=1.05 min; [M+H]=527).

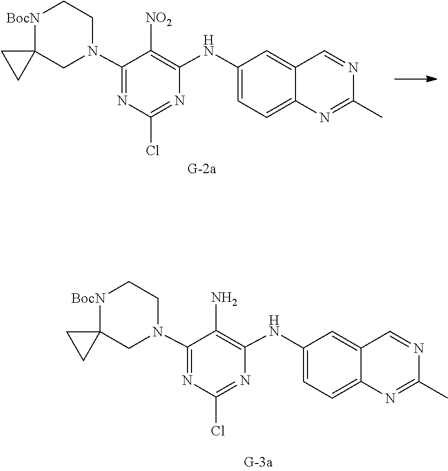 [SMILES: Cc1ncc2cc(Nc3nc(Cl)nc(N4CCN(C(=O)OC(C)(C)C)C5(CC5)C4)c3[N+](=O)[O-])ccc2n1 ; Cc1ncc2cc(Nc3nc(Cl)nc(N4CCN(C(=O)OC(C)(C)C)C5(CC5)C4)c3N)ccc2n1]

To a suspension of G-2a (100 mg, 0.18 mmol, 1.0 equiv.) in THF (20 mL), sponge nickel catalyst (50% aq. slurry, 21 mg, 0.18 mmol, 1.0 equiv.) is added. The reaction is flushed 3 times with N₂, then filled with 3 bar H₂ and stirred for 4 h at room temperature. The reaction is filtered over celite and concentrated under reduced pressure to afford G-3a without further purification. (HPLC method B, tᵣₑₜ=1.02 min; [M+H]=497).

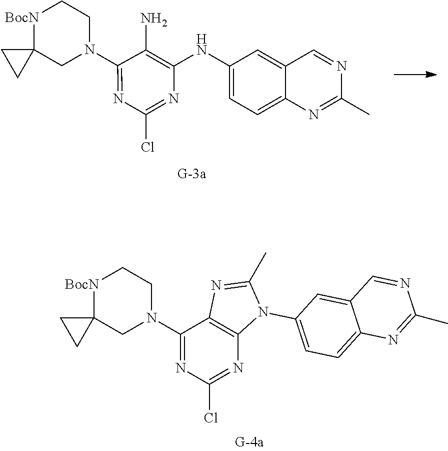 [SMILES: Cc1ncc2cc(Nc3nc(Cl)nc(N4CCN(C(=O)OC(C)(C)C)C5(CC5)C4)c3N)ccc2n1 ; Cc1ncc2cc(-n3c(C)nc4c(N5CCN(C(=O)OC(C)(C)C)C6(CC6)C5)nc(Cl)nc43)ccc2n1]

A solution of G-3a (170 mg, 0.33 mmol, 1.0 equiv.) in acetic acid (1.9 mL) is treated with trimethyl orthoacetate (0.16 mL, 1.27 mmol, 3.9 equiv.) and stirred at room temperature for 18 hours.

The reaction mixture is directly loaded onto Isolute and purified with NP chromatography to give G-4a (HPLC method A, tᵣₑₜ=1.51 min; [M+H]=522).

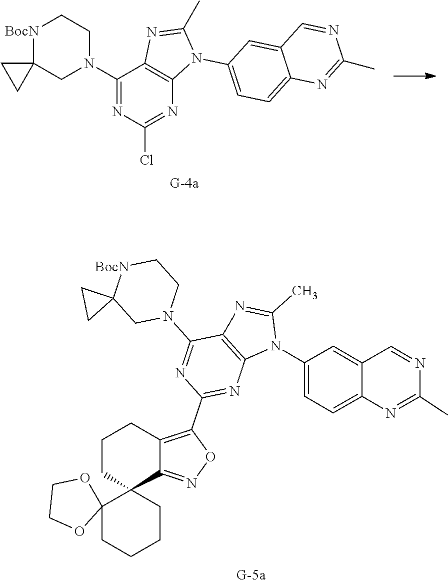 [SMILES: Cc1ncc2cc(-n3c(C)nc4c(N5CCN(C(=O)OC(C)(C)C)C6(CC6)C5)nc(Cl)nc43)ccc2n1 ; Cc1ncc2cc(-n3c(C)nc4c(N5CCN(C(=O)OC(C)(C)C)C6(CC6)C5)nc(-c5onc6c5CCC[C@@]65CCCCC56OCCO6)nc43)ccc2n1]

A solution of G-4a (200 mg, 0.365 mmol, 1.0 equiv.), D-3a (200 mg, 0.461 mmol, 1.3 equiv.), XPhos Pd G3 (40.0 mg, 0.046 mmol, 0.13 equiv.) and XPhos (20 mg, 0.041 mmol, 0.11 equiv.) in dioxane (5 mL) is degassed with argon and heated in a microwave at 90 °C overnight.

The reaction mixture is then concentrated under reduced pressure, dissolved in DCM, filtered, and purified by NP chromatography. The product fractions are then concentrated under reduced pressure and lyophilized to afford G-5a (HPLC method A, tᵣₑₜ=1.78 min; [M+H]=743)

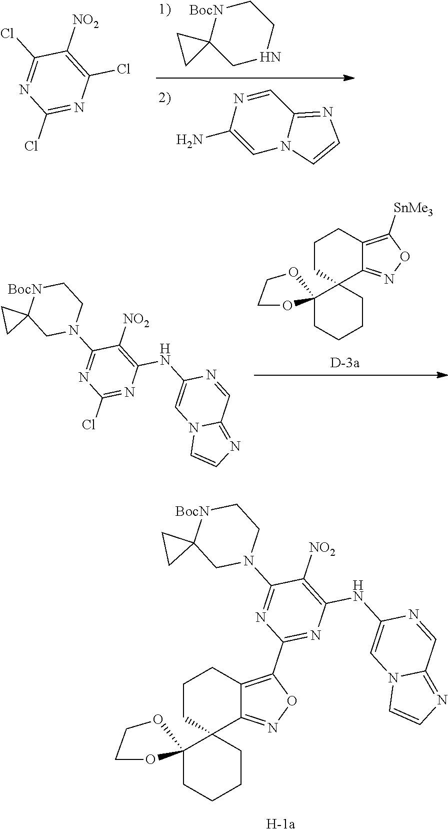

A solution of 2,4,6-trichloro-5-nitropyrimidine (3.00 g, 13.1 mmol, 1.0 equiv.) in DCM (20 mL) is cooled to −10 °C. To this, a solution of tert-butyl 4,7-diazaspiro[2.5]octane-4-carboxylate (2.51 g, 11.8 mmol, 0.90 equiv.) and DIPEA (2.04 g, 15.8 mmol, 1.2 equiv.) in CH₂Cl₂ (10 mL) is added slowly. The reaction mixture is stirred at 0 °C for 1 hour and then at room temperature for 2 hours. The reaction mixture is then diluted with additional DCM and extracted with an aqueous solution of ammonium chloride. The organic phase is dried with magnesium sulfate, filtered and the solvents are removed under reduced pressure. The crude monosubstituted nitropyrimidine (1.30 g, 3.22 mmol, 1 equiv) is redissolved in THF (11.5 mL) and imidazo[1,2-a]pyrazin-6-amine (500 mg, 3.54 mmol, 1.1 equiv) is added and cooled to 0 °C. LiHMDS solution in THF (6.4 mL, 6.43 mmol, 2.0 equiv) is added and the reaction mixture is stirred at room temperature for 1 h. The reaction mixture was diluted with CH₂Cl₂ and aqeuous NaHCO₃ solution and extracted with CH₂Cl₂ three times. The combined extracts were dried with Na₂SO₄ and evaporated to dryness.

The disubstituted pyrimidine (30 mg, 0.0587 mmol, 1.0 equiv.), D-3a (34.18 mg, 0.0763 mmol, 1.3 equiv.), XPHOS Pd G4 (10.31 mg, 0.0117 mmol, 0.20 equiv.), and XPHOS (5.71 mg, 0.0117 mmol, 0.20 equiv.) is placed in a microwave vial. To this, 1,4-dioxane (1 mL) is added. The reaction mixture is flushed with argon for 5 minutes (in an ultrasound bath) and then heated to 120 °C in a microwave for 25 minutes.

The reaction mixture is then directly loaded onto an Isolute column and purified by reverse phase chromatography to give the desired product product H-1a.

The following intermediate compounds H-1 (Table 12) are obtained analogously.

TABLE 12

| | | tᵣₑₜ | | |
|---|---|---|---|---|
| # | structure | [min] | [M + H]⁺ | method |
| H-1a | 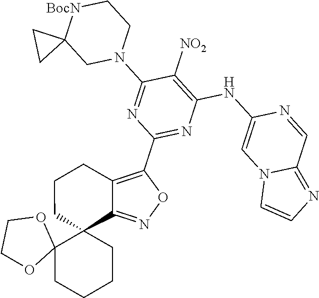 [SMILES: CC(C)(C)OC(=O)N1CCN(c2nc(-c3onc4c3CCC[C@@]43CCCCC34OCCO4)nc(Nc3cn4ccnc4cn3)c2[N+](=O)[O-])CC12CC2] | 1.22 | 715 | A |
| H-1b | 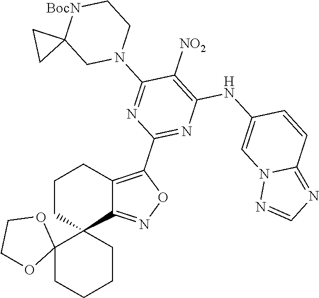 [SMILES: CC(C)(C)OC(=O)N1CCN(c2nc(-c3onc4c3CCC[C@@]43CCCCC34OCCO4)nc(Nc3ccc4ncnn4c3)c2[N+](=O)[O-])CC12CC2] | 1.22 | 715 | A |
| H-1c | 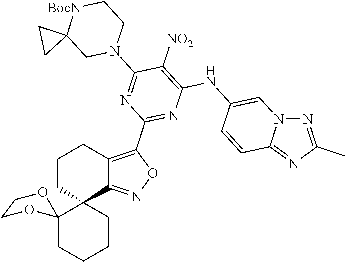 [SMILES: Cc1nc2ccc(Nc3nc(-c4onc5c4CCC[C@@]54CCCCC45OCCO5)nc(N4CCN(C(=O)OC(C)(C)C)C5(CC5)C4)c3[N+](=O)[O-])cn2n1] | 1.23 | 729 | C |

### Synthesis of Purine Intermediates H-3a

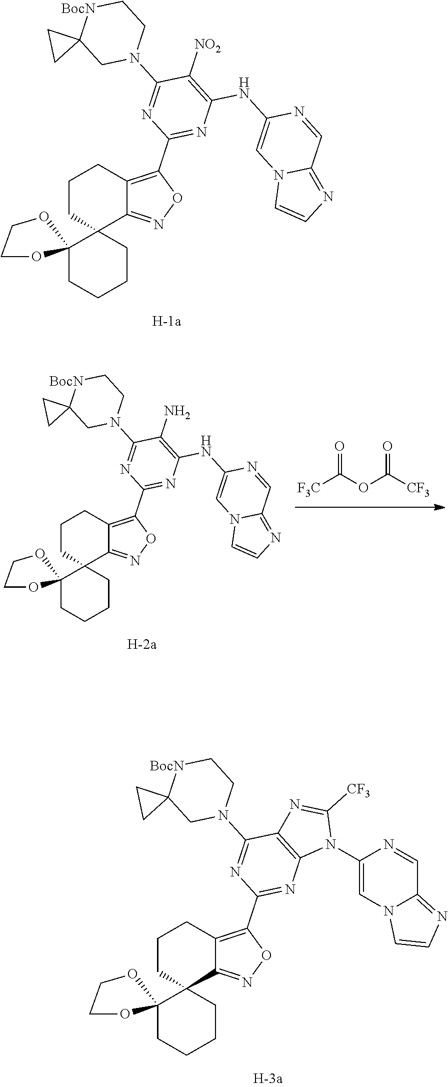

H-1a (1.40 g, 1.96 mmol, 1.0 equiv) and Pd/C 10% are suspended in THF (200 mL) and stirred under hydrogen pressure (5 bar) for 4.5 hour at room temperature. The reaction mixture is filtered over celite and evaporated to dryness. H-2a is directly used in the next step without purification.

H-2a (800 mg, 1.17 mmol, 1.0 equiv) was dissolved in CH₂Cl₂ (20 mL) and cooled to −20 °C. Triethyl amine (0.49 mL, 3.50 mmol, 3.0 equiv) and trifluoroacetic anhydride (0.34 mL, 2.45 mmol, 2.0 equiv) were added sequentially and the reaction mixture is stirred for 20 min at −20 °C. The mixture was quenched with water and then extracted with CH₂Cl₂ three times. The combined extracts were dried with Na₂SO₄ and evaporated to dryness. The crude product was purified by RP-chromatography to give the desired product product H-3a.

The following intermediate compounds H-3 (Table 13) are obtained analogously. For H-3e Difluoric anhydride is used.

TABLE 13

| | | tᵣₑₜ | | HPLC |
|---|---|---|---|---|
| # | structure | [min] | [M + H]⁺ | method |
| H-3a | 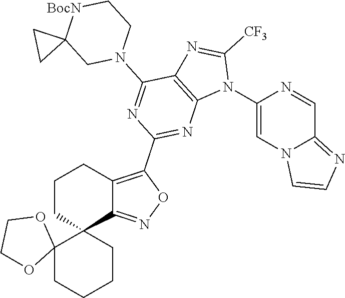 [SMILES: CC(C)(C)OC(=O)N1CCN(c2nc(-c3onc4c3CCC[C@@]43CCCCC34OCCO4)nc3c2nc(C(F)(F)F)n3-c2cn3ccnc3cn2)CC12CC2] | 1.22 | 763 | A |
| H-3b | 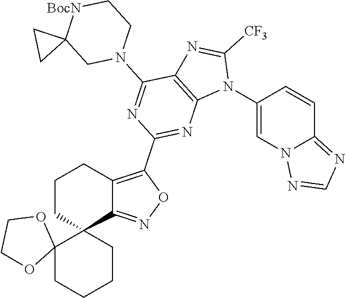 [SMILES: CC(C)(C)OC(=O)N1CCN(c2nc(-c3onc4c3CCC[C@@]43CCCCC34OCCO4)nc3c2nc(C(F)(F)F)n3-c2ccc3ncnn3c2)CC12CC2] | 1.24 | 763 | A |
| H-3c | 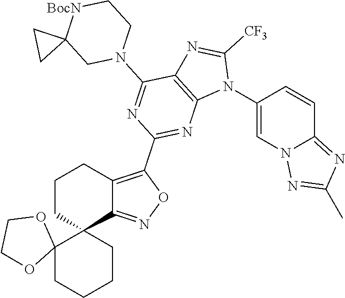 [SMILES: Cc1nc2ccc(-n3c(C(F)(F)F)nc4c(N5CCN(C(=O)OC(C)(C)C)C6(CC6)C5)nc(-c5onc6c5CCC[C@@]65CCCCC56OCCO6)nc43)cn2n1] | 1.26 | 777 | C |
| H-3d | 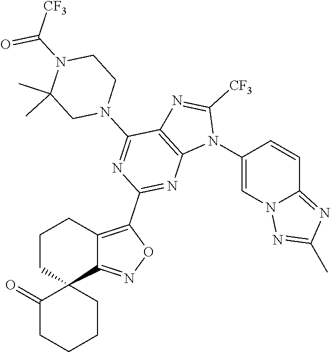 [SMILES: Cc1nc2ccc(-n3c(C(F)(F)F)nc4c(N5CCN(C(=O)C(F)(F)F)C(C)(C)C5)nc(-c5onc6c5CCC[C@@]65CCCCC5=O)nc43)cn2n1] | 1.13 | 731 | C |
| H-3e | 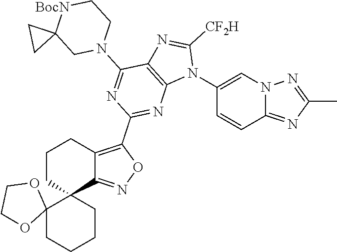 [SMILES: Cc1nc2ccc(-n3c(C(F)F)nc4c(N5CCN(C(=O)OC(C)(C)C)C6(CC6)C5)nc(-c5onc6c5CCC[C@@]65CCCCC56OCCO6)nc43)cn2n1] | 1.26 | 759 | C |

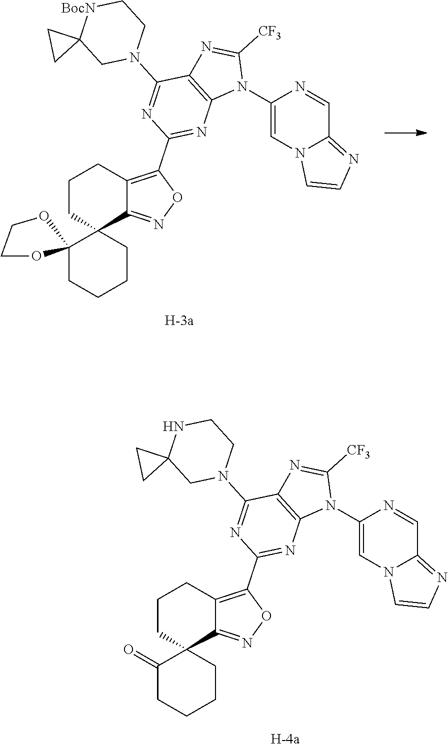

H-3a (1.00 g, 1.18 mmol, 1.0 equiv) is dissolved in dioxane (20 mL), followed by addition of 6 M HCl in dioxane (6.8 mL, 40.8 mmol, 34.6 equiv). The reaction mixture is stirred for 90 min at 60 °C, before it is quenched with NaHCO₃ solution. The suspension is filtered over celite and extracted with CH₂Cl₂ three times. The combined extracts are dried with Na₂SO₄ and evaporated to dryness. The crude product is purified by RP-chromatography to give the desired product H-4a.

The following intermediate compounds G-5 and H-4 ( ) are obtained analogously. H-4e is stirred at 95 °C overnight.

TABLE 14

| | | tᵣₑₜ | | HPLC |
|---|---|---|---|---|
| # | structure | [min] | [M + H]⁺ | method |
| H-4a | 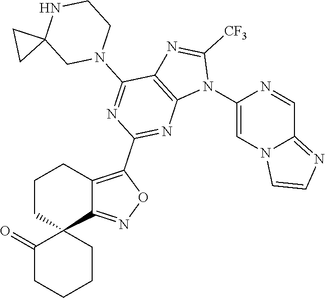 [SMILES: O=C1CCCC[C@@]12CCCc1c2noc1-c1nc(N2CCNC3(CC3)C2)c2nc(C(F)(F)F)n(-c3cn4ccnc4cn3)c2n1] | 0.94 | 619 | A |
| H-4b | 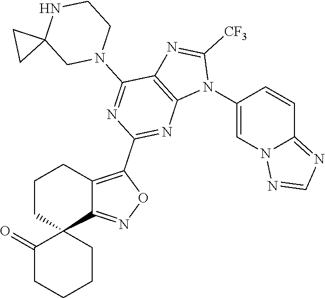 [SMILES: O=C1CCCC[C@@]12CCCc1c2noc1-c1nc(N2CCNC3(CC3)C2)c2nc(C(F)(F)F)n(-c3ccc4ncnn4c3)c2n1] | 0.94 | 619 | A |
| H-4c | 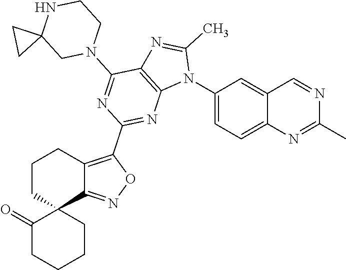 [SMILES: Cc1ncc2cc(-n3c(C)nc4c(N5CCNC6(CC6)C5)nc(-c5onc6c5CCC[C@@]65CCCCC5=O)nc43)ccc2n1] | 1.26 | 590 | A |
| H-4d | 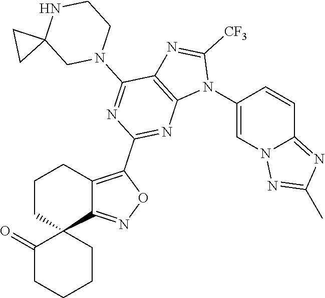 [SMILES: Cc1nc2ccc(-n3c(C(F)(F)F)nc4c(N5CCNC6(CC6)C5)nc(-c5onc6c5CCC[C@@]65CCCCC5=O)nc43)cn2n1] | 0.97 | 633 | C |
| H-4e | 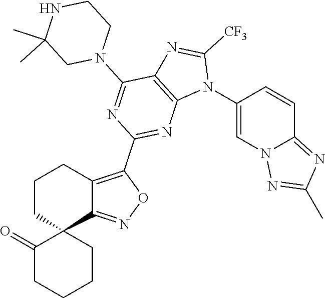 [SMILES: Cc1nc2ccc(-n3c(C(F)(F)F)nc4c(N5CCNC(C)(C)C5)nc(-c5onc6c5CCC[C@@]65CCCCC5=O)nc43)cn2n1] | 0.99 | 635 | C |
| H-4f | 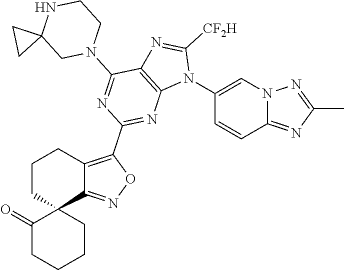 [SMILES: Cc1nc2ccc(-n3c(C(F)F)nc4c(N5CCNC6(CC6)C5)nc(-c5onc6c5CCC[C@@]65CCCCC5=O)nc43)cn2n1] | 1.00 | 615 | A |

### Reductive Amination

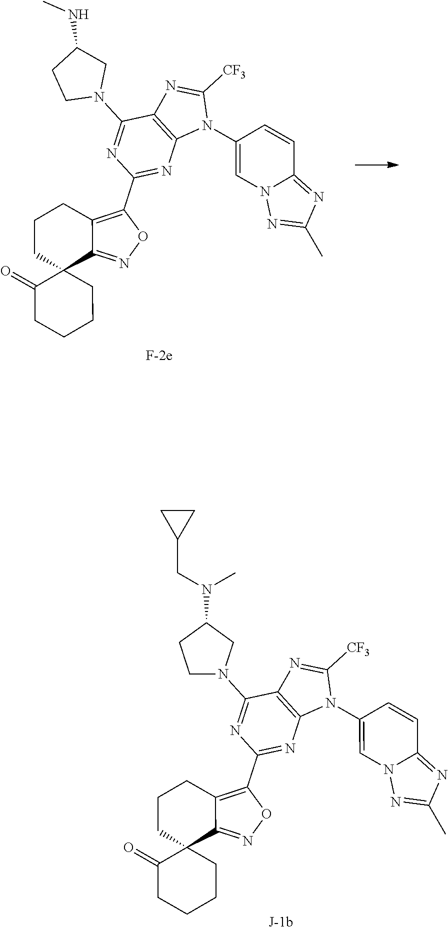 [SMILES: CN[C@H]1CCN(c2nc(-c3onc4c3CCC[C@@]43CCCCC3=O)nc3c2nc(C(F)(F)F)n3-c2ccc3nc(C)nn3c2)C1 ; Cc1nc2ccc(-n3c(C(F)(F)F)nc4c(N5CC[C@H](N(C)CC6CC6)C5)nc(-c5onc6c5CCC[C@@]65CCCCC5=O)nc43)cn2n1]

A solution of F-2e (70 mg, 0.11 mmol, 1.0 equiv.) in methanol is stirred with cyclopropanecarboxaldehyde (16.3 mg, 0.23 mmol, 2.0 equiv.) and a drop of acetic acid for 5 minutes. Sodium triacetoxy borohydride (49.3 mg, 0.23 mmol, 2.0 equiv.) is added and the reaction is allowed to proceed for 30 minutes. The reaction mixture is diluted with an aqueous NaHCO₃-solution and then extracted with 0H₂Cl₂ three times. The combined extracts were dried with Na₂SO₄ and evaporated to dryness. The crude product was purified by RP-chromatography to give the desired product J-1b.

The following intermediate compounds F-2 and J-1 (Table 15) are obtained analogously using the indicated carbonyl component.

TABLE 15

| | | tᵣₑₜ | | HPLC | Carbonyl |
|---|---|---|---|---|---|
| # | structure | [min] | [M + H]⁺ | method | component |
| J-1a | 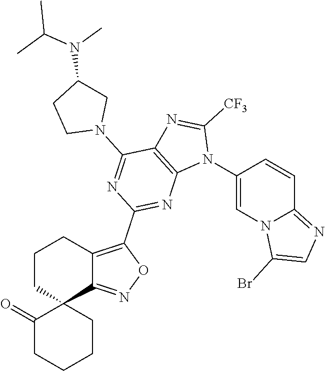 [SMILES: CC(C)N(C)[C@H]1CCN(c2nc(-c3onc4c3CCC[C@@]43CCCCC3=O)nc3c2nc(C(F)(F)F)n3-c2ccc3ncc(Br)n3c2)C1] | 1.17 | 726 | C | 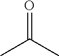 |
| J-1b | 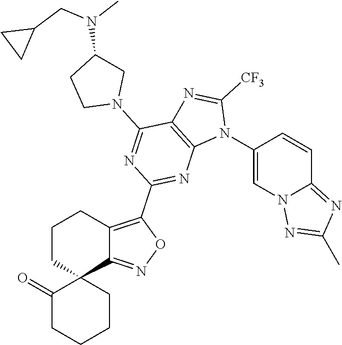 [SMILES: Cc1nc2ccc(-n3c(C(F)(F)F)nc4c(N5CC[C@H](N(C)CC6CC6)C5)nc(-c5onc6c5CCC[C@@]65CCCCC5=O)nc43)cn2n1] | 1.17 | 675 | A | 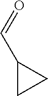 |
| J-1c | 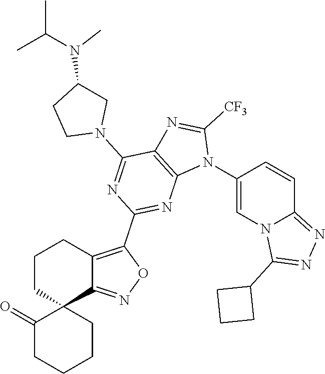 [SMILES: CC(C)N(C)[C@H]1CCN(c2nc(-c3onc4c3CCC[C@@]43CCCCC3=O)nc3c2nc(C(F)(F)F)n3-c2ccc3nnc(C4CCC4)n3c2)C1] | 1.11 | 703 | A | 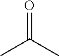 |
| J-1d | 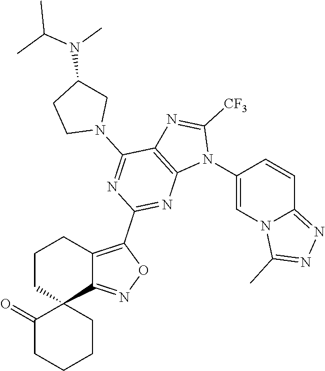 [SMILES: Cc1nnc2ccc(-n3c(C(F)(F)F)nc4c(N5CC[C@H](N(C)C(C)C)C5)nc(-c5onc6c5CCC[C@@]65CCCCC5=O)nc43)cn12] | 1.10 | 663 | A | 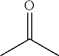 |
| J-1e | 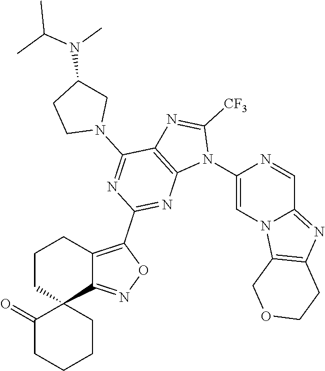 [SMILES: CC(C)N(C)[C@H]1CCN(c2nc(-c3onc4c3CCC[C@@]43CCCCC3=O)nc3c2nc(C(F)(F)F)n3-c2cn3c4c(nc3cn2)CCOC4)C1] | 1.13 | 705 | A | 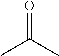 |
| J-1f | 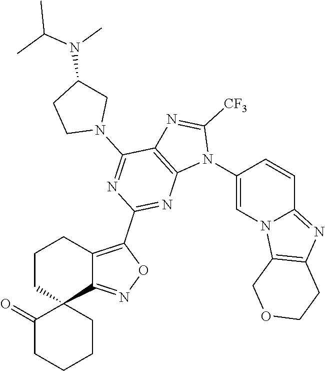 [SMILES: CC(C)N(C)[C@H]1CCN(c2nc(-c3onc4c3CCC[C@@]43CCCCC3=O)nc3c2nc(C(F)(F)F)n3-c2ccc3nc4c(n3c2)COCC4)C1] | 1.09 | 704 | A | 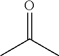 |
| J-1g | 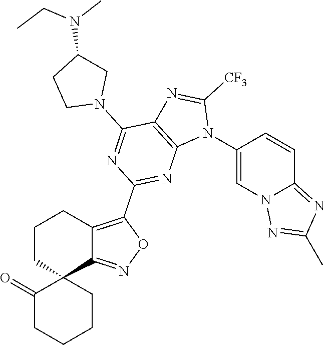 [SMILES: CCN(C)[C@H]1CCN(c2nc(-c3onc4c3CCC[C@@]43CCCCC3=O)nc3c2nc(C(F)(F)F)n3-c2ccc3nc(C)nn3c2)C1] | 1.09 | 649 | A | 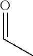 |
| J-1h | 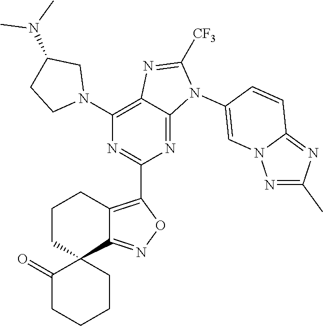 | 1.03 | 635 | C | 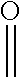 |

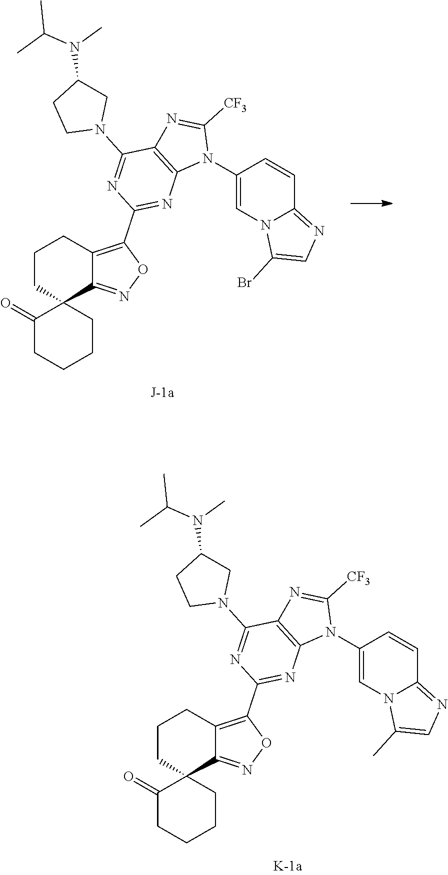 [SMILES: CC(C)N(C)[C@H]1CCN(c2nc(-c3onc4c3CCC[C@@]43CCCCC3=O)nc3c2nc(C(F)(F)F)n3-c2ccc3ncc(Br)n3c2)C1 ; Cc1cnc2ccc(-n3c(C(F)(F)F)nc4c(N5CC[C@H](N(C)C(C)C)C5)nc(-c5onc6c5CCC[C@@]65CCCCC5=O)nc43)cn12]

J-1a (50 mg, 0.07 mmol, 1.0 equiv.), sodium carbonate (15 mg, 0.14 mmol, 2.2 equiv.) and Tetrakis(triphenylphosphine)palladium(0) (5 mg, 0.004 mmol, 0.06 equiv.) are dissolved in a mixture of dioxane (1 mL) and water (0.5 mL). Trimethylboroxine (0.01 mL, 0.07 mmol, 1.1 equiv.) is then added. The orange/brown reaction mixture is degassed with argon and heated in the microwave to 120 °C for 1 hour. After filtration, the crude product was purified by RP-chromatography to give the desired product K-1a (HPLC method A, tᵣₑₜ=1.08 min; [M+H]=661).

### Synthesis of Final Compounds II

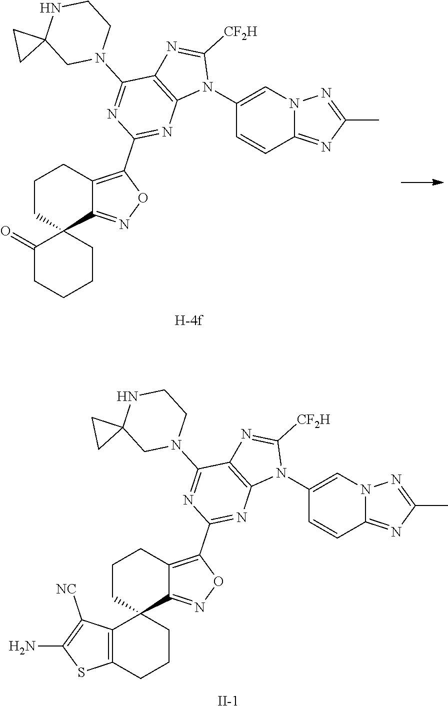 [SMILES: Cc1nc2ccc(-n3c(C(F)F)nc4c(N5CCNC6(CC6)C5)nc(-c5onc6c5CCC[C@@]65CCCCC5=O)nc43)cn2n1 ; Cc1nc2ccc(-n3c(C(F)F)nc4c(N5CCNC6(CC6)C5)nc(-c5onc6c5CCC[C@@]65CCCc6sc(N)c(C#N)c65)nc43)cn2n1 ; "II-1"]

A solution of H-4f (116 mg, 0.19 mmol, 1.0 equiv.) in dry ethanol (2.75 mL) is added to a mixture of malononitrile (25.4 mg, 0.38 mmol, 2.0 equiv.), sulfur (12.1 mg, 0.38 mmol, 2.0 equiv.), and ammonium acetate (29.1 mg, 0.38 mmol, 2.0 equiv.). The reaction mixture is stirred at 60 °C for 4.75 hours. HPLC-MS shows full conversion of the starting material to the product after 3 hours. The reaction mixture is directly loaded onto Isolute and purified by RP-chromatography to give the desired product I-1.

The following final compounds II (Table 16) are obtained analogously from the respective compounds F-2, H-4, C-5, J-1, or K-1. The crude product is purified by chromatography if necessary.

TABLE 16

| | | tᵣₑₜ | | HPLC |
|---|---|---|---|---|
| # | structure | [min] | [M + H]⁺ | method |
| II-1 | 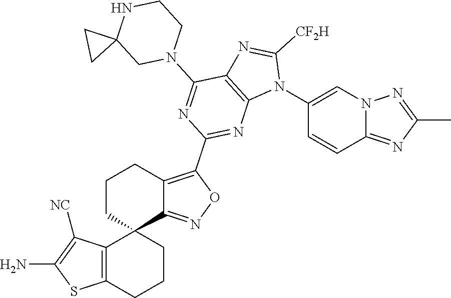 [SMILES: Cc1nc2ccc(-n3c(C(F)F)nc4c(N5CCNC6(CC6)C5)nc(-c5onc6c5CCC[C@@]65CCCc6sc(N)c(C#N)c65)nc43)cn2n1] | 1.00 | 695 | A |
| II-2 | 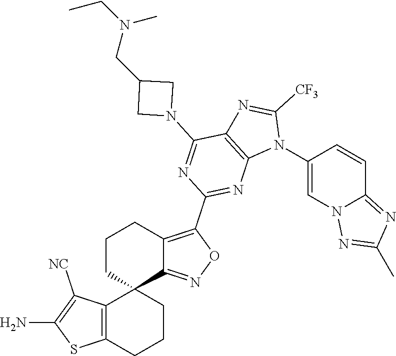 [SMILES: CCN(C)CC1CN(c2nc(-c3onc4c3CCC[C@@]43CCCc4sc(N)c(C#N)c43)nc3c2nc(C(F)(F)F)n3-c2ccc3nc(C)nn3c2)C1] | 1.10 | 729 | A |
| II-3 | 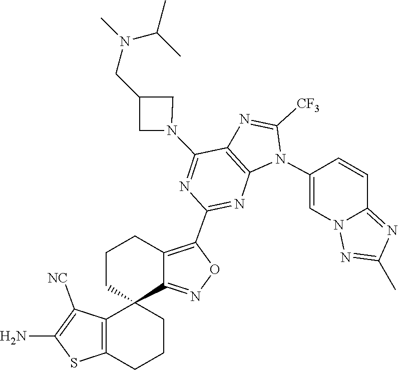 [SMILES: Cc1nc2ccc(-n3c(C(F)(F)F)nc4c(N5CC(CN(C)C(C)C)C5)nc(-c5onc6c5CCC[C@@]65CCCc6sc(N)c(C#N)c65)nc43)cn2n1] | 1.05 | 743 | A |
| II-4 | 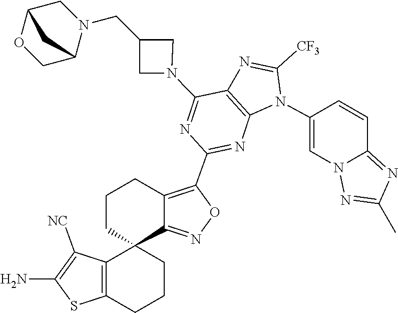 [SMILES: Cc1nc2ccc(-n3c(C(F)(F)F)nc4c(N5CC(CN6C[C@@H]7C[C@H]6CO7)C5)nc(-c5onc6c5CCC[C@@]65CCCc6sc(N)c(C#N)c65)nc43)cn2n1] | 0.93 | 769 | A |
| II-5 | 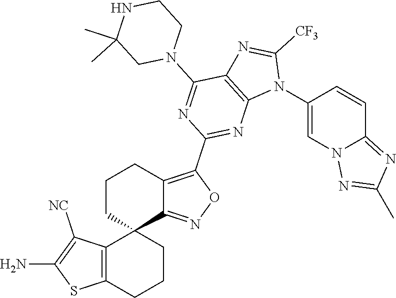 [SMILES: Cc1nc2ccc(-n3c(C(F)(F)F)nc4c(N5CCNC(C)(C)C5)nc(-c5onc6c5CCC[C@@]65CCCc6sc(N)c(C#N)c65)nc43)cn2n1] | 1.40 | 715 | A |
| II-6 | 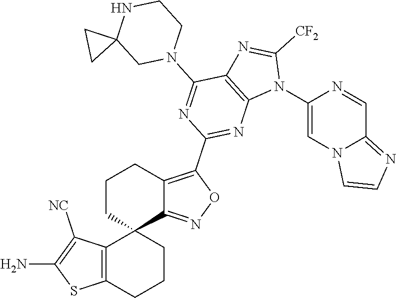 | 1.33 | 699 | A |
| II-7 | 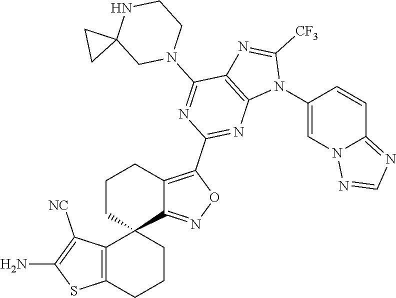 [SMILES: N#Cc1c(N)sc2c1[C@@]1(CCC2)CCCc2c1noc2-c1nc(N2CCNC3(CC3)C2)c2nc(C(F)(F)F)n(-c3ccc4ncnn4c3)c2n1] | 1.34 | 699 | A |
| II-8 | 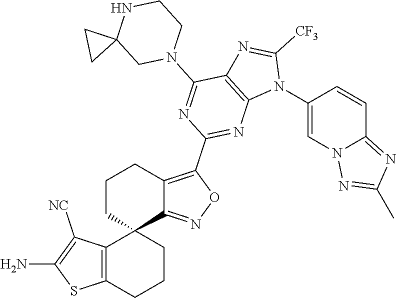 [SMILES: Cc1nc2ccc(-n3c(C(F)(F)F)nc4c(N5CCNC6(CC6)C5)nc(-c5onc6c5CCC[C@@]65CCCc6sc(N)c(C#N)c65)nc43)cn2n1] | 1.37 | 713 | A |
| II-9 | 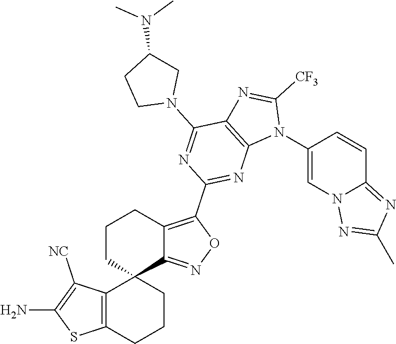 [SMILES: Cc1nc2ccc(-n3c(C(F)(F)F)nc4c(N5CC[C@H](N(C)C)C5)nc(-c5onc6c5CCC[C@@]65CCCc6sc(N)c(C#N)c65)nc43)cn2n1] | 1.46 | 715 | A |
| II-10 | 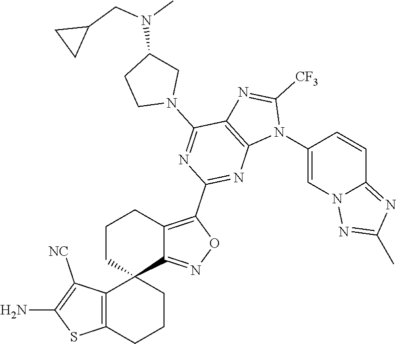 [SMILES: Cc1nc2ccc(-n3c(C(F)(F)F)nc4c(N5CC[C@H](N(C)CC6CC6)C5)nc(-c5onc6c5CCC[C@@]65CCCc6sc(N)c(C#N)c65)nc43)cn2n1] | 1.60 | 755 | A |
| II-11 | 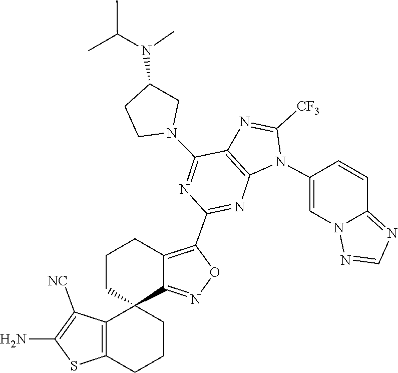 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3ncnn3c2)C1] | 1.59 | 729 | A |
| II-12 | 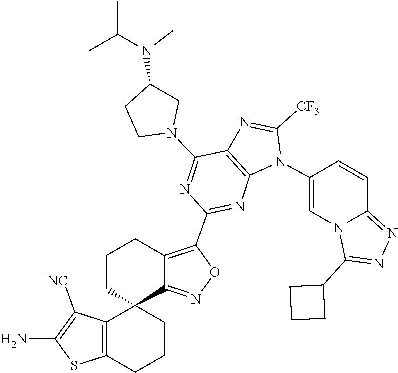 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C4CCC4)n3c2)C1] | 1.61 | 783 | A |
| II-13 | 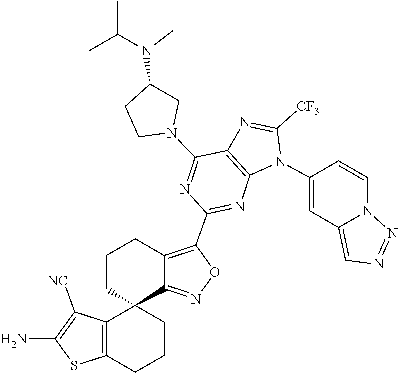 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccn3nncc3c2)C1] | 1.62 | 729 | A |
| II-14 | 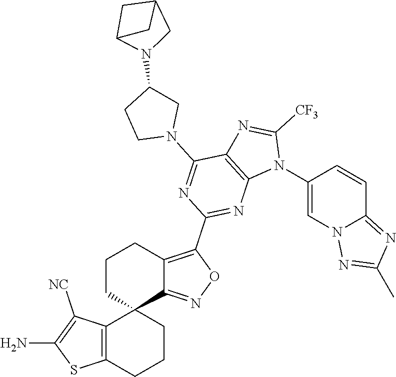 [SMILES: Cc1nc2ccc(-n3c(C(F)(F)F)nc4c(N5CC[C@H](N6CC7CC6C7)C5)nc(-c5onc6c5CCC[C@@]65CCCc6sc(N)c(C#N)c65)nc43)cn2n1] | 1.55 | 753 | A |
| II-15 | 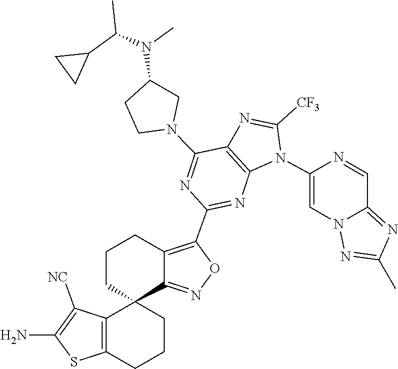 [SMILES: Cc1nc2cnc(-n3c(C(F)(F)F)nc4c(N5CC[C@H](N(C)[C@@H](C)C6CC6)C5)nc(-c5onc6c5CCC[C@@]65CCCc6sc(N)c(C#N)c65)nc43)cn2n1] | 1.74 | 770 | A |
| II-16 | 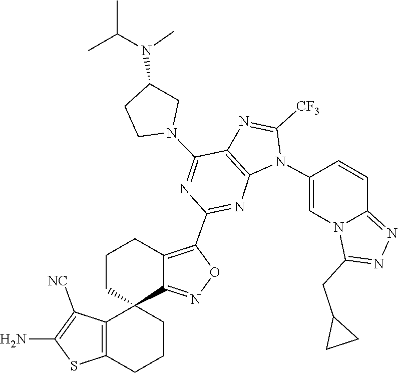 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(CC4CC4)n3c2)C1] | 1.61 | 783 | A |
| II-17 | 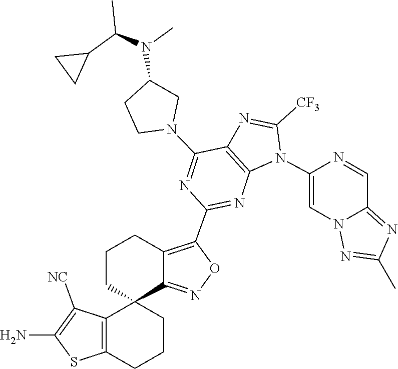 [SMILES: Cc1nc2cnc(-n3c(C(F)(F)F)nc4c(N5CC[C@H](N(C)[C@H](C)C6CC6)C5)nc(-c5onc6c5CCC[C@@]65CCCc6sc(N)c(C#N)c65)nc43)cn2n1] | 1.74 | 770 | A |
| II-18 | 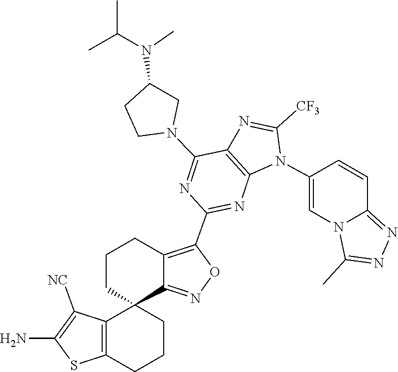 [SMILES: Cc1nnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.53 | 743 | A |
| II-19 | 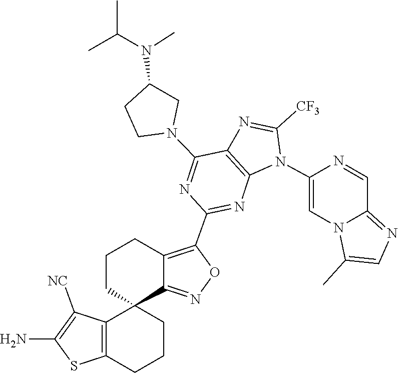 [SMILES: Cc1cnc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.62 | 743 | A |
| II-20 | 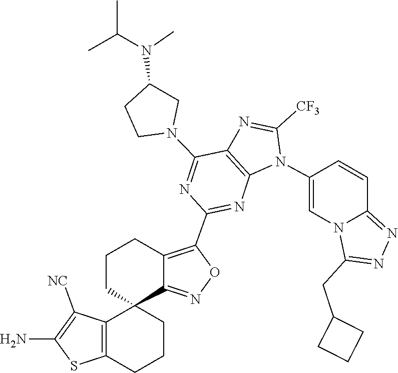 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(CC4CCC4)n3c2)C1] | 1.66 | 797 | A |
| II-21 | 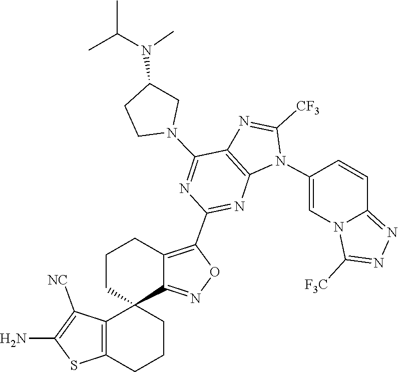 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1] | 1.66 | 797 | A |
| II-22 | 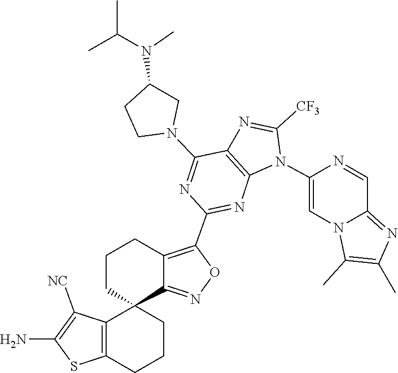 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2c1C] | 1.63 | 757 | A |
| II-23 | 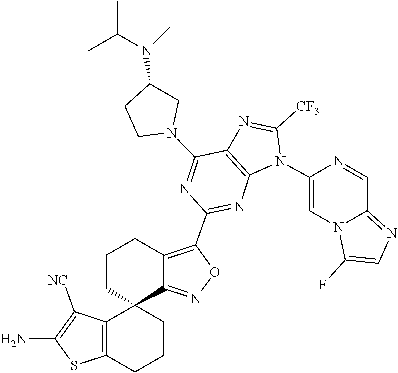 [SMILES: CC(C)N(C)[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1] | 1.63 | 747 | A |
| II-24 | 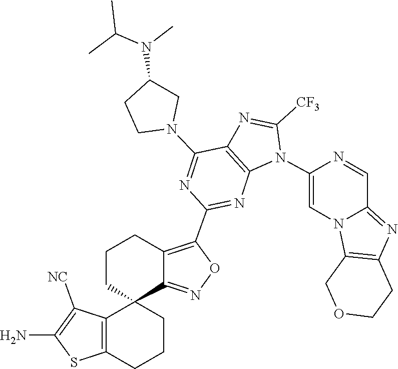 [SMILES: CC(C)N(C)[C@H]1CCN(c2nc(-c3onc4c3CCC[C@@]43CCCc4sc(N)c(C#N)c43)nc3c2nc(C(F)(F)F)n3-c2cn3c4c(nc3cn2)CCOC4)C1] | 1.60 | 785 | A |
| II-25 | 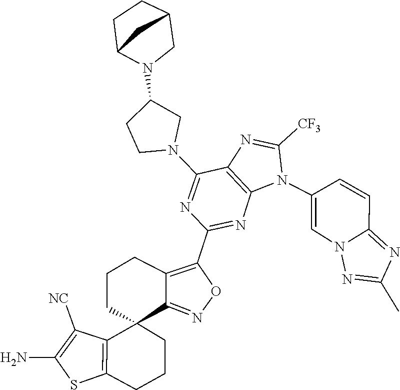 [SMILES: Cc1nc2ccc(-n3c(C(F)(F)F)nc4c(N5CC[C@H](N6C[C@H]7CC[C@@H]6C7)C5)nc(-c5onc6c5CCC[C@@]65CCCc6sc(N)c(C#N)c65)nc43)cn2n1] | 1.65 | 767 | A |
| II-26 | 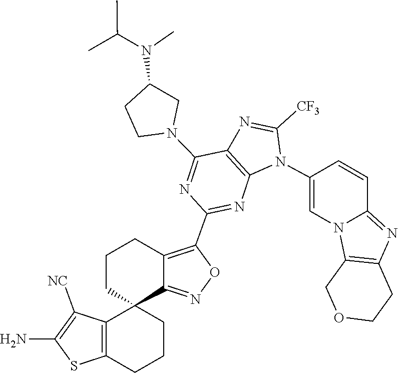 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nc4c(n3c2)COCC4)C1] | 1.60 | 784 | A |
| II-27 | 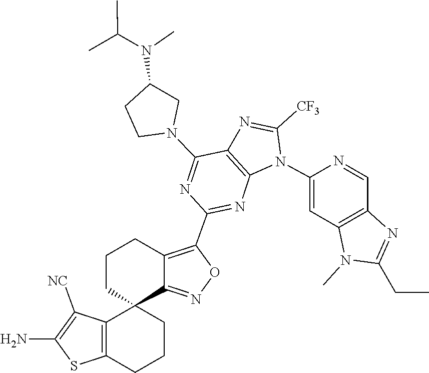 [SMILES: CCc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C] | 1.62 | 771 | A |
| II-28 | 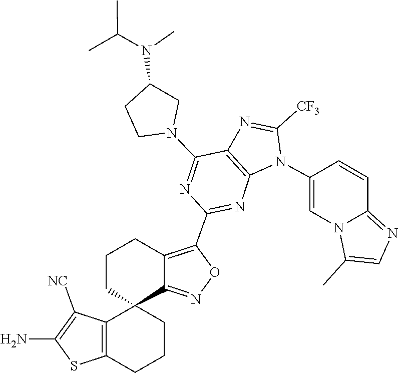 [SMILES: Cc1cnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.63 | 742 | A |
| II-29 | 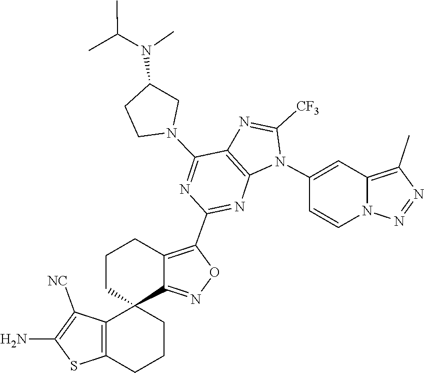 [SMILES: Cc1nnn2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cc12] | 1.64 | 743 | A |
| II-30 | 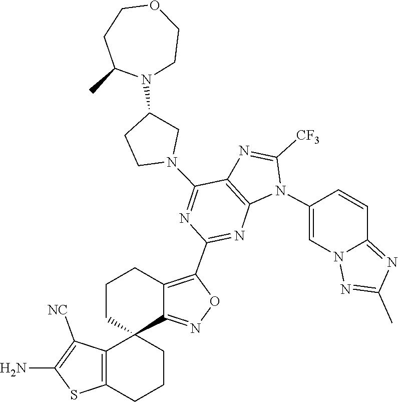 [SMILES: Cc1nc2ccc(-n3c(C(F)(F)F)nc4c(N5CC[C@H](N6CCOCC[C@@H]6C)C5)nc(-c5onc6c5CCC[C@@]65CCCc6sc(N)c(C#N)c65)nc43)cn2n1] | 1.59 | 785 | A |
| II-31 | 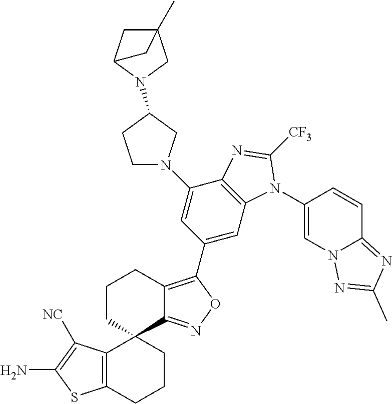 | 1.64 | 767 | A |
| II-32 | 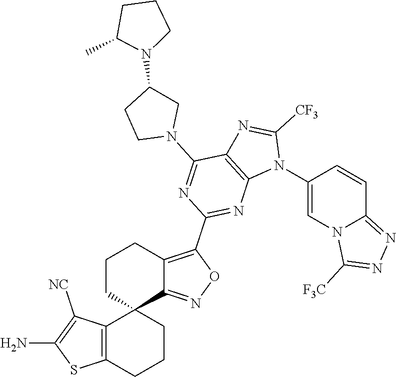 [SMILES: C[C@@H]1CCCN1[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1] | 1.67 | 809 | A |
| II-33 | 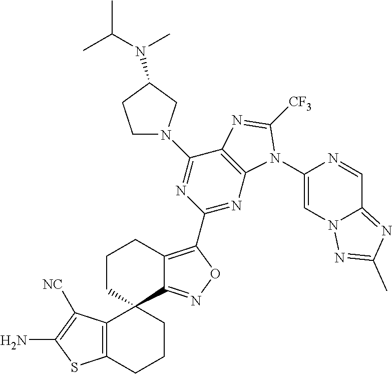 | 1.65 | 744 | A |
| II-34 | 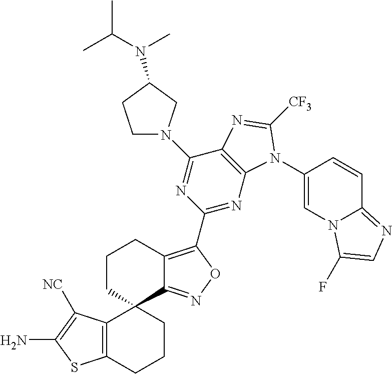 | 1.65 | 746 | A |
| II-35 | 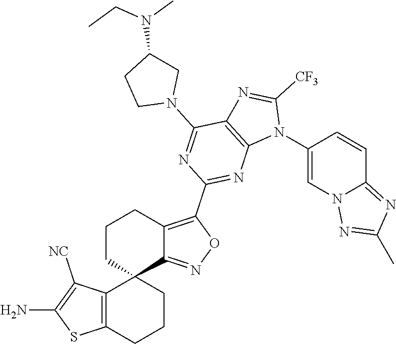 [SMILES: CCN(C)[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1] | 1.50 | 729 | A |
| II-36 | 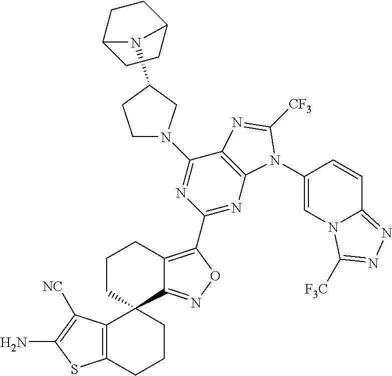 | 1.68 | 821 | A |
| II-37 | 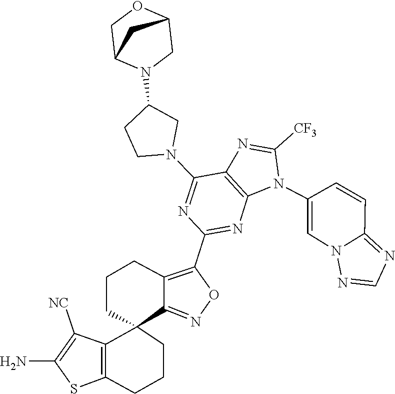 [SMILES: N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3ccc4ncnn4c3)c2n1] | 1.38 | 755 | A |
| II-38 | 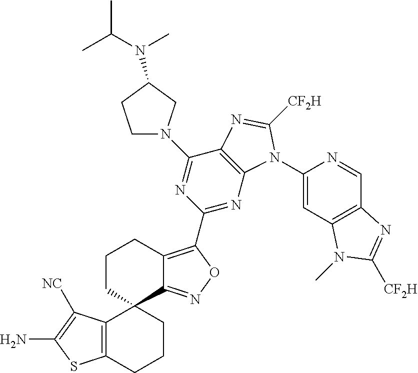 [SMILES: CC(C)N(C)[C@H]1CCN(c2nc(-c3onc4c3CCC[C@@]43CCCc4sc(N)c(C#N)c43)nc3c2nc(C(F)F)n3-c2cc3c(cn2)nc(C(F)F)n3C)C1] | 1.62 | 775 | A |
| II-39 | 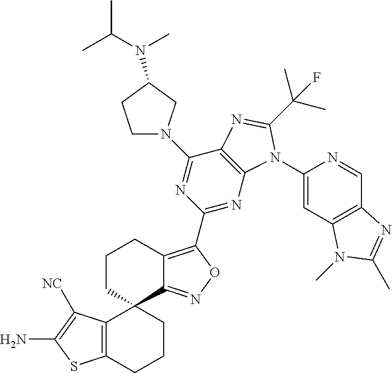 [SMILES: Cc1nc2cnc(-n3c(C(C)(C)F)nc4c(N5CC[C@H](N(C)C(C)C)C5)nc(-c5onc6c5CCC[C@@]65CCCc6sc(N)c(C#N)c65)nc43)cc2n1C] | 1.56 | 749 | A |
| II-40 | 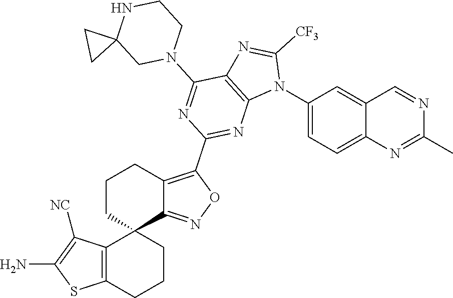 [SMILES: Cc1ncc2cc(-n3c(C(F)(F)F)nc4c(N5CCNC6(CC6)C5)nc(-c5onc6c5CCC[C@@]65CCCc6sc(N)c(C#N)c65)nc43)ccc2n1] | 1.30 | 670 | A |
| II-41 | 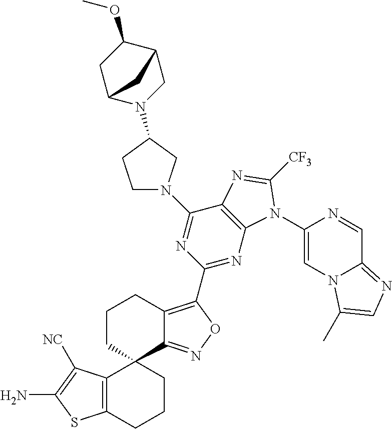 | 1.53 | 797 | A |
| II-42 | 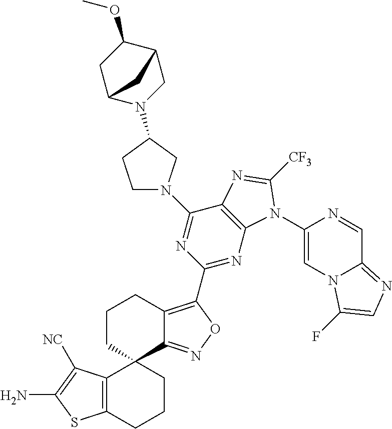 | 1.56 | 801 | A |
| II-43 | 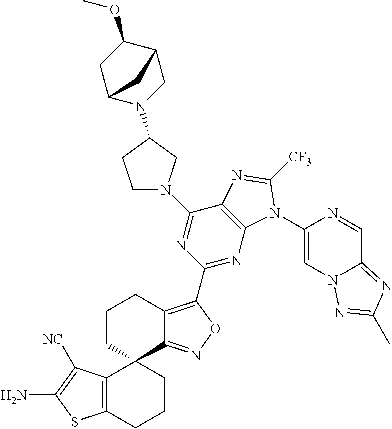 | 1.58 | 798 | A |
| II-44 | 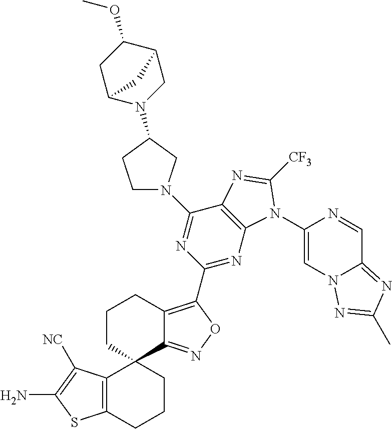 | 1.58 | 798 | A |
| II-45 | 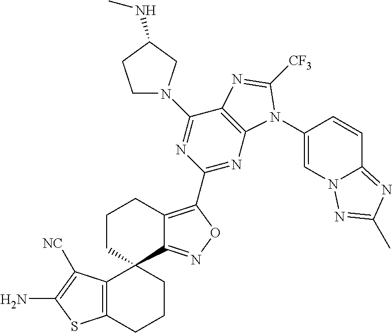 | 1.01 | 701 | C |
| II-46 | 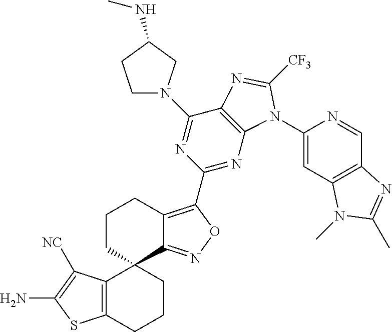 [SMILES: CN[C@H]1CCN(c2nc(-c3onc4c3CCC[C@@]43CCCc4sc(N)c(C#N)c43)nc3c2nc(C(F)(F)F)n3-c2cc3c(cn2)nc(C)n3C)C1] | 1.01 | 715 | C |

### Reductive Amination

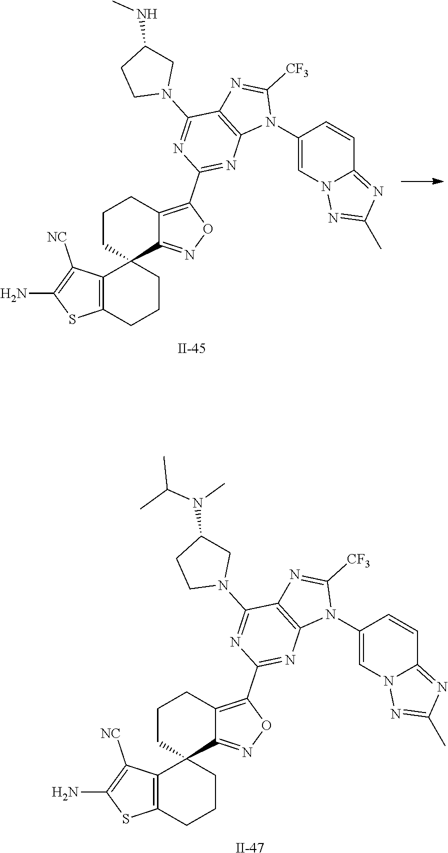

A solution of 11-45 (90 mg, 0.13 mmol, 1.0 equiv.) in dry methanol (1 mL) is stirred with acetone (19.2 μL, 0.26 mmol, 2.0 equiv.) and a drop of acetic acid (3.7 μL, 0.01 mmol, 0.5 equiv.) for 5 minutes. Sodium cyanoborohydride solution (0.26 mL, 0.24 mmol, 2.0 equiv.) is added and the reaction is allowed to proceed at 60 °C overnight. The reaction mixture is basified with ammonia, filtered and purified by chromatography to give the desired product II-47.

The following final compounds II (Table 17) are obtained analogously using the indicated carbonyl component. The crude product is purified by chromatography if necessary.

TABLE 17

| | | tᵣₑₜ | | HPLC | Carbonyl |
|---|---|---|---|---|---|
| # | structure | [min] | [M + H]⁺ | method | component |
| II-47 | 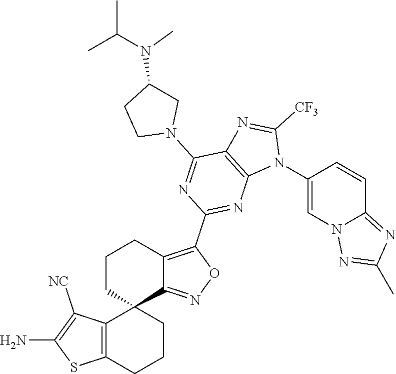 [SMILES: Cc1nc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1] | 1.61 | 743 | A | 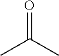 |
| II-48 | 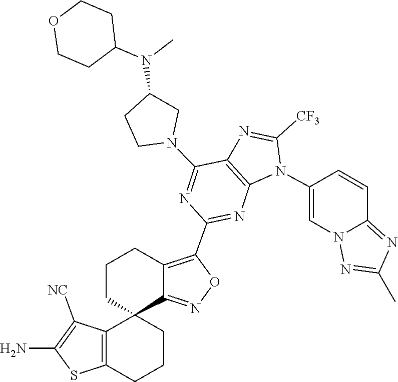 [SMILES: Cc1nc2ccc(-n3c(C(F)(F)F)nc4c(N5CC[C@H](N(C)C6CCOCC6)C5)nc(-c5onc6c5CCC[C@@]65CCCc6sc(N)c(C#N)c65)nc43)cn2n1] | 1.47 | 785 | A | 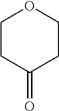 |
| II-49 | 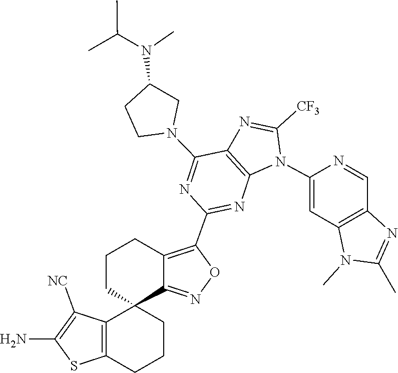 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C] | 1.58 | 757 | A | 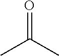 |

The following compounds (Table 18) were prepared according to the respective references cited below.

TABLE 18

| | | Synthesis according |
|---|---|---|
| # | | to |
| II-50 | 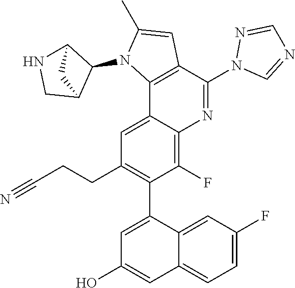 [SMILES: Cc1cc2c(-n3cncn3)nc3c(F)c(-c4cc(O)cc5ccc(F)cc45)c(CCC#N)cc3c2n1[C@H]1[C@H]2CN[C@@H]1C2] | WO2023064857 Example 26 |
| II-51 | 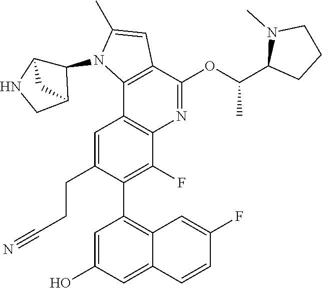 [SMILES: Cc1cc2c(O[C@@H](C)[C@@H]3CCCN3C)nc3c(F)c(-c4cc(O)cc5ccc(F)cc45)c(CCC#N)cc3c2n1[C@H]1[C@H]2CN[C@@H]1C2] | WO2023064857 Example 22 |
| MRTX1133 II-52 | 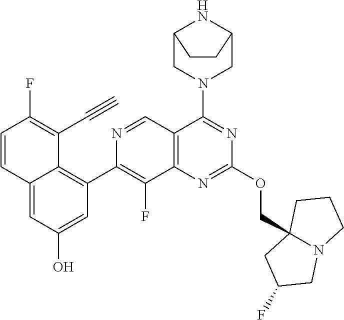 [SMILES: C#Cc1c(F)ccc2cc(O)cc(-c3ncc4c(N5CC6CCC(C5)N6)nc(OC[C@@]56CCCN5C[C@H](F)C6)nc4c3F)c12] | Wang, X. et al. J Med Chem 2022 65(4), 3123-3133 |
| II-53 | 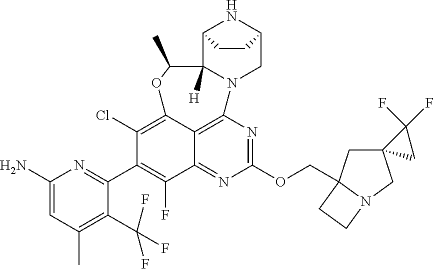 | WO2022173678 Example 21 |
| II-54 | 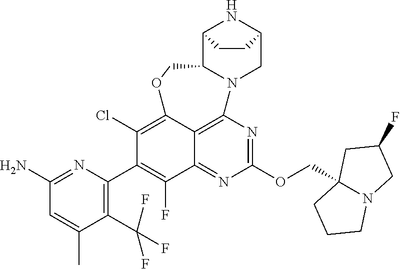 [SMILES: Cc1cc(N)nc(-c2c(Cl)c3c4c(nc(OC[C@@]56CCCN5C[C@H](F)C6)nc4c2F)N2C[C@H]4CC[C@H](N4)[C@H]2CO3)c1C(F)(F)F] | WO2022173678 Example 28 |
| RMC-9805 Zoldonrasib TCI-1 | 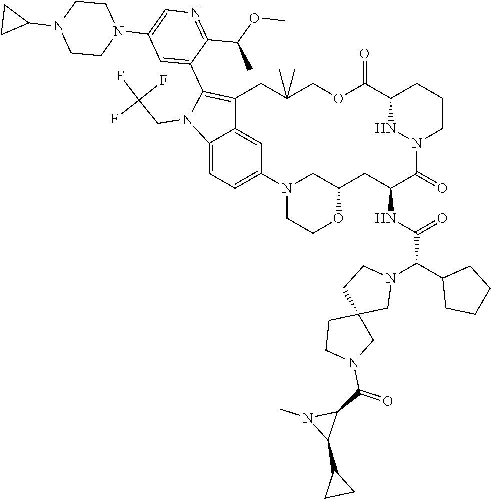 [SMILES: CO[C@@H](C)c1ncc(N2CCN(C3CC3)CC2)cc1-c1c2c3cc(ccc3n1CC(F)(F)F)N1CCO[C@@H](C[C@H](NC(=O)[C@H](C3CCCC3)N3CC[C@]4(CCN(C(=O)[C@H]5[C@@H](C6CC6)N5C)C4)C3)C(=O)N3CCC[C@H](N3)C(=O)OCC(C)(C)C2)C1] | WO2023060253 Example A26 |
| RMC-6236 Daraxonrasib TCI-2 | 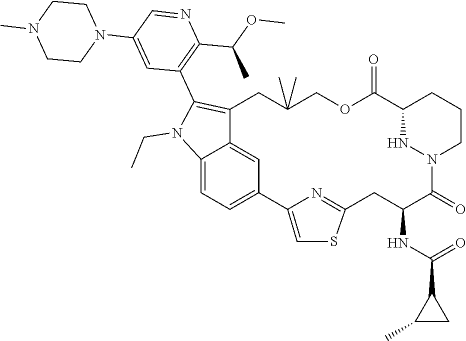 | WO2022060836 Example A293 |
| TCI-3 | 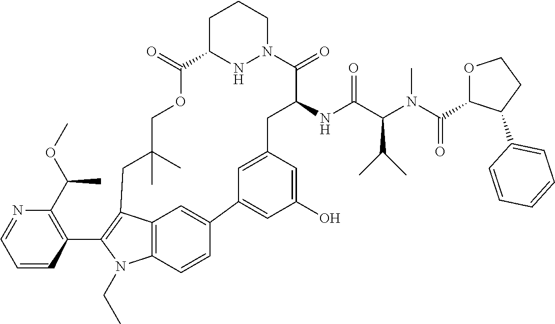 | WO2021091956 Example A416 |

### Assay Description and Data

### KRAS TR-FRET Assays

This assay measures the inhibitory effect of compounds on KRAS::CRAF protein::protein interactions in the presence of GTP using Time-resolved fluorescence energy transfer (TR-FRET).

The following wildtype or mutant alleles of KRAS and CRAF protein are used at the given concentrations:

KRAS (WT) 1-169, N-terminal 6His-tag, C-terminal avi-tag (Boehringer Ingelheim in house protein prep); final assay concentration 15 nM.

KRAS (G12D) 1-169, N-terminal 6His-tag, C-terminal avi-tag (Xtal BioStructures, Inc.); final assay concentration 15 nM,

CRAF Ras binding domain, GST-tag, TEV cleavage site (Boehringer Ingelheim in house protein prep); final assay concentration 15 nM.

Test compounds dissolved in DMSO are dispensed onto assay plates (Proxiplate 384 PLUS, white, PerkinElmer; 6008289) using an Access Labcyte Workstation with the Labcyte Echo 55x. For the chosen highest assay concentration of 100 μM, 150 nl of compound solution is transferred from a 10 mM DMSO compound stock solution. A series of eleven fivefold dilutions per compound is transferred to the assay plate, compound dilutions are tested in duplicates. DMSO is added as backfill to a total volume of 150 nl.

The assay runs on a fully automated robotic system. To 150 nl of compound dilution 15 μl of a mix including the respective KRAS allele, CRAF (e.g. KRAS (G12D)::CRAF; final assay concentrations see above), GTP nucleotide (Sigma G8877; final assay concentration 10 μM), Lance Eu-W1024 labeled Streptavidin (PerkinElmer, Cat No AD0063; final assay concentration 1.5 nM) and Anti-GST surelight APC (PerkinElmer, Cat No AD0059G; final assay concentration 30 nM) in assay buffer (1×PBS, 0.1% BSA, 0.05% Tween 20) are added into columns 1-23 and 15 μL of the solution without the respective KRAS allele are added to row 24.

Plates are kept at room temperature in a darkened incubator. After 60 minutes incubation time the signal is measured in a PerkinElmer Envision HTS Multilabel Reader using the TR-FRET LANCE Ultra specs from PerkinElmer.

Each plate contains 16 wells of a negative control (diluted DMSO instead of test compound; w respective KRAS allele; column 23) and 16 wells of a positive control (diluted DMSO instead of test compound; w/o respective KRAS allele; column 24).

IC50 values are calculated and analyzed with Boehringer Ingelheim's MEGALAB IC50 application using a 4 parametric logistic model.

### Ba/F3 Cell Model Generation

Ba/F3 cells are ordered from DSMZ (ACC300, Lot17) and grown in RPMI-1640 (ATCC 30-2001)+10% FCS+10 ng/mL IL-3 at 37 °C in 5% CO₂ atmosphere. Plasmids containing the KRAS_G12D or KRAS_G12D-Y71H sequences are obtained from GeneScript. To generate a KRAS_G12D- or KRAS_G12D-Y71H-dependent Ba/F3 models, Ba/F3 cells are transduced with a retrovirus containing a vector that harbors the KRAS_G12D or KRAS_G12D-Y71H isoforms. Platinum-E cells (Cell Biolabs RV101) are used for retrovirus packaging. Retrovirus is added to Ba/F3 cells. To ensure infection, 4 μg/mL polybrene is added and cells are spinfected. Infection efficiency is confirmed by measuring GFP-positive cells using a cell analyzer. Cells with an infection efficiency of 10% to 20% are further cultivated and puromycin selection with 1 μg/mL is initiated. As a control, parental Ba/F3 cells are used to show selection status. Selection is considered successful when parental Ba/F3 cells cultures died. To evaluate the transforming potential of the KRAS_G12D and KRAS_G12D-Y71H mutations, the growth medium is no longer supplemented with IL-3. Before conducting the experiments, puromycin is left out.

### Ba/F3 KRAS_G12D Proliferation Assay

For proliferation assays, Ba/F3 cells are seeded into 384-well plates at 1.5×10³ cells/60 μL in growth media (RPMI-1640+10% FCS). Compounds (10 mM stock in DMSO) are added at logarithmic dose series using the ECHO acoustic liquid handler system (Beckman Coulter), normalizing for added DMSO and including DMSO controls. All compound treatments are performed in technical duplicates. For the TO time point measurement, untreated cells are analyzed at the time of compound addition. Treated cells are incubated for 72 h at 37 °C with 5% CO₂ and cell viability is measured in the PerkinElmer Envision Multimode Reader using AlamarBlue™ (ThermoFisher) viability stain. Viability (stated as percent of control) is defined as relative fluorescence units RFU of each well divided by the RFU of cells in DMSO controls. IC₅₀ values are determined from viability measurements by non-linear regression using a four-parameter model.

IC₅₀ values of representative compounds according to the invention measured with this assay are presented in Table 19.

Additional Proliferation Assays with Mutant Cancer Cell Lines

### AsPC-1 CTG Proliferation Assay (120 h) (Pancreatic Cancer, KRAS G12D Mutant)

AsPC-1 cells (ATCC CRL-1682) are dispensed into black 384-well plates, flat and clear bottom (Greiner, PNr. 781091) at a density of 500 cells per well in 60 μl RPMI ATCC-Formulation (PAN P04-18047)+10% FCS (fetal calf serum, HyClone, SH30084.03). Cells are incubated overnight at 37 °C in a humidified tissue culture incubator at 5% CO₂. Compounds (10 mM stock in DMSO) are added at logarithmic dose series using the ECHO acoustic liquid handler system (Beckman Coulter), normalizing for added DMSO and including DMSO controls. All compound treatments are performed in technical duplicates. For the TO time point measurement, untreated cells are analyzed at the time of compound addition. Plates are incubated for 120 h, and cell viability is measured using CellTiter-Glo® 2.0 Cell Viability Reagent (Promega G9243). Viability (stated as percent of control) is defined as relative luminescence units RLU of each well divided by the RLU of cells in DMSO controls. IC₅₀ values are determined from viability measurements by non-linear regression using a four-parameter model.

### GP2d CTG Proliferation Assay (120 h) (Adenocarcinoma, KRAS G12D Mutant)

GP2d cells (ECACC No. 95090714) are dispensed into black 384-well plates, flat and clear bottom (Greiner, PNr. 781091) at a density of 500 cells per well in 60 μl DMEM (Sigma D6429)+10% FCS (fetal calf serum, HyClone, SH30084.03). Cells are incubated overnight at 37 °C in a humidified tissue culture incubator at 5% CO₂. Compounds (10 mM stock in DMSO) are added at logarithmic dose series using the ECHO acoustic liquid handler system (Beckman Coulter), normalizing for added DMSO and including DMSO controls. All compound treatments are performed in technical duplicates. For the TO time point measurement, untreated cells are analyzed at the time of compound addition. Plates are incubated for 120 h, and cell viability is measured using CellTiter-Glo® 2.0 Cell Viability Reagent (Promega G9243). Viability (stated as percent of control) is defined as relative luminescence units RLU of each well divided by the RLU of cells in DMSO controls. IC₅₀ values are determined from viability measurements by non-linear regression using a four-parameter model.

Table of example compounds disclosed herein contain IC₅₀ values determined using the above assays (see)

TABLE 19

| | BAF3_{—} | | KRAS_{—} | | |
|---|---|---|---|---|---|
| | KRAS_{—} | KRASG12D::CRAF | WT::CRAF | ASPC-1 | GP2D |
| | G12D | GTP IC₅₀ | GTP IC₅₀ | IC₅₀ | IC50 |
| # | IC₅₀ (nM) | (nM) | (nM) | (nM) | (nM) |
| II-1 | 2.6 | 20.7 | 78 | 26 | 2 |
| II-2 | 3.0 | 12.6 | 469 | 43 | 14 |
| II-3 | 2.4 | 11.5 | 699 | 31 | 3 |
| II-4 | 1.8 | 12.6 | 862 | 30 | 3 |
| II-5 | 3.0 | 10.9 | 33 | 56 | 3 |
| II-6 | 4.0 | 36.1 | 115 | 284 | 9 |
| II-7 | 2.8 | 20.3 | 79 | 35 | 3 |
| II-8 | 2.8 | 18.5 | 75 | 23 | 3 |
| II-9 | 7.0 | 21.2 | 798 | 55 | 23 |
| II-10 | 5.9 | 26.8 | 1606 | 51 | 11 |
| II-11 | 2.1 | 10.6 | 1170 | 9 | 3 |
| II-12 | 3.6 | 14.2 | 1436 | 15 | 3 |
| II-13 | 1.5 | 31.0 | 3777 | 17 | 5 |
| II-14 | 2.4 | 4.9 | 1195 | 17 | 2 |
| II-15 | 1.4 | 18.3 | 4843 | 19 | 3 |
| II-16 | 1.4 | 9.6 | 1324 | 20 | 3 |
| II-17 | 1.3 | 17.9 | 5020 | 20 | 3 |
| II-18 | 3.6 | 9.0 | 799 | 20 | 2 |
| II-19 | 2.5 | 10.8 | 870 | 21 | 4 |
| II-20 | 2.1 | 8.0 | 2482 | 25 | 4 |
| II-21 | 1.9 | 11.8 | 2336 | 25 | 3 |
| II-22 | 4.2 | 12.6 | 1080 | 29 | 6 |
| II-23 | 4.9 | 13.3 | 1122 | 29 | 7 |
| II-24 | 1.6 | 19.2 | 1226 | 30 | 2 |
| II-25 | 2.4 | 11.4 | 2003 | 31 | 1 |
| II-26 | 0.9 | 19.9 | 16617 | 31 | 2 |
| II-27 | 1.6 | 13.2 | 817 | 33 | 3 |
| II-28 | 1.6 | 21.0 | 9588 | 35 | 4 |
| II-29 | 3.0 | 18.3 | 4512 | 35 | 4 |
| II-30 | 2.0 | 41.6 | >20000 | 38 | 11 |
| II-31 | 4.0 | 11.1 | 1647 | 42 | 2 |
| II-32 | 2.1 | 20.7 | 3094 | 45 | 4 |
| II-33 | 4.0 | 14.0 | 2257 | 54 | 9 |
| II-34 | 2.6 | 6.4 | 2563 | 58 | 5 |
| II-35 | 4.5 | 13.3 | 871 | 61 | 43 |
| II-36 | 4.3 | 29.6 | 2126 | 84 | 6 |
| II-37 | 3.4 | 49.8 | 3171 | 119 | 10 |
| II-38 | 4.2 | 34.1 | >4000 | 21 | 17 |
| II-39 | 3.9 | 10.9 | 883 | 41 | 1 |
| II-40 | 16.8 | 50.1 | 450 | 108 | 8 |
| II-41 | | 18.8 | 1051 | 7 | 2 |
| II-42 | | 10.3 | 1474 | 18 | 2 |
| II-43 | 2.0 | 16.3 | 3148 | 20 | 4 |
| II-44 | 1.9 | 15.1 | 2109 | 30 | 5 |
| II-47 | 1.1 | 9.7 | 1582 | 17 | 3 |
| II-48 | 1.3 | 19.3 | 4853 | 18 | 4 |
| II-49 | 2.7 | 13.6 | 647 | 9 | 2 |

### Tri-Complex Inhibitor Resistant GP2d (GP2d KRAS_71H) Cell Generation

GP2d cells (ECACC No. 95090714) are cultured at 37 °C in a humidified tissue culture incubator at 5% CO₂, in DMEM (Sigma D6429)+10% FBS (GIBCO, 26140-079) and treated with 10× of IC50 of TCI-3 for several weeks in small, separated cell pools with constant medium and compound renewal. After 7 weeks of treatment, when cells recovered and started growing again, a CTG proliferation assay is performed to confirm loss of sensitivity (an IC50 shift compared to the parental cell line). Cells are then seeded for single cell cloning and individual clones analyzed by Sanger sequencing. An additional KRAS_Y71H mutation was identified in tri-complex resistant GP2d cells.

### GP2d KRA S_Y71H/GP2d Ratio Determination

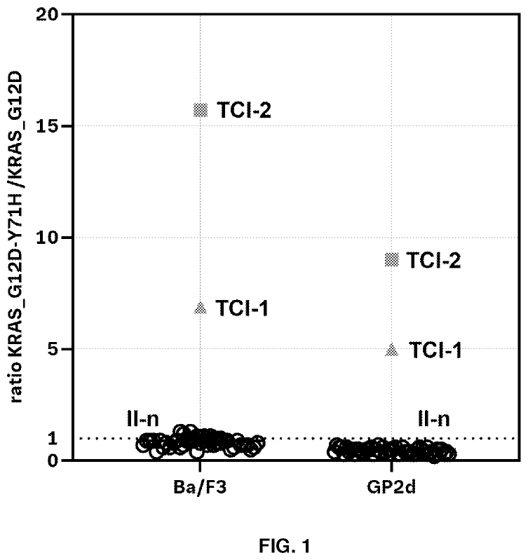

GP2d KRAS_Y71H and GP2d cells are dispensed into black 384-well plates, flat and clear bottom (Greiner, PNr. 781091) at a density of 600 cells per well in 50 μl DMEM (Sigma D6429)+10% FBS (GIBCO, 26140-079). Cells are incubated overnight at 37 °C in a humidified tissue culture incubator at 5% CO₂. Compounds, 1:30 diluted in DMSO (10 mM stock in DMSO) are added a start concentration of 1 μM with 10×1:3 dilutions using the D300e Digital Dispenser (HP Development Company). All compound treatments are performed in technical triplicates. Plates are incubated for 96 h-120 h, and cell viability is measured using CellTiter-Glo® 2.0 Cell Viability Reagent (Promega G9243). Viability is defined as relative luminescence units (RLU) of each well divided by the RLU of cells in DMSO controls. IC₅₀ values are determined from viability measurements by non-linear regression with sigmoidal curve fitting and variable slope. The ratio is determined by dividing the GP2d KRAS_Y71H IC₅₀ by the GP2d IC₅₀. A ratio of 1 indicates the same sensitivity in both cell lines, whereas an increased ratio indicates a reduced sensitivity in the GP2d KRAS_Y71H. Ratios of representative compounds according to the invention measured with this assay are presented in Table 20 and in FIG. 1.

### Ba/F3 KRAS_G12D-Y71H/Ba/F3 KRAS_G12D Ratio Determination

Ba/F3 KRAS_G12D-Y71H and Ba/F3 KRAS_G12D cells are dispensed into black 384-well plates, flat and clear bottom (Greiner, PNr. 781091) at a density of 1000 cells per well in 50 μl RPM11640 ATCC formulation (PAN Biotech, P04-18047)+Glutamax (GIBCO, 35050-038)+10% FBS (GIBCO, 26140-076)). Compounds, 1:30 diluted in DMSO (10 mM stock in DMSO) are added at a start concentration of 1 μM with 10×1:3 dilutions using the D300e Digital Dispenser (HP Development Company). All compound treatments are performed in technical triplicates. Plates are incubated for 96 h, and cell viability is measured using CellTiter-Glo® 2.0 Cell Viability Reagent (Promega G9243). Viability is defined as relative luminescence units RLU of each well divided by the RLU of cells in DMSO controls. IC₅₀ values are determined from viability measurements by non-linear regression with sigmoidal curve fitting and variable slope. The ratio is determined by dividing the Ba/F3 KRAS_G12D-Y71H IC₅₀ by the Ba/F3 KRAS_G12D IC₅₀. A ratio of 1 indicates the same sensitivity in both cell lines, whereas an increased ratio indicates a reduced sensitivity in the Ba/F3 KRAS_G12D-Y71H. Ratios of representative compounds according to the invention measured with this assay are presented in Table 20 and in FIG. 1.

TABLE 20

| | GP2d | Ba/F3 KRAS_G12D- |
|---|---|---|
| | KRAS_Y71H/ | Y71H/Ba/F3 |
| # | GP2d ratio | KRAS_G12D ratio |
| II-1 | 0.4 | 0.9 |
| II-2 | 0.5 | 1.1 |
| II-3 | 0.5 | 1.0 |
| II-4 | 0.4 | 1.0 |
| II-5 | 0.4 | 0.8 |
| II-6 | 0.3 | 0.7 |
| II-8 | 0.5 | 0.9 |
| II-9 | 0.3 | 0.6 |
| II-10 | 0.4 | 0.5 |
| II-11 | 0.6 | 1.0 |
| II-13 | 0.6 | 0.9 |
| II-14 | 0.4 | 1.0 |
| II-15 | 0.7 | 0.7 |
| II-16 | 0.5 | 1.1 |
| II-17 | 0.4 | 0.8 |
| II-18 | 0.5 | 1.1 |
| II-19 | 0.5 | 0.9 |
| II-20 | 0.7 | 1.3 |
| II-21 | 0.5 | 0.9 |
| II-22 | 0.5 | 0.6 |
| II-23 | 0.4 | 0.7 |
| II-24 | 0.5 | 0.9 |
| II-25 | 0.6 | 0.9 |
| II-26 | 0.6 | 0.9 |
| II-27 | 0.7 | 0.9 |
| II-28 | 0.4 | 0.8 |
| II-29 | 0.6 | 0.9 |
| II-30 | 0.5 | 0.9 |
| II-31 | 0.3 | 0.6 |
| II-32 | 0.6 | 1.0 |
| II-33 | 0.6 | 0.8 |
| II-34 | 0.6 | 0.9 |
| II-35 | 0.3 | 0.8 |
| II-36 | 0.3 | 0.7 |
| II-37 | 0.3 | 0.7 |
| II-38 | 0.4 | 0.6 |
| II-39 | 0.5 | 0.9 |
| II-40 | 0.2 | 0.5 |
| II-41 | 0.3 | 0.8 |
| II-42 | 0.3 | 0.7 |
| II-43 | 0.4 | 0.4 |
| II-47 | 0.3 | 0.8 |
| II-48 | 0.5 | 0.7 |
| II-49 | 0.4 | 0.8 |
| II-50 | 0.3 | 0.4 |
| II-51 | 0.6 | 1.2 |
| II-52 | 0.4 | 1.0 |
| II-53 | 0.3 | 1.0 |
| II-54 | 0.4 | 1.3 |
| TCI-1 | 5.0 | 6.9 |
| TCI-2 | 9.0 | 15.7 |

1. A method of treating and/or preventing cancer, the method comprising administering to a patient a therapeutically effective amount of a KRAS G12D switch II inhibitor, wherein the cancer is resistant to treatment with a tri-complex inhibitor.

2. The method according to claim 1, wherein the cancer is resistant to treatment with tri-complex inhibitors after earlier treatment with tri-complex inhibitors.

3. The method according to claim 2, wherein the cancer is resistant to treatment with Daraxonrasib (RMC-6236) and/or Zoldonrasib (RMC-9805).

4. The method according to claim 1 wherein the cancer cells of the cancer exhibit a primary KRAS G12D mutation and further exhibit a secondary KRAS Y71X mutation.

5. The method according to claim 4, wherein the secondary KRAS Y71X mutation is Y71H.

6. The method according to claim 1, wherein the KRAS G12D switch II inhibitor is a compound according to formula (I) 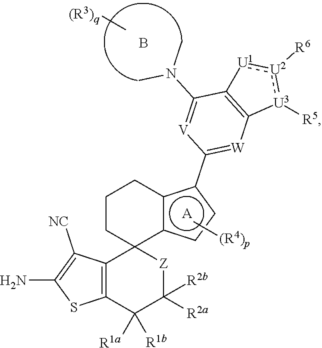
wherein
R^{1a} and R^{1b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
R^{2a} and R^{2b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
and/or, optionally, one of R^{1a} or R^{1b} and one of R^{2a} or R^{2b} together with the carbon atoms they are attached to form a cyclopropane ring;
Z is —(CR^{7a}R^{7b})ₙ—;
each R^{7a} and R^{7b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂,
C₃₋₅cycloalkyl and 3-5 membered heterocyclyl;
or R^{7a} and R^{7b} together with the carbon atom they are attached to form a cyclopropane ring;
n is selected from the group consisting of 0, 1 and 2;
W is nitrogen (—N═) or —CH═;
V is nitrogen (—N═) or —CH═;
ring A is a ring selected from the group consisting of pyrrole, furan, thiophene, imidazole, pyrazole, oxazole, isoxazole, thiazole, isothiazole and triazole;
each R⁴, if present, is independently selected from the group consisting of C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocyclyl;
p is selected from the group consisting of 0, 1, 2 and 3;
U¹ is nitrogen (—N═) or sulphur (—S—);
U² is nitrogen (—N═) or —CR⁵═;
U³ is nitrogen (—NR⁵—) or —CR⁵═;
ring B is a 4-11 membered heterocycle;
each R³, if present, is independently selected from the group consisting of R⁸ and R⁹;
each R⁸ is independently selected from the group consisting of —OR⁹, —NR⁹R⁹, halogen, and —CN;
each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the
C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
each R¹⁰ is independently selected from the group consisting of —OR¹¹, —NR¹¹R¹¹ and halogen;
each R¹¹ is independently selected from the group consisting of C₁₋₆alkyl, C₃₋₁₀cycloalkyl, and 3-11 membered heterocyclyl; wherein the C₁₋₆alkyl, C₃₋₁₀cycloalkyl, and 3-11 membered heterocyclyl are all optionally substituted with one or more identical or different R¹²;
each R¹² is independently selected from the group consisting of halogen, C₁₋₆alkyl, C₁₋₆alkoxy, C₁₋₆haloalkyl;
q is selected from the group consisting of 0, 1, 2, and 3;
R⁵ is selected from the group consisting of C₆₋₁₀aryl, and 5-13 membered heteroaryl;
wherein the C₆₋₁₀aryl, and 5-13 membered heteroaryl are all optionally substituted with one or more, identical or different R¹³
each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl, wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
each R¹⁴ is independently selected from the group consisting of halogen, —CN, C₁₋₆alkyl, C₁₋₆haloalkyl, and C₃₋₁₀cycloalkyl;
R⁶ is a selected from the group consisting of hydrogen, C₁₋₆alkyl, C₁₋₆haloalkyl, C₃₋₁₀cycloalkyl and 3-11 membered heterocyclyl; wherein C₁₋₆alkyl, C₁₋₆haloalkyl, C₃₋₁₀cycloalkyl and 3-11 membered heterocyclyl are all optionally substituted with one or more, identical or different R¹⁵;
each R¹⁵ is independently selected from the group consisting of halogen, C₁₋₆alkyl, and C₁₋₆haloalkyl;
or a salt thereof.

7. The method according to claim 1, wherein the KRAS G12D switch II inhibitor is a compound according to formula (IIa) or the salt thereof 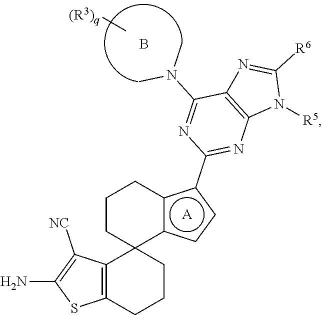
wherein
ring A is a ring selected from the group consisting of pyrrole, furan, thiophene, imidazole, pyrazole, oxazole, isoxazole, thiazole, isothiazole and triazole;
ring B is a 4-11 membered heterocycle;
each R³, if present, is independently selected from the group consisting of R⁸ and R⁹;
each R⁸ is independently selected from the group consisting of —OR⁹, —NR⁹R⁹, halogen, and —CN;
each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
each R¹⁰ is independently selected from the group consisting of —OR¹¹, —NR¹¹R¹¹ and halogen;
each R¹¹ is independently selected from the group consisting of C₁₋₆alkyl, C₃₋₁₀cycloalkyl, and 3-11 membered heterocyclyl; wherein the C₁₋₆alkyl, C₃₋₁₀cycloalkyl, and 3-11 membered heterocyclyl are all optionally substituted with one or more identical or different R¹²;
each R¹² is independently selected from the group consisting of halogen, C₁₋₆alkyl, C₁₋₆alkoxy, C₁₋₆haloalkyl;
q is selected from the group consisting of 0, 1, 2, and 3;
R⁵ is selected from the group consisting of C₆₋₁₀aryl, and 5-13 membered heteroaryl;
wherein the C₆₋₁₀aryl, and 5-13 membered heteroaryl are all optionally substituted with one or more, identical or different R¹³;
each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl, wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
each R¹⁴ is independently selected from the group consisting of halogen, —CN, C₁₋₆alkyl, C₁₋₆haloalkyl, and C₃₋₁₀cycloalkyl;
R⁶ is a selected from the group consisting of hydrogen, C₁₋₆alkyl, C₁₋₆haloalkyl, C₃₋₁₀cycloalkyl and 3-11 membered heterocyclyl; wherein C₁₋₆alkyl, C₁₋₆haloalkyl, C₃₋₁₀cycloalkyl and 3-11 membered heterocyclyl are all optionally substituted with one or more, identical or different R¹⁵; and
each R¹⁵ is independently selected from the group consisting of halogen, C₁₋₆alkyl, and C₁₋₆haloalkyl.

8. The method according to claim 6, wherein ring A is selected from the group consisting of 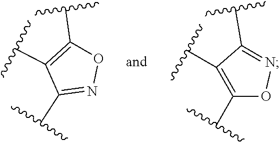
and
wherein R⁶ is a selected from the group consisting of C₁₋₆alkyl, C₁₋₆haloalkyl, and C₃₋₁₀cycloalkyl.

9. The method according to claim 7, wherein ring A is selected from the group consisting of 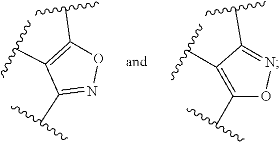
and
wherein R⁶ is a selected from the group consisting of C₁₋₆alkyl, C₁₋₆haloalkyl, and C₃₋₁₀cycloalkyl.

10. The method according to claim 6, wherein ring B—(R³)q is selected from the group consisting of

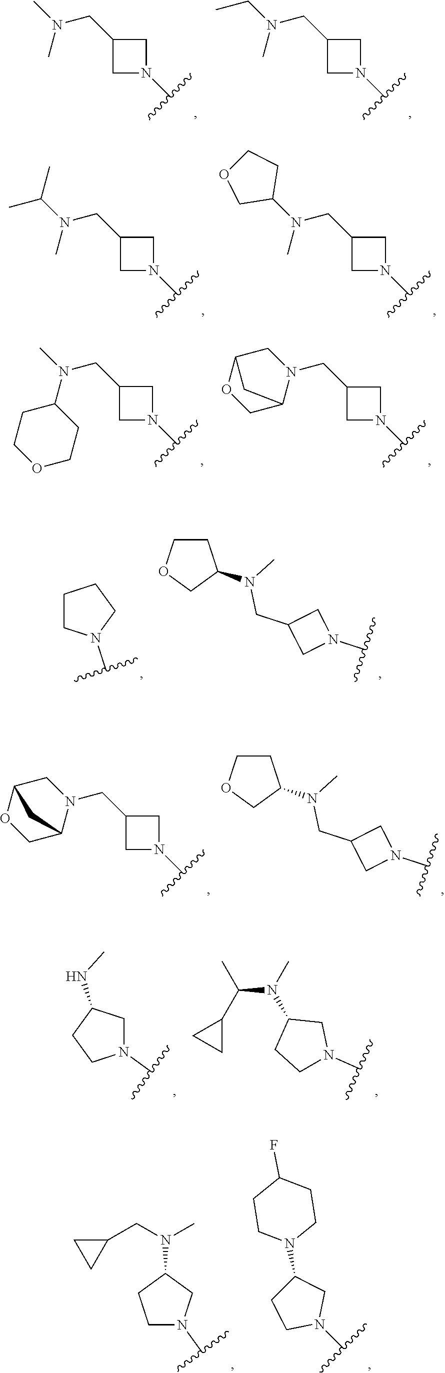

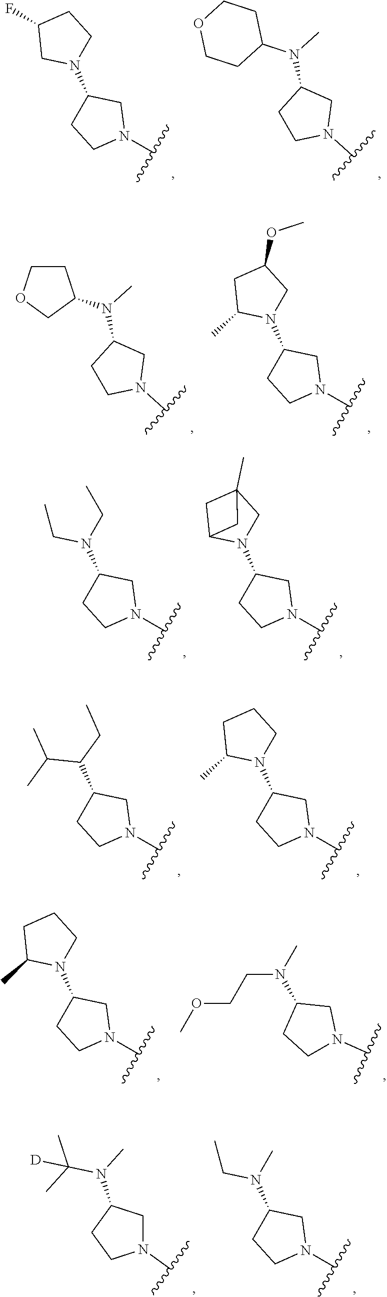

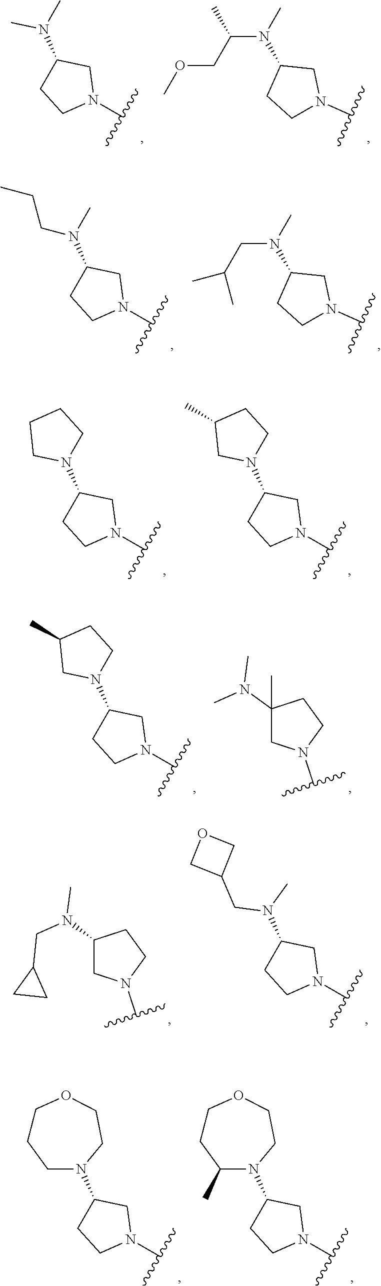

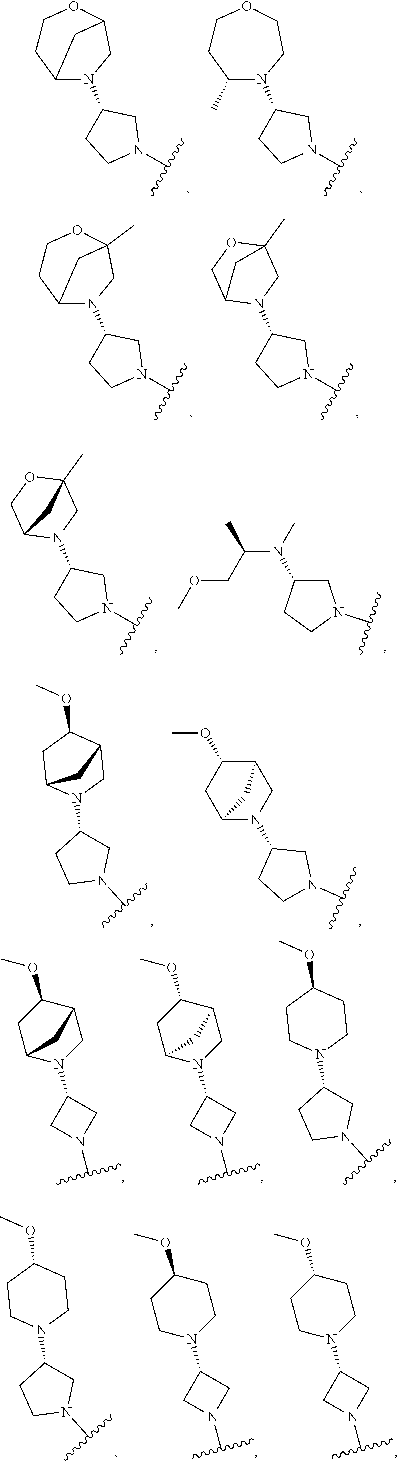

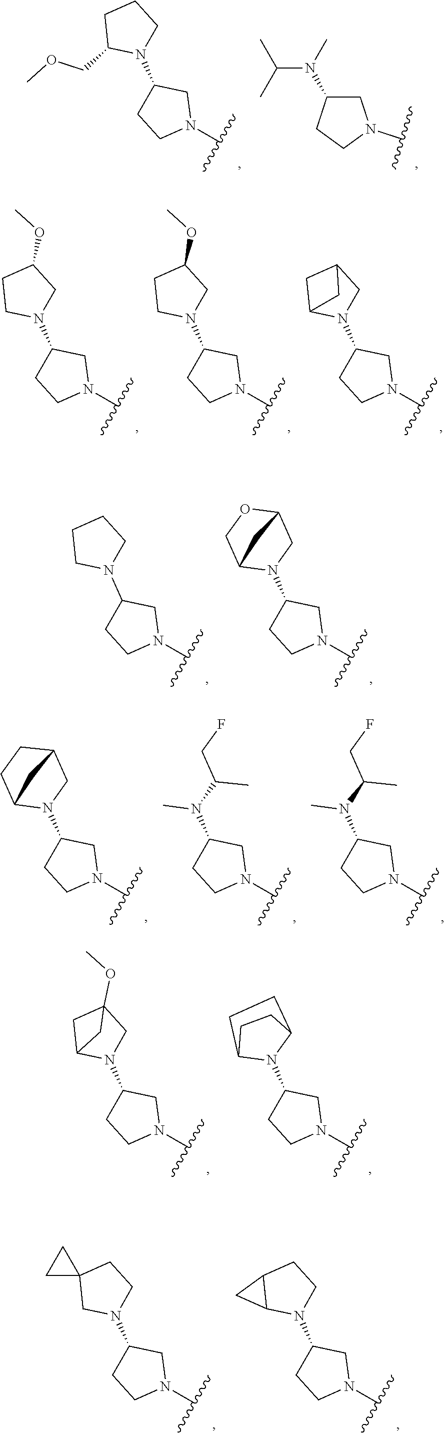

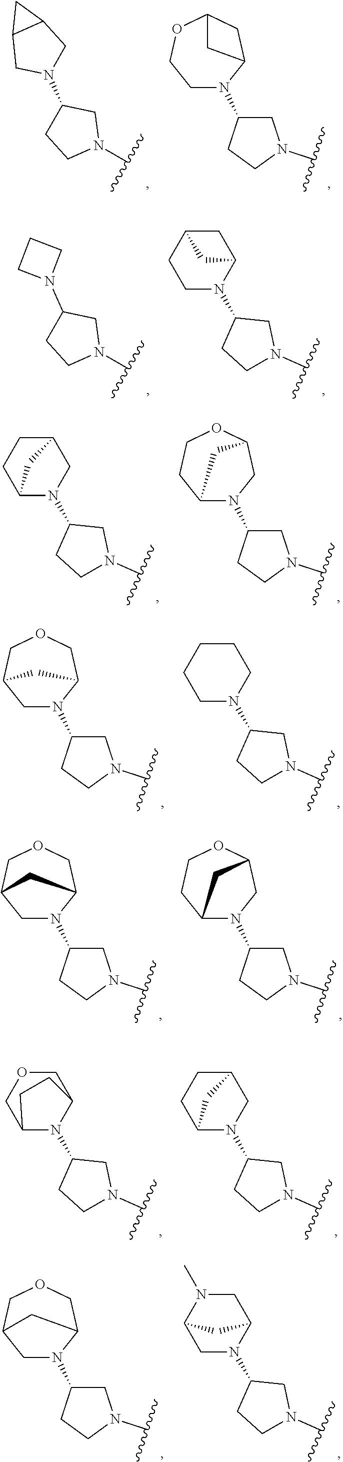

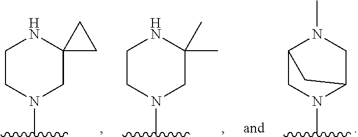

11. The method according to claim 6, wherein R⁵ is selected from the group consisting of

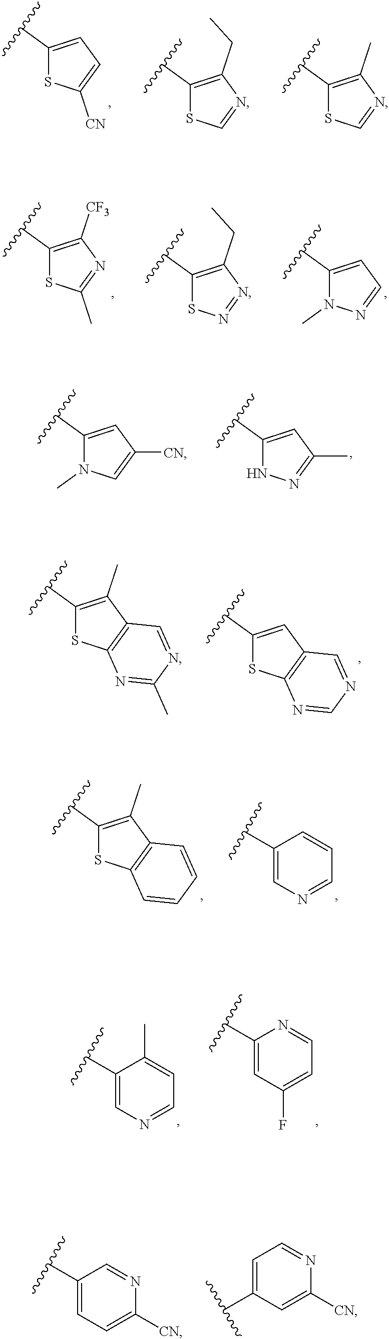

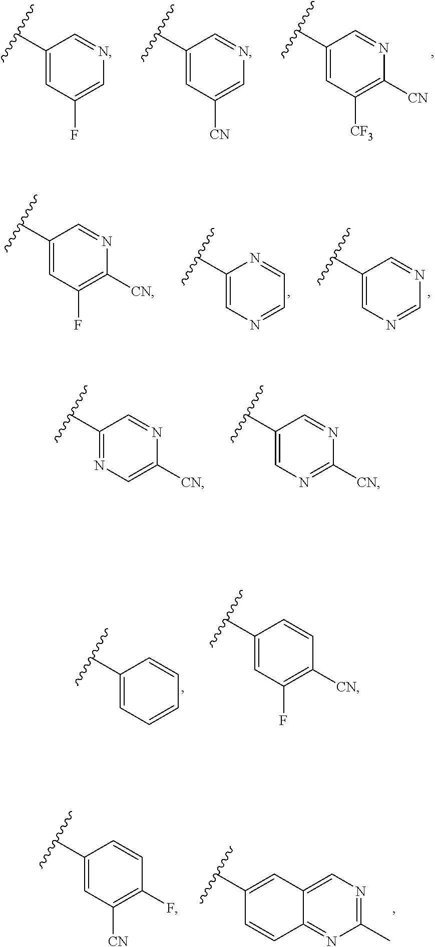

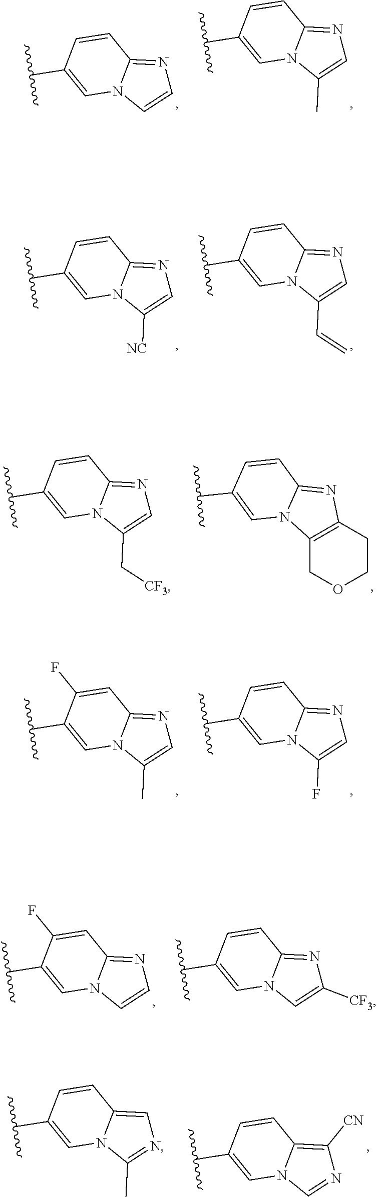

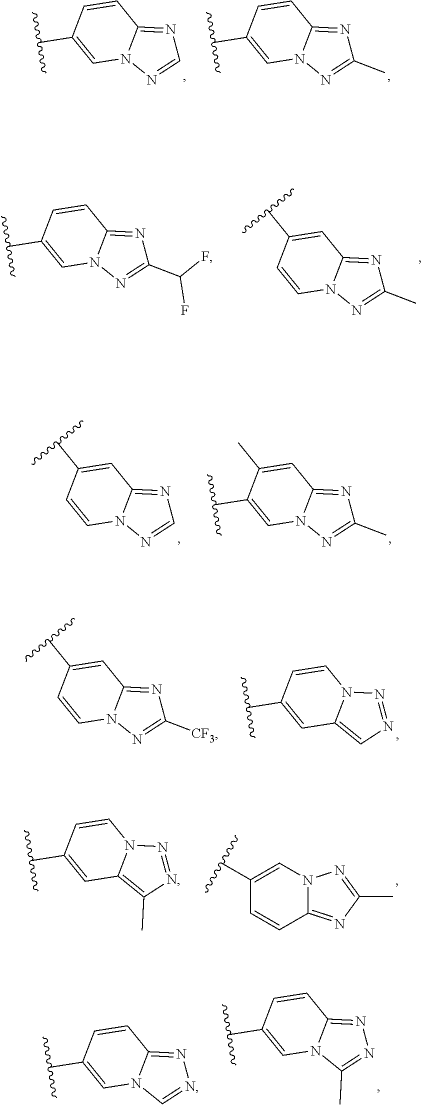

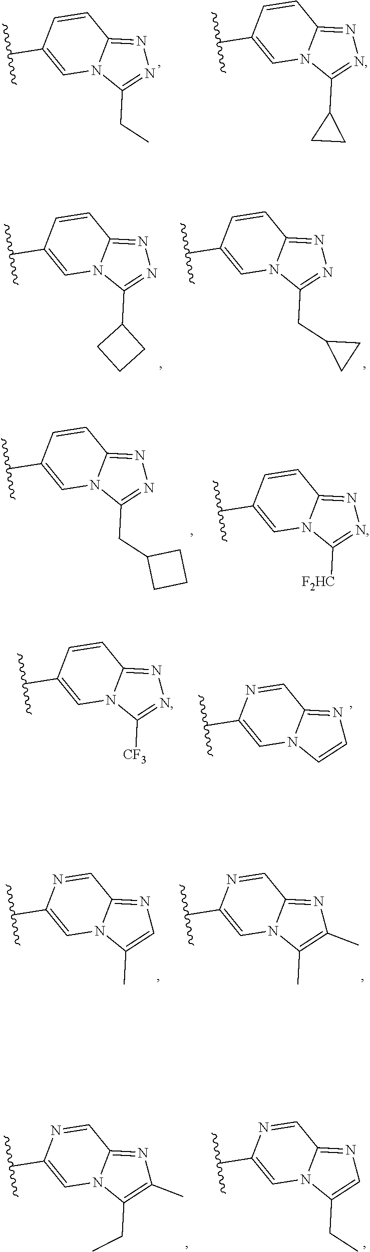

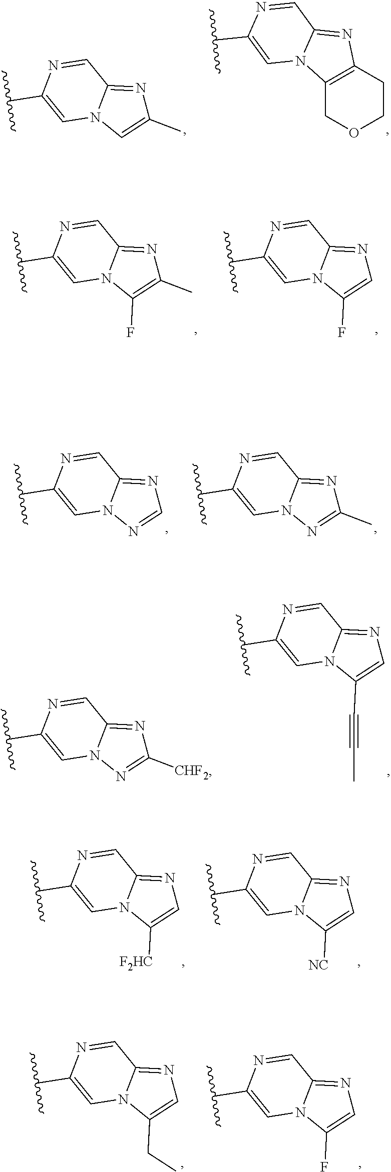

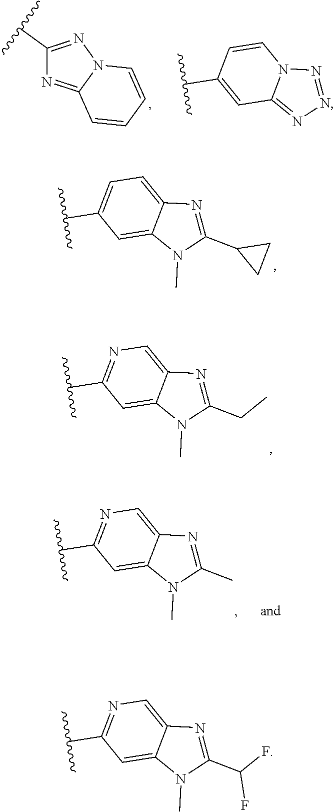

12. The method according to claim 1, wherein the KRAS G12D switch II inhibitor is selected from the group consisting of the following compounds or a pharmaceutically acceptable salt thereof:

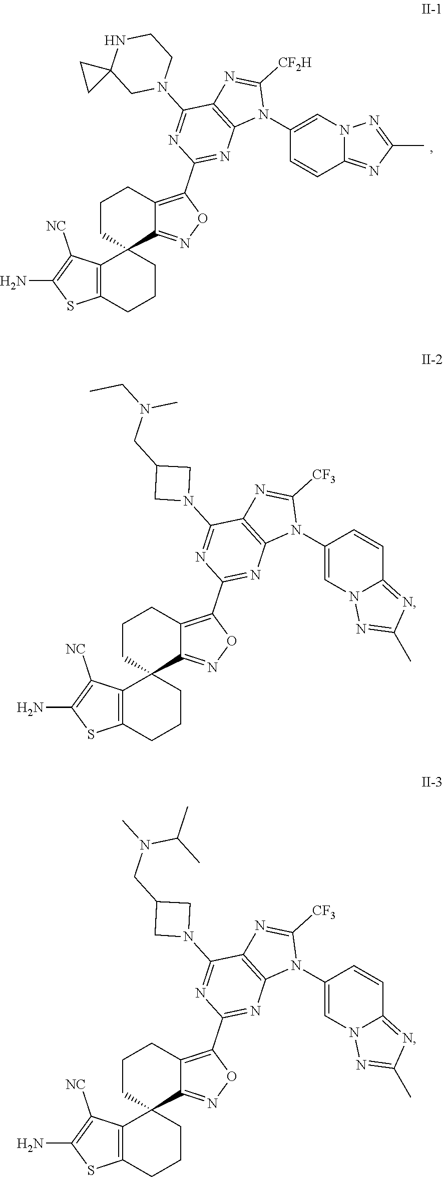 [SMILES: Cc1nc2ccc(-n3c(C(F)F)nc4c(N5CCNC6(CC6)C5)nc(-c5onc6c5CCC[C@@]65CCCc6sc(N)c(C#N)c65)nc43)cn2n1 ; CCN(C)CC1CN(c2nc(-c3onc4c3CCC[C@@]43CCCc4sc(N)c(C#N)c43)nc3c2nc(C(F)(F)F)n3-c2ccc3nc(C)nn3c2)C1 ; Cc1nc2ccc(-n3c(C(F)(F)F)nc4c(N5CC(CN(C)C(C)C)C5)nc(-c5onc6c5CCC[C@@]65CCCc6sc(N)c(C#N)c65)nc43)cn2n1]

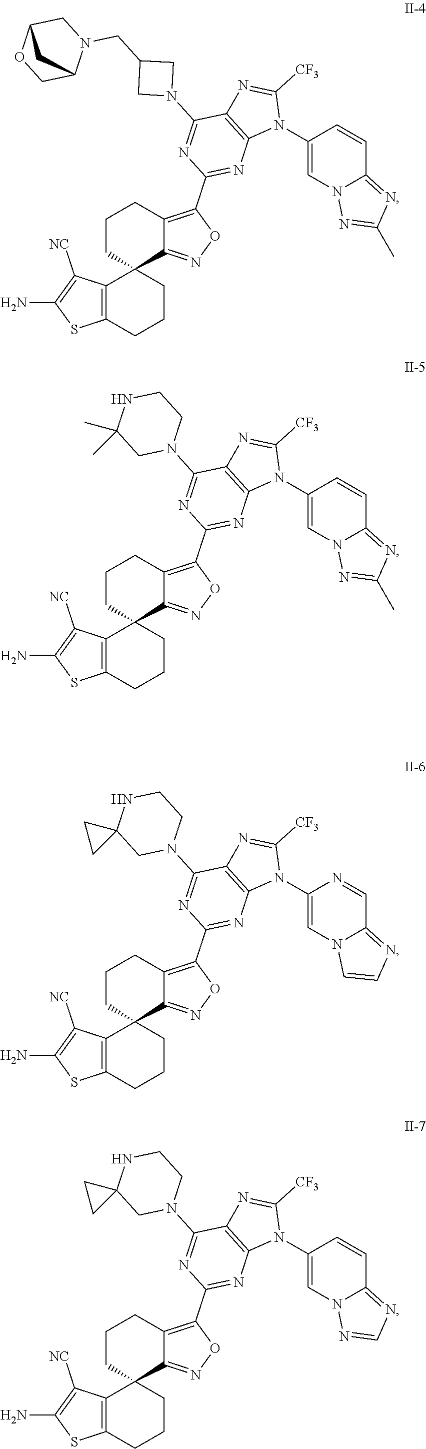 [SMILES: Cc1nc2ccc(-n3c(C(F)(F)F)nc4c(N5CC(CN6C[C@@H]7C[C@H]6CO7)C5)nc(-c5onc6c5CCC[C@@]65CCCc6sc(N)c(C#N)c65)nc43)cn2n1 ; Cc1nc2ccc(-n3c(C(F)(F)F)nc4c(N5CCNC(C)(C)C5)nc(-c5onc6c5CCC[C@@]65CCCc6sc(N)c(C#N)c65)nc43)cn2n1 ; N#Cc1c(N)sc2c1[C@@]1(CCC2)CCCc2c1noc2-c1nc(N2CCNC3(CC3)C2)c2nc(C(F)(F)F)n(-c3cn4ccnc4cn3)c2n1 ; N#Cc1c(N)sc2c1[C@@]1(CCC2)CCCc2c1noc2-c1nc(N2CCNC3(CC3)C2)c2nc(C(F)(F)F)n(-c3ccc4ncnn4c3)c2n1]

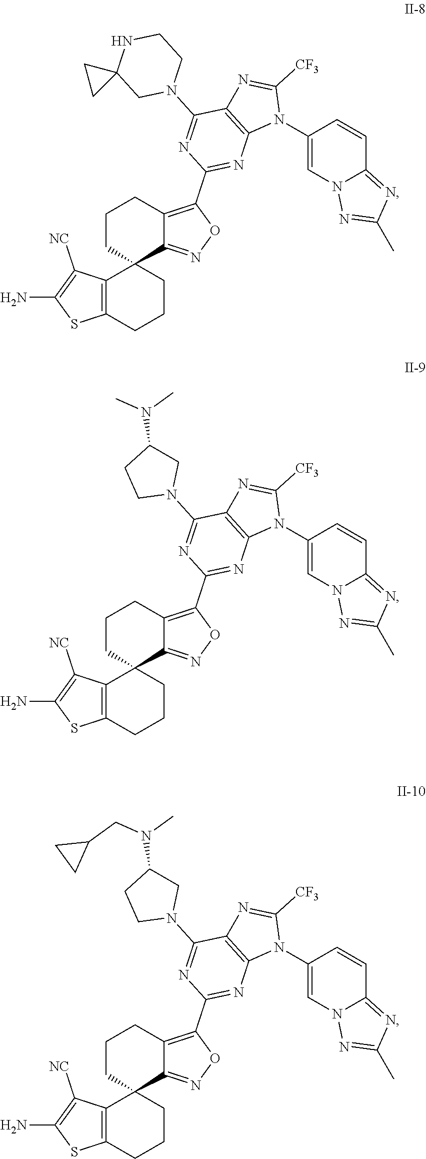 [SMILES: Cc1nc2ccc(-n3c(C(F)(F)F)nc4c(N5CCNC6(CC6)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N(C)C)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N(C)CC6CC6)C5)nc(-c5onc6c5CCC[C@@]65CCCc6sc(N)c(C#N)c65)nc43)cn2n1]

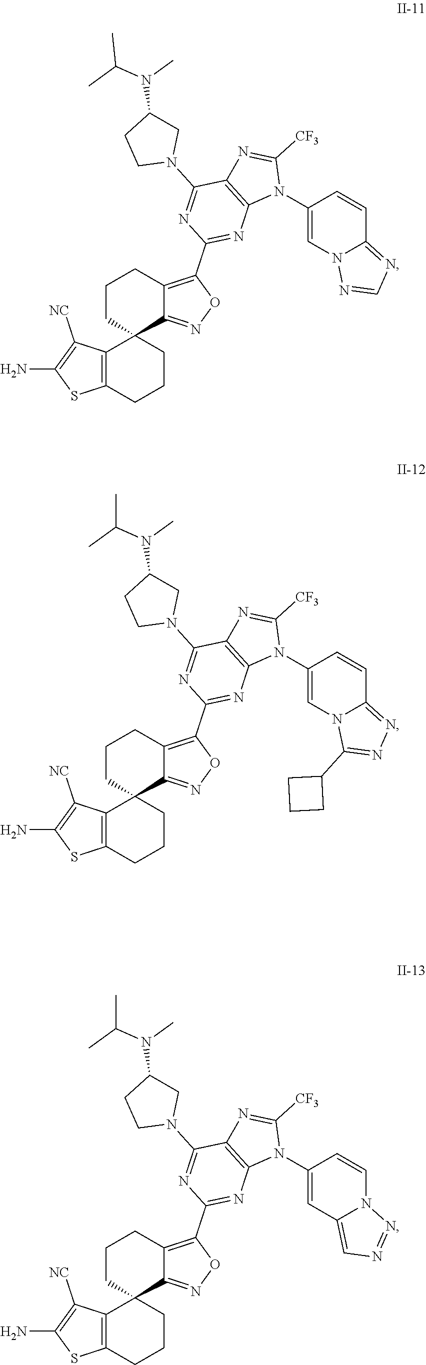 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3ncnn3c2)C1 ; CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C4CCC4)n3c2)C1 ; CC(C)N(C)[C@H]1CCN(c2nc(-c3onc4c3CCC[C@@]43CCCc4sc(N)c(C#N)c43)nc3c2nc(C(F)(F)F)n3-c2ccn3nncc3c2)C1]

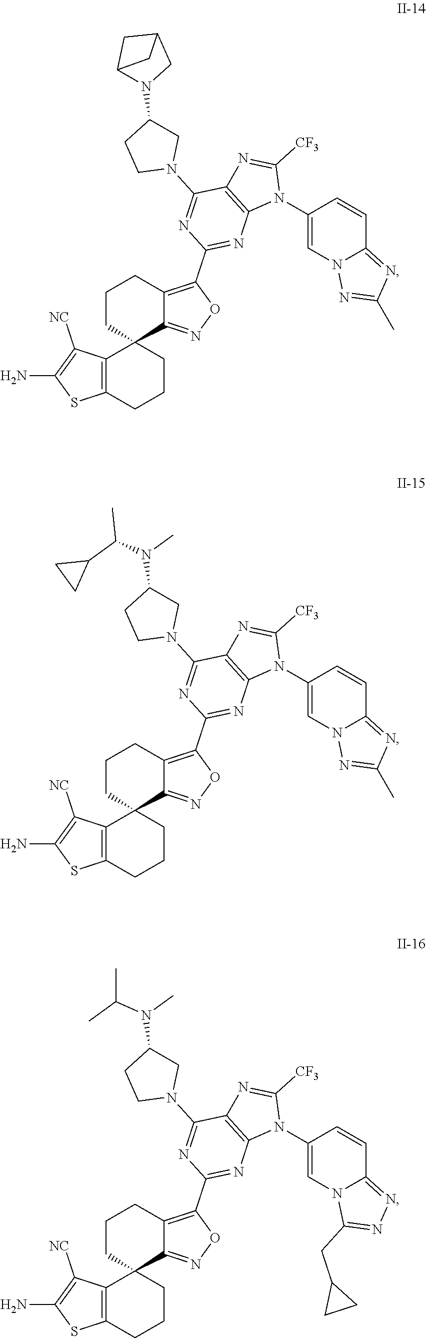 [SMILES: Cc1nc2ccc(-n3c(C(F)(F)F)nc4c(N5CC[C@H](N6CC7CC6C7)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N(C)[C@@H](C)C6CC6)C5)nc(-c5onc6c5CCC[C@@]65CCCc6sc(N)c(C#N)c65)nc43)cn2n1 ; CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(CC4CC4)n3c2)C1]

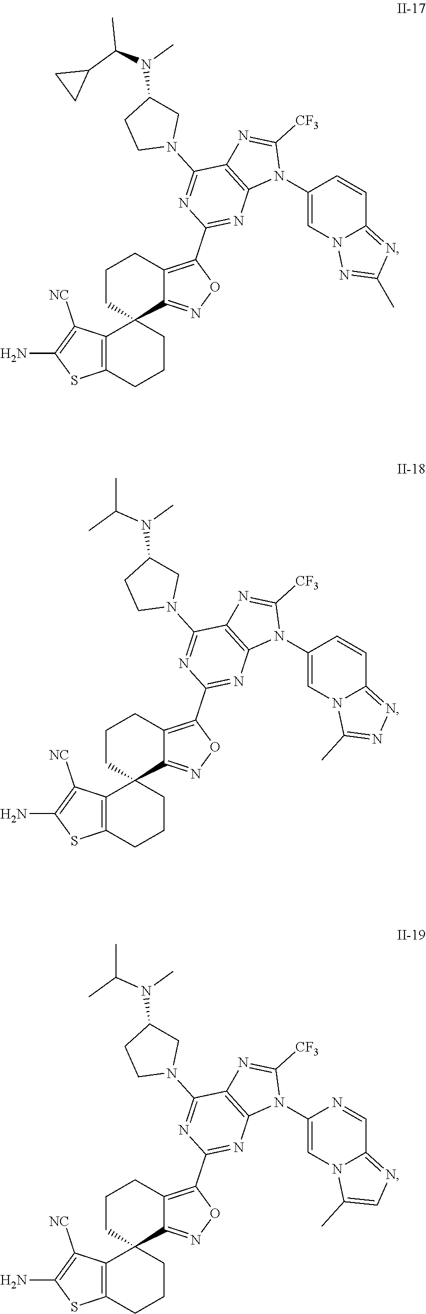 [SMILES: Cc1nc2ccc(-n3c(C(F)(F)F)nc4c(N5CC[C@H](N(C)[C@H](C)C6CC6)C5)nc(-c5onc6c5CCC[C@@]65CCCc6sc(N)c(C#N)c65)nc43)cn2n1 ; Cc1nnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12 ; Cc1cnc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn12]

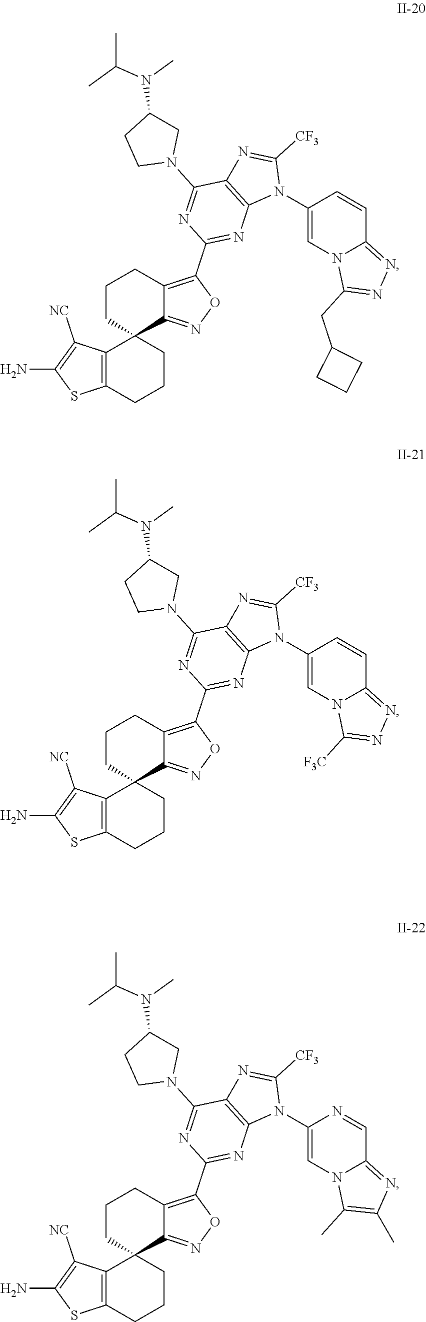 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(CC4CCC4)n3c2)C1 ; CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1 ; Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2c1C]

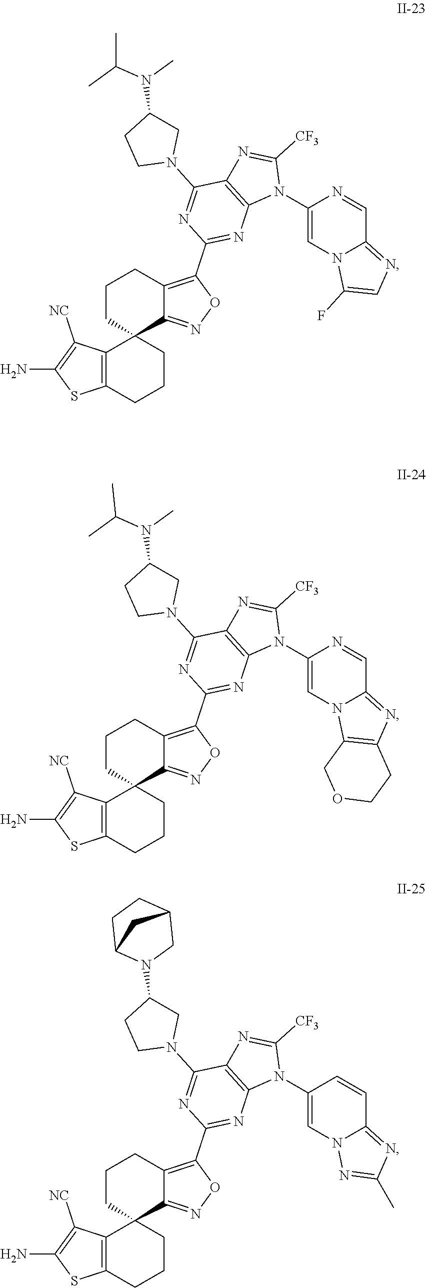

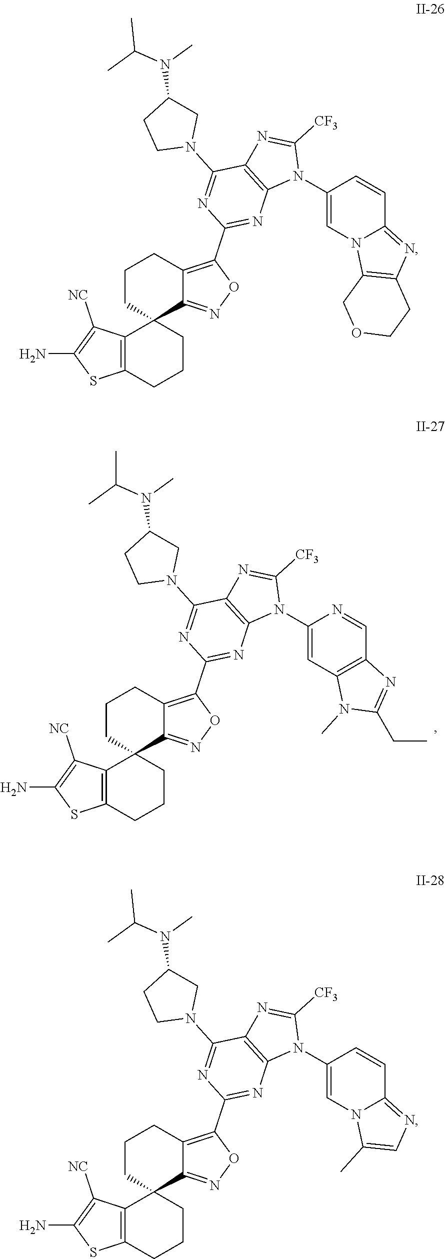 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nc4c(n3c2)COCC4)C1 ; CCc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C ; Cc1cnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12]

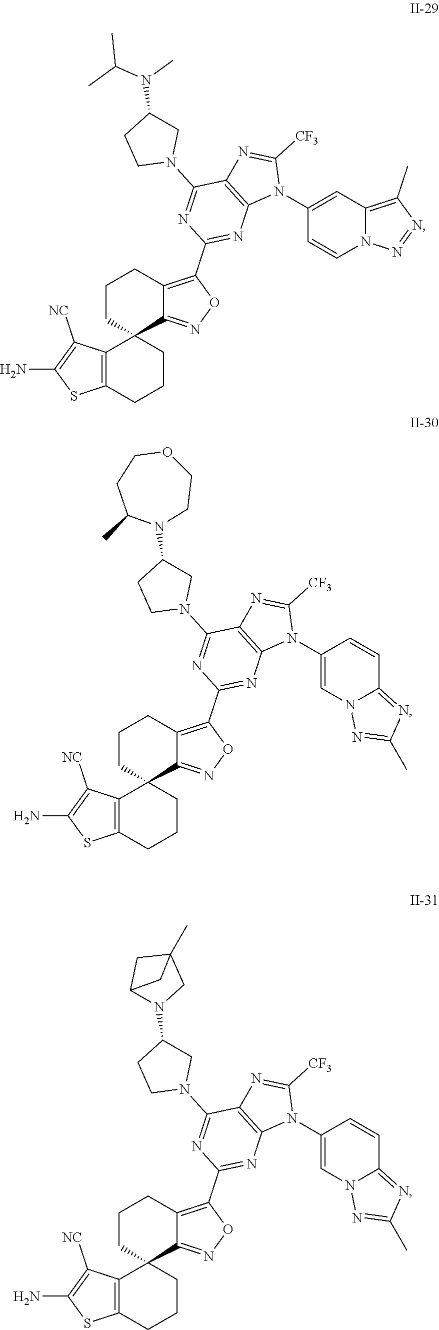 [SMILES: Cc1nnn2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cc12 ; Cc1nc2ccc(-n3c(C(F)(F)F)nc4c(N5CC[C@H](N6CCOCC[C@@H]6C)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N6CC7(C)CC6C7)C5)nc(-c5onc6c5CCC[C@@]65CCCc6sc(N)c(C#N)c65)nc43)cn2n1]

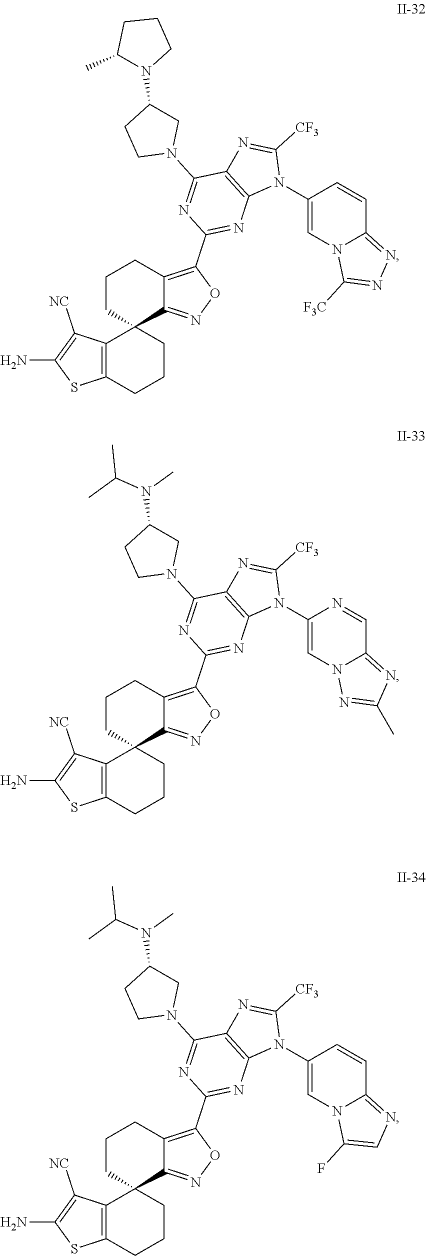 [SMILES: C[C@@H]1CCCN1[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1 ; Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1 ; CC(C)N(C)[C@H]1CCN(c2nc(-c3onc4c3CCC[C@@]43CCCc4sc(N)c(C#N)c43)nc3c2nc(C(F)(F)F)n3-c2ccc3ncc(F)n3c2)C1]

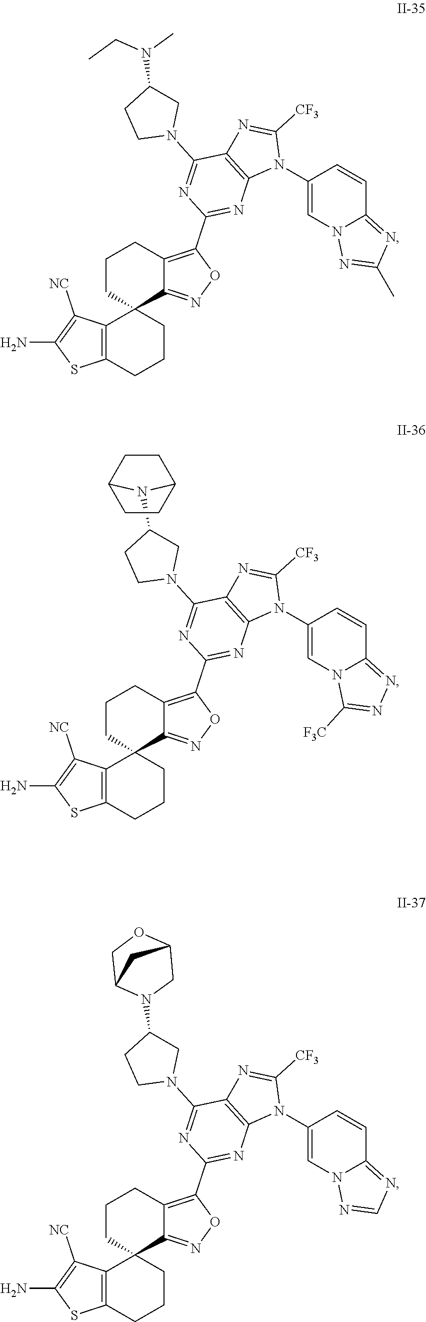 [SMILES: CCN(C)[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1 ; N#Cc1c(N)sc2c1[C@@]1(CCC2)CCCc2c1noc2-c1nc(N2CC[C@H](N3C4CCC3CC4)C2)c2nc(C(F)(F)F)n(-c3ccc4nnc(C(F)(F)F)n4c3)c2n1 ; N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3ccc4ncnn4c3)c2n1]

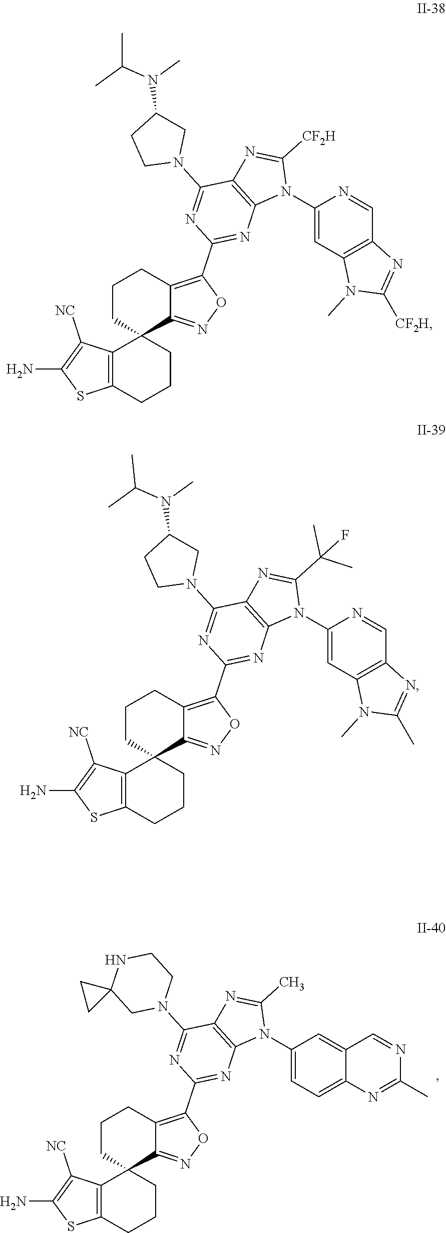 [SMILES: CC(C)N(C)[C@H]1CCN(c2nc(-c3onc4c3CCC[C@@]43CCCc4sc(N)c(C#N)c43)nc3c2nc(C(F)F)n3-c2cc3c(cn2)nc(C(F)F)n3C)C1 ; Cc1nc2cnc(-n3c(C(C)(C)F)nc4c(N5CC[C@H](N(C)C(C)C)C5)nc(-c5onc6c5CCC[C@@]65CCCc6sc(N)c(C#N)c65)nc43)cc2n1C ; Cc1ncc2cc(-n3c(C)nc4c(N5CCNC6(CC6)C5)nc(-c5onc6c5CCC[C@@]65CCCc6sc(N)c(C#N)c65)nc43)ccc2n1]

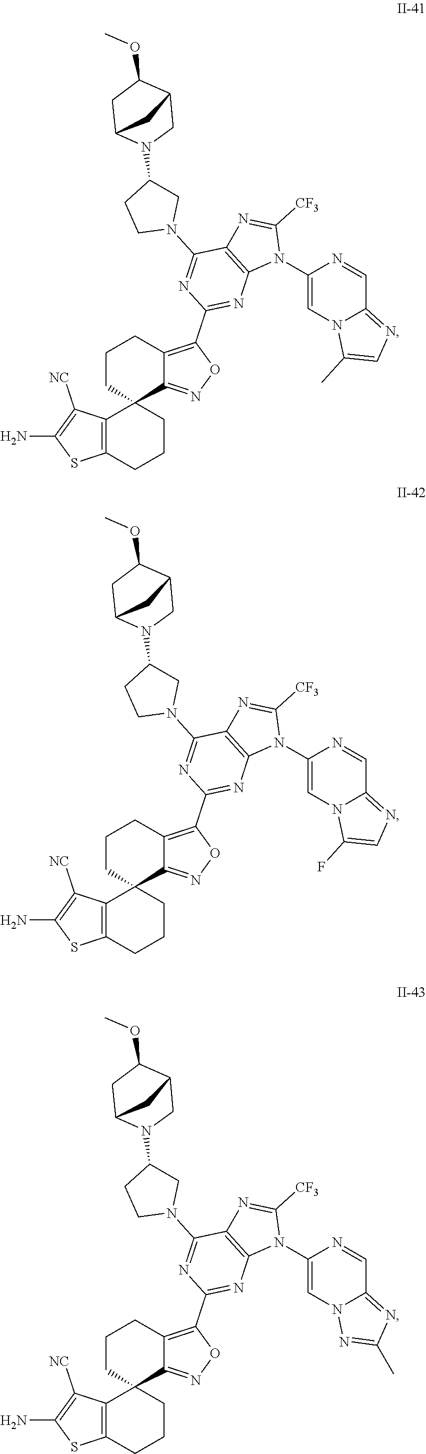 [SMILES: CO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1 ; CO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1 ; CO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3nc(C)nc3cn2)C1]

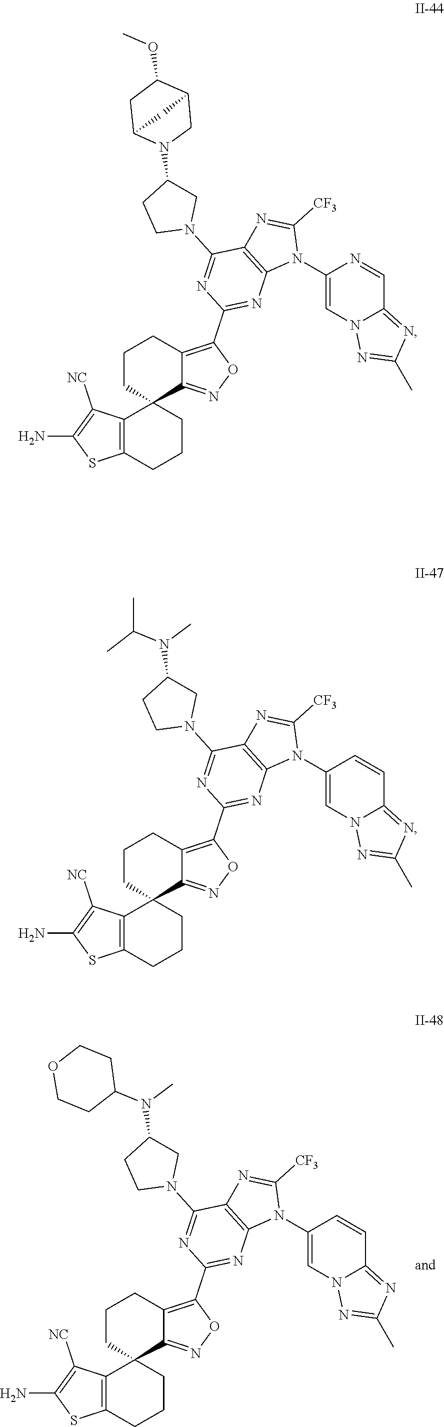 [SMILES: CO[C@H]1C[C@H]2C[C@@H]1CN2[C@H]1CCN(c2nc(-c3onc4c3CCC[C@@]43CCCc4sc(N)c(C#N)c43)nc3c2nc(C(F)(F)F)n3-c2cn3nc(C)nc3cn2)C1 ; Cc1nc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N(C)C6CCOCC6)C5)nc(-c5onc6c5CCC[C@@]65CCCc6sc(N)c(C#N)c65)nc43)cn2n1]

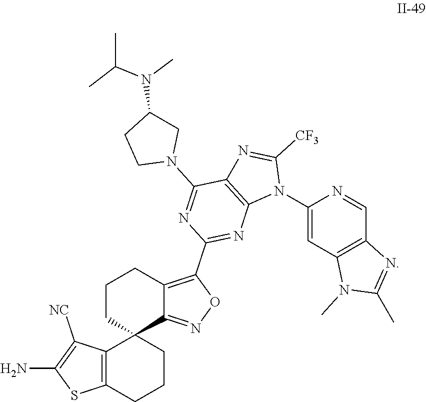 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C]

13. The method according to claim 1, wherein the cancer is selected from the group consisting of pancreatic cancer, lung cancer, colorectal cancer, cholangiocarcinoma, uterine cancer, endometrial cancer, thyroid cancer, acute myeloid leukaemia, bladder cancer, urothelial cancer, gastric cancer, cervical cancer, oesophageal cancer, and ovarian cancer.
